SEQUENCE LISTING (198 sequences)

SEQ ID NO: 1 - DNA, 305 bp, Artificial Sequence
  Description of Artificial Sequence Synthetic polynucleotide
gcgaggggtctgcctgggccagccgctccctctgaacacgggacgcgtggtccaattcgggcttcgggaccctttggcggtttgaacgcctgggagagggcgcccgcgagcctggggaccccggcaacggcttccccagagcctgccttgcaatctcgcgcgtcctctccctcagcacgtggcggttccacgtgtggtcgggcgtcccggactagctcacgtcgtgacctagcttaatgaacccagccgggcctgcagcaccaccttagaggttttgattatttgattagaccaatctattcacc

SEQ ID NO: 2 - DNA, 305 bp, Artificial Sequence
  Description of Artificial Sequence Synthetic polynucleotide
ggcgaatagattggtataatgaaataatcaaaacctcttaggcggtgctacaggcccggctgggttcattaagctaggtcacgacgcgagctagtccgggaagcccgaccacacgtggaaccgccacgtgctgagggagaggacgcgcgagattgcaaggcaggctctggggaagccgttgccggggtccccaggctcgcgggcgccccatccctggcgttcaaaccgccaaagggtcccgaagcccgaattggaccacgcgtcccgtgtttagagggagcggctggcccaggcagacccctcgc

SEQ ID NO: 3 - DNA, 305 bp, Artificial Sequence
  Description of Artificial Sequence Synthetic polynucleotide
ggtgaatagattggtctaatcaaataatcaaaacctctaaggtggtgctgcaggcccggctgggttcattaagctaggtcacgacgtgagctagtccgggacgcccgaccacacgtggaaccgccacgtgctgagggagaggacgcgcgagattgcaaggcaggctctggggaagccgttgccggggtccccaggctcgcgggcgccctctcccaggcgttcaaaccgccaaagggtcccgaagcccgaattggaccacgcgtcccgtgttcagagggagcggctggcccaggcagacccctcgc

SEQ ID NO: 4 - DNA, 1322 bp, Artificial Sequence
  Description of Artificial Sequence Synthetic polynucleotide
gtgatgggttctttagacgatccagcccaggatcatgtgttgcccacatggagcctatccacgctggcctagaaggcaagcacatttcaaggtgaacccacgtccatggagcgatggcgccaatatctcgcctctagaccaagcggttctcaccccaactgcgtcatttgtatgtatggctgcaaagttgtcggtacgatagaggccgccaacctggcggcgagggcgaggagctggttgccgatctgtgcccaagcatgtgtcggagctcggctgtctcggcagcgagctcctgtgcaaggggcttgcatcgagaatgtcaggcgatagacactgcacgttggggacacggaggtgcccctgtggcgtgtcctggatgccctcgggtccgtcgcgagaagctctggcgaccagcacccggccacaaccgcagcaggcgttcacccacaagaatcttccagatcgtgatgcgcatgtatcgtgacacgattggcgaggtccgcaggacgcacacggactcgtccactcatcagaactggtcagggcacccatctgcgtcccttttcaggaaccacccaccgctgccaggcaccttcgccagcggcggactccacacagagaatgccttgctgtgagagaccatggccggcaagtgctgtcggatctgcccgcatacggtcagtccccagcacaaggaagccaagagtacaggctgttggtgtcgatggaggagtggccgttcccacaagtagtgagcggcagctgctcaacggcttccccctgttcatcttggcaaagccagtgacttcctacaagtatgtgatgcagatcggcactgcaatctgtcggcatgcgtacagaacatcggctcgccagggcagcgttgctcgctctggatgagctgcttgggaggaatcatcggcacacgcccgtgccgtgcccgcgccccgcgcccgtcgggaaaggcccccggttaggacactgccgcgtcagccagtcgtgggatcgatcggacgtggcgaatcctcgcccggacaccctcatcacaccccacatttccctgcaagcaatcttgccgacaaaatagtcaagatccattgggtttagggaacacgtgcgagactgggcagctgtatctgtccttgccccgcgtcaaattcctgggcgtgacgcagtcacaggagaatctattagaccctggacttgcagctcagtcatgggcgtgagtggctaaagcacctaggtcaggcgagtaccgccccttccccaggattcactcttctgcgattgacgttgagcctgcatcgggctgcttcgtcacc

SEQ ID NO: 5 - DNA, 841 bp, Artificial Sequence
  Description of Artificial Sequence Synthetic polynucleotide
tcggagctaaagcagagactggacaagacttgcgttcgcatactggtgacacagaatagctcccatctattcatacgcctttgggaaaaggaacgagccttgtggcctctgcattgctgcctgctttgaggccgaggacggtgcgggacgctcagatccatcagcgatcgccccaccctcagagcacctccgatccaaggcaatactatcaggcaaagtttccaaattcaaacattccaaaatcacgccagggactggatcacacacgcagatcagcgccgttttgctctttgcctacgggcgactgtgccacttgtcgacccctggtgacgggagggaccacgcctgcggttggcatccacttcgacggacccagggacggtttctcatgccaaacctgagatttgagcacccagatgagcacattatgcgttttaggatgcctgagcagcgggcgtgcaggaatctggtctcgccagattcaccgaagatgcgcccatcggagcgaggcgagggctttgtgaccacgcaaggcagtgtgaggcaaacacatagggacacctgcgtctttcaatgcacagacatctatggtgcccatgtatataaaatgggctacttctgagtcaaaccaacgcaaactgcgctatggcaaggccggccaaggttggaatcccggtctgtctggatttgagtttgtgggggctatcacgtgacaatccctgggattgggcggcagcagcgcacggcctgggtggcaatggcgcactaatactgctgaaagcacggctctgcatccctttctcttgacctgcgattggtccttttcgcaagcgtgatcatc

SEQ ID NO: 6 - DNA, 841 bp, Artificial Sequence
  Description of Artificial Sequence Synthetic polynucleotide
tcggagctaaagcagaaactgaacaagacttgcgttcgcatacttgtgacactgaataggttcaatctattcatacgcctttgggaaactgaacgagccttgtggcctctgcattgctgcctgctttgaggccgaggacggcgcggaacgcacagatccatcagcgatcgccccaccctcagagtacatccgatccaaggcaatactatcaggcaaagtttccaaattcaaacattccaaaattacgtcagggactggatcacacacgcagatcagcgccgttttgctctttgcctacgggcgactgtgccacttgtcgacgcctggtgacgggagggaccacgcctgcggttggcatccacttcgacggacccagggacggtctcacatgccaaacctgagatttgagcaccaagatgagcacattatgcgtttttggatgcctgagcagcgggcgtgcaggaatctggtctcgccagattcaccgaagatgcggccatcggagcgaggcgagggctgtgtggccacgccaggcagtgtgaggcaaacacacagggacatctgcttctttcgatgcacagacatctatgttgcccgtgcatataaaatgggctacttctgaatcaaaccaacgcaaacttcgctatggcaaggccggccaaggttggaatcccggtctgtctggatttgagtttgtgggggctatcacgtgacaatccctgggattgggcggcagcagcgcacggcctggatggcaatggcgcactaatactgctgaaagcacggctctgcatccctttctcttgacctgcgattggtccttttcgcaagcgtgatcatc

SEQ ID NO: 7 - DNA, 512 bp, Artificial Sequence
  Description of Artificial Sequence Synthetic polynucleotide
caccgatcactccgtcgccgcccaagagaaatcaacctcgatggagggcgaggtggatcagaggtattggttatcgttcgttcttagtctcaatcaatcgtacaccttgcagttgcccgagtttctccacacatacagcacctcccgctcccagcccattcgagcgacccaatccgggcgatcccagcgatcgtcgtcgcttcagtgctgaccggtggaaagcaggagatctcgggcgagcaggaccacatccagcccaggatcttcgactggctcagagctgaccctcacgcggcacagcaaaagtagcacgcacgcgttatgcaaactggttacaacctgtccaacagtgttgcgacgttgactggctacattgtctgtctgtcgcgagtgcgcctgggcccttacggtgggacactggaactccgccccgagtcgaacacctagggcgacgcccgcagcttggcatgacagctctccttgtgttctaaataccttgcgcgtgtgggaga

SEQ ID NO: 8 - DNA, 516 bp, Artificial Sequence
  Description of Artificial Sequence Synthetic polynucleotide
atccaccgatcactccgtcgccgcccaagagaattcaacctcgatggagggcaaggtggatcagaggtattggttatcgttcgctattagtctcaatcaatcgtgcaccttgcagttgctcgagtttctccacacatacagcacctcccgctcccagcccattcgagcgacccaatccgggcgatcccagcgatcgtcgtcgcttcagtgctgaccggtggaaagcaggagatctcgggcgagcaggaccacatccagcacaggatcttcgactggctcagagctgaccctcacgcggcacagcaaaagtagcccgcacgcgttatgcaaacaggttacaacctgtccaacactgttgcgacgttgactggctacattgtctgtctgtcgcgagtacgcctggacccttacggtgggacactggaactccgccccgagtcgaacacctagggcgacgcccgcagcttggcatgacagctctccttgtattctaaatacctcgcgcgtgtgggagaa

SEQ ID NO: 9 - DNA, 335 bp, Artificial Sequence
  Description of Artificial Sequence Synthetic polynucleotide
atgatgcgcgtgtacgactatcaaggaagaaagaggacttaatttcttaccttctaaccaccatattctttttgctggatgcttgctcgtctcgatgacaattgtgaacctcttgtgtgaccctgaccctgctgcaaggctctccgaccgcacgcaaggcgcagccggcgcgtccggaggcgatcggatccaatccagtcgtcctcccgcagcccgggcacgtttgcccatgcaggcccttccacaccgctcaagagactcccgaacaccgcccactcggcactcgcttcggctgccgagtgcgcgtttgagtttgccctgccacagaagacacc

SEQ ID NO: 10 - DNA, 335 bp, Artificial Sequence
  Description of Artificial Sequence Synthetic polynucleotide
atgatgcgcgtgtacgactatcaaggaagaaagaggacttaatttcttaccttctaaccaccatattctttttgctggatgcttgctcgtctcgatgacaattgtgaacctcttgtgtgaccctgaccctgctgcaaggctctccgaccgcacgcaaggcgcagccggcgcgtccggaggcgatcggatccaatccagtcgtcctcccgcagcccgggcacgtttgcccatgcaggcccttccacaccgctcaagagactcccgaacaccgcccactcggcactcgcttcggctgccgagtgcgcgtttgagtttgccctgccacaggagacatc

SEQ ID NO: 11 - DNA, 1097 bp, Artificial Sequence
  Description of Artificial Sequence Synthetic polynucleotide
cccgggcgagctgtacgcctacggagcgaggcctggtgtgaccgttgcgatctcgccagcagacgtcgcggagcctcgtcccaaaggccctttctgatcgagcttgtcgtccactggacgctttaagttgcgcgcgcgatgggataaccgagctgatctgcactcagattttggtttgttttcgcgcatggtgcagcgaggggaggtactacgctggggtacgagatcctccggattcccagaccgtgttgccggcatttacccggtcatcgccagcgattcgggacgacaaggccttatcctgtgctgagacgctcgagcacgtttataaaattgtgggtaccgcggtatgcacagcgttcaacacgcgccacgccgaaattggttggtgggggagcacgtatgggactgacgtatggccagcagcgaacactcaccgaacaagtgccaatgtataccttgcatcaatgatgctccggcagcttcgattgactgtctcgaaaaagtgtgagcaagcagatcatgtggccgctctgtcgcgcagcacctgacgcattcgacacccacggcaatgcccaggccagggaatagagagtaagacaactcccattgttcagcaaaacattgcactgcagtgccttcacaactatacaatgaatgggagggaatatgggctctgcatgggacagcttagctgggacattcggctactgaacaagaaaaccccacgagaaccaattggcgaaacctgccgggaggaggtgatcgtttctgtaaatggcttacgcattcccccccggcggctcacgaggggtgtggtgaaccctgccagctgatcaagtgcttgctgacgtcggccagggaggtgtatgtgattgggccgtggggcgtgagttatcctaccgccggacccgcgaagtcacatgacgaatggccgtgcgggatgacgagagcacgactcgctctttcttcgccggcccggcttcatggaggacaataataaagggtggccaccggcaacagccctccatacctgaaccgattccagacccaaacctcttgaattttgagggatccagttcaccggtatagtcacg

SEQ ID NO: 12 - DNA, 1105 bp, Artificial Sequence
  Description of Artificial Sequence Synthetic polynucleotide
atccccgggcgagctgtacgcctacggagcgaggcctggtgtgaccgttgcgatctcgccagcagacgtcgcggagcctcgtcccaaaggccctttctgatcgagcttgtcgtccactggacgctttaagttgcgcgcgcgatgggataaccgagctgatctgcactcagattttggtttgttttcgcgcatggtgcagcgaggggaggtactacgctggggtacgagatcctccggattcccagaccgtgttgccggcatttacccggtcatcgccagcgattcgggacgacaaggccttatcctgtgctgagacgctcgagcacgtttataaaattgtggtcaccgtggtacgcacagcgtccaacacgcgccacgccgaaattcgttggtgggggagcacgtatcggactgacgtatggccagcagcgaacactcaccaaacaggtgccaatgtatagcttgcatcaatgatgctctggcagcttcgattgactgtctcgaaaaagtgtgtgcaaacagattatgtggccgctctgtggccgcgcagcacctgacgcactcgacacccacggcaatgcccaggccaaggaacagagagtaagacaactcccattgttcagtaaaacattgcactgcagtgccttcacaaacatacaacgaatgggagggaatatgggcttcgaatgggacagcttagctgggacattcggttactgaacaagaaaaccccacgagaaccaactggcgaaacctgccgggaggaggtgatcgtttttgtaaatggcttacgcattccccccccggcggctcacggggggtgtggtgaaccctgccagctgatcaagtgcttgctgacgtcggccagggaggtgtatgtgatttggccgtggggcgtgagttatcctaccgccggacccgcgaagtcacatgacgaatggccgtgcgggatgacgagagcagggctcgctctttcttcgccggcccggcttcatggaggacaataataaagggtggccaccggcaacagccctccatacctgaaccgattccagacccaaacctcttgaattttgagggatccagttcaccggtatagtcacga

SEQ ID NO: 13 - DNA, 754 bp, Artificial Sequence
  Description of Artificial Sequence Synthetic polynucleotide
gcgagtggttttgctgccgggaagggagtggggagcgtcgagcgagggacgcggcgctcgaggcgcacgtcgtctgtcaacgcgcgcggccctcgcggcccgcggccccacccagctctaatcatcgaaaactaagaggctccacacgcctgtcgtagaatgcatgggattcgccagtagaccacgatctgcgccgaagaagctggtctacccgacgttttttgttgctcctttattctgaatgatatgaagatagtgtgcgcagtgccacgcataggcatcaggagcaagggaggacgggtcaacttgaaagaaccaaaccatccatccgagaaatgcgcatcatctttgtagtaccatcaaacgccttggccaatgtcttctgcatggacaacacaacctgctcctggccacacggtcgacttggagcgccccatgcgcccaggtcgccacgacccgcggcccagcgcgcggcgattcgcctcacgagatcccggcggacccggcacgcccgcgggccgacggtgcgcttggcgatgctgctcattaacccacggccgtcacccgatccacatgctctttttcaacacatccacattggaatagagctctaccagggtgagtactgcattctttggggctgggaggaccccactcgacacctggtccttcatcggccgaaagcccgaacctgagcgcttccccgccccgttcctcatccccgactttccgatggcccattgcagtttcaaac

SEQ ID NO: 14 - DNA, 318 bp, Artificial Sequence
  Description of Artificial Sequence Synthetic polynucleotide
atctgggtggaggactgggagtaagatgtaaggatattaattaaacattctagtttgttgatggcacaacagtcaatgcatttcagtcgtcttgctccttataacctatgcgtgtgccatcgccggccatgcacctgtggcgtggtaccgaccatcggggagaggcccgagattcggaggtacctcccgccctgggcgagcccttcacgtgacggcacaagtcccttgcatcggcccgcgagcacggaatacagagccccgtgccccccacgggccctcacatcatccactccattgttcttgccacaccgatcagca

SEQ ID NO: 15 - DNA, 316 bp, Artificial Sequence
  Description of Artificial Sequence Synthetic polynucleotide
tgggtggaggactgggaagaagatgtaaggatatcaatttaacattctagtttgttgatggcacaacagtcactgaataccgggcgtctggctgctaaaatagccggagcgtgtgccatcgccggccatgcatctgtggcgtggtaccgaccatcagggagaggcccgagattcggaggtacctcccgccctgggcgagcccttcacgtgacggcacaagtcccttgcatcggcccgcgagcacggaatacagagccccgtgctccccacgggccctcacatcatccactccattgttcttgccacaccgatcagc

SEQ ID NO: 16 - DNA, 350 bp, Artificial Sequence
  Description of Artificial Sequence Synthetic polynucleotide
ataacgaggcacaatgatcgatatttctatcgaacaactgtatttagccctgtacgtaccccgctcttgggccagcccgtccgtgcttgccttcggaaaattgcatggcgcctcatgcaaactcgcgctctcacagcagatctcgcccagctcccgggagagcaatcgcgggtggggcccggggcgaatccaggacgcgccccgcggggccgctccactcgccagggccaatgggcggcttatagtcctggcatgggctctgcatgcacagtatcgcagtttgggcgaggtgttgcccccgcgatttcgaatacgcgacgcccggtactcgtgcgagaacagggttcttg

SEQ ID NO: 17 - DNA, 772 bp, Prototheca moriformis
tcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcggggctggcgggaaaacaggcttcgtgtgctcaggttatgggaggtgcaggacagctcattaaacgccaacaatcgcacaattcatggcaagctaatcagttatttcccattaacgagctataattgtcccaaaattctggtctaccgggggtgatccttcgtgtacgggcccttccctcaaccctaggtatgcgcacatgcggtcgccgcgcaacgcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggtgtaggatttcgtaatcattcgtcctgatggggagctaccgactgccctagtatcagcccgactgcctgacgccagcgtccacttttgtgcacacattccattcgtgcccaagacatttcattgtggtgcgaagcgtccccagttacgctcacctgatccccaacctccttattgttctgtcgacagagtgggcccagaggccggtcgcagcc

SEQ ID NO: 18 - DNA, 1065 bp, Prototheca moriformis
ggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagcc

SEQ ID NO: 19 - DNA, 6332 bp, Artificial Sequence
  Description of Artificial Sequence Synthetic polynucleotide
gctcttccgctaacggaggtctgtcaccaaatggaccccgtctattgcgggaaaccacggcgatggcacgtttcaaaacttgatgaaatacaatattcagtatgtcgcgggcggcgacggcggggagctgatgtcgcgctgggtattgcttaatcgccagcttcgcccccgtcttggcgcgaggcgtgaacaagccgaccgatgtgcacgagcaaatcctgacactagaagggctgactcgcccggcacggctgaattacacaggcttgcaaaaataccagaatttgcacgcaccgtattcgcggtattttgttggacagtgaatagcgatgcggcaatggcttgtggcgttagaaggtgcgacgaaggtggtgccaccactgtgccagccagtcctggcggctcccagggccccgatcaagagccaggacatccaaactacccacagcatcaacgccccggcctatactcgaaccccacttgcactctgcaatggtatgggaaccacggggcagtcttgtgtgggtcgcgcctatcgcggtcggcgaagaccgggaaggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacgtactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccactagtatggccatccccgccgccgccgtgatcttcctgttcggcctgctgttcttcacctccggcctgatcatcaacctgttccaggccctgtgcttcgtgctggtgtggcccctgtccaagaacgcctaccgccgcatcaaccgcgtgttcgccgagctgctgctgtccgagctgctgtgcctgttcgactggtgggccggcgccaagctgaagctgttcaccgaccccgagaccttccgcctgatgggcaaggagcacgccctggtgatcatcaaccacatgaccgagctggactggatgctgggctgggtgatgggccagcacctgggctgcctgggctccatcctgtccgtggccaagaagtccaccaagttcctgcccgtgctgggctggtccatgtggttctccgagtacctgtacatcgagcgctcctgggccaaggaccgcaccaccctgaagtcccacatcgagcgcctgaccgactaccccctgcccttctggatggtgatcttcgtggagggcacccgcttcacccgcaccaagctgctggccgcccagcagtacgccgcctcctccggcctgcccgtgccccgcaacgtgctgatcccccgcaccaagggcttcgtgtcctgcgtgtcccacatgcgctccttcgtgcccgccgtgtacgacgtgaccgtggccttccccaagacctcccccccccccaccctgctgaacctgttcgagggccagtccatcgtgctgcacgtgcacatcaagcgccacgccatgaaggacctgcccgagtccgacgacgccgtggcccagtggtgccgcgacaagttcgtggagaaggacgccctgctggacaagcacaacgccgaggacaccttctccggccaggaggtgcaccgcaccggctcccgccccatcaagtccctgctggtggtgatctcctgggtggtggtgatcaccttcggcgccctgaagttcctgcagtggtcctcctggaagggcaaggccttctccgtgatcggcctgggcatcgtgaccctgctgatgcacatgctgatcctgtcctcccaggccgagcgctcctccaaccccgccaaggtggcccaggccaagctgaagaccgagctgtccatctccaagaaggccaccgacaaggagaactgactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgagctcagcggcgacggtcctgctaccgtacgacgttgggcacgcccatgaaagtttgtataccgagcttgttgagcgaactgcaagcgcggctcaaggatacttgaactcctggattgatatcggtccaataatggatggaaaatccgaacctcgtgcaagaactgagcaaacctcgttacatggatgcacagtcgccagtccaatgaacattgaagtgagcgaactgttcgcttcggtggcagtactactcaaagaatgagctgctgttaaaaatgcactctcgttctctcaagtgagtggcagatgagtgctcacgccttgcacttcgctgcccgtgtcatgccctgcgccccaaaatttgaaaaaagggatgagattattgggcaatggacgacgtcgtcgctccgggagtcaggaccggcggaaaataagaggcaacacactccgcttcttagctcttc

SEQ ID NO: 20 - DNA, 1188 bp, Artificial Sequence
  Description of Artificial Sequence Synthetic polynucleotide
actagtatggccatcccctccgccgccgtggtgttcctgttcggcctgctgttcttcacctccggcctgatcatcaacctgttccaggccttctgcttcgtgctgatctcccccctgtccaagaacgcctaccgccgcatcaaccgcgtgttcgccgagctgctgcccctggagttcctgtggctgttccactggtgcgccggcgccaagctgaagctgttcaccgaccccgagaccttccgcctgatgggcaaggagcacgccctggtgatcatcaaccacaagatcgagctggactggatggtgggctgggtgctgggccagcacctgggctgcctgggctccatcctgtccgtggccaagaagtccaccaagttcctgcccgtgttcggctggtccctgtggttctccggctacctgttcctggagcgctcctgggccaaggacaagatcaccctgaagtcccacatcgagtccctgaaggactaccccctgcccttctggctgatcatcttcgtggagggcacccgcttcacccgcaccaagctgctggccgcccagcagtacgccgcctcctccggcctgcccgtgccccgcaacgtgctgatcccccacaccaagggcttcgtgtcctccgtgtcccacatgcgctccttcgtgcccgccatctacgacgtgaccgtggccttccccaagacctcccccccccccaccatgctgaagctgttcgagggccagtccgtggagctgcacgtgcacatcaagcgccacgccatgaaggacctgcccgagtccgacgacgccgtggcccagtggtgccgcgacaagttcgtggagaaggacgccctgctggacaagcacaactccgaggacaccttctccggccaggaggtgcaccacgtgggccgccccatcaaggccctgctggtggtgatctcctgggtggtggtgatcatcttcggcgccctgaagttcctgctgtggtcctccctgctgtcctcctggaagggcaaggccttctccgtgatcggcctgggcatcgtggccggcatcgtgaccctgctgatgcacatcctgatcctgtcctcccaggccgagggctccaaccccgtgaaggccgcccccgccaagctgaagaccgagctgtcctcctccaagaaggtgaccaacaaggagaactgactcgag

SEQ ID NO: 21 - DNA, 1122 bp, Artificial Sequence
  Description of Artificial Sequence Synthetic polynucleotide
actagtatggccatcgccgccgccgccgtgatcttcctgttcggcctgctgttcttcgcctccggcatcatcatcaacctgttccaggccctgtgcttcgtgctgatctggcccctgtccaagaacgtgtaccgccgcatcaaccgcgtgttcgccgagctgctgctgatggacctgctgtgcctgttccactggtgggccggcgccaagatcaagctgttcaccgaccccgagaccttccgcctgatgggcatggagcacgccctggtgatcatgaaccacaagaccgacctggactggatggtgggctggatcctgggccagcacctgggctgcctgggctccatcctgtccatcgccaagaagtccaccaagttcatccccgtgctgggctggtccgtgtggttctccgagtacctgttcctggagcgctcctgggccaaggacaagtccaccctgaagtcccacatggagaagctgaaggactaccccctgcccttctggctggtgatcttcgtggagggcacccgcttcacccgcaccaagctgctggccgcccagcagtacgccgcctcctccggcctgcccgtgccccgcaacgtgctgatcccccacaccaagggcttcgtgtcctgcgtgtccaacatgcgctccttcgtgcccgccgtgtacgacgtgaccgtggccttccccaagtcctcccccccccccaccatgctgaagctgttcgagggccagtccatcgtgctgcacgtgcacatcaagcgccacgccctgaaggacctgcccgagtccgacgacgccgtggcccagtggtgccgcgacaagttcgtggagaaggacgccctgctggacaagcacaacgccgaggacaccttctccggccaggaggtgcaccacatcggccgccccatcaagtccctgctggtggtgatcgcctgggtggtggtgatcatcttcggcgccctgaagttcctgcagtggtcctccctgctgtccacctggaagggcaaggccttctccgtgatcggcctgggcatcgccaccctgctgatgcacatgctgatcctgtcctcccaggccgagcgctccaaccccgccaaggtggccaagtgactcgag

SEQ ID NO: 22 - DNA, 1188 bp, Artificial Sequence
  Description of Artificial Sequence Synthetic polynucleotide
actagtatggccatcccctccgccgccgtggtgttcctgttcggcctgctgttcttcacctccggcctgatcatcaacctgttccaggccttctgcttcgtgctgatctcccccctgtccaagaacgcctaccgccgcatcaaccgcgtgttcgccgagctgctgcccctggagttcctgtggctgttccactggtgcgccggcgccaagctgaagctgttcaccgaccccgagaccttccgcctgatgggcaaggagcacgccctggtgatcatcaaccacaagatcgagctggactggatggtgggctgggtgctgggccagcacctgggctgcctgggctccatcctgtccgtggccaagaagtccaccaagttcctgcccgtgttcggctggtccctgtggttctccgagtacctgttcctggagcgctcctgggccaaggacaagatcaccctgaagtcccacatcgagtccctgaaggactaccccctgcccttctggctgatcatcttcgtggagggcacccgcttcacccgcaccaagctgctggccgcccagcagtacgccgcctcctccggcctgcccgtgccccgcaacgtgctgatcccccacaccaagggcttcgtgtcctccgtgtcccacatgcgctccttcgtgcccgccatctacgacgtgaccgtggccttccccaagacctcccccccccccaccatgctgaagctgttcgagggccagtccgtggagctgcacgtgcacatcaagcgccacgccatgaaggacctgcccgagtccgacgacgccgtggcccagtggtgccgcgacaagttcgtggagaaggacgccctgctggacaagcacaactccgaggacaccttctccggccaggaggtgcaccacgtgggccgccccatcaaggccctgctggtggtgatctcctgggtggtggtgatcatcttcggcgccctgaagttcctgctgtggtcctccctgctgtcctcctggaagggcaaggccttctccgtgatcggcctgggcatcgtggccggcatcgtgaccctgctgatgcacatcctgatcctgtcctcccaggccgagggctccaaccccgtgaaggccgcccccgccaagctgaagaccgagctgtcctcctccaagaaggtgaccaacaaggagaactgactcgag

SEQ ID NO: 23 - protein, 385 aa, Artificial Sequence
  Description of Artificial Sequence Synthetic polypeptide
MAIPAAAVIFLFGLLFFTSGLIINLFQALCFVLVWPLSKNAYRRINRVFAELLLSELLCLFDWWAGAKLKLFTDPETFRLMGKEHALVIINHMTELDWMLGWVMGQHLGCLGSILSVAKKSTKFLPVLGWSMWFSEYLYIERSWAKDRTTLKSHIERLTDYPLPFWMVIFVEGTRFTRTKLLAAQQYAASSGLPVPRNVLIPRTKGFVSCVSHMRSFVPAVYDVTVAFPKTSPPPTLLNLFEGQSIVLHVHIKRHAMKDLPESDDAVAQWCRDKFVEKDALLDKHNAEDTFSGQEVHRTGSRPIKSLLVVISWVVVITFGALKFLQWSSWKGKAFSVIGLGIVTLLMHMLILSSQAERSSNPAKVAQAKLKTELSISKKATDKEN

SEQ ID NO: 24 - protein, 384 aa, Artificial Sequence
  Description of Artificial Sequence Synthetic polypeptide
MAIPAAAVIFLFGLIFFASGLIINLFQALCFVLIWPISKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWNKDKSTLKSHIERLKDYPLPFWLVIFAEGTRFTQTKLLAAQQYAASSGLPVPRNVLIPRTKGFVSCVSHMRSFVPAVYDLTVAFPKTSPPPTLLNLFEGQSVVLHVHIKRHAMKDLPESDDEVAQWCRDKFVEKDALLDKHNAEDTFSGQELQHTGRRPIKSLLVVISWVVVIAFGALKFLQWSSWKGKAFSVIGLGIVTLLMHMLILSSQAERSKPAKVAQAKLKTELSISKTVTDKEN

SEQ ID NO: 25 - protein, 278 aa, Artificial Sequence
  Description of Artificial Sequence Synthetic polypeptide
MAIPAAAVIFLFGLIFFASGLIINLFQALCFVLIWPISKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWNKDKSTLKSHIERLKDYPLPFWLVIFAEGTRFTQTKLLAAQQYAASSGLPVPRNVLIPRTKGFVSCVSHMRSFVPAVYDLTVAFPKTSPPPTLLNLFEGQSVVLHVHIKRHAMKDLPESDDEVAQWCRDKFVEK

SEQ ID NO: 26 - protein, 366 aa, Artificial Sequence
  Description of Artificial Sequence Synthetic polypeptide
IVNLVQAVCFVLVRPLSKNTYRRINRVVAELLWLELVWLIDWWAGVKIKVFTDHETFHLMGKEHALVICNHKSDIDWLVGWVLAQRSGCLGSTLAVMKKSSKFLPVIGWSMWFSEYLFLERNWAKDESTLKSGLNRLKDYPLPFWLALFVEGTRFTRAKLLAAQQYAASSGLPVPRNVLIPRTKGFVSSVSHMRSFVPAIYDVTVAIPKTSPPPTLIRMFKGQSSVLHVHLKRHVMKDLPESDDAVAQWCRDIFVEKDALLDKHNADDTFSGQELQDTGRPIKSLLVVISWAVLEVFGAVKFLQWSSLLSSWKGLAFSGIGLGIITLLMHILILFSQSERSTPAKVAPAKAKIEGESSKTEMEKEK

SEQ ID NO: 27 - protein, 287 aa, Artificial Sequence
  Description of Artificial Sequence Synthetic polypeptide
IVNLVQAVCFVLVRPLSKNTYRRINRVVAELLWLELVWLIDWWAGVKIKVFTDHETFHLMGKEHALVICNHKSDIDWLVGWVLAQRSGCLGSTLAVMKKSSKFLPVIGWSMWFSEYLFLERNWAKDESTLKSGLNRLKDYPLPFWLALFVEGTRFTRAKLLAAQQYAASSGLPVPRNVLIPRTKGFVSSVSHMRSFVPAIYDVTVAIPKTSPPPTLIRMFKGQSSVLHVHLKRHVMKDLPESDDAVAQWCRDIFVEKDALLDKHNADDTFSGQELQDTGRPIKSLLV

SEQ ID NO: 28 - protein, 391 aa, Artificial Sequence
  Description of Artificial Sequence Synthetic polypeptide
MAIPSAAVVFLFGLLFFTSGLIINLFQAFCFVLISPLSKNAYRRINRVFAELLPLEFLWLFHWCAGAKLKLFTDPETFRLMGKEHALVIINHKIELDWMVGWVLGQHLGCLGSILSVAKKSTKFLPVFGWSLWFSGYLFLERSWAKDKITLKSHIESLKDYPLPFWLIIFVEGTRFTRTKLLAAQQYAASSGLPVPRNVLIPHTKGFVSSVSHMRSFVPAIYDVTVAFPKTSPPPTMLKLFEGQSVELHVHIKRHAMKDLPESDDAVAQWCRDKFVEKDALLDKHNSEDTFSGQEVHHVGRPIKALLVVISWVVVIIFGALKFLLWSSLLSSWKGKAFSVIGLGIVAGIVTLLMHILILSSQAEGSNPVKAAPAKLKTELSSSKKVTNKEN

SEQ ID NO: 29 - protein, 391 aa, Artificial Sequence
  Description of Artificial Sequence Synthetic polypeptide
MAIPSAAVVFLFGLLFFTSGLIINLFQAFCFVLISPLSKNAYRRINRVFAELLPLEFLWLFHWCAGAKLKLFTDPETFRLMGKEHALVIINHKIELDWMVGWVLGQHLGCLGSILSVAKKSTKFLPVFGWSLWFSEYLFLERSWAKDKITLKSHIESLKDYPLPFWLIIFVEGTRFTRTKLLAAQQYAASSGLPVPRNVLIPHTKGFVSSVSHMRSFVPAIYDVTVAFPKTSPPPTMLKLFEGQSVELHVHIKRHAMKDLPESDDAVAQWCRDKFVEKDALLDKHNSEDTFSGQEVHHVGRPIKALLVVISWVVVIIFGALKFLLWSSLLSSWKGKAFSVIGLGIVAGIVTLLMHILILSSQAEGSNPVKAAPAKLKTELSSSKKVTNKEN

SEQ ID NO: 30 - protein, 376 aa, Artificial Sequence
  Description of Artificial Sequence Synthetic polypeptide
LSLLFFVSGLIVNLVQAVCFVLIRPLSKNTYRRINRVVAELLWLELVWLIDWWAGVKIKVFTDHETFNLMGKEHALVVCNHKSDIDWLVGWVLAQRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKTSVPPTMLRIFKGQSSVLHVHLKRHLMKDLPESDDAVAQWCRDIFVEKDALLDKHNAEDTFSGQELQDIGRPIKSLLVVISWAVLVIFGAVKFLQWSSLLSSWKGLAFSGIGLGIVTLLMHILILFSQSERSTPAKVAPAKPKNEGESSKTEMEKEH

SEQ ID NO: 31 - protein, 320 aa, Artificial Sequence
  Description of Artificial Sequence Synthetic polypeptide
QIKVFTDHETFNLMGKEHALVVCNHKSDIDWLVGWVLAQWSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKTSVPPTMLRIFKGQSSVLHVHLKRHLMKDLPESDDAVAQWCRDIFVEKDALLDKHNAEDTFSGQELQDIGRPIKSLLVVISWAVLVIFGAVKFLQWSSLLSSWKGLAFSGIGLGIVTLLMHILILFSQSERSTPAKVAPAKLKKEGESSKPETDKQN

SEQ ID NO: 32 - protein, 376 aa, Artificial Sequence
  Description of Artificial Sequence Synthetic polypeptide
LSLLFFVSGLIVNLVQAVCFVLIRPLLKNTYRRINRVVAELLWLELVWLIDWWAGIKIKVFTDHETFHLMGKEHALVICNHKSDIDWLVGWVLAQRSGCLGSTLAVMKKSSKFLPVIGWSMWFSEYLFLERNWAKDESTLKSGLNRLKDYPLPFWLALFVEGTRFTRAKLLAAQQYAASSGLPVPRNVLIPRTKGFVSSVSQMRSFVPAIYDVTVAIPKTSPPPTLLRMFKGQSSVLHVHLKRHLMNDLPESDDAVAQWCRDIFVEKDALLDKHNAEDTFSGQELQDTGRPIKSLLVVISWATLVVFGAVKFLQWSSLLSSWKGLAFSGIGLGIITLLMHILILFSQSERSTPAKVAPAKPKNEGESSKTEMEKEH

SEQ ID NO: 33 - protein, 376 aa, Artificial Sequence
  Description of Artificial Sequence Synthetic polypeptide
LSLLFFVSGLIVNLVQAVCFVLIRPLLKNTYRRINRVVAELLWLELVWLIDWWAGIKIKVFTDHETFHLMGKEHALVICNHKSDIDWLVGWVLAQRSGCLGSTLAVMKKSSKFLPVIGWSMWFSEYLFLERNWAKDESTLKSGLNRLKDYPLPFWLALFVEGTRFTRAKLLAAQQYAASSGLPVPRNVLIPRTKGFVSSVSQMRSFVPAIYDVTVAIPKTSPPPTLLRMFKGQSSVLHVHLKRHLMNDLPESDDAVAQWCRDIFVEKDALLDKHNAEDTFSGQELQDIGRPIKSLLVVISWAVLEIFGAVKFLQWSSLLSSWKGLAFSGIGLGIVTLLMHILILFSQSERSTPAKVAPAKPKKEGESSKPETDKEN

SEQ ID NO: 34 - protein, 369 aa, Artificial Sequence
  Description of Artificial Sequence Synthetic polypeptide
MAIAAAAVIFLFGLLFFASGIIINLFQALCFVLIWPLSKNVYRRINRVFAELLLMDLLCLFHWWAGAKIKLFTDPETFRLMGMEHALVIMNHKTDLDWMVGWILGQHLGCLGSILSIAKKSTKFIPVLGWSVWFSEYLFLERSWAKDKSTLKSHMEKLKDYPLPFWLVIFVEGTRFTRTKLLAAQQYAASSGLPVPRNVLIPHTKGFVSCVSNMRSFVPAVYDVTVAFPKSSPPPTMLKLFEGQSIVLHVHIKRHALKDLPESDDAVAQWCRDKFVEKDALLDKHNAEDTFSGQEVHHIGRPIKSLLVVIAWVVVIIFGALKFLQWSSLLSTWKGKAFSVIGLGIATLLMHMLILSSQAERSNPAKVAK

SEQ ID NO: 35 - protein, 388 aa, Artificial Sequence
  Description of Artificial Sequence Synthetic polypeptide
MAIAAAAVIFLFGLLFFASGIIINLFQALCFVLIWPLSKNVYRRINRVFAELLLMDLLCLFHWWAGAKIKLFTDPETFRLMGMEHALVIMNHKTDLDWMVGWILGQHLGCLGSILSIAKKSTKFIPVLGWSVWFSEYLFLERSWAKDESTLKSGLNRLKDYPLPFWLALFVEGTRFTRAKLLAAQQYAASSGLPVPKNVLIPRTKGFVSSVSHMRSFVPAIYDVTVAIPKTSAPPTLLRMFKGQSSVLHVHLKRHLMKDLPESDDAVAQWCRDIFVEKDALLDKHNAEDTFSGQELHDIGRPVKSLLVVISWAMLVVFGAVKFLQWSSLLSSWKGLAFSGIGLGIITLLMHILILFSQSERSTPAKVAPAKQKNNEGESSKTEMEKEH

SEQ ID NO: 36 - protein, 354 aa, Artificial Sequence
  Description of Artificial Sequence Synthetic polypeptide
SGLVVNLIQAFFFVLVRPFSKNAYRKINRVVAELLWLELIWLIDWWAGVKIQLYTDPETFKLMGKEHALVICNHKSDIDWLVGWILAQRSGCLGSALAVMKKSSKFLPVIGWSMWFSEYLFLERSWAKDENTLKSGFQRLRDFPHAFWLALFVEGTRFTQAKLLAAQEYASSMGLPAPRNVLIPRTKGFVTAVTHMRPFVPAVYDVTLAIPKTSPPPTMLRLFKGQSSVVHIHLKRHLMSDLPKSDDSVAQWCKDAFVVKDNLLDKHKENDSFGDGVLQDTGRPLNSLVVVISWACLLIFGALKFFQWSSILSSWKGLAFSAVGLGIVTVLMQILIQFSQSERSNRPMPSKHAK

SEQ ID NO: 37 - protein, 369 aa, Artificial Sequence
  Description of Artificial Sequence Synthetic polypeptide
MAIPTAAYVVPLGAIFFFSGLLVNLIQAFFFITVWPLSKKTYIRINKVIVELLWLEFVWLADWWAGLKIEVYADAETFQLMGKEHALVICNHKSDIDWLVGWILAQRAGCLGSSFAVTKKSARYLPVVGWSIWFSGAIFLERSWEKDENTLKAGFQRLREFPCAFWLGLFVEGTRFTQAKLLAAQEYASTMGLPFPRNVLIPRTKGFIAAVNHMREFVPAIYDLTFAFPKDSPPPTMLRLLKGQPSVVHVHIKRHLMKDLPEKNEAVAQWCKDVFLVKDKLLDKHKDDGSFGDGELHEIGRPLKSLVVVTTWACLLILGTLKFLLWSSLLSSWKGLIFSATGLAVLTVLMQFLIQSTQSERSNPASLSK

SEQ ID NO: 38 - protein, 315 aa, Artificial Sequence
  Description of Artificial Sequence Synthetic polypeptide
LGLLFFISGLAVNLIQAVCFVFLRPLSKNTYRKINRVLAELLWLQLVWLVDWWAGVKIKVFADRESFNLMGKEHALVICNHKSDIDWLVGWVLAQRSGCLGSSLAVMKKSSKFLPVIGWSMWFSEYLFLERSWAKDESTLKEGLRRLKDFPRPFWLALFVEGTRFTQAKLLAAQEYATSQGLPVPRNVLIPRTKVHVHVKRHLMKELPETDEAVAQWCKDLFVEKDKLLDKHVAEDTFSDQPLQDIGRPVKPLLVVSSWACLVAYGALKFLQWSSLLSSWKGIAVSAVALAIVTILMQIMILFSQSERSIPAKVA

SEQ ID NO: 39 - protein, 357 aa, Artificial Sequence
  Description of Artificial Sequence Synthetic polypeptide
LGLLFFISGLAVNLIQAVCFVFLRPLSKNTYRKINRVLAELLWLQLVWLVDWWAGVKIKVFADRESFNLMGKEHALVICNHKSDIDWLVGWVLAQRSGCLGSSLAVMKKSSKFLPVIGWSMWFSEYLFLERSWAKDESTLKEGLRRLKDFPRPFWLALFVEGTRFTQAKLLAAQEYATSQGLPVPRNVLIPRTKGFVSAVSHMRSFVPAVYDMTVAIPKSSPSPTMLRLFKGQSSVVHVHVKRHLMKELPETDEAVAQWCKDLFVEKDKLLDKHVAEDTFSDQPLQDIGRPVKPLLVVSSWACLVAYGALKFLQWSSLLSSWKGIAVSAVALAIVTILMQIMILFSQSERSIPTKVA

SEQ ID NO: 40 - protein, 345 aa, Artificial Sequence
  Description of Artificial Sequence Synthetic polypeptide
MAIAAAAVVFLFGLLFFTSGLIINLAQAVCFVLIWPLSKNAYRRINRVFAELLLLELLWLFHWRAGAKLKLFADPETFRLFGKEHALVICNHRTDLDWMVGWVLGQHFGCLGSILSVAKKSTKFLPVLGWSMWFSEYLFLERSWAKDKSTLKSHTERLKDYPLPFWLGIFVEGTRFTRAKLLAAQQYAASSGLPVPRNVLIPHTKLHVHIKRYAMKDLPESDDAVAQWCRDIYVEKDAFLDKHNAEDTFSGQEVHHIGRPIKSLLVVISWVVVIIFGALKFLRWSSLLSSWKGKAFSVIGLGIVTLLVNILILSSQAERSNPAKVAPAKLKTELSPSKKVTNKEN

SEQ ID NO: 41 - protein, 387 aa, Artificial Sequence
  Description of Artificial Sequence Synthetic polypeptide
MAIAAAAVVFLFGLLFFTSGLIINLAQAVCFVLIWPLSKNAYRRINRVFAELLLLELLWLFHWRAGAKLKLFADPETFRLFGKEHALVICNHRTDLDWMVGWVLGQHFGCLGSILSVAKKSTKFLPVLGWSMWFSEYLFLERSWAKDKSTLKSHTERLKDYPLPFWLGIFVEGTRFTRAKLLAAQQYAASSGLPVPRNVLIPHTKGFVSSMSHMRSFVPAVYDLTVAFPKTSPPPTLLKLFEGQSVVLHVHIKRYAMKDLPESDDAVAQWCRDIYVEKDAFLDKHNAEDTFSGQEVHHIGRPIKSLLVVISWVVVIIFGALKFLRWSSLLSSWKGKAFSVIGLGIVTLLVNILILSSQAERSNPAKVAPAKLKTELSPSKKVTNKEN

SEQ ID NO: 42 - protein, 382 aa, Artificial Sequence
  Description of Artificial Sequence Synthetic polypeptide
AAAVIVPLGILFFISGLVVNLLQAICYVLIRPLSKNTYRKINRVVAETLWLELVWIVDWWAGVKIQVFADNETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQQEQNIGRPIKSLAVVLSWSCLLILGAMKFLHWSNLFSSWKGIAFSALGLGIITLCMQILIRSSQSERSTPAKVVPAKPKDNHNDSGSSSQTE

SEQ ID NO: 43 - protein, 382 aa, Artificial Sequence
  Description of Artificial Sequence Synthetic polypeptide
AAAVIVPLGILFFISGLVVNLLQAVCYVLVRPMSKNTYRKINRVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQQEQNIGRPIKSLAVVLSWSCLLILGAMKFLHWSNLFSSWKGIAFSALGLGIITLCMQILIRSSQSERSTPAKVVPAKPKDNHNDSGSSSQTE

SEQ ID NO: 44 - protein, 385 aa, Artificial Sequence
  Description of Artificial Sequence Synthetic polypeptide
MAIAAAVIVPLGLLFFISGLLMNLLQAICYVLVRPLSKNTYRKINRVVAETLWLELVWIVDWWAGVKIKVFADNETFSRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQQEQNIGRPIKSLAVVLSWSCLLILGAMKFLHWSNLFSSWKGIAFSALGLGIITLCMQILIRSSQSERSTPAKVVPAKPKDNHNDSGSSSQTE

SEQ ID NO: 45 - protein, 352 aa, Artificial Sequence
  Description of Artificial Sequence Synthetic polypeptide
INLVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQKEQNIGRPIKSLAVSLIKTFPWLHPHQLTNIFVLFQVVVSWACLLTLGAMKFLHWSNLFSSWKGIALSAFGLGIITLCMQILIRSSQSERSTPAKVAPAKPK

SEQ ID NO: 46 - protein, 329 aa, Artificial Sequence
  Description of Artificial Sequence Synthetic polypeptide
INLVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPEPEDEIAQWCRDQFVAKDALLDKHIAADTFPGQKEQNIGRPIKSLAVVVSWACLLTLGAMKFLHWSNLFSSWKGIALSAFGLGIITLCMQILIRSSQSERSTPAKVAPAKPK

SEQ ID NO: 47 - protein, 342 aa, Artificial Sequence
  Description of Artificial Sequence Synthetic polypeptide
INLVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQQEQNIGRPIKSLAVVLSWSCLLILGAMKFLHWSNLFSSWKGIAFSALGLGIITLCMQILIRSSQSERSTPAKVVPAKPKDNHNDSGSSSQTE

SEQ ID NO: 48 - protein, 267 aa, Artificial Sequence
  Description of Artificial Sequence Synthetic polypeptide
INLVVAETLWLELVWIVDWWAGVKIQVFADDETFNRMGKEHALVVCNHRSDIDWLVGWILAQRSGCLGSALAVMKKSSKFLPVIGWSMWFSEYLFLERNWAKDESTLKSGLQRLNDFPRPFWLALFVEGTRFTEAKLKAAQEYAASSELPVPRNVLIPRTKGFVSAVSNMRSFVPAIYDMTVAIPKTSPPPTMLRLFKGQPSVVHVHIKCHSMKDLPESDDAIAQWCRDQFVAKDALLDKHIAADTFPGQQEQNIGRPIKSLAVSLS

SEQ ID NO: 49 - protein, 288 aa, Artificial Sequence
  Description of Artificial Sequence Synthetic polypeptide
MAIGVAAIVVPLGLLFILSGLMVNLIQAICFILVRPLSKNMYRRVNRVVVELLWLELIWLIDWWGGVKVDVYADSETFQSLGKEHALVVSNHRSDIDWLVGWVLAQRSGCLGSTLAVMKKSSKFLPVIGWSMWFSEYVFLERSWAKDESTLKSGLRRLKDFPRPFWLALFVEGTRFTQAKLLAAREYAASTGLPIPRNVLIPRTKGFVSAVSNMRSFVPAIYDVTVAIPKTQPSPTMLRIFNRQPSVVHVHIKRHSMNQLPQTDEGVGQWCKDIFVAKDALLDRHLAE

SEQ ID NO: 50 - protein, 375 aa, Artificial Sequence
  Description of Artificial Sequence Synthetic polypeptide
MAIGVAAIVVPLGLLFILSGLMVNLIQAICFILVRPLSKNMYRRVNRVVVELLWLELIWLIDWWGGVKVDVYADSETFQSLGKEHALVVSNHRSDIDWLVGWVLAQRSGCLGSTLAVMKKSSKFLPVIGWSMWFSEYVFLERSWAKDESTLKSGLRRLKDFPRPFWLALFVEGTRFTQAKLLAAREYAASTGLPIPRNVLIPRTKGFVSAVSNMRSFVPAIYDVTVAIPKTQPSPTMLRIFNRQPSVVHVHIKRHSMNQLPQTDEGVAQWCKDIFVAKDALLDRHLAEGKFDEKEFKRIRRPIKSLLVISSWSFLLMFGVFKFLKWSALLSTWKGVAVSTTVLLLVTVVMYMFILFSQSERSSPRKVAPSGPENG

SEQ ID NO: 51 - protein, 375 aa, Artificial Sequence
  Description of Artificial Sequence Synthetic polypeptide
MAIGVAAIVVPLGLLFILSGLIINLIQAICFILVRPLSKNMYRKVNRVVVELLWLELIWLIDWWGGVKVDVYADSETFQSLGKEHALVVSNHRSDIDWLVGWVLAQRSGCLGSTLAVMKKSSKFLPVIGWSMWFSEYVFLERSWAKDESTLKSGLQRLKDFPRPFWLALFVEGTRFTQAKLLAAQEYAASTGLPIPRNVLIPRTKGFVSAVSNMRSFVPAIYDVTVAIPKTQPSPTMLRIFNRQPSVVHVHIKRHSMNQLPQTDEGVAQWCKDIFVAKDALLDRHLAEGKFDEKEFKLIRRPIKSLLVISSWSFLLMFGVFKFLKWSALLSTWKGVAVSTAVLLLVTVVMYMFILFSQSERSSPRKVAPIGPENG

SEQ ID NO: 52 - protein, 288 aa, Artificial Sequence
  Description of Artificial Sequence Synthetic polypeptide
MAIGVAAIVVPLGLLFILSGLIINLIQAICFILVRPLSKNMYRKVNRVVVELLWLELIWLIDWWGGVKVDVYADSETFQSLGKEHALVVSNHRSDIDWLVGWVLAQRSGCLGSTLAVMKKSSKFLPVIGWSMWFSEYVFLERSWAKDESTLKSGLQRLKDFPRPFWLALFVEGTRFTQAKLLAAQEYAASTGLPIPRNVLIPRTKGFVSAVSNMRSFVPAIYDVTVAIPKTQPSPTMLRIFNRQPSVVHVHIKRHSMNQLPQTDEGVAQWCKDIFVAKDALLDRHLAE

SEQ ID NO: 53 - protein, 354 aa, Artificial Sequence
  Description of Artificial Sequence Synthetic polypeptide
SLLFFMSGLVVNFIQAVFYVLVRPISKNTYRRINTLVAELLWLELVWVIDWWAGVKVQLYTDTESFRLMGKEHALLICNHRSDIDWLIGWVLAQRCGCLSSSIAVMKKSSKFLPVIGWSMWFSEYLFLERNWAKDENTLKSGLQRLNDFPKPFWLALFVEGTRFTKAKLLAAQEYAASAGLPVPRNVLIPRTKGFVSAVSNMRSFVPAIYDLTVAIPKTTEQPTMLRLFRGKSSVVHVHLKRHLMKDLPKTDDGVAQWCKDQFISKDALLDKHVAEDTFSGLEVQDIGRPMKSLVVVVSWMCLLCLGLVKFLQWSALLSSWKGMMITTFVLGIVTVLMHILIRSSQSEHSTPAK

SEQ ID NO: 54 - protein, 282 aa, Artificial Sequence
  Description of Artificial Sequence Synthetic polypeptide
QRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKMSTPPTMLRIFKGQSSVLHVHLKRHLMKDLPESDDAVAQWCRDIFVEKDALLDKHNAEDTFSGQELQDIGRPVKSLLVVISWAVLVIFGAVKFLQWSSLLSSWKGLAFSGIGLGIVTLLMHILILFSQSERSTPAKVAPAKPKKEGESSKTETEKEN

SEQ ID NO: 55 - protein, 247 aa, Artificial Sequence
  Description of Artificial Sequence Synthetic polypeptide
DWWAGVKIKVFTDHETLSLMGKEHALVISNHKSDIDWLVGWVLAQRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKMSTPPTMLRIFKGQSSVLHVHLKRHLMKDLPESDDAVAQWCRDIFVEKDALLDKHNAEDTFSGQELQDIGRPVKSLLV

SEQ ID NO: 56 - protein, 326 aa, Artificial Sequence
  Description of Artificial Sequence Synthetic polypeptide
DWWAGVKIKVFTDHETLSLMGKEHALVISNHKSDIDWLVGWVLAQRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKMSTPPTMLRIFKGQSSVLHVHLKRHLMKDLPESDDAVAQWCRDIFVEKDALLDKHNAEDTFSGQELQDIGRPVKSLLVVISWAVLVIFGAVKFLQWSSLLSSWKGLAFSGIGLGIVTLLMHILILFSQSERSTPAKVAPAKPKKEGESSKTETEKEN

SEQ ID NO: 57 - protein, 203 aa, Artificial Sequence
  Description of Artificial Sequence Synthetic polypeptide
QRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKMSTPPTMLRIFKGQSSVLHVHLKRHLMKDLPESDDAVAQWCRDIFVEKDALLDKHNAEDTFSGQELQDIGRPVKSLLV

SEQ ID NO: 58 - protein, 376 aa, Artificial Sequence
  Description of Artificial Sequence Synthetic polypeptide
LSLLFFVSGLFVNLVQAVCFVLIRPFSKNTYRRINRVVAELLWLELVWLIDWWAGVKIKVFTDHETLSLMGKEHALVISNHKSDIDWLVGWVLAQRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKMSTPPTMLRIFKGQSSVLHVHLKRHLMKDLPESDDAVAQWCRDIFVEKDALLDKHNAEDTFSGQELQDIGRPVKSLLVVISWAVLVIFGAVKFLQWSSLLSSWKGLAFSGIGLGIVTLLMHILILFSQSERSTPAKVAPAKPKKEGESSKTETEKEN

SEQ ID NO: 59 - protein, 361 aa, Artificial Sequence
  Description of Artificial Sequence Synthetic polypeptide
QAVCFVLIRPFSKNTYRRINRVVAELLWLELVWLIDWWAGVKIKVFTDHETLSLMGKEHALVISNHKSDIDWLVGWVLAQRSGCLGSTLAVMKKSSKFLPVIGWSMWFSEYLFLERSWAKDESTLKSGLKRLKDYPLPFWLALFVEGTRFTQAKLLAAQQYAASSGLPVPRNVLIPRTKGFVSSVSHMRSFVPAIYDVTVAIPKMSTPPTMLRIFKGQSSVLHVHLKRHLMKDLPESDDAVAQWCRDIFVEKDALLDKHNAEDTFSGQELQDIGRPVKSLLVVISWAVLVIFGAVKFLQWSSLLSSWKGLAFSGIGLGIVTLLMHILILFSQSERSTPAKVAPAKPKKEGESSKTETEKEN

SEQ ID NO: 60 - protein, 387 aa, Artificial Sequence
  Description of Artificial Sequence Synthetic polypeptide
MAIAAAAVVFLFGLLFFTSGLIINLAQAVCFVLIWPLSKNAYRRINRVFAELLLLELLWLFHWRAGAKLKLFADPETFRLFGKEHALVICNHRTDLDWMVGWVLGQHFGCLGSILSVAKKSTKFLPVLGWSMWFSEYLFLERSWAKDKSTLKSHTERLKDYPLPFWLGIFVEGTRFTRAKLLAAQQYAASSGLPVPRNVLIPHTKGFVSSMSHMRSFVPAVYDLTVAFPKTSPPPTLLKLFEGQSVVLHVHIKRYAMKDLPESDDAVAQWCRDIYVEKDAFLDKHNAEDTFSGQEVHHIGRPIKSLLVVISWVVVIIFGALKFLRWSSLLSSWKGKAFSVIGLGIVTLLVNILILSSQAERSNPAKVVPAKLKTELSPSKKVTNKEN

SEQ ID NO: 61 - protein, 386 aa, Artificial Sequence
  Description of Artificial Sequence Synthetic polypeptide
MAIPSAAVVFLFGLLFFASGLIINLVQAVCFVLIWPLSKNTCRRINIVFQDMLLSELLWLFHWRAGAKLKFFTDPETYRHMGKEHALVITNHRTDLDWMIGWVLGEHLGCLGSILSVVKKSTKFLPVLGWSMWFSEYLFLERNWAKDKSTFKSHIERLEDFPQPFWFGIFVEGTRFTRAKLLAAQQYAASSGLPVPRNVLIPHTKGFVSSVSHMRSFVPAVYETTMTFPKTSPPPTLLKLFEGQPLVLHIHMKRHAMKDIPESDDAVAQWCRDKFVEKDALLDKHNAEDTFGGLEVHIGRSIKSLMVVICWVVVIIFGALKFLQWSSLLSSWKGIAFIGIGLGIVNLLVHVLILSSQAERSAPTKVAPAKLKTKLLSSKKITNKEN

SEQ ID NO: 62 - protein, 387 aa, Artificial Sequence
  Description of Artificial Sequence Synthetic polypeptide
MAIPSAAVVFLFGLLFFASGLIINLVQAVCFVLIWPLSKNTCRRINIVFQDMLLSELLWLFHWRAGAKLKFFTDPETYRHMGKEHALVITNHRTDLDWMIGWVLGEHLGCLGSILSVVKKSTKFLPVLGWSMWFSEYLFLERNWAKDKSTFKSHIERLEDFPQPFWFGIFVEGTRFTRAKLLAAQQYAASSGLPVPRNVLIPRTKGFVSSVSHMRSFVPAIYDVTVAIPKTSPPPTMLRMFKGQSSVLHVHLKRHLMKDLPESDDAVAQWCRDIFVEKDALLDKHNAEDTFSGQELQDIGRPIKSLVVVISWAALVVFGAVKFLQWSSLLSSWKGLAFSGIGLGIITLLMHILILFSQSERSTPAKVAPAKPKREGESSKTEMDKEN

SEQ ID NO: 63 - protein, 360 aa, Artificial Sequence
  Description of Artificial Sequence Synthetic polypeptide
MAIPAAAVVFLFGLLFFPSGLIINLFQAVCFVLIWPFSRNTCRRINIVFQEMLLSELLWLFHWRAGAKLKLFADPETYRHMGKEHALLITNHRTDLDWMIGWALGQHLGCLGSILSVVKKSTKFLPSHIERLEDFPQPFWMAIFVEGTRFTRAKLLAAQQYAASSGLPVPRNVLIPRTKGFVSCVSHMRSFVPAVYETTMTFPKTSPPPTLLKLFEGQPIVLHVHMKRHAMKDIPESDEAVAQWCRDKFVEKDSLLDKHNAGDTFSCQEIHIGRPIKSLMVVISWVVVIIFGALKFLQWSSLLSSWKGIAFSGIGLGIVTLLVHILILSSQAERSTPAKVAPAKLKTELSSSTKVTNKEN

SEQ ID NO: 64 - protein, 386 aa, Artificial Sequence
  Description of Artificial Sequence Synthetic polypeptide
MAIPAAAVVFLFGLLFFPSGLIINLFQAVCFVLIWPFSRNTCRRINIVFQEMLLSELLWLFHWRAGAKLKLFADPETYRHMGKEHALLITNHRTDLDWMIGWALGQHLGCLGSILSVVKKSTKFLPVLGWSMWFSEYLFLERNWAKDKSTFKSHIERLEDFPQPFWMAIFVEGTRFTRAKLLAAQQYAASSGLPVPRNVLIPRTKGFVSCVSHMRSFVPAVYETTMTFPKTSPPPTLLKLFEGQPIVLHVHMKRHAMKDIPESDEAVAQWCRDKFVEKDSLLDKHNAGDTFSCQEIHIGRPIKSLMVVISWVVVIIFGALKFLQWSSLLSSWKGIAFSGIGLGIVTLLVHILILSSQAERSTPAKVAPAKLKTELSSSTKVTNKEN

SEQ ID NO: 65 - protein, 376 aa, Artificial Sequence
  Description of Artificial Sequence Synthetic polypeptide
LSLLFFVSGLIVNLVQAVCFVLIRPLSKNTYRRINRVVAELLWLELVWLIDWWAGVKIKVFTDHETFRLMGTEHALVISNHKSDIDWLVGWVLAQRSGCLGSTLAVMKKSSKFLPVIGWSMWFSEYLFLERSWAKDESTLKSGLNRLKDYPLPFWLALFVEGTRFTRAKLLAAQQYAASSGLPVPRNVLIPRTKGFVSSVSHMRSFVPAIYDVTVAIPKTSPPPTMLRMFKGQSSVLHVHLKRHLMKDLPESDDAVAQWCRDIFVEKDALLDKHNAEDTFSGQELQDIGRPIKSLVVVISWAALVVFGAVKFLQWSSLLSSWKGLAFSGIALGIITLLMHILILFSQSERSTPAKVAPAKPKKEGESSKTETDKEN

SEQ ID NO: 66 - protein, 386 aa, Artificial Sequence
  Description of Artificial Sequence Synthetic polypeptide
MAIPAAAVIFLFSILFFASGLIINLVQAVCFVLIWPLSKNTCRRINLVFQEMLLSELLGLFHWRAGAKLKLYTDPETYPLLGKEHALLMINHRTDLDWMIGWVLGQHLGCLGSILSVVKKSTKFLPVLGWSMWFSEYLFLERNWAKDKSTFKSHIERLEDFPQPFWMAIFVEGTRFTRAKLLAAQQYAASSGLPVPRNVLIPHTKGFVSTVSHMRSFVPAVYDTTLTFPKTSPPPTLLNLFAGQPIVLHIHIKRHAMKDIPESDDAVAQWCRDKFVEKDALLDKHNAEDAFSDQEFPISRSIKSLMVVISWVMVIIFGALKFLQWSSLLSSWKGKAFSVIAVGIVTLLMHMSILSSQAERSNPAKVALPKLKTELPSSKKVLNKEN

SEQ ID NO: 67 - protein, 386 aa, Artificial Sequence
  Description of Artificial Sequence Synthetic polypeptide
MAIPAAAVIFLFSILFFASGLIINLVQAVCFVLIWPLSKNTCRRINLVFQEMLLSELLGLFHWRAGAKLKLYTDPETYPLLGKEHALLMINHRTDLDWMIGWVLGQHLGCLGSILSVVKKSTKFLPVLGWSMWFSEYLFLERNWAKDKSTFKSHIERLEDFPQPFWMAIFVEGTRFTRAKLLAAQQYAASSGLPVPRNVLIPHTKGFVSTVSHMRSFVPAVYDTTLTFPKTSPPPTLLNLFAGQPIVLHIHIKRHAMKDIPESDDAVAQWCRDKFVEKDALLDKHNAEDAFSDQEFPISRSIKSLMVVISWVMVIIFGALKFLQWSSLLSSWKGIAFSGIGLGIVTLLMHILILSSQAERSTPAKVAQAKVKTELPSSTKVTNKGN

SEQ ID NO: 68 - protein, 386 aa, Artificial Sequence
  Description of Artificial Sequence Synthetic polypeptide
MAIPAAAVIFLFGILFFASGLIINLVQAVCFVLIWPLSKNTCRRINLVFQEMLLSELLWLFHWRAGAELKLFTDPETYRLLGKEHALVMTNHRTDLDWMIGWVTGQHLGCLGSILSIAKKSTKFLPVLGWSMWFSEYLFLERNWAKDKSTFKSHIERLEDFPQPFWMAIFVEGTRFTRAKLLAAQQYAASSGLPVPRNVLIPHTKGFVSSVCHMRSFVPAVYDTTLTFPKNSPPPTLLNLFAGQPIVLHIHIKRHAMKDMPKSDDAVAQWCRDKFVKKDALLDKHNTEDTFSDQEFPIGRPIKSLMVVISWVVVIIFGTLKFLQWSSLLSSWKGIAFSGIGLGIVTLLVHILILSSQAERSTPPKVAPAKLKTELSSTTKVINKGN

SEQ ID NO: 69 - protein, 345 aa, Artificial Sequence
  Description of Artificial Sequence Synthetic polypeptide
LGLLFFVSGLIVNLVQAVCFVLIRPLSKNTYRRLNRVVAELLWLELVWLIDWWAGVKIKVFTDHETFHLMGKEHALVICNHKSDIDWLVGWVLAQRSGCLGSTLAVMKKSSKFLPVIGWSMWFSEYLFLERSWAKDESTLKSGLNRLKDYPLPFWLALFVEGTRFTRAKLLAAQQYAASSGLPVPRNVLIPRTKGFVSSVSHMRSFVPAIYDVTVAIPKTSPPPTMLRMFKGQSSVDALLDKHNADDTFSGQELHDIGRPIKSLLVVISWAVLVVFGAVKFLQWSSLLSSWKGIAFSGIGLGIVTLLVHILILSSQAERSTSAKVAQAKVKTELSSSKKVKNKGN

SEQ ID NO: 70 - protein, 376 aa, Artificial Sequence
  Description of Artificial Sequence Synthetic polypeptide
LGLLFFVSGLIVNLVQAVCFVLIRPLSKNTYRRLNRVVAELLWLELVWLIDWWAGVKIKVFTDHETFHLMGKEHALVICNHKSDIDWLVGWVLAQRSGCLGSTLAVMKKSSKFLPVIGWSMWFSEYLFLERSWAKDESTLKSGLNRLKDYPLPFWLALFVEGTRFTRAKLLAAQQYAASSGLPVPRNVLIPRTKGFVSSVSHMRSFVPAIYDVTVAIPKTSPPPTMLRMFKGQSSVLHVHLKRHLMKDLPESDDAVAQWCRDIFVEKDVLLDKHNAEDTFSGQELQDIGRPVKSLLVVISWTLLVIFGAVKFLQWSSLLSSWKGLAFSGIGLGIVTLLMHILILFSQSERSTPAKVAPAKPKKEGESSKMETDKEN

SEQ ID NO: 71 - protein, 288 aa, Artificial Sequence
  Description of Artificial Sequence Synthetic polypeptide
LAGWMGSSSGCLGSTLAVMKKSSKFLPVIGWSMWFSEYLFLERSWAKDESTLKSGLNRLKDYPLPFWLALFVEGTRFTRAKLLAAQQYAASLGLPVPRNVLIPRTKGFVSSVSHMRSFVPAIYDVTVAIPKTSPPPTMIRMFKGQSSVLHVHLKRHVMKDLPESDDAVAQWCRDIFVEKDALLDKHNAEDTFSGQELQDTGRPIKSLLVVISWAVLEVFGAVKFLQWSSLLSSWKGLAFSGIGLGIITLLMHILILFSQSERSTPAKVAPAKPKNEGESSKAEMEKEK

SEQ ID NO: 72 - protein, 313 aa, Artificial Sequence
  Description of Artificial Sequence Synthetic polypeptide
LAGWMGSSSGCLGSTLAVMKKSSKFLPVIGWSMWFSEYLFLERSWAKDESTLKSGLNRLKDYPLPFWLALFVEGTRFTRAKLLAAQQYAASLGLPVPRNVLIPRTKGFVSSVSHMRSFVPAIYDVTVAIPKTSPPPTMIRMFKGQSSVLHVHLKRHVMKDLPESDDAVAQWCRDIFVEKDALLDKHNAEDTFSGQELQDTGRPIKSLLVRCFLVLSLIYLNGIMLKLRGPCLQVVISWAVLEVFGAVKFLQWSSLLSSWKGLAFSGIGLGIITLLMHILILFSQSERSTPAKVAPAKPKNEGESSKAEMEKEK

SEQ ID NO: 73 - protein, 288 aa, Artificial Sequence
  Description of Artificial Sequence Synthetic polypeptide
LAGWMGSSSGCLGSTLAVMKKSSKFLPVIGWSMWFSEYLFLERSWAKDESTLKSGLNRLKDYPLPFWLALFVEGTRFTRAKLLAAQQYAASLGLPVPRNVLIPRTKGFVSSVSHMRSFVPAIYDVTVAIPKTSPPPTMIRMFKGQSSVLHVHLKRHVMKDLPESDDAVAQWCRDIFVEKDALLDKHNAEDTFSGQELQDTGRPIKSLLVVTSWAVLVISGAVKFLQWSSLLSSWKGLAFSGIGLGIVTLLMHILILFSQSERSTPAKVAPAKPKKEGESSKTEKDKEN

SEQ ID NO: 74 - protein, 376 aa, Artificial Sequence
  Description of Artificial Sequence Synthetic polypeptide
LGLLFFVSGLIVNLVQAVCFVLIRPLSKNTYRRINRVVAELLWLELVWLIDWWAGVKIKVFTDHETLSLMGKEHALVICNHKSDIDWLVGWVLAQRSGCLGSTLAVMKKSSKFLPVIGWSMWFSEYLFLERSWAKDENTLKSGLNRLKDYPLPFWLALFVEGTRFTRAKLLAAQQYATSSGLPVPRNVLIPRTKGFVSSVSHMRSFVPAIYDVTVAIPKTSPPPTMLRMFKGQSSVLHVHLKRHLMKDLPESDDAVAQWCRDIFVEKDALLDKHNAEDTFSGQELQDTGRPIKSLLVVISWAVLVIFGAVKFLQWSSLLSSWKGLAFSGVGLGIITLLMHILILFSQSERSTPAKVAPAKPKKDGESSKTEIEKEN

SEQ ID NO: 75 - protein, 387 aa, Artificial Sequence
  Description of Artificial Sequence Synthetic polypeptide
MAIAAAAVIVPVSLLFFVSGLIVNLVQAVCFVLIRPLFKNTYRRINRVVAELLWLELVWLIDWWAGVKIKVFTDHETFHLMGKEHALVICNHKSDIDWLVGWVLAQRSGCLGSTLAVMKKSSKFLPVIGWSMWFSEYLFLERNWAKDESTLKSGLNRLKDYPLPFWLALFVEGTRFTRAKLLAAQQYAASSGLPVPRNVLIPRTKGFVSSVSHMRSFVPAIYDVTVAIPKTSPPPTLLRMFKGQSSVLHVHLKRHQMNDLPESDDAVAQWCRDIFVEKDALLDKHNAEDTFSGQELQDTGRPIKSLLIVISWAVLVVFGAVKFLQWSSLLSSWKGLAFSGIGLGVITLLMHILILFSQSERSTPAKVAPAKPKIEGESSKTEMEKEH

SEQ ID NO: 76 - protein, 387 aa, Artificial Sequence
  Description of Artificial Sequence Synthetic polypeptide
MTIASAAVVFLFGILLFTSGLIINLFQAFCSVLVWPLSKNAYRRINRVFAEFLPLEFLWLFHWWAGAKLKLFTDPETFRLMGKEHALVIINHKIELDWMVGWVLGQHLGCLGSILSVAKKSTKFLPVFGWSLWFSEYLFLERNWAKDKKTLKSHIERLKDYPLPFWLIIFVEGTRFTRTKLLAAQQYAASAGLPVPRNVLIPHTKGFVSSVSHMRSFVPAIYDVTVAFPKTSPPPTMLKLFEGHFVELHVHIKRHAMKDLPESEDAVAQWCRDKFVEKDALLDKHNAEDTFSGQEVHHVGRPIKSLLVVISWVVVIIFGALKFLQWSSLLSSWKGIAFSVIGLGTVALLMQILILSSQAERSIPAKETPANLKTELSSSKKVTNKEN

SEQ ID NO: 77 - protein, 375 aa, Artificial Sequence
  Description of Artificial Sequence Synthetic polypeptide
MAIGAAAIVVPLGLLFMLSGLMVNLIQAICFILVRPLSKNMYRRVNRVVVELLWLELIWLIDWWGGVKVDVYADSETFQSLGKEHALVVSNHKSDIDWLVGWVLAQRSGCLGSTLAVMKKSSKFLPVIGWSMWFSEYVFLERSWAKDESTLKSGLQRLKDFPRPFWLALFVEGTRFTQAKLLAAQEYAASTGLPIPRNVLIPRTKGFVSAVSNMRSFVPAIYDVTVAIPKTQPSPTMLRIFNRQPSVVHVRIKRHSMNQLPPTDEGVAQWCKDIFVAKDALLDRHLAEGKFDEKEFKRIRRPIKSLLVISSWSFLLLFGVFKFLKWSALLSTWKGVAVSTAVLLLVTVVMYMFILFSQSERSSPRKVAPSGPENG

SEQ ID NO: 78 - protein, 384 aa, Artificial Sequence
  Description of Artificial Sequence Synthetic polypeptide
MAIPAAVVIFLFGLLFFSSGLIINLFQALCFVLIWPLSKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWDKDKSTLKSHIERLKDYPLPFWLVIFAEGTRFTRTKLLAAQQYAASSGLPVPRNVLIPRTKGFVSCVNHMRSFVPAVYDLTVAFPKTSPPPTLLNLFEGQSVVLHVHIKRHAMKDLPESDDAVAQWCRDKFVEKDALLDKHNAEDTFSSQEVHHTGSRPIKSLLVVISWVVVITFGALKFLQWSSWKGKAFSVIGLGIVTLLMHMLILSSQAERSKPAKVTQAKLKTELSISKKVTDKEN

SEQ ID NO: 79 - protein, 384 aa, Artificial Sequence
  Description of Artificial Sequence Synthetic polypeptide
MAIAAAAVIFLFGLLFFASGLIINLFQALCFVLIRPLSKNAYRRINRVFAELLLSELLCLFDWWAGAKLKLFTDPETLRLMGKEHALIIINHMTELDWMVGWVMGQHFGCLGSIISVAKKSTKFLPVLGWSMWFSEYLYLERSWAKDKSTLKSHIERLKDYPLPFWLVIFVEGTRFTRTKLLAAQEYAASSGLPVPRNVLIPRTKGFVSCVNHMRSFVPAVYDVTVAFPKTSPQPTLLNLFEGRSIVLHVHIKRHAMKDLPESDDAVAQWCRDKFVEKDALLDKHNAEDTFSGQEVHHTGRRPIKSLLVVMSWVVVTTFGALKFLQWSSWKGKAFSVIGLGIVTLLMHVLILSSQAERSNPAKVVQAELNTELSISKKVTNKGN

SEQ ID NO: 80 - protein, 373 aa, Artificial Sequence
  Description of Artificial Sequence Synthetic polypeptide
MAIPAAAVIFLFGLIFFASGLIINLFQALCFVLIWPLWKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWDKDKSTLKSHIERLKDYPLPFWLVIFAEGTRFTRTKLLAAQQYAASSGLPVPRNVLIPRTKGFVSSVSHMRSFVPAIYDVTVAIPKTSPPPTLIRMFKGQSSVLHVHLKRHVMKDLPESDDAVAQWCRDIFVEKDALLDKHNAEDTFSGQELQDTGRPIKSLLVVISWAVLEVFGAVKFLQWSSLLSSWKGLAFSGIGLGIITLLMHILILFSQSERSTPAKVAPAKAK

SEQ ID NO: 81 - protein, 398 aa, Artificial Sequence
  Description of Artificial Sequence Synthetic polypeptide
MAIPAAAVIFLFGLIFFASGLIINLFQALCFVLIWPLWKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWDKDKSTLKSHIERLKDYPLPFWLVIFAEGTRFTRTKLLAAQQYAASSGLPVPRNVLIPRTKGFVSSVSHMRSFVPAIYDVTVAIPKTSPPPTLIRMFKGQSSVLHVHLKRHVMKDLPESDDAVAQWCRDIFVEKDALLDKHNAEDTFSGQELQDTGRPIKSLLVRCFLVLSLIYLNGIILKLCGLCLQVVISWAVLEVFGAVKFLQWSSLLSSWKGLAFSGIGLGIITLLMHILILFSQSERSTPAKVAPAKAK

SEQ ID NO: 82 - protein, 387 aa, Artificial Sequence
  Description of Artificial Sequence Synthetic polypeptide
MAIPAAAVIFLFGLIFFASGLIINLFQALCFVLIWPLWKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWDKDKSTLKSHIERLKDYPLPFWLVIFAEGTRFTRTKLLAAQQYAASSGLPVPRNVLIPRTKGFVSSVSHMRSFVPAIYDVTVAIPKTSPPPTLIRMFKGQSSVLHVHLKRHVMKDLPESDDAVAQWCRDIFVEKDALLDKHNAEDTFSGQELQDTGRPIKSLLVVISWAVLEVFGAVKFLQWSSLLSSWKGLAFSGIGLGIITLLMHILILFSQSERSTPAKVAPAKAKMEGESSKTEMEMEK

SEQ ID NO: 83 - protein, 412 aa, Artificial Sequence
  Description of Artificial Sequence Synthetic polypeptide
MAIPAAAVIFLFGLIFFASGLIINLFQALCFVLIWPLWKNAYRRINRVFAELLLSELLCLFDWWAGAKLKLFTDPETFRLMGKEHALVIINHMTELDWMVGWVMGQHFGCLGSILSVAKKSTKFLPVLGWSMWFTEYLYIERSWDKDKSTLKSHIERLKDYPLPFWLVIFAEGTRFTRTKLLAAQQYAASSGLPVPRNVLIPRTKGFVSSVSHMRSFVPAIYDVTVAIPKTSPPPTLIRMFKGQSSVLHVHLKRHVMKDLPESDDAVAQWCRDIFVEKDALLDKHNAEDTFSGQELQDTGRPIKSLLVRCFLVLSLIYLNGIILKLCGLCLQVVISWAVLEVFGAVKFLQWSSLLSSWKGLAFSGIGLGIITLLMHILILFSQSERSTPAKVAPAKAKMEGESSKTEMEMEK

SEQ ID NO: 84 - protein, 387 aa, Artificial Sequence
  Description of Artificial Sequence Synthetic polypeptide
MAIAAAPVIFLFGLLFFASGLIINLFQAICFVLIWPLSKNAYRRINRVFAELLLSELLCLFDWWAGAKLKLFTDPETFRLMGKEHALVITNHKIDLDWMIGWILGQHFGCLGSVISIAKKSTKFLPIFGWSLWFSEYLFLERNWAKDKRTLKSHIERMKDYPLPLWLILFVEGTRFTRTKLLAAQQYAASSGLPVPRNVLIPHTKGFVSSVSHMRSFVPAIYDVTVAIPKTSPPPTLIRMFKGQSSVLHVHLKRHLMKDLPESDDAVAQWCRDIFVEKDALLDKHNAEDTFSGQELQETGRPIKSLLVVISWAVLEVYGAVKFLQWSSLLSSWKGLAFSGIGLGLITLLMHILILFSQSERSTPAKVAPAKPKKEGESSKTEMEKEK

SEQ ID NO: 85 - protein, 382 aa, Artificial Sequence
  Description of Artificial Sequence Synthetic polypeptide
MHVLLEMVTFRFSSFFVFDNVQALCFVLIWPLSKSAYRKINRVFAELLLSELLCLFDWWAGAKLKLFTDPETFRLMGKEHALVITNHKIDLDWMIGWILGQHFGCLGSVISIAKKSTKFLPIFGWSLWFSEYLFLERNWAKDKRTLKSHIERMKDYPLPLWLILFVEGTRFTRTKLLAAQQYAASSGLPVPRNVLIPHTKGFVSSVSHMRSFVPAVYDVTVAFPKTSPPPTMLSLFEGQSVVLHVHIKRHAMKDLPDSDDAVAQWCRDKFVEKDALLDKHNAEDTFSGQEVHHVGRPIKSLLVVISWMVVIIFGALKFLQWSSLLSSWKGKAFSAIGLGIATLLMHVLVVFSQADRSNPAKVPPAKLNTELSSSKKVTNKEN

SEQ ID NO: 86 - DNA, 7194 bp, Artificial Sequence
  Description of Artificial Sequence Synthetic polynucleotide
gtttaaacgccggtcaccacccgcatgctcgtactacagcgcacgcaccgcttcgtgatccaccgggtgaacgtagtcctcgacggaaacatctggttcgggcctcctgcttgcactcccgcccatgccgacaacctttctgctgttaccacgacccacaatgcaacgcgacacgaccgtgtgggactgatcggttcactgcacctgcatgcaattgtcacaagcgcttactccaattgtattcgtttgttttctgggagcagttgctcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtatgcagaccgcccaccagcgcccccccaccgagggccactgcttcggcgcccgcctgcccaccgcctcccgccgcgccgtgcgccgcgcctggtcccgcatcgcccgcgggcgcgccgccgccgccgccgacgccaaccccgcccgccccgagcgccgcgtggtgatcaccggccagggcgtggtgacctccctgggccagaccatcgagcagttctactcctccctgctggagggcgtgtccggcatctcccagatccagaagttcgacaccaccggctacaccaccaccatcgccggcgagatcaagtccctgcagctggacccctacgtgcccaagcgctgggccaagcgcgtggacgacgtgatcaagtacgtgtacatcgccggcaagcaggccctggagtccgccggcctgcccatcgaggccgccggcctggccggcgccggcctggaccccgccctgtgcggcgtgctgatcggcaccgccatggccggcatgacctccttcgccgccggcgtggaggccctgacccgcggcggcgtgcgcaagatgaaccccttctgcatccccttctccatctccaacatgggcggcgccatgctggccatggacatcggcttcatgggccccaactactccatctccaccgcctgcgccaccggcaactactgcatcctgggcgccgccgaccacatccgccgcggcgacgccaacgtgatgctggccggcggcgccgacgccgccatcatcccctccggcatcggcggcttcatcgcctgcaaggccctgtccaagcgcaacgacgagcccgagcgcgcctcccgcccctgggacgccgaccgcgacggcttcgtgatgggcgagggcgccggcgtgctggtgctggaggagctggagcacgccaagcgccgcggcgccaccatcctggccgagctggtgggcggcgccgccacctccgacgcccaccacatgaccgagcccgacccccagggccgcggcgtgcgcctgtgcctggagcgcgccctggagcgcgcccgcctggcccccgagcgcgtgggctacgtgaacgcccacggcacctccacccccgccggcgacgtggccgagtaccgcgccatccgcgccgtgatcccccaggactccctgcgcatcaactccaccaagtccatgatcggccacctgctgggcggcgccggcgccgtggaggccgtggccgccatccaggccctgcgcaccggctggctgcaccccaacctgaacctggagaaccccgcccccggcgtggaccccgtggtgctggtgggcccccgcaaggagcgcgccgaggacctggacgtggtgctgtccaactccttcggcttcggcggccacaactcctgcgtgatcttccgcaagtacgacgagatggactacaaggaccacgacggcgactacaaggaccacgacatcgactacaaggacgacgacgacaagtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctaggtgatatccatcttaaggatctaagtaagattcgaagcgctcgaccgtgccggacggactgcagccccatgtcgtagtgaccgccaatgtaagtgggctggcgtttccctgtacgtgagtcaacgtcactgcacgcgcaccaccctctcgaccggcaggaccaggcatcgcgagatacagcgcgagccagacacggagtgccgagctatgcgcacgctccaactaggtaccagtttaggtccagcgtccgtggggggggacgggctgggagcttgggccgggaagggcaagacgatgcagtccctctggggagtcacagccgactgtgtgtgttgcactgtgcggcccgcagcactcacacgcaaaatgcctggccgacaggcaggccctgtccagtgcaacatccacggtccctctcatcaggctcaccttgctcattgacataacggaatgcgtaccgctctttcagatctgtccatccagagaggggagcaggctccccaccgacgctgtcaaacttgcttcctgcccaaccgaaaacattattgtttgagggggggggggggggggcagattgcatggcgggatatctcgtgaggaacatcactgggacactgtggaacacagtgagtgcagtatgcagagcatgtatgctaggggtcagcgcaggaagggggcctttcccagtctcccatgccactgcaccgtatccacgactcaccaggaccagcttcttgatcggcttccgctcccgtggacaccagtgtgtagcctctggactccaggtatgcgtgcaccgcaaaggccagccgatcgtgccgattcctgggtggaggatatgagtcagccaacttggggctcagagtgcacactggggcacgatacgaaacaacatctacaccgtgtcctccatgctgacacaccacagcttcgctccacctgaatgtgggcgcatgggcccgaatcacagccaatgtcgctgctgccataatgtgatccagaccctctccgcccagatgccgagcggatcgtgggcgctgaatagattcctgtttcgatcactgtttgggtcctttccttttcgtctcggatgcgcgtctcgaaacaggctgcgtcgggctttcggatcccttttgctccctccgtcaccatcctgcgcgcgggcaagttgcttgaccctgggctgataccagggttggagggtattaccgcgtcaggccattcccagcccggattcaattcaaagtctgggccaccaccctccgccgctctgtctgatcactccacattcgtgcatacactacgttcaagtcctgatccaggcgtgtctcgggacaaggtgtgcttgagtttgaatctcaaggacccactccagcacagctgctggttgaccccgccctcgcaatctagaatggccgcgtccgtccactgcaccctgatgtccgtggtctgcaacaacaagaaccactccgcccgccccaagctgcccaactcctccctgctgcccggcttcgacgtggtggtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcctgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgacaattgacggagcgtcgtgcgggagggagtgtgccgagcggggagtcccggtctgtgcgaggcccggcagctgacgctggcgagccgtacgccccgagggtccccctcccctgcaccctcttccccttccctctgacggccgcgcctgttcttgcatgttcagcgacggatcctagggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtcctgggcgaagaacgagggaatttgtgggtaaaacaagcatcgtctctcaggccccggcgcagtggccgttaaagtccaagaccgtgaccaggcagcgcagcgcgtccgtgtgcgggccctgcctggcggctcggcgtgccaggctcgagagcagctccctcaggtcgccttggacggcctctgcgaggccggtgagggcctgcaggagcgcctcgagcgtggcagtggcggtcgtatccgggtcgccggtcaccgcctgcgactcgccatccgaagagcgtttaaac

SEQ ID NO: 87 - DNA, 7081 bp, Artificial Sequence
  Description of Artificial Sequence Synthetic polynucleotide
gctcttcgccgccgccactcctgctcgagcgcgcccgcgcgtgcgccgccagcgccttggccttttcgccgcgctcgtgcgcgtcgctgatgtccatcaccaggtccatgaggtctgccttgcgccggctgagccactgcttcgtccgggcggccaagaggagcatgagggaggactcctggtccagggtcctgacgtggtcgcggctctgggagcgggccagcatcatctggctctgccgcaccgaggccgcctccaactggtcctccagcagccgcagtcgccgccgaccctggcagaggaagacaggtgaggggggtatgaattgtacagaacaaccacgagccttgtctaggcagaatccctaccagtcatggctttacctggatgacggcctgcgaacagctgtccagcgaccctcgctgccgccgcttctcccgcacgcttctttccagcaccgtgatggcgcgagccagcgccgcacgctggcgctgcgcttcgccgatctgaggacagtcggggaactctgatcagtctaaacccccttgcgcgttagtgttgccatcctttgcagaccggtgagagccgacttgttgtgcgccaccccccacaccacctcctcccagaccaattctgtcacctttttggcgaaggcatcggcctcggcctgcagagaggacagcagtgcccagccgctgggggttggcggatgcacgctcaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcctgaagaatgggaggcaggtgttgttgattatgagtgtgtaaaagaaaggggtagagagccgtcctcagatccgactactatgcaggtagccgctcgcccatgcccgcctggctgaatattgatgcatgcccatcaaggcaggcaggcatttctgtgcacgcaccaagcccacaatcttccacaacacacagcatgtaccaacgcacgcgtaaaagttggggtgctgccagtgcgtcatgccaggcatgatgtgctcctgcacatccgccatgatctcctccatcgtctcgggtgtttccggcgcctggtccgggagccgttccgccagatacccagacgccacctccgacctcacggggtacttttcgagcgtctgccggtagtcgacgatcgcgtccaccatggagtagccgaggcgccggaactggcgtgacggagggaggagagggaggagagagaggggggggggggggggggatgattacacgccagtctcacaacgcatgcaagacccgtttgattatgagtacaatcatgcactactagatggatgagcgccaggcataaggcacaccgacgttgatggcatgagcaactcccgcatcatatttcctattgtcctcacgccaagccggtcaccatccgcatgctcatattacagcgcacgcaccgcttcgtgatccaccgggtgaacgtagtcctcgacggaaacatctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctcttgttttccagaaggagttgctccttgagcctttcattctcagcctcgataacctccaaagccgctctaattgtggagggggttcgaatttaaaagcttggaatgttggttcgtgcgtctggaacaagcccagacttgttgctcactgggaaaaggaccatcagctccaaaaaacttgccgctcaaaccgcgtacctctgctttcgcgcaatctgccctgttgaaatcgccaccacattcatattgtgacgcttgagcagtctgtaattgcctcagaatgtggaatcatctgccccctgtgcgagcccatgccaggcatgtcgcgggcgaggacacccgccactcgtacagcagaccattatgctacctcacaatagttcataacagtgaccatatttctcgaagctccccaacgagcacctccatgctctgagtggccaccccccggccctggtgcttgcggagggcaggtcaaccggcatggggctaccgaaatccccgaccggatcccaccacccccgcgatgggaagaatctctccccgggatgtgggcccaccaccagcacaacctgctggcccaggcgagcgtcaaaccataccacacaaatatccttggcatcggccctgaattccttctgccgctctgctacccggtgcttctgtccgaagcaggggttgctagggatcgctccgagtccgcaaacccttgtcgcgtggcggggcttgttcgagcttgaagagc

SEQ ID NO: 88 - DNA, 6029 bp, Artificial Sequence
  Description of Artificial Sequence Synthetic polynucleotide
gctcttccgctaacggaggtctgtcaccaaatggaccccgtctattgcgggaaaccacggcgatggcacgtttcaaaacttgatgaaatacaatattcagtatgtcgcgggcggcgacggcggggagctgatgtcgcgctgggtattgcttaatcgccagcttcgcccccgtcttggcgcgaggcgtgaacaagccgaccgatgtgcacgagcaaatcctgacactagaagggctgactcgcccggcacggctgaattacacaggcttgcaaaaataccagaatttgcacgcaccgtattcgcggtattttgttggacagtgaatagcgatgcggcaatggcttgtggcgttagaaggtgcgacgaaggtggtgccaccactgtgccagccagtcctggcggctcccagggccccgatcaagagccaggacatccaaactacccacagcatcaacgccccggcctatactcgaaccccacttgcactctgcaatggtatgggaaccacggggcagtcttgtgtgggtcgcgcctatcgcggtcggcgaagaccgggaaggtaccgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacgtactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagaattcctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccatggccgccgccgtgatcgtgcccctgggcatcctgttcttcatctccggcctggtggtgaacctgctgcaggccatctgctacgtgctgatccgccccctgtccaagaacacctaccgcaagatcaaccgcgtggtggccgagaccctgtggctggagctggtgtggatcgtggactggtgggccggcgtgaagatccaggtgttcgccgacaacgagaccttcaaccgcatgggcaaggagcacgccctggtggtgtgcaaccaccgctccgacatcgactggctggtgggctggatcctggcccagcgctccggctgcctgggctccgccctggccgtgatgaagaagtcctccaagttcctgcccgtgatcggctggtccatgtggttctccgagtacctgttcctggagcgcaactgggccaaggacgagtccaccctgaagtccggcctgcagcgcctgaacgacttcccccgccccttctggctggccctgttcgtggagggcacccgcttcaccgaggccaagctgaaggccgcccaggagtacgccgcctcctccgagctgcccgtgccccgcaacgtgctgatcccccgcaccaagggcttcgtgtccgccgtgtccaacatgcgctccttcgtgcccgccatctacgacatgaccgtggccatccccaagacctcccccccccccaccatgctgcgcctgttcaagggccagccctccgtggtgcacgtgcacatcaagtgccactccatgaaggacctgcccgagtccgacgacgccatcgcccagtggtgccgcgaccagttcgtggccaaggacgccctgctggacaagcacatcgccgccgacaccttccccggccagcaggagcagaacatcggccgccccatcaagtccctggccgtggtgctgtcctggtcctgcctgctgatcctgggcgccatgaagttcctgcactggtccaacctgttctcctcctggaagggcatcgccttctccgccctgggcctgggcatcatcaccctgtgcatgcagatcctgatccgctcctcccagtccgagcgctccacccccgccaaggtggtgcccgccaagcccaaggacaaccacaacgactccggctcctcctcccagaccgaggtggagaagcagaagtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttaattaagagctcagcggcgacggtcctgctaccgtacgacgttgggcacgcccatgaaagtttgtataccgagcttgttgagcgaactgcaagcgcggctcaaggatacttgaactcctggattgatatcggtccaataatggatggaaaatccgaacctcgtgcaagaactgagcaaacctcgttacatggatgcacagtcgccagtccaatgaacattgaagtgagcgaactgttcgcttcggtggcagtactactcaaagaatgagctgctgttaaaaatgcactctcgttctctcaagtgagtggcagatgagtgctcacgccttgcacttcgctgcccgtgtcatgccctgcgccccaaaatttgaaaaaagggatgagattattgggcaatggacgacgtcgtcgctccgggagtcaggaccggcggaaaataagaggcaacacactccgcttcttagctcttc

SEQ ID NO: 89 - DNA, 1176 bp, Artificial Sequence
  Description of Artificial Sequence Synthetic polynucleotide
atggccatggccgccgccgccgtgatcgtgcccctgggcatcctgttcttcatctccggcctggtggtgaacctgctgcaggccgtgtgctacgtgctgatccgccccctgtccaagaacacctaccgcaagatcaaccgcgtggtggccgagaccctgtggctggagctggtgtggatcgtggactggtgggccggcgtgaagatccaggtgttcgccgacgacgagaccttcaaccgcatgggcaaggagcacgccctggtggtgtgcaaccaccgctccgacatcgactggctggtgggctggatcctggcccagcgctccggctgcctgggctccgccctggccgtgatgaagaagtcctccaagttcctgcccgtgatcggctggtccatgtggttctccgagtacctgttcctggagcgcaactgggccaaggacgagtccaccctgaagtccggcctgcagcgcctgaacgacttcccccgccccttctggctggccctgttcgtggagggcacccgcttcaccgaggccaagctgaaggccgcccaggagtacgccgcctcctcccagctgcccgtgccccgcaacgtgctgatcccccgcaccaagggcttcgtgtccgccgtgtccaacatgcgctccttcgtgcccgccatctacgacatgaccgtggccatccccaagacctcccccccccccaccatgctgcgcctgttcaagggccagccctccgtggtgcacgtgcacatcaagtgccactccatgaaggacctgcccgagtccgacgacgccatcgcccagtggtgccgcgaccagttcgtggccaaggacgccctgctggacaagcacatcgccgccgacaccttccccggccagaaggagcacaacatcggccgccccatcaagtccctggccgtggtggtgtcctgggcctgcctgctgaccctgggcgccatgaagttcctgcactggtccaacctgttctcctccctgaagggcatcgccctgtccgccctgggcctgggcatcatcaccctgtgcatgcagatcctgatccgctcctcccagtccgagcgctccacccccgccaaggtggcccccgccaagcccaaggacaagcaccagtccggctcctcctcccagaccgaggtggaggagaagcagaagtga

SEQ ID NO: 90 - DNA, 1164 bp, Artificial Sequence
  Description of Artificial Sequence Synthetic polynucleotide
atggccatcgccgccgccgccgtgatcgtgcccctgggcctgctgttcttcatctccggcctggtggtgaacctgatccaggccctgtgcttcgtgctgatccgccccctgtccaagaacacctaccgcaagatcaaccgcgtggtggccgagctgctgtggctggagctgatctggctggtggactggtgggccggcgtgaagatcaaggtgttcatggaccccgagtccttcaacctgatgggcaaggagcacgccctggtggtggccaaccaccgctccgacatcgactggctggtgggctggctgctggcccagcgctccggctgcctgggctccgccctggccgtgatgaagaagtcctccaagttcctgcccgtgatcggctggtccatgtggttctccgagtacctgttcctggagcgctcctgggccaaggacgagaacaccctgaaggccggcctgcagcgcctgaaggacttcccccgccccttctggctggccttcttcgtggagggcacccgcttcacccaggccaagttcctggccgcccaggagtacgccgcctcccagggcctgcccatcccccgcaacgtgctgatcccccgcaccaagggcttcgtgtccgccgtgtcccacatgcgctccttcgtgcccgccatctacgacatgaccgtggccatccccaagtcctccccctcccccaccatgctgcgcctgttcaagggccagccctccgtggtgcacgtgcacatcaagcgctgcctgatgaaggagctgcccgagaccgacgaggccgtggcccagtggtgcaaggacatgttcgtggagaaggacaagctgctggacaagcacatcgccgaggacaccttctccgaccagcccatgcaggacctgggccgccccatcaagtccctgctggtggtggcctcctgggcctgcctgatggcctacggcgccctgaagttcctgcagtgctcctccctgctgtcctcctggaagggcatcgccttcttcctggtgggcctggccatcgtgaccatcctgatgcacatcctgatcctgttctcccagtccgagcgctccacccccgccaaggtggcccccggcaagcccaagaacgacggcgagacctccgaggcccgccgcgacaagcagcagtga

SEQ ID NO: 91 - DNA, 1164 bp, Artificial Sequence
  Description of Artificial Sequence Synthetic polynucleotide
atggccatccccgccgccatcgtgatcgtgcccgtgggcctgctgttcttcatctccggcctgatcgtgaacctgctgcaggccctgtgcttcgtgctgatccgccccctgtccaagtccgcctaccgcaccatcaaccgccagctggtggagctgctgtggctggagctggtgtgcatcgtggactggtgggcccgcgtgaagatccagctgttcaccgacaaggagaccctgaactccatgggcaaggagcacgccctggtgatgtgcaaccaccgctccgacatcgactggctggtgggctggatcctggcccagcgctccggctgcctgggctccaccgtggccgtgatgaagaagtcctccaaggtgctgcccgtgatcggctggtccatgtggttctccgagtacctgttcctggagcgcaactgggccaaggacgagtccaccctgaagtccggcctgcagcgcctgcgcgacttcccccgccccttctggctggccctgttcgtggagggcacccgcttcacccagcccaagctgctggccgcccaggagtacgccgcctccaccggcctgcccatcccccgcaacgtgctgatcccccgcaccaagggcttcgtgtccgccgtgtccatcacccgctccttcgtgcccgtgatctacgacatcaccgtggccatccccaagtcctccccccagcccaccatgctgcgcctgttcaagggccagtcctccgtggtgcacgtgcacctgaagcgccacctgatgaaggacctgcccgagtccgacgacgacgtggcccagtggtgccgcgaccagttcgtggtgaaggactccctgctggacaagcacatcgccgaggacaccttctccgaccaggagctgcaggacatcggccgccccatcaagtccctggtggtgttcacctcctgggtgtgcatcatcaccttcggcgccctgaagttcctgcagtggtcctccctgctgcactcctggaagggcatcgccatctccgcctccggcctggccatcgtgaccgtgctgatgcacatcctgatccgcttctcccagtccgagcactccacctccgccaagatcgccgccgagaagcacaagaacggcggcgtgtcccaggagatgggccgcgagaagcagcactga

SEQ ID NO: 92 - DNA, 1164 bp, Artificial Sequence
  Description of Artificial Sequence Synthetic polynucleotide
atggagatccccgccgtggccgtgatcgtgcccatcggcatcctgttcttcatctccggcctgatcgtgaacctgatgcaggccatctgcttcttcctgatccgccccctgtccaagaacacccaccgcatcgtgaaccgccagctggccgagctgctgtggctggagctgatctggatcgtggactggtgggccggcgtgaagatccagctgttcaccgacaaggagaccctgcacctgatgggcaaggagcacgccctggtgatctgcaaccactcctccgacatcgactggctggtgggctggctgctgtgccagcgctccggctgcctgggctccgccctggccgtgatgaagtcctcctccaaggtgctgcccgtgatcggctggtccatgtggttctccgagtacctgttcctggagcgctcctgggccaaggacgagtccaccctgaagtccggcctgcagcgcctgaaggacttcccccgccccttctggctggccctgttcgtggagggcacccgcttcacccaggccaagctgctggccgcccaggagtacgccatgtccgccggcctgcccgtgccccgcaacgtgctgatcccccgcaccaagggcttcgtgtccgccgtgtccaacatgcgctccttcgtgcccgccatctacgacgtgaccgtggccatccccaagtcctccgtgcagcccaccatgctgcgcctgttcaagggccagtcctccgtggtgcaggtgcacctgaagcgccactccatgaaggacctgcccgagtccgaggacgacgtggcccagtggtgccgcgaccgcttcgtggtgaaggactccctgctggacaagcacaaggtggaggacaccttcaccgaccaggagctgcaggacctgggccgccccatcaagtccctggtggtggtgacctgctgggcctgcatcatcatcttcggcatcctgaagttcctgcagtggtcctccctgctgtactcctggaagggcatggccatctccgcctccggcctggccgtggtgaccttcctgatgcagatcctgatccgcttctcccagtccgagcgctccacccccgccaagatcgcccccgccaagcccaacaaggccggcaactcctccgagaccgtgcgcgacaagcaccagtga

SEQ ID NO: 93 - DNA, 1137 bp, Artificial Sequence
  Description of Artificial Sequence Synthetic polynucleotide
atggccatccccgccgccatcatcatcgtgcccctgggcctgatcttcttcacctccggcctgatcatcaacctgatccaggccgtgtgctacgtgctgatccgccccctgtccaagtccaccttccgccgcatcaaccgcgagctggccgagctgctgtggctggagctggtgtgggtggtggactggtgggccggcgtgaagatccagctgttcaccgacaaggagaccctgcactccatgggcaaggagcacgccctggtgatctgcaaccaccgctccgacatcgactggctggtgggctggatcctggcccagcgctccggctgcctgggctccgccctggccgtgatgaagaagtcctccaaggtgctgcccgtgatcggctggtccatgtggttctccgagtacttcttcctggagcgcaactgggccatggacgagtccaccctgaagtccggcctgcagcgcctgaaggacttcccccagcccttctggctggccctgttcgtggagggcacccgcttcacccagcccaagctgctggccgcccaggagtacgccgcctccgccggcctgcccatcccccgcaacgtgctgatcccccgcaccaagggcttcgtgtccgccgtgaacatcatgcgctccttcgtgcccgccatctacgacgtgaccgtggccatccccaagtcctccccccagcccaccatgctgcgcctgttcaagggccagtcctccgtggtgcacgtgcacctgaagcgccacctgatggaggacctgcccgagaccgacgacgacgtggcccagtggtgccgcgaccgcttcgtggtgaaggactccctgctggacaagtacgtggccgaggacaccttctccgaccaggagctgcaggacctgggccgccccatcaagtccctggtggtggtgacctcctgggtgtgcatcatcgccttcggctccctgaagttcctgcagtggtcctccctgctgtactcctggaagggcatcgtgatctccgccgcctccctggccgtggtgaccgtgctgatgcagatcctgatccgcttctcccagtccgagcgctccacctccgccaagatcgccgccgccaagcgcaagaacgtgggcgagcactga

SEQ ID NO: 94 - DNA, 1164 bp, Artificial Sequence
  Description of Artificial Sequence Synthetic polynucleotide
atggccatccccgtggtggtggtgatcgtgcccgtgggcctgctgttcttcatctccggcctgatcgtgaacctgctgcaggccctgtgcttcgtgctgatccgccccctgtccaagtccgcctaccgcaccatcaaccgccagctggtggagctgctgtggctggagctggtgtgcatcgtggactggtgggcccgcgtgaagatccagctgttcatcgacaaggagaccctgaactccatgggcaaggagcacgccctggtgatgtgcaaccaccgctcctacatcgactggctggtgggctggatcctggcccagcgctccggctgcctgggctccaccgtggccgtgatgaagaagtcctccaaggtgctgcccgtgatcggctggtccatgtggttctccgagtacctgttcctggagcgcaactgggccaaggacgagtccaccctgaagtccggcctgcagcgcctgcgcgacttcccccgccccttctggctggccctgttcgtggagggcacccgcttcacccagcccaagctgctggccgcccaggagtacgccgcctccaccggcctgcccatcccccgcaacgtgctgatcccccgcaccaagggcttcgtgtccgccgtgtccatcacccgctccttcgtgcccgtgatctacgacatcaccgtggccatccccaagtcctcctcccagcccaccatgctgaagctgttcaagggccagtcctccgtggtgcacgtgcacctgaagcgccacctgatgaaggacctgcccgagtccgacgacgacgtggcccagtggtgccgcgcccagttcgtggtgaaggactccctgctggacaagcacatcgccgaggacaccttctccgaccaggagctgcaggacatcggccgccccatcaagtccctggtggtgttcacctcctgggtgtgcatcatcaccttcggcgccctgaagttcctgcagtggtcctccctgctgcactcctggaagggcatcgccatctccgcctccggcctggccatcgtgaccgtgctgatgcacatcctgatccgcttctcccagtccgagcactccacctccgccaagatcgccgccgagaagcacaagaacggcggcgtgtcccaggagatgggccgcgagaagcagcactga

SEQ ID NO: 95 - DNA, 1164 bp, Artificial Sequence
  Description of Artificial Sequence Synthetic polynucleotide
atgggcatccccgccgtggccgtgatcgtgcccatcggcatcctgttcttcatctccggcttcatcgtgaacctgatgcaggccatctgcttcgtgctgatccgccccctgtccaagaacacctaccgcatcgtgaaccgccagctggccgagttcctgtggctggagctgatctgggtggtggactggtgggccggcgtgaagatccagctgttcaccgacaaggagaccctgcacctgatgggcaaggagcacgccctggtgatctgcaaccaccgctccgacatcgactggctggtgggctggctgctgtgccagcgctccggctgcctgggctccgccctggccgtgatgaagtcctcctccaaggtgctgcccgtgatcggctggtccatgtggttctccgagtacctgttcctggagcgctcctgggccaaggacgagtccaccctgaagctgggcctgcagcgcctgaaggacttcccccgccccttctggctggccctgttcgtggagggcacccgcttcacccaggccaagctgctggccgcccaggagtacgccatgtccgccggcctgcccgtgccccgcaacgtgctgatcccccgcaccaagggcttcgtgtccgccgtgtccaacatgcgctccttcgtgcccgccatctacgacgtgaccgtggccatccccaagtcctccgtgcagcccaccatgctgggcctgttcaagggccagtcctgcgtggtgcaggtgcacctgaagcgccacctgatgaaggacctgcccgagtccgaggacgacgtggcccagtggtgccgcgagcgcttcgtggtgaaggactccctgctggacaagcacaaggtggaggacaccttctccgaccaggagctgcaggacctgggccgccccatcaagtccctggtggtggtgatctcctgggcctgcatcctgatcttctggatcctgaagttcctgcagtggtcctccctgctgtactcctggaagggcatcgccatctccgcctgcgccatggccgtgatcgccttcctgatgcagatcctgctgcgcttctcccagtccgagcgctccacccccgccaagatcgcccccgccaagcccaacaacgcccgcaactcctccgagaccgtgcgcgacaagcaccagtga

SEQ ID NO: 96 - DNA, 1137 bp, Artificial Sequence
  Description of Artificial Sequence Synthetic polynucleotide
atggccatccccgccgccatcatcatcgtgcccctgggcctgatcttcttcacctccggcttcatcatcaacctgatccaggccgtgtgctacgtgctgatccgccccctgtccaagtccaccttccgccgcatcaaccgccagctggccgagctgctgtggctggagctggtgtgggtggtggactggtgggccggcgtgaagatccagctgttcaccaacaaggagaccctgcactccatcggcaaggagcacgccctggtgatctgcaaccagcgctccgacatcgactggctggtgggctggatcctggcccagcgctccggctgcctgggctccgccctggccgtgatgaagaagtcctccaaggtgctgcccgtgatcggctggtccatgtggttctccgagtacctgttcctggagcgcaactgggccatggacgagtccaccctgaagtccggcctgcagtggctgaaggacttcccccagcccttctggctggccctgttcgtggagggcacccgcttcacccagcccaagctgctggccgcccaggagtacgccgcctccgccggcctgcccatcccccgcaacgtgctgatcccccgcaccaagggcttcgtgtccgccgtgaacatcatgcgctccttcgtgcccgccgtgtacgacgtgaccgtggccatccccaagtcctccccccagcccaccatgctgcgcctgttcaagggccagtcctccgtggtgcacgtgcacctgaagcgccacctgatggaggacctgcccgagaccgacgacgacgtggcccagtggtgccgcgaccgcttcgtggtgaaggactccctgctggacaagcacctggccgaggacaccttctccgaccaggagctgcaggacctgggccgccccatcaagtccctggtggtggtgacctcctgggtgtgcatcatcgccttcggcgccctgaagttcctgcagtggtcctccctgctgtactcctggaagggcatcgtgatctccgccgcctccctggccgtggtgaccgtgctgatgcagatcctgatccgcttctcccagtccgagcgctccacctccgccaaggtggtggccgagaagcgcaagaacgtgggcgagcactga

SEQ ID NO: 97 - DNA, 5674 bp, Artificial Sequence
  Description of Artificial Sequence Synthetic polynucleotide
gtttaaacgccggtcaccacccgcatgctcgtactacagcgcacgcaccgcttcgtgatccaccgggtgaacgtagtcctcgacggaaacatctggttcgggcctcctgcttgcactcccgcccatgccgacaacctttctgctgttaccacgacccacaatgcaacgcgacacgaccgtgtgggactgatcggttcactgcacctgcatgcaattgtcacaagcgcttactccaattgtattcgtttgttttctgggagcagttgctcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaggtaccatggacgcctccggcgcctcctccttcctgcgcggccgctgcctggagtcctgcttcaaggcctccttcggctacgtaatgtcccagcccaaggacgccgccggccagccctcccgccgccccgccgacgccgacgacttcgtggacgacgaccgctggatcaccgtgatcctgtccgtggtgcgcatcgccgcctgcttcctgtccatgatggtgaccaccatcgtgtggaacatgatcatgctgatcctgctgccctggccctacgcccgcatccgccagggcaacctgtacggccacgtgaccggccgcatgctgatgtggattctgggcaaccccatcaccatcgagggctccgagttctccaacacccgcgccatctacatctgcaaccacgcctccctggtggacatcttcctgatcatgtggctgatccccaagggcaccgtgaccatcgccaagaaggagatcatctggtatcccctgttcggccagctgtacgtgctggccaaccaccagcgcatcgaccgctccaacccctccgccgccatcgagtccatcaaggaggtggcccgcgccgtggtgaagaagaacctgtccctgatcatcttccccgagggcacccgctccaagaccggccgcctgctgcccttcaagaagggcttcatccacatcgccctccagacccgcctgcccatcgtgccgatggtgctgaccggcacccacctggcctggcgcaagaactccctgcgcgtgcgccccgcccccatcaccgtgaagtacttctcccccatcaagaccgacgactgggaggaggagaagatcaaccactacgtggagatgatccacgccctgtacgtggaccacctgcccgagtcccagaagcccctggtgtccaagggccgcgacgcctccggccgctccaactcctgattaattaactcgagatgtggagatgtagggtggtcgactcgttggaggtgggtgtttttttttatcgagtgcgcggcgcggcaaacgggtccctttttatcgaggtgttcccaacgccgcaccgccctcttaaaacaacccccaccaccacttgtcgaccttctcgtttgttatccgccacggcgccccggaggggcgtcgtctggccgcgcgggcagctgtatcgccgcgctcgctccaatggtgtgtaatcttggaaagataataatcgatggatgaggaggagagcgtgggagatcagagcaaggaatatacagttggcacgaagcagcagcgtactaagctgtagcgtgttaagaaagaaaaactcgctgttaggctgtattaatcaaggagcgtatcaataattaccgaccctatacctttatctccaacccaatcgcggcctaggtgcggtgagaatcgaaaatgcatcgtttctaggttcggagacggtcaattccctgctccggcgaatctgtcggtcaagctggccagtggacaatgttgctatggcagcccgcgcacatgggcctcccgacgcggccatcaggagcccaaacagcgtgtcagggtatgtgaaactcaagaggtccctgctgggcactccggccccactccgggggcgggacgccaggcattcgcggtcggtcccgcgcgacgagcgaaatgatgattcggttacgagaccaggacgtcgtcgaggtcgagaggcagcctcggacacgtctcgctagggcaacgccccgagtccccgcgagggccgtaaacattgtttctgggtgtcggagtgggcattttgggcccgatccaatcgcctcatgccgctctcgtctggtcctcacgttcgcgtacggcctggatcccggaaagggcggatgcacgtggtgttgccccgccattggcgcccacgtttcaaagtccccggccagaaatgcacaggaccggcccggctcgcacaggccatgctgaacgcccagatttcgacagcaacaccatctagaataatcgcaaccatccgcgttttgaacgaaacgaaacggcgctgtttagcatgtttccgacatcgtgggggccgaagcatgctccggggggaggaaagcgtggcacagcggtagcccattctgtgccacacgccgacgaggaccaatccccggcatcagccttcatcgacggctgcgccgcacatataaagccggacgcctaaccggtttcgtggttatgactagtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcagatatcaagctccatcgagctccagccacggcaacaccgcgcgccttgcggccgagcacggcgacaagaacctgagcaagatctgcgggctgatcgccagcgacgagggccggcacgagatcgcctacacgcgcatcgtggacgagttcttccgcctcgaccccgagggcgccgtcgccgcctacgccaacatgatgcgcaagcagatcaccatgcccgcgcacctcatggacgacatgggccacggcgaggccaacccgggccgcaacctcttcgccgacttctccgcggtcgccgagaagatcgacgtctacgacgccgaggactactgccgcatcctggagcacctcaacgcgcgctggaaggtggacgagcgccaggtcagcggccaggccgccgcggaccaggagtacgtcctgggcctgccccagcgcttccggaaactcgccgagaagaccgccgccaagcgcaagcgcgtcgcgcgcaggcccgtcgccttctcctggatctccgggcgcgagatcatggtctagggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactcctgaagagcgtttaaac

SEQ ID NO: 98 - DNA, 1170 bp, Artificial Sequence
  Description of Artificial Sequence Synthetic polynucleotide
ggtaccatggccatccccgccgccgccgtgatcttcctgttcggcctgctgttcttcacctccggcctgatcatcaacctgttccaggccctgtgcttcgtgctggtgtggcccctgtccaagaacgcctaccgccgcatcaaccgcgtgttcgccgagctgctgctgtccgagctgctgtgcctgttcgactggtgggccggcgccaagctgaagctgttcaccgaccccgagaccttccgcctgatgggcaaggagcacgccctggtgatcatcaaccacatgaccgagctggactggatgctgggctgggtgatgggccagcacctgggctgcctgggctccatcctgtccgtggccaagaagtccaccaagttcctgcccgtgctgggctggtccatgtggttctccgagtacctgtacatcgagcgctcctgggccaaggaccgcaccaccctgaagtcccacatcgagcgcctgaccgactaccccctgcccttctggatggtgatcttcgtggagggcacccgcttcacccgcaccaagctgctggccgcccagcagtacgccgcctcctccggcctgcccgtgccccgcaacgtgctgatcccccgcaccaagggcttcgtgtcctgcgtgtcccacatgcgctccttcgtgcccgccgtgtacgacgtgaccgtggccttccccaagacctcccccccccccaccctgctgaacctgttcgagggccagtccatcgtgctgcacgtgcacatcaagcgccacgccatgaaggacctgcccgagtccgacgacgccgtggcccagtggtgccgcgacaagttcgtggagaaggacgccctgctggacaagcacaacgccgaggacaccttctccggccaggaggtgcaccgcaccggctcccgccccatcaagtccctgctggtggtgatctcctgggtggtggtgatcaccttcggcgccctgaagttcctgcagtggtcctcctggaagggcaaggccttctccgtgatcggcctgggcatcgtgaccctgctgatgcacatgctgatcctgtcctcccaggccgagcgctcctccaaccccgccaaggtggcccaggccaagctgaagaccgagctgtccatctccaagaaggccaccgacaaggagaactgactcgag

SEQ ID NO: 99 - DNA, 1167 bp, Artificial Sequence
  Description of Artificial Sequence Synthetic polynucleotide
ggtaccatggccatccccgccgccgccgtgatcttcctgttcggcctgatcttcttcgcctccggcctgatcatcaacctgttccaggccctgtgcttcgtgctgatctggcccatctccaagaacgcctaccgccgcatcaaccgcgtgttcgccgagctgctgctgtccgagctgctgtgcctgttcgactggtgggccggcgccaagctgaagctgttcaccgaccccgagaccttccgcctgatgggcaaggagcacgccctggtgatcatcaaccacatgaccgagctggactggatggtgggctgggtgatgggccagcacttcggctgcctgggctccatcctgtccgtggccaagaagtccaccaagttcctgcccgtgctgggctggtccatgtggttcaccgagtacctgtacatcgagcgctcctggaacaaggacaagtccaccctgaagtcccacatcgagcgcctgaaggactaccccctgcccttctggctggtgatcttcgccgagggcacccgcttcacccagaccaagctgctggccgcccagcagtacgccgcctcctccggcctgcccgtgccccgcaacgtgctgatcccccgcaccaagggcttcgtgtcctgcgtgtcccacatgcgctccttcgtgcccgccgtgtacgacctgaccgtggccttccccaagacctcccccccccccaccctgctgaacctgttcgagggccagtccgtggtgctgcacgtgcacatcaagcgccacgccatgaaggacctgcccgagtccgacgacgaggtggcccagtggtgccgcgacaagttcgtggagaaggacgccctgctggacaagcacaacgccgaggacaccttctccggccaggagctgcagcacaccggccgccgccccatcaagtccctgctggtggtgatctcctgggtggtggtgatcgccttcggcgccctgaagttcctgcagtggtcctcctggaagggcaaggccttctccgtgatcggcctgggcatcgtgaccctgctgatgcacatgctgatcctgtcctcccaggccgagcgctccaagcccgccaaggtggcccaggccaagctgaagaccgagctgtccatctccaagaccgtgaccgacaaggagaactgactcgag

SEQ ID NO: 100 - DNA, 1188 bp, Artificial Sequence
  Description of Artificial Sequence Synthetic polynucleotide
ggtaccatggccatcccctccgccgccgtggtgttcctgttcggcctgctgttcttcacctccggcctgatcatcaacctgttccaggccttctgcttcgtgctgatctcccccctgtccaagaacgcctaccgccgcatcaaccgcgtgttcgccgagctgctgcccctggagttcctgtggctgttccactggtgcgccggcgccaagctgaagctgttcaccgaccccgagaccttccgcctgatgggcaaggagcacgccctggtgatcatcaaccacaagatcgagctggactggatggtgggctgggtgctgggccagcacctgggctgcctgggctccatcctgtccgtggccaagaagtccaccaagttcctgcccgtgttcggctggtccctgtggttctccggctacctgttcctggagcgctcctgggccaaggacaagatcaccctgaagtcccacatcgagtccctgaaggactaccccctgcccttctggctgatcatcttcgtggagggcacccgcttcacccgcaccaagctgctggccgcccagcagtacgccgcctcctccggcctgcccgtgccccgcaacgtgctgatcccccacaccaagggcttcgtgtcctccgtgtcccacatgcgctccttcgtgcccgccatctacgacgtgaccgtggccttccccaagacctcccccccccccaccatgctgaagctgttcgagggccagtccgtggagctgcacgtgcacatcaagcgccacgccatgaaggacctgcccgagtccgacgacgccgtggcccagtggtgccgcgacaagttcgtggagaaggacgccctgctggacaagcacaactccgaggacaccttctccggccaggaggtgcaccacgtgggccgccccatcaaggccctgctggtggtgatctcctgggtggtggtgatcatcttcggcgccctgaagttcctgctgtggtcctccctgctgtcctcctggaagggcaaggccttctccgtgatcggcctgggcatcgtggccggcatcgtgaccctgctgatgcacatcctgatcctgtcctcccaggccgagggctccaaccccgtgaaggccgcccccgccaagctgaagaccgagctgtcctcctccaagaaggtgaccaacaaggagaactgactcgag

SEQ ID NO: 101 - DNA, 1188 bp, Artificial Sequence
  Description of Artificial Sequence Synthetic polynucleotide
ggtaccatggccatcccctccgccgccgtggtgttcctgttcggcctgctgttcttcacctccggcctgatcatcaacctgttccaggccttctgcttcgtgctgatctcccccctgtccaagaacgcctaccgccgcatcaaccgcgtgttcgccgagctgctgcccctggagttcctgtggctgttccactggtgcgccggcgccaagctgaagctgttcaccgaccccgagaccttccgcctgatgggcaaggagcacgccctggtgatcatcaaccacaagatcgagctggactggatggtgggctgggtgctgggccagcacctgggctgcctgggctccatcctgtccgtggccaagaagtccaccaagttcctgcccgtgttcggctggtccctgtggttctccgagtacctgttcctggagcgctcctgggccaaggacaagatcaccctgaagtcccacatcgagtccctgaaggactaccccctgcccttctggctgatcatcttcgtggagggcacccgcttcacccgcaccaagctgctggccgcccagcagtacgccgcctcctccggcctgcccgtgccccgcaacgtgctgatcccccacaccaagggcttcgtgtcctccgtgtcccacatgcgctccttcgtgcccgccatctacgacgtgaccgtggccttccccaagacctcccccccccccaccatgctgaagctgttcgagggccagtccgtggagctgcacgtgcacatcaagcgccacgccatgaaggacctgcccgagtccgacgacgccgtggcccagtggtgccgcgacaagttcgtggagaaggacgccctgctggacaagcacaactccgaggacaccttctccggccaggaggtgcaccacgtgggccgccccatcaaggccctgctggtggtgatctcctgggtggtggtgatcatcttcggcgccctgaagttcctgctgtggtcctccctgctgtcctcctggaagggcaaggccttctccgtgatcggcctgggcatcgtggccggcatcgtgaccctgctgatgcacatcctgatcctgtcctcccaggccgagggctccaaccccgtgaaggccgcccccgccaagctgaagaccgagctgtcctcctccaagaaggtgaccaacaaggagaactgactcgag

SEQ ID NO: 102 - DNA, 1122 bp, Artificial Sequence
  Description of Artificial Sequence Synthetic polynucleotide
ggtaccatggccatcgccgccgccgccgtgatcttcctgttcggcctgctgttcttcgcctccggcatcatcatcaacctgttccaggccctgtgcttcgtgctgatctggcccctgtccaagaacgtgtaccgccgcatcaaccgcgtgttcgccgagctgctgctgatggacctgctgtgcctgttccactggtgggccggcgccaagatcaagctgttcaccgaccccgagaccttccgcctgatgggcatggagcacgccctggtgatcatgaaccacaagaccgacctggactggatggtgggctggatcctgggccagcacctgggctgcctgggctccatcctgtccatcgccaagaagtccaccaagttcatccccgtgctgggctggtccgtgtggttctccgagtacctgttcctggagcgctcctgggccaaggacaagtccaccctgaagtcccacatggagaagctgaaggactaccccctgcccttctggctggtgatcttcgtggagggcacccgcttcacccgcaccaagctgctggccgcccagcagtacgccgcctcctccggcctgcccgtgccccgcaacgtgctgatcccccacaccaagggcttcgtgtcctgcgtgtccaacatgcgctccttcgtgcccgccgtgtacgacgtgaccgtggccttccccaagtcctcccccccccccaccatgctgaagctgttcgagggccagtccatcgtgctgcacgtgcacatcaagcgccacgccctgaaggacctgcccgagtccgacgacgccgtggcccagtggtgccgcgacaagttcgtggagaaggacgccctgctggacaagcacaacgccgaggacaccttctccggccaggaggtgcaccacatcggccgccccatcaagtccctgctggtggtgatcgcctgggtggtggtgatcatcttcggcgccctgaagttcctgcagtggtcctccctgctgtccacctggaagggcaaggccttctccgtgatcggcctgggcatcgccaccctgctgatgcacatgctgatcctgtcctcccaggccgagcgctccaaccccgccaaggtggccaagtgactcgag

SEQ ID NO: 103 - DNA, 1176 bp, Artificial Sequence
  Description of Artificial Sequence Synthetic polynucleotide
ggtaccatgaccatcgcctccgccgccgtggtgttcctgttcggcatcctgctgttcacctccggcctgatcatcaacctgttccaggccttctgctccgtgctggtgtggcccctgtccaagaacgcctaccgccgcatcaaccgcgtgttcgccgagttcctgcccctggagttcctgtggctgttccactggtgggccggcgccaagctgaagctgttcaccgaccccgagaccttccgcctgatgggcaaggagcacgccctggtgatcatcaaccacaagatcgagctggactggatggtgggctgggtgctgggccagcacctgggctgcctgggctccatcctgtccgtggccaagaagtccaccaagttcctgcccgtgttcggctggtccctgtggttctccgagtacctgttcctggagcgcaactgggccaaggacaagaagaccctgaagtcccacatcgagcgcctgaaggactaccccctgcccttctggctgatcatcttcgtggagggcacccgcttcacccgcaccaagctgctggccgcccagcagtacgccgcctccgccggcctgcccgtgccccgcaacgtgctgatcccccacaccaagggcttcgtgtcctccgtgtcccacatgcgctccttcgtgcccgccatctacgacgtgaccgtggccttccccaagacctcccccccccccaccatgctgaagctgttcgagggccacttcgtggagctgcacgtgcacatcaagcgccacgccatgaaggacctgcccgagtccgaggacgccgtggcccagtggtgccgcgacaagttcgtggagaaggacgccctgctggacaagcacaacgccgaggacaccttctccggccaggaggtgcaccacgtgggccgccccatcaagtccctgctggtggtgatctcctgggtggtggtgatcatcttcggcgccctgaagttcctgcagtggtcctccctgctgtcctcctggaagggcatcgccttctccgtgatcggcctgggcaccgtggccctgctgatgcagatcctgatcctgtcctcccaggccgagcgctccatccccgccaaggagacccccgccaacctgaagaccgagctgtcctcctccaagaaggtgaccaacaaggagaactgactcgag

SEQ ID NO: 104 - DNA, 1176 bp, Artificial Sequence
  Description of Artificial Sequence Synthetic polynucleotide
ggtaccatggccatcgccgccgccgccgtgatcgtgcccgtgtccctgctgttcttcgtgtccggcctgatcgtgaacctggtgcaggccgtgtgcttcgtgctgatccgccccctgttcaagaacacctaccgccgcatcaaccgcgtggtggccgagctgctgtggctggagctggtgtggctgatcgactggtgggccggcgtgaagatcaaggtgttcaccgaccacgagaccttccacctgatgggcaaggagcacgccctggtgatctgcaaccacaagtccgacatcgactggctggtgggctgggtgctggcccagcgctccggctgcctgggctccaccctggccgtgatgaagaagtcctccaagttcctgcccgtgatcggctggtccatgtggttctccgagtacctgttcctggagcgcaactgggccaaggacgagtccaccctgaagtccggcctgaaccgcctgaaggactaccccctgcccttctggctggccctgttcgtggagggcacccgcttcacccgcgccaagctgctggccgcccagcagtacgccgcctcctccggcctgcccgtgccccgcaacgtgctgatcccccgcaccaagggcttcgtgtcctccgtgtcccacatgcgctccttcgtgcccgccatctacgacgtgaccgtggccatccccaagacctcccccccccccaccctgctgcgcatgttcaagggccagtcctccgtgctgcacgtgcacctgaagcgccaccagatgaacgacctgcccgagtccgacgacgccgtggcccagtggtgccgcgacatcttcgtggagaaggacgccctgctggacaagcacaacgccgaggacaccttctccggccaggagctgcaggacaccggccgccccatcaagtccctgctgatcgtgatctcctgggccgtgctggtggtgttcggcgccgtgaagttcctgcagtggtcctccctgctgtcctcctggaagggcctggccttctccggcatcggcctgggcgtgatcaccctgctgatgcacatcctgatcctgttctcccagtccgagcgctccacccccgccaaggtggcccccgccaagcccaagatcgagggcgagtcctccaagaccgagatggagaaggagcactgactcgag

SEQ ID NO: 105 - DNA, 1176 bp, Artificial Sequence
  Description of Artificial Sequence Synthetic polynucleotide
ggtaccatggccatcgccgccgccgccgtgatcgtgcccctgggcctgctgttcttcgtgtccggcctgatcgtgaacctggtgcaggccgtgtgcttcgtgctgatccgccccctgtccaagaacacctaccgccgcatcaaccgcgtggtggccgagctgctgtggctggagctggtgtggctgatcgactggtgggccggcgtgaagatcaaggtgttcaccgaccacgagaccctgtccctgatgggcaaggagcacgccctggtgatctgcaaccacaagtccgacatcgactggctggtgggctgggtgctggcccagcgctccggctgcctgggctccaccctggccgtgatgaagaagtcctccaagttcctgcccgtgatcggctggtccatgtggttctccgagtacctgcccgagtccgacgacgccgtggcccagtggtgccgcgacatcttcgtggagaaggacgccctgctggacaagcacaacgccgaggacaccttctccggccaggagctgcaggacaccggccgccccatcaagtccctgctggtggtgatctcctgggccgtgctggtgatcttcggcgccgtgaagttcctgcagtggtcctccctgctgtcctcctggaagggcctggccttctccggcgtgggcctgggcatcatcaccctgctgatgcacatcctgatcctgttctcccagtccgagcgctccacccccgccaaggtggcccccgccaagcccaagaaggacggcgagtcctccaagaccgagatcgagaaggagaacgttcctggagcgctcctgggccaaggacgagaacaccctgaagtccggcctgaaccgcctgaaggactaccccctgcccttctggctggccctgttcgtggagggcacccgcttcacccgcgccaagctgctggccgcccagcagtacgccacctcctccggcctgcccgtgccccgcaacgtgctgatcccccgcaccaagggcttcgtgtcctccgtgtcccacatgcgctccttcgtgcccgccatctacgacgtgaccgtggccatccccaagacctcccccccccccaccatgctgcgcatgttcaagggccagtcctccgtgctgcacgtgcacctgaagcgccacctgatgaaggaccttgactcgag

SEQ ID NO: 106 - DNA, 1167 bp, Artificial Sequence
  Description of Artificial Sequence Synthetic polynucleotide
ggtaccatggccatcgccgccgccgccgtgatcttcctgttcggcctgatcttcttcgcctccggcctgatcatcaacctgttccaggccctgtgcttcgtgctgatccgccccctgtccaagaacgcctaccgccgcatcaaccgcgtgttcgccgagctgctgctgtccgagctgctgtgcctgttcgactggtgggccggcgccaagctgaagctgttcaccgaccccgagaccttccgcctgatgggcaaggagcacgccctggtgatcatcaaccacatgaccgagctggactggatggtgggctgggtgatgggccagcacttcggctgcctgggctccatcatctccgtggccaagaagtccaccaagttcctgcccgtgctgggctggtccatgtggttctccgagtacctgtacctggagcgctcctgggccaaggacaagtccaccctgaagtcccacatcgagcgcctgatcgactaccccctgcccttctggctggtgatcttcgtggagggcacccgcttcacccgcaccaagctgctggccgcccagcagtacgccgtgtcctccggcctgcccgtgccccgcaacgtgctgatcccccgcaccaagggcttcgtgtcctgcgtgtcccacatgcgctccttcgtgcccgccgtgtacgacgtgaccgtggccttccccaagacctcccccccccccaccctgctgaacctgttcgagggccagtccatcatgctgcacgtgcacatcaagcgccacgccatgaaggacctgcccgagtccgacgacgccgtggccgagtggtgccgcgacaagttcgtggagaaggacgccctgctggacaagcacaacgccgaggacaccttctccggccaggaggtgtgccactccggctcccgccagctgaagtccctgctggtggtgatctcctgggtggtggtgaccaccttcggcgccctgaagttcctgcagtggtcctcctggaagggcaaggccttctccgccatcggcctgggcatcgtgaccctgctgatgcacgtgctgatcctgtcctcccaggccgagcgctccaaccccgccgaggtggcccaggccaagctgaagaccggcctgtccatctccaagaaggtgaccgacaaggagaactgactcgag

SEQ ID NO: 107 - DNA, 1155 bp, Artificial Sequence
  Description of Artificial Sequence Synthetic polynucleotide
ggtaccatggccatccccgccgccgtggccgtgatccccatcggcctgctgttcatcatctccggcctgatcgtgaacctgatccaggccgtggtgtacgtgctgatccgccccctgtccaagaacctgcaccgcaagatcaacaagcccatcgccgagctgctgtggctggagctgatctggctggtggactggtgggccggcatcaaggtggaggtgtacgccgactcccagaccctggagctgatgggcaaggagcacgccctgctgatctgcaaccaccgctccgacatcgactggctggtgggctgggtgctggcccagcgcgcccgctgcctgggctccgccctggccatcatgaagaagtccgccaagttcctgcccgtgatcggctggtccatgtggttctccgactacatcttcctggaccgcacctgggccaaggacgagaagaccctgaagtccggcttcgagcgcctggccgacttccccatgcccttctggctggccctgttcgtggagggcacccgcttcaccaaggccaagctgctggccgcccaggagtacgccgcctcccgcggcctgcccgtgccccagaacgtgctgatcccccgcaccaagggcttcgtgaccgccgtgacccacatgcgctcctacgtgcccgccatctacgactgcaccgtggacatctccaaggcccaccccgccccctccatcctgcgcctgatccgcggccagtcctccgtggtgaaggtgcagatcacccgccactccatgcaggagctgcccgagaccgccgacggcatctcccagtggtgcatggacctgttcgtgaccaaggacggcttcctggagaagtaccactccaaggacatcttcggctccctgcccgtgcagaacatcggccgccccgtgaagtccctgatcgtggtgctgtgctggtactgcctgatggccttcggcctgttcaagttcttcatgtggtcctccctgctgtcctcctgggagggcatcctgtccctgggcctgatcctgctggccgtggccatcgtgatgcagatcctgatccagtccaccgagtccgagcgctccacccccgtgaagtccatccagaaggacccctccaaggagaccctgctgcagaactgactcgag

SEQ ID NO: 108 - DNA, 1161 bp, Artificial Sequence
  Description of Artificial Sequence Synthetic polynucleotide
ggtaccatgcacgtgctgctggagatggtgaccttccgcttctcctccttcttcgtgttcgacaacgtgcaggccctgtgcttcgtgctgatctggcccctgtccaagtccgcctaccgcaagatcaaccgcgtgttcgccgagctgctgctgtccgagctgctgtgcctgttcgactggtgggccggcgccaagctgaagctgttcaccgaccccgagaccttccgcctgatgggcaaggagcacgccctggtgatcaccaaccacaagatcgacctggactggatgatcggctggatcctgggccagcacttcggctgcctgggctccgtgatctccatcgccaagaagtccaccaagttcctgcccatcttcggctggtccctgtggttctccgagtacctgttcctggagcgcaactgggccaaggacaagcgcaccctgaagtcccacatcgagcgcatgaaggactaccccctgcccctgtggctgatcctgttcgtggagggcacccgcttcacccgcaccaagctgctggccgcccagcagtacgccgcctcctccggcctgcccgtgccccgcaacgtgctgatcccccacaccaagggcttcgtgtcctccgtgtcccacatgcgctccttcgtgcccgccgtgtacgacgtgaccgtggccttccccaagacctcccccccccccaccatgctgtccctgttcgagggccagtccgtggtgctgcacgtgcacatcaagcgccacgccatgaaggacctgcccgactccgacgacgccgtggcccagtggtgccgcgacaagttcgtggagaaggacgccctgctggacaagcacaacgccgaggacaccttctccggccaggaggtgcaccacgtgggccgccccatcaagtccctgctggtggtgatctcctggatggtggtgatcatcttcggcgccctgaagttcctgcagtggtcctccctgctgtcctcctggaagggcaaggccttctccgccatcggcctgggcatcgccaccctgctgatgcacgtgctggtggtgttctcccaggccgaccgctccaaccccgccaaggtgccccccgccaagctgaacaccgagctgtcctcctccaagaaggtgaccaacaaggagaactgactcgag

SEQ ID NO: 109 - DNA, 1155 bp, Artificial Sequence
  Description of Artificial Sequence Synthetic polynucleotide
ggtaccatggccatccccgccgccgtggccgtgatccccatcggcctgctgttcatcatctccggcctgatcgtgaacctgatccaggccgtggtgtacgtgctgatccgccccctgtccaagaacctgtaccgcaagatcaacaagcccatcgccgagctgctgtggctggagctgatctggctggtggactggtgggccggcatcaaggtggaggtgtacgccgactccgagaccctggagtccatgggcaaggagcacgccctgctgatctgcaaccaccgctccgacatcgactggctggtgggctgggtgctggcccagcgcgcccgctgcctgggctccgccctggccatcatgaagaagtccgccaagttcctgcccgtgatcggctggtccatgtggttctccgactacatcttcctggaccgcacctgggagaaggacgagaagaccctgaagtccggcttcgagcgcctggccgacttccccatgcccttctggctggccctgttcgtggagggcacccgcttcaccaaggccaagctgctggccgcccaggagttcgccgcctcccgcggcctgcccgtgccccagaacgtgctgatcccccgcaccaagggcttcgtgaccgccgtgacccacatgcgctcctacgtgcccgccatctacgactgcaccgtggacatctccaaggcccaccccgccccctccatcctgcgcctgatccgcggccagtcctccgtggtgaaggtgcagatcacccgccactccatgcaggagctgcccgagacccccgacggcatctcccagtggtgcatggacctgttcgtgaccaaggacgccttcctggagaagtaccactccaaggacatcttcggctccctgcccgtgcacgacatcggccgccccgtgaagtccctgatcgtggtgctgtgctggtactccctgatggccttcggcttctacaagttcttcatgtggtcctccctgctgtcctcctgggagggcatcctgtccctgggcctggtgctgatcgtgatcgccatcgtgatgcagatcctgatccagtcctccgagtccgagcgctccacccccgtgaagtccgtgcagaaggacccctccaaggagaccctgctgcagaactgactcgag

SEQ ID NO: 110 - DNA, 1137 bp, Artificial Sequence
  Description of Artificial Sequence Synthetic polynucleotide
ggtaccatggccaccggcggctccctgaagccctcctcctccgacctggacctggaccaccccaacatcgaggactacctgccctccggctcctccatcaacgagcccgccggcaagctgcgcctgcgcgacctgctggacatctcccccaccctgaccgaggccgccggcgccatcgtggacgactccttcacccgctgcttcaagtccatcccccgcgagccctggaactggaacctgtacctgttccccctgtggtgcatcggcgtgctgatccgctacttcatcctgttccccggccgcgtgatcgtgctgaccatgggctggatcaccgtgatctcctccttcatcgccgtgcgcgtgctgctgaagggccacgacgccctgcagatcaagctggagcgcctgatcgtgcagctgctgtgctcctccttcgtggcctcctggaccggcgtggtgaagtaccacggcccccgcccctccatccgccccaagcaggtgtacgtggccaaccacacctccatgatcgacttcttcatcctggaccagatgaccgtgttctccgtgatcatgcagaagcaccccggctgggtgggcctgctgcagtccaccctgctggagtccgtgggctgcatctggttcgaccgcgccgaggccaaggaccgcggcatcgtggccaagaagctgtgggaccacgtgcacggcgagggcaacaaccccctgctgatcttccccgagggcacctgcgtgaacaacaactactccgtgatgttcaagaagggcgccttcgagctgggctgcaccgtgtgccccgtggccatcaagtacaacaagatcttcgtggacgccttctggaactccaagaagcagtccttcacccgccacctgctgcagctgatgacctcctgggccgtggtgtgcgacgtgtggtacttggagccccagaccctgaagcccggcgagacccccatcgagttcgccgagcgcgtgcgcgacatcatctccgcccgcgccggcctgaagaaggtgccctgggacggctacctgaagtactcccgcccctcccccaagcaccgcgagcgcaagcagcagaccttcgccgagtccgtgctgcagcgcctggaggagtgactcgag

SEQ ID NO: 111 - DNA, 1140 bp, Artificial Sequence
  Description of Artificial Sequence Synthetic polynucleotide
ggtaccatggccaccgccggctccctgaagccctcccgctccgagctggacttcgaccgccccaacatcgaggactacctgccctccggctcctccatcatcgagcccgccggcaagctgcgcctgcgcgacctgctggacatctcccccaccctgaccgaggccgccggcgccatcgtggacgactccttcacccgctgcttcaagtccaacccccccgagccctggaactggaacatctacctgttccccctgtggtgcttcggcgtgctgatccgctacctgatcctgttccccgcccgcgtgatcgtgctgaccatcggctggatcatcttcctgtcctccttcatccccgtgcacctgctgctgaagggccacgacgccctgcgcatcaagctggagcgcctgctggtggagctgatctgctccttcttcgtggcctcctggaccggcgtggtgaagtaccacggcccccgcccctccatccgccccaagcaggtgtacgtggccaaccacacctccatgatcgacttcttcatcctggaccagatgaccgtgttctccgtgatcatgcagaagcaccccggctgggtgggcctgctgcagtccaccctgctggagtccgtgggctgcatctggttcgaccgcgccgaggccaaggaccgcggcatcgtggccaagaagctgtgggaccacgtgcacggcgagggcaacaaccccctgctgatcttccccgagggcacctgcgtgaacaacaactactccgtgatgttcaagaagggcgccttcgagctgggctgcaccgtgtgccccgtggccatcaagtacaacaagatcttcgtggacgccttctggaactccaagaagcagtccttcacccgccacctgctgcagctgatgacctcctgggccgtggtgtgcgacgtgtggtacttggagccccagaccctgaagcccggcgagacccccatcgagttcgccgagcgcgtgcgcgacatcatctccgtgcgcgccggcctgaagaaggtgccctgggacggctacctgaagtactcccgcccctcccccaagcacaccgagcgcaagcagcagaacttcgccgagtccgtgctgcagcgcctggagaagaagtgactcgag

SEQ ID NO: 112 - DNA, 1140 bp, Artificial Sequence
  Description of Artificial Sequence Synthetic polynucleotide
ggtaccatggccaccggcggccgcctgaagccctcctcctccgagctggacctggaccgcgccaacaccgaggactacctgccctccggctcctccatcaacgagcccgtgggcaagctgcgcctgcgcgacctgctggacatctcccccaccctgaccgaggccgccggcgccatcgtggacgactccttcacccgctgcttcaagtccatcccccccgagccctggaactggaacatctacctgttccccctgtggtgcttcggcgtgctgatccgctacttcatcctgttccccgcccgcgtgatcgtgctgaccatcggctggatcaccgtgatctcctccttcaccgccgtgcgcttcctgctgaagggccacaacgccctgcagatcaagctggagcgcctgatcgtgcagctgctgtgctcctccttcgtggcctcctggaccggcgtggtgaagtaccacggcccccgcccctccatccgccccaagcaggtgtacgtggccaaccacacctccatgatcgacttcctgatcctggaccagatgaccgtgttctccgtgatcatgcagaagcaccccggctgggtgggcctgctgcagtccaccctgctggagtccgtgggctgcatctggttcaaccgcgccgaggccaaggaccgcgagatcgtggccaagaagctgtgggaccacgtgcacggcgagggcaacaaccccctgctgatcttccccgagggcacctgcgtgaacaaccactactccgtgatgttcaagaagggcgccttcgagctgggctgcaccgtgtgccccgtggccatcaagtacaacaagatcttcgtggacgccttctggaactcccgcaagcagtccttcaccatgcacctgctgcagctgatgacctcctgggccgtggtgtgcgacgtgtggtacttggagccccagaccctgaagcccggcgagaccgccatcgagttcgccgagcgcgtgcgcgacatcatctccgtgcgcgccggcctgaagaaggtgccctgggacggctacctgaagtactcccgcccctcccccaagcaccgcgagtccaagcagcagtccttcgccgagtccgtgctgcgccgcctggaggagaagtgactcgag

SEQ ID NO: 113 - DNA, 1140 bp, Artificial Sequence
  Description of Artificial Sequence Synthetic polynucleotide
ggtaccatggccaccggcggccgcctgaagccctcctcctccgagctggacctggaccgcgccaacaccgaggactacctgccctccggctcctccatcaacgagcccgtgggcaagctgcgcctgcgcgacctgctggacatctcccccaccctgaccgaggccgccggcgccatcgtggacgactccttcacccgctgcttcaagtccatcccccccgagccctggaactggaacatctacctgttccccctgtggtgcttcggcgtgctgatccgctacttcatcctgttccccgcccgcgtgatcgtgctgaccatcggctggatcaccgtgatctcctccttcaccgccgtgcgcttcctgctgaagggccacaacgccctgcagatcaagctggagcgcctgatcgtgcagctgctgtgctcctccttcgtggcctcctggaccggcgtggtgaagtaccacggcccccgcccctccatccgccccaagcaggtgtacgtggccaaccacacctccatgatcgacttcctgatcctggaccagatgaccgtgttctccgtgatcatgcagaagcaccccggctgggtgggcctgctgcagtccaccctgctggagtccgtgggctgcatctggttcaaccgcgccgaggccaaggaccgcgagatcgtggccaagaagctgtgggaccacgtgcacggcgagggcaacaaccccctgctgatcttccccgagggcacctgcgtgaacaaccactactccgtgatgttcaagaagggcgccttcgagctgggctgcaccgtgtgccccgtggccatcaagtacaacaagatcttcgtggacgccttctggaactccaagaagcactccttcacccgccacctgctgcagctgatgacctcctgggccgtggtgtgcgacgtgtggtacttggagccccagaccctgaagcccggcgagacccccatcgagttcgccgagcgcgtgcgcgacatcatctccgtgcgcgccgacctgaagaaggtgccctgggacggctacctgaagtactcccgcccctcccccaagcaccgcgagcgcaagcagcagaagttcgccgagtccgtgctgcgccgcctggaggagaagtgactcgag

SEQ ID NO: 114 - DNA, 1146 bp, Artificial Sequence
  Description of Artificial Sequence Synthetic polynucleotide
ggtaccatggccaccgccggccgcctgaagccctcctcctccgagctggagctggacctggaccgccccaacatcgaggactacctgccctccggctcctccatcaacgagcccgccggcaagctgcgcctgcgcgacctgctggacatctcccccatgctgaccgaggccgccggcgccatcgtggacgactccttcacccgctgcttcaagtccatcccccccgagccctggaactggaacatctacctgttccccctgtggtgcttcggcgtgctgatccgctacctgatcctgttccccgcccgcgtgatcgtgctgaccgtgggctggatcaccgtgatctcctccttcatcaccgtgcgcttcctgctgaagggccacgactccctgcgcatcaagctggagcgcctgatcgtgcagctgttctgctcctccttcgtggcctcctggaccggcgtggtgaagtaccacggcccccgcccctccatccgcccccagcaggtgtacgtggccaaccacacctccatgatcgacttcatcatcctgaaccagatgaccgtgttctccgccatcatgcagaagcaccccggctgggtgggcctgatccagtccaccatcctggagtccgtgggctgcatctggttcaaccgcgccgaggccaaggaccgcgagatcgtggccaagaagctgctggaccacgtgcacggcgagggcaacaaccccctgctgatcttccccgagggcacctgcgtgaacaaccactactccgtgatgttcaagaagggcgccttcgagctgggctgcaccgtgtgccccgtggccatcaagtacaacaagatcttcgtggacgccttctggaactccaagaagcagtccttcaccatgcacctgctgcagctgatgacctcctgggccgtggtgtgcgacgtgtggtacttggagccccagaccctgaagcccggcgagacccccatcgagttcgccgagcgcgtgcgcgacatcatctccgtgcgcgccggcctgaagaaggtgccctgggacggctacctgaagtactcccgcccctcccccaagcaccgcgagcgcaagcagcagtccttcgccgagtccgtgctgcgccgcctggagaagcgctgactcgag

SEQ ID NO: 115 - DNA, 1170 bp, Artificial Sequence
  Description of Artificial Sequence Synthetic polynucleotide
ggtaccatggccaccgccggccgcctgaagccctcctcctccgagctggagctggacctggaccgccccaacatcgaggactacctgccctccggctcctccatcaacgagcccgccggcaagctgcgcctgcgcgacctgctggacatctcccccatgctgaccgaggccgccggcgccatcgtggacgactccttcacccgctgcttcaagtccatcccccccgagccctggaactggaacatctacctgttccccctgtggtgcttcggcgtgctgatccgctacctgatcctgttccccgcccgcgtgatcgtgctgaccgtgggctggatcaccgtgatctcctccttcatcaccgtgcgcttcctgctgaagggccacgactccctgcgcatcaagctggagcgcctgatcgtgcagctgttctgctcctccttcgtggcctcctggaccggcgtggtgaagtaccacggcccccgcccctccatccgcccccagcaggtgtacgtggccaaccacacctccatgatcgacttcatcatcctgaaccagatgaccgtgttctccgccatcatgcagaagcaccccggctgggtgggcctgatccagtccaccatcctggagtccgtgggctgcatctggttcaaccgcgccgaggccaaggaccgcgagatcgtggccaagaagctgctggaccacgtgcacggcgagggcaacaaccccctgctgatcttccccgagggcacctgcgtgaacaaccactactccgtgatgttcaagaagggcgccttcgagctgggctgcaccgtgtgccccgtggccatcaagtacaacaagatcttcgtggacgccttctggaactccaagaagctgtccttcaccatgcacctgctgcagctgatgacctcctgggccgtggtgtgcgacgtgtggtacttggagccccagaccctgaagcccggcgagacccccatcgagttcgccgagcgcgtgcgcgacatcatctccgtgcgcgccggcctgaagaaggtgccctgggacggctacctgaagtactcccgcccctcccccaagcaccgcgagcgcaagcagcagaccttcgccgagtccgtgctgcgccgcctggaggagaagggcaacgtggtgcccaccgtgaactgactcgag

SEQ ID NO: 116 - DNA, 1587 bp, Artificial Sequence
  Description of Artificial Sequence Synthetic polynucleotide
ggtaccatggccatcgccgacggcggcatcatcggcgccgccggctccatctccgccctgaccgccgacaccgaccccccctccctgcgccgccgcaacgtgcccgccggccaggcctccgccgtgtccgccttctccaccgagtccatggccaagcacctgtgcgacccctcccgcgagccctccccctcccccaagtcctccgacgacggcaaggaccccgacatcggctccgtggactccctgaacgagaagccctcctcccccgccgccggcaagggccgcctgcagcacgacctgcgcttcacctaccgcgcctcctcccccgcccaccgcaaggtgaaggagtcccccctgtcctcctccaacatcttcaagcagtcccacgccggcctgttcaacctgtgcgtggtggtgctggtggccgtgaactcccgcctgatcatcgagaacctgatgaagtacggcctgctgatcaagaccggcttctggttctcctcccgctccctgcgcgactggcccctgttcatgtgctgcctgtccctgcccatcttccccctggccgccttcctggtggagaagctggcccagaagaaccgcctgcaggagcccaccgtggtgtgctgccacgtgctgatcacctccgtgtccatcctgtaccccgtgctggtgatcctgcgctgcgactccgccgtgctgtccggcgtggccctgatgctgttcgcctgcatcgtgtggctgaagctggtgtcctacgcccactccaactacgacatgcgctacgtggccaagtccctggacaagggcgagcccgtggtggactccgtgatcgccgaccacccctaccgcgtggactacaaggacctggtgtacttcatggtggcccccaccctgtgctaccagctgtcctaccccctgaccccctgcgtgcgcaagtcctggatcgcccgccaggtgatgaagctggtgctgttcaccggcgtgatgggcttcatcgtggagcagtacatcaaccccatcgtgcagaactccaagcaccccctgaagggcgacctgctgtacgccatcgagcgcgtgctgaagctgtccgtgcccaacctgtacgtgtggctgtgcatgttctactgcttcttccacctgtggctgaacatcctggccgagctgatctgcttcggcgaccgcgagttctacaaggactggtggaacgccaagaccgtggaggagtactggcgcatgtggaacatgcccgtgcacaagtggatggtgcgccacatctacttcccctgcctgcgcaacggcatcccccgcggcgtggccgtgctgatcgccttcctggtgtccgccgtgttccacgagctgtgcatcgccgtgccctgccacgtgttcaagctgtgggccttcatcggcatcatgttccaggtgcccctggtgctggtgtccaactgcctgcagaagaagttccagtcctccatggccggcaacatgttcttctggttcatcttctgcatcttcggccagcccatgtgcgtgctgctgtactaccacgacctgatgaaccgcaagggctcccgcatcgactgactcgag

SEQ ID NO: 117 - DNA, 1599 bp, Artificial Sequence
  Description of Artificial Sequence Synthetic polynucleotide
ggtaccatggccatcgccgacggcggctccgccggcgccgccggctccatctccggctccgacccctccccctccaccgccccctccctgcgccgccgcaacgcctccgccggccaggccttctccaccgagtccatggcccgcgacctgtgcgacccctcccgcgagccctccctgtcccccaagtcctccgacgacggcaaggaccccgccgacgacatcggcgccgccgactccgtggactccggcggcgtgaaggacgagaagccctcctcccaggccgccgccaaggcccgcctggagcacgacctgcgcttcacctaccgcgcctcctcccccgcccaccgcaaggtgaaggagtcccccctgtcctcctccaacatcttcaagcagtcccacgccggcctgttcaacctgtgcgtggtggtgctggtggccgtgaactcccgcctgatcatcgagaacctgatgaagtacggcctgctgatcaagaccggcttctggttctcctcccgctccctgcgcgactggcccctgttcatgtgctgcctgtccctgcccatcttccccctggccgccttcctggtggagaagctggcccagaagaaccgcctgcaggagcccaccgtggtgtgctgccacgtgatcatcacctccgtgtccatcctgtaccccgtgctggtgatcctgcgctgcgactccgccgtgctgtccggcgtggccctgatgctgttcgcctgcatcgtgtggctgaagctggtgtcctacgcccacgccaactacgacatgcgctccgtggccaagtccctggacaagggcgagaccgtggccgactccgtgatcgtggaccacccctaccgcgtggactacaaggacctggtgtacttcatggtggcccccaccctgtgctaccagctgtcctaccccctgaccccctacgtgcgcaagtcctgggtggcccgccaggtgatgaagctggtgctgttcaccggcgtgatgggcttcatcgtggagcagtacatcaaccccatcgtgcagaactccaagcaccccctgaagggcgacctgctgtacgccatcgagcgcgtgctgaagctgtccgtgcccaacctgtacgtgtggctgtgcatgttctactgcttcttccacctgtggctgaacatcctggccgagctgacctgcttcggcgaccgcgagttctacaaggactggtggaacgccaagaccgtggaggagtactggcgcatgtggaacatgcccgtgcacaagtggatggtgcgccacatctacttcccctgcctgcgcaacggcatcccccgcggcgtggccgtgctgatcgccttcctggtgtccgccgtgttccacgagctgtgcatcgccgtgccctgccacgtgttcaagctgtgggccttcatcggcatcatgttccaggtgcccctggtgctggtgtccaactgcctgcagaagaagttccagtcctccatggccggcaacatgttcttctggttcatcttctgcatcttcggccagcccatgtgcgtgctgctgtactaccacgacctgatgaaccgcaagggctcccgcatcgactgactcgag

SEQ ID NO: 118 - DNA, 1404 bp, Artificial Sequence
  Description of Artificial Sequence Synthetic polynucleotide
ggtaccatgggcctggtgtccgtggccgccgccatcggcgtgtccgtgcccgtggcccgcttcctgctgtgcttcctggccaccatccccgtgtccttcctgtggcgcctggtgcccggccgcctgcccaagcacctgtactccgccgcctccggcgccatcctgtcctacctgtccttcggcgcctcctccaacctgcacttcatcgtgcccatgaccctgggctacctgtccatgctgttcttccgccccttctccggcctgctgaccttcttcctgggcttcggctacctgatcggctgccacgtgtactacatgtccggcgacgcctggaaggagggcggcatcgacgccaccggcgccctgatggtgctgaccctgaaggtgatctcctgctccatgaactacaacgacggcctgctgaaggaggagggcctgcgcgagtcccagaagaagaaccgcctgaccaagatgccctccctgatcgagtacttcggctactgcctgtgctgcggctcccacttcgccggccccgtgtacgagatgaaggactacctggagtggaccgagggcaagggcatctggtcccgctcccagaaggagcccaagccctcccccttcggcggcgccctgcgcgccatcatccaggccgccgtgtgcatggccatgtacctgtacctggtgccccaccaccccctgacccgcttcaccgagcccgtgtactacgagtggggcttcttccgccgcctgtcctaccagtacatggccgccctgaccgcccgctggaagtactacttcatctggtccatctccgaggcctccctgatcatctccggcctgggcttctccggctggaccgagtcctccccccccaagccccgctgggaccgcgccaagaacgtggacatcatcggcgtggagttcgccaagtcctccgtgcagctgcccctggtgtggaacatccaggtgtccatctggctgcgccactacgtgtacgaccgcctggtgcagaacggcaagcgccccggcttcttccagctgctggccacccagaccgtgtccgccgtgtggcacggcctgtaccccggctacatcatcttcttcgtgcagtccgccctgatgatcgccggctcccgcgtgatctaccgctggcagcaggccgtgccccccaagatgggcctggtgaagaacatcttcgtgttcttcaacttcgcctacaccctgctggtgctgaactactccgccgtgggcttcatggtgctgtccatgcacgagaccctggcctcctacggctccgtgtactacatcggcaccatcctgcccatcaccctgatcctgctgtcctacgtgatcaagcccggcaagcccgcccgctccaaggcccacaaggagcagtgactcgag

SEQ ID NO: 119 - DNA, 1404 bp, Artificial Sequence
  Description of Artificial Sequence Synthetic polynucleotide
ggtaccatggagctgggctccgtggccgccgccatcggcgtgtccgtgcccgtggcccgcttcctgctgtgcttcctggccaccatccccgtgtccttcctgtggcgcctggtgcccggccgcctgcccaagcacctgtactccgccgcctccggcgccatcctgtcctacctgtccttcggcccctcctccaacctgcacttcatcgtgcccatgaccctgggctacctgtccatgctgttcttccgccccttctccggcctgctgaccttcttcctgggcttcggctacctgatcggctgccacgtgtactacatgtccggcgacgcctggaaggagggcggcatcgacgccaccggcgccctgatggtgctgaccctgaaggtgatctcctgctccatcaactacaacgacggcctgctgaaggaggagggcctgcgcgagtcccagaagaagaaccgcctgaccaagatgccctccctgatcgagtacatcggctactgcctgtgctgcggctcccacttcgccggccccgtgtacgagatgaaggactacctggagtggaccgagggcaagggcgtgtggtcccactccgagaaggagcccaagccctcccccttcggcggcgccctgcgcgccatcatccaggccgccgtgtgcatggccatgtacatgtacctggtgccccaccaccccctgtcccgcttcaccgagcccgtgtactacgagtggggcttcttccgccgcctgtcctaccagtacatggccggcctgaccgcccgctggaagtactacttcatctggtccatctccgaggcctccctgatcatctccggcctgggcttctccggctggaccgagtcctccccccccaagccccgctgggaccgcgccaagaacgtggacatcatcggcgtggagttcgccaagtcctccgtgcagctgcccctggtgtggaacatccaggtgtccacctggctgcgccactacgtgtacgaccgcctggtgcagaacggcaagcgccccggcttcttccagctgctggccacccagaccgtgtccgccatctggcacggcctgtaccccggctacatcatcttcttcgtgcagtccgccctgatgatcgccggctcccgcgtgatctaccgctggcagcaggccgtgccccccaagatgggcctggtgaagaacatcttcgtgttcttcaacttcgcctacaccctgctggtgctgaactactccgccgtgggcttcatggtgctgtccatgcacgagaccctggcctcctacggctccgtgtactacatcggcaccatcctgcccatcaccctgatcctgctgtcctacgtgatcaagcccggcaagcccgcccgctccaaggcccacaaggagcagtgactcgag

SEQ ID NO: 120 - DNA, 1410 bp, Artificial Sequence
  Description of Artificial Sequence Synthetic polynucleotide
ggtaccatggagctggagatcggctccgtggccgccgccatcggcgtgtccgtgcccgtggcccgcttcctgctgtgcttcctggccaccatccccgtgtccttcctgtgccgcctgctgcccgcccgcctgcccaagcacctgtactccgccgcctccggcgccatcctgtcctacctgtccttcggcccctcctccaacctgcacttcatcgtgcccatgtccctgggctacctgtccatgctgttcttccgccccttctccggcctgctgaccttcttcctgggcttcggctacctgatcggctgccacgtgtactacatgtccggcgacgcctggaaggagggcggcatcgacgccaccggcgccctgatggtgctgaccctgaaggtgatctcctgctccatcaactacaacgacggcctgctgaaggaggagggcctgcgcgagtcccagaagaagaaccgcctgaccaagatgccctccctgatcgagtacttcggctactgcctgtgctgcggctcccacttcgccggccccgtgtacgagatgaaggactacctggagtggaccgagggcaagggcatctggtcccgctccgagaaggaccccaagccctcccccttcggcggcgccctgcgcgccatcatccaggccgccgtgtgcatggccatgcacatgtacctggtgccccaccaccccctgacccgcttcaccgagcccgtgtactacgagtggggcttcttccgccgcctgtcctaccagtacatggccgcccagaccgcccgctggaagtactacttcatctggtccatctccgaggcctccctgatcatctccggcctgggcttctccggctggaccgagtcctccccccccaagccccgctgggacaaggccaagaacgtggacatcatcggcgtggagttcgccaagtcctccgtgcagctgcccctggtgtggaacatccaggtgtccacctggctgcgccactacgtgtacgaccgcctggtgcagaacggcaagcgccccggcttcttccagctgctggccacccagaccgtgtccgccgtgtggcacggcctgtaccccggctacatcatcttcttcgtgcagtccgccctgatgatcgccggctcccgcgtgatctaccgctggcagcaggccgtgccccagaagatgggcctggtgaagaacatcttcgtgttcttcaacttcgcctacaccctgctggtgctgaactactccgccgtgggcttcatggtgctgtccatgcacgagaccctggcctcctacggctccgtgtactacatcggcaccatcctgcccatcaccctgatcctgctgtcctacgtgatcaagcccggcaagcccacccgctccaaggtgcacaaggagcagtgactcgag

SEQ ID NO: 121 - DNA, 1410 bp, Artificial Sequence
  Description of Artificial Sequence Synthetic polynucleotide
ggtaccatggagctggagatggagcccctggccgccgccatcggcgtgtccgtggccgtgttccgcttcctggtgtgcttcatcgccaccatccccgtgtccttcatctgccgcctggtgcccggcggcctgccccgccacctgttctccgccgcctccggcgccgtgctgtcctacctgtccttcggcttctcctccaacctgcacttcctggtgcccatgaccctgggctacctgtccatgatcctgttccgccgcttctgcggcatcctgaccttcttcctgggcttcggctacctgatcggctgccacgtgtactacatgtccggcgacgcctggaaggagggcggcatcgacgccaccggcgccctgatggtgctgaccctgaaggtgatctcctgctccatcaactacaacgacggcctgctgaaggaggagggcctgcgcgagtcccagaagaagaaccgcctgatccgcctgccctccctgatcgagtacttcggctactgcctgtgctgcggctcccacttcgccggccccgtgtacgagatgaaggactacctggactggaccgagggcaagggcatctggtcccactccgagaagggccccaagccctcccccctgcgcgccgccctgcgcgccatcatccaggccggcttctgcatggccatgtacctgtacctggtgccccactaccccctgacccgcttcaccgaccccgtgtactacgagtggggcatcctgcgccgcctgtcctaccagtacatggcctccttcaccgcccgctggaagtactacttcatctggtccatctccgaggcctccctgatcatctccggcctgggcttctccggctggaccgagtcctccccccccaagccccgctgggaccgcgccaagaacgtggacatcctgggcgtggagctggccaagtcctccgtgcagatccccctggtgtggaacatccaggtgtccacctggctgcgccactacgtgtacgaccgcctggtgcagaacggcaagcgccccggcttcctgcagctgctggccacccagaccgtgtccgccatctggcacggcgtgtaccccggctacctgatcttcttcgtgcagtccgccctgatgatcgccggctcccgcgccatctaccgctggcagcaggccgtgccccccaagatgtccctggtgaagaacaccctggtgttcttcaacttcgcctacaccctgctggtgctgaactactccgccgtgggcttcatggtgctgtccatgcacgagaccctggcctcctacggctccgtgtactacgtgggcaccatcctgcccgtgaccctgatcctgctgggctacgtgatcaagcccggcaagtccccccgctccaaggcctccaaggagcagtgactcgag

SEQ ID NO: 122 - DNA, 750 bp, Artificial Sequence
  Description of Artificial Sequence Synthetic polynucleotide
ggtaccatgaacttcgacttcctgtccaacatcccctggttcggcgccaaggcctccgacaacgccggctcctccttcggctccgccaccatcgtgatccagcagcccccccccgtgtcccgcggcttcgacatccgccactggggctggccctggtccgtgctgtccgtgctgccctggggcaagcccggctgcgacgagctgcgcgccccccccaccaccatcaaccgccgcctgaagcgcaacgccacctccatgcactcctccgccgtgcgcggcaacgccgaggccgcccgcgtgcgcttccgcccctacgtgtccaaggtgccctggcacaccggcttccgcggcctgctgtcccagctgttcccccgctacggccactactgcggccccaactggtcctccggcaagaacggcggctcccccgtgtgggaccagcgccccatcgactggctggactactgctgctactgccacgacatcggctacgacacccacgaccaggccaagctgctggaggccgacctggccttcctggagtgcctggagcgcccctcctaccccaccaagggcgacgcccacgtggcccacatgtacaagaccatgtgcgtgaccggcctgcgcaacgtgctgatcccctaccgcacccagctgctgcgcctgaactcccgccagcccctgatcgacttcggctggctgtccaacgccgcctggaagggctggaacgcccagaagtcctgactcgag

SEQ ID NO: 123 - DNA, 723 bp, Artificial Sequence
  Description of Artificial Sequence Synthetic polynucleotide
ggtaccatgaacctggacttcctgtccaagatcccctggttcgaggccaaggcctccgagaaccccggcctgaacctgggctccaccaccatcgtgatcaagcagccccgccagggcttcgacatccgccactggggctggccctggtccgtgctgacctggggcaaccgcgtgaccgacgaggtgcacgccccccccaccaccatcaaccgccgcctgaagcgcaacgccaccggccccgccgtgcagggcgacaccgaggccgcccgcctgcgcttccgcccctacgtgtccaaggtgccctggcacaccggcttccgcggcctgctgtcccagctgttcccccgctacggccactactgcggccccaactggtcctccggcaagaacggcggctcccccgtgtgggaccagcgccccatcgactggctggactactgctgctactgccacgacatcggctacgacacccacgaccaggccaagctgctggaggccgacctggccttcctggagtgcctggagcgcccctcctaccccaccaccggcgacgcccacgtggcccacatgtacaagaccatgtgcgtgaccggcctgcgcaacgtgctgatcccctaccgcacccagctgctgcgcctgaacttccgccagcccctgatcgacttcggctggctgtccaacgccgcctggaagggctggtccgcccagaagacctgactcgag

SEQ ID NO: 124 - DNA, 489 bp, Artificial Sequence
  Description of Artificial Sequence Synthetic polynucleotide
ggtaccatggtgcacctgccccacaccctgaagctgggcctggtgatcgccatctccatctccggcctgtgcttctcctccacccccgcccgcgccctgaacgtgggcatccaggccgccggcgtgaccgtgtccgtgggcaagggctgctcccgcaagtgcgagtccgacttctgcaaggtgccccccttcctgcgctacggcaagtactgcggcctgatgtactccggctgccccggcgagaagccctgcgacggcctggacgcctgctgcatgaagcacgacgcctgcgtgcaggccaagaacaacgactacctgtcccaggagtgctcccagaacctgctgaactgcatggcctccttccgcatgtccggcggcaagcagttcaagggctccacctgccaggtggacgaggtggtggacgtgctgaccgtggtgatggaggccgccctgctggccggccgctacctgcacaagccctgactcgag

SEQ ID NO: 125 - DNA, 489 bp, Artificial Sequence
  Description of Artificial Sequence Synthetic polynucleotide
ggtaccatggtgcacctgccccacaccctgaagctgggcctggtgatcgccatctccatctccggcctgtgcctgtcctccacccccgcccgcgccctgaacgtgggcatccaggccgccggcgtgaccgtgtccgtgggcaagggctgctcccgcaagtgcgagtccgacttctgcaaggtgccccccttcctgcgctacggcaagtactgcggcctgatgtactccggctgccccggcgagaagccctgcgacggcctggacgcctgctgcatgaagcacgacgcctgcgtgcaggccaagaacgacgactacctgtcccaggagtgctcccagaacctgctgaactgcatggcctccttccgcatgtccggcggcaagcagttcaagggctccacctgccaggtggacgaggtggtggacgtgctgaccgtggtgatggaggccgccctgctggccggccgctacctgcacaagccctgactcgag

SEQ ID NO: 126 - DNA, 7557 bp, Artificial Sequence
  Description of Artificial Sequence Synthetic polynucleotide
gtttaaacgccggtcaccacccgcatgctcgtactacagcgcacgcaccgcttcgtgatccaccgggtgaacgtagtcctcgacggaaacatctggttcgggcctcctgcttgcactcccgcccatgccgacaacctttctgctgttaccacgacccacaatgcaacgcgacacgaccgtgtgggactgatcggttcactgcacctgcatgcaattgtcacaagcgcttactccaattgtattcgtttgttttctgggagcagttgctcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtatgcagaccgcccaccagcgcccccccaccgagggccactgcttcggcgcccgcctgcccaccgcctcccgccgcgccgtgcgccgcgcctggtcccgcatcgcccgcgggcgcgccgccgccgccgccgacgccaaccccgcccgccccgagcgccgcgtggtgatcaccggccagggcgtggtgacctccctgggccagaccatcgagcagttctactcctccctgctggagggcgtgtccggcatctcccagatccagaagttcgacaccaccggctacaccaccaccatcgccggcgagatcaagtccctgcagctggacccctacgtgcccaagcgctgggccaagcgcgtggacgacgtgatcaagtacgtgtacatcgccggcaagcaggccctggagtccgccggcctgcccatcgaggccgccggcctggccggcgccggcctggaccccgccctgtgcggcgtgctgatcggcaccgccatggccggcatgacctccttcgccgccggcgtggaggccctgacccgcggcggcgtgcgcaagatgaaccccttctgcatccccttctccatctccaacatgggcggcgccatgctggccatggacatcggcttcatgggccccaactactccatctccaccgcctgcgccaccggcaactactgcatcctgggcgccgccgaccacatccgccgcggcgacgccaacgtgatgctggccggcggcgccgacgccgccatcatcccctccggcatcggcggcttcatcgcctgcaaggccctgtccaagcgcaacgacgagcccgagcgcgcctcccgcccctgggacgccgaccgcgacggcttcgtgatgggcgagggcgccggcgtgctggtgctggaggagctggagcacgccaagcgccgcggcgccaccatcctggccgagctggtgggcggcgccgccacctccgacgcccaccacatgaccgagcccgacccccagggccgcggcgtgcgcctgtgcctggagcgcgccctggagcgcgcccgcctggcccccgagcgcgtgggctacgtgaacgcccacggcacctccacccccgccggcgacgtggccgagtaccgcgccatccgcgccgtgatcccccaggactccctgcgcatcaactccaccaagtccatgatcggccacctgctgggcggcgccggcgccgtggaggccgtggccgccatccaggccctgcgcaccggctggctgcaccccaacctgaacctggagaaccccgcccccggcgtggaccccgtggtgctggtgggcccccgcaaggagcgcgccgaggacctggacgtggtgctgtccaactccttcggcttcggcggccacaactcctgcgtgatcttccgcaagtacgacgagatggactacaaggaccacgacggcgactacaaggaccacgacatcgactacaaggacgacgacgacaagtgaatcgatgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggagagctccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctaggtgatatcgaattcctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacactagtatggctatcaagacgaacaggcagcctgtggagaagcctccgttcacgatcgggacgctgcgcaaggccatccccgcgcactgtttcgagcgctcggcgcttcgtagcagcatgtacctggcctttgacatcgcggtcatgtccctgctctacgtcgcgtcgacgtacatcgaccctgcaccggtgcctacgtgggtcaagtacggcatcatgtggccgctctactggttcttccaggtgtgtttgagggttttggttgcccgtattgaggtcctggtggcgcgcatggaggagaaggcgcctgtcccgctgacccccccggctaccctcccggcaccttccagggcgcgtacgggaagaaccagtagagcggccacatgatgccgtacttgacccacgtaggcaccggtgcagggtcgatgtacgtcgacgcgacgtagagcagggacatgaccgcgatgtcaaaggccaggtacatgctgctacgaagcgccgagcgctcgaaacagtgcgcggggatggccttgcgcagcgtcccgatcgtgaacggaggcttctccacaggctgcctgttcgtcttgatagccatctcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagctgtagagctcgatctaagtaagattcgaagcgctcgaccgtgccggacggactgcagccccatgtcgtagtgaccgccaatgtaagtgggctggcgtttccctgtacgtgagtcaacgtcactgcacgcgcaccaccctctcgaccggcaggaccaggcatcgcgagatacagcgcgagccagacacggagtgccgagctatgcgcacgctccaactaggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaggatccttagggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtcctgggcgaagaacgagggaatttgtgggtaaaacaagcatcgtctctcaggccccggcgcagtggccgttaaagtccaagaccgtgaccaggcagcgcagcgcgtccgtgtgcgggccctgcctggcggctcggcgtgccaggctcgagagcagctccctcaggtcgccttggacggcctctgcgaggccggtgagggcctgcaggagcgcctcgagcgtggcagtggcggtcgtatccgggtcgccggtcaccgcctgcgactcgccatccgaagagcgtttaaac

SEQ ID NO: 127 - DNA, 8094 bp, Artificial Sequence
  Description of Artificial Sequence Synthetic polynucleotide
gaagagcgcccaatgtttaaacctcttttgctgcgtctcctcaggcttgggggcctccttgggcttgggtgccgccatgatctgcgcgcatcagagaaacgttgctggtaaaaaggagcgcccggctgcgcaatatatatataggcatgccaacacagcccaacctcactcgggagcccgtcccaccacccccaagtcgcgtgccttgacggcatactgctgcagaagcttcatgagaatgatgccgaacaagaggggcacgaggacccaatcccggacatccttgtcgataatgatctcgtgagtccccatcgtccgcccgacgctccggggagcccgccgatgctcaagacgagagggccctcgaccaggaggggctggcccgggcgggcactggcgtcgaaggtgcgcccgtcgttcgcctgcagtcctatgccacaaaacaagtcttctgacggggtgcgtttgctcccgtgcgggcaggcaacagaggtattcaccctggtcatggggagatcggcgatcgagctgggataagagatacggtcccgcgcaaggatcgctcatcctggtctgagccggacagtcattctggcaagcaatgacaacttgtcaggaccggaccgtgccatatatttctcacctagcgccgcaaaacctaacaatttgggagtcactgtgccactgagttcgactggtagctgaatggagtcgctgctccactaaacgaattgtcagcaccgccagccggccgaggacccgagtcatagcgagggtagtagcgcgccactagtatggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccgccggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtgaatcgatggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctaggtacgccgctcagcctacacgtcttctccgatacctttccctcattgcattttatgccagactgggtcccagcctgggtgggtgctcccgctcgattgctcgtgtcggaggcggggcacccccgctctctctatttatcactgcctctccccgaccaaccctgacgactgtaaccctgccagaaacaattcagcctcatcaaaccgagttgtgcacaagggcgactaattttttagtcgggaaacaacccgcttccagaagcatccggacgggggtagcgaggctgtgtcgagcgccgtggggatctggccggtgaggtgcccgaaatccgtgtacagctcagcggctgggatcatcgacccccgggatcatcgaccccgtgggccgggcccccggaccctataactaaaagccgacgccagtgcaaaaccacaaacatttactccttaatcctccctcctccttcatacacacccacaagtaatcaactcacccatatggccatcgccgccgccgccgtgatcgtgcccctgggcctgctgttcttcatctccggcctggtggtgaacctgatccaggccctgtgcttcgtgctgatccgccccctgtccaagaacacctaccgcaagatcaaccgcgtggtggccgagctgctgtggctggagctgatctggctggtggactggtgggccggcgtgaagatcaaggtgttcatggaccccgagtccttcaacctgatgggcaaggagcacgccctggtggtggccaaccaccgctccgacatcgactggctggtgggctggctgctggcccagcgctccggctgcctgggctccgccctggccgtgatgaagaagtcctccaagttcctgcccgtgatcggctggtccatgtggttctccgagtacctgttcctggagcgctcctgggccaaggacgagaacaccctgaaggccggcctgcagcgcctgaaggacttcccccgccccttctggctggccttcttcgtggagggcacccgcttcacccaggccaagttcctggccgcccaggagtacgccgcctcccagggcctgcccatcccccgcaacgtgctgatcccccgcaccaagggcttcgtgtccgccgtgtcccacatgcgctccttcgtgcccgccatctacgacatgaccgtggccatccccaagtcctccccctcccccaccatgctgcgcctgttcaagggccagccctccgtggtgcacgtgcacatcaagcgctgcctgatgaaggagctgcccgagaccgacgaggccgtggcccagtggtgcaaggacatgttcgtggagaaggacaagctgctggacaagcacatcgccgaggacaccttctccgaccagcccatgcaggacctgggccgccccatcaagtccctgctggtggtggcctcctgggcctgcctgatggcctacggcgccctgaagttcctgcagtgctcctccctgctgtcctcctggaagggcatcgccttcttcctggtgggcctggccatcgtgaccatcctgatgcacatcctgatcctgttctcccagtccgagcgctccacccccgccaaggtggcccccggcaagcccaagaacgacggcgagacctccgaggcccgccgcgacaagcagcagtgaatgcatatgtggagatgtagggtggtcgactcgttggaggtgggtgtttttttttatcgagtgcgcggcgcggcaaacgggtccctttttatcgaggtgttcccaacgccgcaccgccctcttaaaacaacccccaccaccacttgtcgaccttctcgtttgttatccgccacggcgccccggaggggcgtcgtctggccgcgcgggcagctgtatcgccgcgctcgctccaatggtgtgtaatcttggaaagataataatcgatggatgaggaggagagcgtgggagatcagagcaaggaatatacagttggcacgaagcagcagcgtactaagctgtagcgtgttaagaaagaaaaactcgctgttaggctgtattaatcaaggagcgtatcaataattaccgaccctatacctttatctccaacccaatcgcggcttaaggatctaagtaagattcgaagcgctcgaccgtgccggacggactgcagccccatgtcgtagtgaccgccaatgtaagtgggctggcgtttccctgtacgtgagtcaacgtcactgcacgcgcaccaccctctcgaccggcaggaccaggcatcgcgagatacagcgcgagccagacacggagtgccgagctatgcgcacgctccaactaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcctgaggacagggtggttggctggatggggaaacgctggtcgcgggattcgatcctgctgcttatatcctccctggaagcacacccacgactctgaagaagaaaacgtgcacacacacaacccaaccggccgaatatttgcttccttatcccgggtccaagagagactgcgatgcccccctcaatcagcatcctcctccctgccgcttcaatcttccctgcttgcctgcgcccgcggtgcgccgtctgcccgcccagtcagtcactcctgcacaggccccttgtgcgcagtgctcctgtaccctttaccgctccttccattctgcgaggccccctattgaatgtattcgttgcctgtgtggccaagcgggctgctgggcgcgccgccgtcgggcagtgctcggcgactttggcggaagccgattgttcttctgtaagccacgcgcttgctgctttgggaagagaagggggggggtactgaatggatgaggaggagaaggaggggtattggtattatctgagttggggaggcagggagagttggaaaatgtaagtggcacgacgggcaaggagaatggtgagcatgtgcatggtgatgtcgttggtcgaggacgatcctgcacgcgtgtatctgatgtagaatacggcaatcaccctagtctacatctataccttctccgtataacgccctttccaaatgccctcccgtttctctcctattcttgatccacatgatgaccctggcactatttcaagggctggagaagagcgtttaaac

SEQ ID NO: 128 - DNA, 10062 bp, Artificial Sequence
  Description of Artificial Sequence Synthetic polynucleotide
gctcttcgcgaaggtcattttccagaacaacgaccatggcttgtcttagcgatcgctcgaatgactgctagtgagtcgtacgctcgacccagtcgctcgcaggagaacgcggcaactgccgagcttcggcttgccagtcgtgactcgtatgtgatcaggaatcattggcattggtagcattataattcggcttccgcgctgtttatgggcatggcaatgtctcatgcagtcgaccttagtcaaccaattctgggtggccagctccgggcgaccgggctccgtgtcgccgggcaccacctcctgccatgagtaacagggccgccctctcctcccgacgttggccaactgaataccgtgtcttggggccctacatgatgggctgcctagtcgggcgggacgcgcaactgcccgcgcaatctgggacgtggtctgaatcctccaggcgggtttccccgagaaagaaagggtgccgatttcaaagcagagccatgtgccgggccctgtggcctgtgttggcgcctatgtagtcaccccccctcacccaattgtcgccagtttgcgcaatccataaactcaaaactgcagcttctgagctgcgctgttcaagaacacctctggggtttgctcacccgcgaggtcgacggtacctccctccgtctctgcactctggcgcccctcctccgtctcgtggactgacggacgagagtctgggcgccgcttttctatccacaccgccctttccgcatcgaagacaccacccatcgtgccgccaggtcttccccaatcacccgccctgtggtcctctctcccagccgtgtttggtcgctgcgtccacatttttccattcgtgccccacgatcctcgcccatcttggcgccttggataggcacccttttttcagcacgccctggtgtgtagcacaacctgacctctctctaccgcatcgcctccctcccacacctcagttgactccctcgtcgcacgttgcacccgcaagctccccatttcatcctattgacaatcgcacactgtacatgtatgctcattattttgcaaaaaaacagggggtcggttcactcctggcagacgacgcggtgctgccgcgcgccgctgaggcggcgtcgcgacggcaacacccatcgcaccgcacgtcgacgagtcaacccaccctgctcaacggtgatctccccatcgcgacaccccccgtgaccgtactatgtgcgtccatacgcaacatgaaaaggaccttggtccccggaggcggcgagctcgtaatcccgaggttggccccgcttccgctggacacccatcgcatcttccggctcgcccgctgtcgagcaagcgccctcgtgcgcgcaacccttgtggtgcctgcccgcagagccgggcataaaggcgagcaccacacccgaaccagtccaatttgctttctgcattcactcaccaacttttacatccacacatcgtactaccacacctgcccagtcgggtttgatttctattgcaaaggtgcgggggggttggcgcactgcgtgggttgtgcagccggccgccgcggctgtacccagcgatcaggtagcttgggctgtatcttctcaagcattaccttgtcctgggcgtaggtttgcctctagaatggccgcgtccgtccactgcaccctgatgtccgtggtctgcaacaacaagaaccactccgcccgccccaagctgcccaactcctccctgctgcccggcttcgacgtggtggtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcctgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgatacgtaacagacgaccttggcaggcgtcgggtagggaggtggtggtgatggcgtctcgatgccatcgcacgcatccaacgaccgtatacgcatcgtccaatgaccgtcggtgtcctctctgcctccgttttgtgagatgtctcaggcttggtgcatcctcgggtggccagccacgttgcgcgtcgtgctgcttgcctctcttgcgcctctgtggtactggaaaatatcatcgaggcccgtttttttgctcccatttcctttccgctacatcttgaaagcaaacgacaaacgaagcagcaagcaaagagcacgaggacggtgaacaagtctgtcacctgtatacatctatttccccgcgggtgcacctactctctctcctgccccggcagagtcagctgccttacgtgacggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctaggctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccatctccgactcccccgagatcctgggctccaccgccaccgtgacctcctcctcccactccgactccgacctgaacctgctgtccatccgccgccgcacctccaccaccgccgccgcccgcgcccccgaccgcgacgactccggcaacggcgaggccgtggacgaccgcgaccgcgtggagtccgccaacctgatgtccaacgtggccgagaacgccaacgagatgcccaactcctccgacacccgcttcacctaccgcccccgcgtgcccgcccaccgccgcatcaaggagtcccccctgtcctccggcgccatcttcaagcagtcccacgccggcctgttcaacctgtgcatcgtggtgctggtggccgtgaactcccgcctgatcatcgagaacctgatgaagtacggctggctgatccgctccggcttctggttctcctcccgctccctgtccgactggcccctgttcatgtgctgcctgaccctgcccatcttccccctggccgccttcgtggtggagaagctggtgcagcgcaactacatctccgagcccgtggtggtgttcctgcacgccatcatctccaccaccgccgtgctgtaccccgtgatcgtgaacctgcgctgcgactccgccttcctgtccggcgtggccctgatgctgttcgcctgcatcgtgtggctgaagctggtgtcctacgcccacaccaacaacgacatgcgcgccctggccaagtccgccgagaagggcgacgtggacccctcctacgacgtgtccttcaagtccctggcctacttcatggtggcccccaccctgtgctaccagcagtcctacccccgcacccccgccgtgcgcaagtcctgggtggtgcgccagttcatcaagctgatcgtgttcaccggcctgatgggcttcatcatcgagcagtacatcaaccccatcgtgcagaactcccagcaccccctgaagggcaacctgctgtacgccatcgagcgcgtgctgaagctgtccgtgcccaacctgtacgtgtggctgtgcatgttctactgcttcttccacctgtggctgaacatcctggccgagctgctgcgcttcggcgaccgcgagttctacaaggactggtggaacgccaagaccgtggaggagtactggcgcatgtggaacatgcccgtgcacaagtggatggtgcgccacatctacttcccctgcctgcgcaacggcatccccaagggcgtggccatcgtgatcgccttcctggtgtccgccgtgttccacgagctgtgcatcgccgtgccctgccacatgttcaagctgtgggccttcatcggcatcatgttccaggtgcccctggtgctgatcaccaactacctgcaggacaagttccgctcctccatggtgggcaacatgatcttctggttcatcttctccatcctgggccagcccatgtgcgtgctgctgtactaccacgacctgatgaaccgcaagggcaaggccgactgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacttaaggatctaagtaagattcgaagcgctcgaccgtgccggacggactgcagccccatgtcgtagtgaccgccaatgtaagtgggctggcgtttccctgtacgtgagtcaacgtcactgcacgcgcaccaccctctcgaccggcaggaccaggcatcgcgagatacagcgcgagccagacacggagtgccgagctatgcgcacgctccaactagatatcatgtggatgatgagcatgaattcgggagcagttgtcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccgccggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtgaatcgatggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctcctcactcagcgcgcctgcgcggggatgcggaacgccgccgccgccttgtcttttgcacgcgcgactccgtcgcttcgcgggtggcacccccattgaaaaaaacctcaattctgtttgtggaagacacggtgtacccccaaccacccacctgcacctctattattggtattattgacgcgggagcgggcgttgtactctacaacgtagcgtctctggttttcagctggctcccaccattgtaaattcttgctaaaatagtgcgtggttatgtgagaggtatggtgtaacagggcgtcagtcatgttggttttcgtgctgatctcgggcacaaggcgtcgtcgacgtgacgtgcccgtgatgagagcaataccgcgctcaaagccgacgcatggcctttactccgcactccaaacgactgtcgctcgtatttttcggatatctattttttaagagcgagcacagcgccgggcatgggcctgaaaggcctcgcggccgtgctcgtggtgggggccgcgagcgcgtggggcatcgcggcagtgcaccaggcgcagacggaggaacgcatggtgagtgcgcatcacaagatgcatgtcttgttgtctgtactataatgctagagcatcaccaggggcttagtcatcgcacctgctttggtcattacagaaattgcacaagggcgtcctccgggatgaggagatgtaccagctcaagctggagcggcttcgagccaagcaggagcgcggcgcatgacgacctacccacatgcgaagagc

SEQ ID NO: 129 - DNA, 9540 bp, Artificial Sequence
  Description of Artificial Sequence Synthetic polynucleotide
gctcttcgcgaaggtcattttccagaacaacgaccatggcttgtcttagcgatcgctcgaatgactgctagtgagtcgtacgctcgacccagtcgctcgcaggagaacgcggcaactgccgagcttcggcttgccagtcgtgactcgtatgtgatcaggaatcattggcattggtagcattataattcggcttccgcgctgtttatgggcatggcaatgtctcatgcagtcgaccttagtcaaccaattctgggtggccagctccgggcgaccgggctccgtgtcgccgggcaccacctcctgccatgagtaacagggccgccctctcctcccgacgttggccaactgaataccgtgtcttggggccctacatgatgggctgcctagtcgggcgggacgcgcaactgcccgcgcaatctgggacgtggtctgaatcctccaggcgggtttccccgagaaagaaagggtgccgatttcaaagcagagccatgtgccgggccctgtggcctgtgttggcgcctatgtagtcaccccccctcacccaattgtcgccagtttgcgcaatccataaactcaaaactgcagcttctgagctgcgctgttcaagaacacctctggggtttgctcacccgcgaggtcgacggtacctccctccgtctctgcactctggcgcccctcctccgtctcgtggactgacggacgagagtctgggcgccgcttttctatccacaccgccctttccgcatcgaagacaccacccatcgtgccgccaggtcttccccaatcacccgccctgtggtcctctctcccagccgtgtttggtcgctgcgtccacatttttccattcgtgccccacgatcctcgcccatcttggcgccttggataggcacccttttttcagcacgccctggtgtgtagcacaacctgacctctctctaccgcatcgcctccctcccacacctcagttgactccctcgtcgcacgttgcacccgcaagctccccatttcatcctattgacaatcgcacactgtacatgtatgctcattattttgcaaaaaaacagggggtcggttcactcctggcagacgacgcggtgctgccgcgcgccgctgaggcggcgtcgcgacggcaacacccatcgcaccgcacgtcgacgagtcaacccaccctgctcaacggtgatctccccatcgcgacaccccccgtgaccgtactatgtgcgtccatacgcaacatgaaaaggaccttggtccccggaggcggcgagctcgtaatcccgaggttggccccgcttccgctggacacccatcgcatcttccggctcgcccgctgtcgagcaagcgccctcgtgcgcgcaacccttgtggtgcctgcccgcagagccgggcataaaggcgagcaccacacccgaaccagtccaatttgctttctgcattcactcaccaacttttacatccacacatcgtactaccacacctgcccagtcgggtttgatttctattgcaaaggtgcgggggggttggcgcactgcgtgggttgtgcagccggccgccgcggctgtacccagcgatcaggtagcttgggctgtatcttctcaagcattaccttgtcctgggcgtaggtttgcctctagaatggccgcgtccgtccactgcaccctgatgtccgtggtctgcaacaacaagaaccactccgcccgccccaagctgcccaactcctccctgctgcccggcttcgacgtggtggtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcctgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgatacgtaacagacgaccttggcaggcgtcgggtagggaggtggtggtgatggcgtctcgatgccatcgcacgcatccaacgaccgtatacgcatcgtccaatgaccgtcggtgtcctctctgcctccgttttgtgagatgtctcaggcttggtgcatcctcgggtggccagccacgttgcgcgtcgtgctgcttgcctctcttgcgcctctgtggtactggaaaatatcatcgaggcccgtttttttgctcccatttcctttccgctacatcttgaaagcaaacgacaaacgaagcagcaagcaaagagcacgaggacggtgaacaagtctgtcacctgtatacatctatttccccgcgggtgcacctactctctctcctgccccggcagagtcagctgccttacgtgacggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctaggctggctcgggcctcgtgctggcactccctcccatgccgacaacctttctgctgtcaccacgacccacgatgcaacgcgacacgacccggtgggactgatcggttcactgcacctgcatgcaattgtcacaagcgcatactccaatcgtatccgtttgatttctgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatgaccggcgaggagatggaggagcgcaaggccaccggctaccgcgagttctccggccgccacgagttcccctccaacaccatgcacgccctgctggccatgggcatctggctgggcgccatccacttcaacgccctgctgctgctgttctccttcctgttcctgcccttctccaagttcctggtggtgttcggcctgctgctgctgttcatgatcctgcccatcgacccctactccaagttcggccgccgcctgtcccgctacatctccaagcacgcctgctcctacttccccatcaccctgcacgtggaggacatccacgccttccaccccgaccgcgcctacgtgttcggcttcgagccccactccgtgctgcccatcggcgtggtggccctggccgacctgaccggcttcatgcccctgcccaagatcaaggtgctggcctcctccgccgtgttctacacccccttcctgcgccacatctggacctggctgggcctgacccccgccaccaagaagaacttctcctccctgctggacgccggctactcctgcatcctggtgcccggcggcgtgcaggagaccttccacatggagcccggctccgagatcgccttcctgcgcgcccgccgcggcttcgtgcgcatcgccatggagatgggctcccccctggtgcccgtgttctgcttcggccagtcccacgtgtacaagtggtggaagcccggcggcaagttctacctgcagttctcccgcgccatcaagttcacccccatcttcttctggggcatcttcggctcccccctgccctaccagcaccccatgcacgtggtggtgggcaagcccatcgacgtgaagaagaacccccagcccatcgtggaggaggtgatcgaggtgcacgaccgcttcgtggaggccctgcaggacctgttcgagcgccacaaggcccaggtgggcttcgccgacctgcccctgaagatcctgtgaatcgatagatctcttaaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacttaaggatctaagtaagattcgaagcgctcgaccgtgccggacggactgcagccccatgtcgtagtgaccgccaatgtaagtgggctggcgtttccctgtacgtgagtcaacgtcactgcacgcgcaccaccctctcgaccggcaggaccaggcatcgcgagatacagcgcgagccagacacggagtgccgagctatgcgcacgctccaactagatatcatgtggatgatgagcatgaattcgggagcagttgtcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccgccggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtgaatcgatggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctcctcactcagcgcgcctgcgcggggatgcggaacgccgccgccgccttgtcttttgcacgcgcgactccgtcgcttcgcgggtggcacccccattgaaaaaaacctcaattctgtttgtggaagacacggtgtacccccaaccacccacctgcacctctattattggtattattgacgcgggagcgggcgttgtactctacaacgtagcgtctctggttttcagctggctcccaccattgtaaattcttgctaaaatagtgcgtggttatgtgagaggtatggtgtaacagggcgtcagtcatgttggttttcgtgctgatctcgggcacaaggcgtcgtcgacgtgacgtgcccgtgatgagagcaataccgcgctcaaagccgacgcatggcctttactccgcactccaaacgactgtcgctcgtatttttcggatatctattttttaagagcgagcacagcgccgggcatgggcctgaaaggcctcgcggccgtgctcgtggtgggggccgcgagcgcgtggggcatcgcggcagtgcaccaggcgcagacggaggaacgcatggtgagtgcgcatcacaagatgcatgtcttgttgtctgtactataatgctagagcatcaccaggggcttagtcatcgcacctgctttggtcattacagaaattgcacaagggcgtcctccgggatgaggagatgtaccagctcaagctggagcggcttcgagccaagcaggagcgcggcgcatgacgacctacccacatgcgaagagc

SEQ ID NO: 130 - DNA, 7158 bp, Artificial Sequence
  Description of Artificial Sequence Synthetic polynucleotide
gctcttcgcgaaggtcattttccagaacaacgaccatggcttgtcttagcgatcgctcgaatgactgctagtgagtcgtacgctcgacccagtcgctcgcaggagaacgcggcaactgccgagcttcggcttgccagtcgtgactcgtatgtgatcaggaatcattggcattggtagcattataattcggcttccgcgctgtttatgggcatggcaatgtctcatgcagtcgaccttagtcaaccaattctgggtggccagctccgggcgaccgggctccgtgtcgccgggcaccacctcctgccatgagtaacagggccgccctctcctcccgacgttggccaactgaataccgtgtcttggggccctacatgatgggctgcctagtcgggcgggacgcgcaactgcccgcgcaatctgggacgtggtctgaatcctccaggcgggtttccccgagaaagaaagggtgccgatttcaaagcagagccatgtgccgggccctgtggcctgtgttggcgcctatgtagtcaccccccctcacccaattgtcgccagtttgcgcaatccataaactcaaaactgcagcttctgagctgcgctgttcaagaacacctctggggtttgctcacccgcgaggtcgacggtacctccctccgtctctgcactctggcgcccctcctccgtctcgtggactgacggacgagagtctgggcgccgcttttctatccacaccgccctttccgcatcgaagacaccacccatcgtgccgccaggtcttccccaatcacccgccctgtggtcctctctcccagccgtgtttggtcgctgcgtccacatttttccattcgtgccccacgatcctcgcccatcttggcgccttggataggcacccttttttcagcacgccctggtgtgtagcacaacctgacctctctctaccgcatcgcctccctcccacacctcagttgactccctcgtcgcacgttgcacccgcaagctccccatttcatcctattgacaatcgcacactgtacatgtatgctcattattttgcaaaaaaacagggggtcggttcactcctggcagacgacgcggtgctgccgcgcgccgctgaggcggcgtcgcgacggcaacacccatcgcaccgcacgtcgacgagtcaacccaccctgctcaacggtgatctccccatcgcgacaccccccgtgaccgtactatgtgcgtccatacgcaacatgaaaaggaccttggtccccggaggcggcgagctcgtaatcccgaggttggccccgcttccgctggacacccatcgcatcttccggctcgcccgctgtcgagcaagcgccctcgtgcgcgcaacccttgtggtgcctgcccgcagagccgggcataaaggcgagcaccacacccgaaccagtccaatttgctttctgcattcactcaccaacttttacatccacacatcgtactaccacacctgcccagtcgggtttgatttctattgcaaaggtgcgggggggttggcgcactgcgtgggttgtgcagccggccgccgcggctgtacccagcgatcaggtagcttgggctgtatcttctcaagcattaccttgtcctgggcgtaggtttgcctctagaatggccgcgtccgtccactgcaccctgatgtccgtggtctgcaacaacaagaaccactccgcccgccccaagctgcccaactcctccctgctgcccggcttcgacgtggtggtccaggccgcggccacccgcttcaagaaggagacgacgaccacccgcgccacgctgacgttcgacccccccacgaccaactccgagcgcgccaagcagcgcaagcacaccatcgacccctcctcccccgacttccagcccatcccctccttcgaggagtgcttccccaagtccacgaaggagcacaaggaggtggtgcacgaggagtccggccacgtcctgaaggtgcccttccgccgcgtgcacctgtccggcggcgagcccgccttcgacaactacgacacgtccggcccccagaacgtcaacgcccacatcggcctggcgaagctgcgcaaggagtggatcgaccgccgcgagaagctgggcacgccccgctacacgcagatgtactacgcgaagcagggcatcatcacggaggagatgctgtactgcgcgacgcgcgagaagctggaccccgagttcgtccgctccgaggtcgcgcggggccgcgccatcatcccctccaacaagaagcacctggagctggagcccatgatcgtgggccgcaagttcctggtgaaggtgaacgcgaacatcggcaactccgccgtggcctcctccatcgaggaggaggtctacaaggtgcagtgggccaccatgtggggcgccgacaccatcatggacctgtccacgggccgccacatccacgagacgcgcgagtggatcctgcgcaactccgcggtccccgtgggcaccgtccccatctaccaggcgctggagaaggtggacggcatcgcggagaacctgaactgggaggtgttccgcgagacgctgatcgagcaggccgagcagggcgtggactacttcacgatccacgcgggcgtgctgctgcgctacatccccctgaccgccaagcgcctgacgggcatcgtgtcccgcggcggctccatccacgcgaagtggtgcctggcctaccacaaggagaacttcgcctacgagcactgggacgacatcctggacatctgcaaccagtacgacgtcgccctgtccatcggcgacggcctgcgccccggctccatctacgacgccaacgacacggcccagttcgccgagctgctgacccagggcgagctgacgcgccgcgcgtgggagaaggacgtgcaggtgatgaacgagggccccggccacgtgcccatgcacaagatccccgagaacatgcagaagcagctggagtggtgcaacgaggcgcccttctacaccctgggccccctgacgaccgacatcgcgcccggctacgaccacatcacctccgccatcggcgcggccaacatcggcgccctgggcaccgccctgctgtgctacgtgacgcccaaggagcacctgggcctgcccaaccgcgacgacgtgaaggcgggcgtcatcgcctacaagatcgccgcccacgcggccgacctggccaagcagcacccccacgcccaggcgtgggacgacgcgctgtccaaggcgcgcttcgagttccgctggatggaccagttcgcgctgtccctggaccccatgacggcgatgtccttccacgacgagacgctgcccgcggacggcgcgaaggtcgcccacttctgctccatgtgcggccccaagttctgctccatgaagatcacggaggacatccgcaagtacgccgaggagaacggctacggctccgccgaggaggccatccgccagggcatggacgccatgtccgaggagttcaacatcgccaagaagacgatctccggcgagcagcacggcgaggtcggcggcgagatctacctgcccgagtcctacgtcaaggccgcgcagaagtgatacgtaacagacgaccttggcaggcgtcgggtagggaggtggtggtgatggcgtctcgatgccatcgcacgcatccaacgaccgtatacgcatcgtccaatgaccgtcggtgtcctctctgcctccgttttgtgagatgtctcaggcttggtgcatcctcgggtggccagccacgttgcgcgtcgtgctgcttgcctctcttgcgcctctgtggtactggaaaatatcatcgaggcccgtttttttgctcccatttcctttccgctacatcttgaaagcaaacgacaaacgaagcagcaagcaaagagcacgaggacggtgaacaagtctgtcacctgtatacatctatttccccgcgggtgcacctactctctctcctgccccggcagagtcagctgccttacgtgacggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggggagcagttgtcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccgccggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtgaatcgatggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctcctcactcagcgcgcctgcgcggggatgcggaacgccgccgccgccttgtcttttgcacgcgcgactccgtcgcttcgcgggtggcacccccattgaaaaaaacctcaattctgtttgtggaagacacggtgtacccccaaccacccacctgcacctctattattggtattattgacgcgggagcgggcgttgtactctacaacgtagcgtctctggttttcagctggctcccaccattgtaaattcttgctaaaatagtgcgtggttatgtgagaggtatggtgtaacagggcgtcagtcatgttggttttcgtgctgatctcgggcacaaggcgtcgtcgacgtgacgtgcccgtgatgagagcaataccgcgctcaaagccgacgcatggcctttactccgcactccaaacgactgtcgctcgtatttttcggatatctattttttaagagcgagcacagcgccgggcatgggcctgaaaggcctcgcggccgtgctcgtggtgggggccgcgagcgcgtggggcatcgcggcagtgcaccaggcgcagacggaggaacgcatggtgagtgcgcatcacaagatgcatgtcttgttgtctgtactataatgctagagcatcaccaggggcttagtcatcgcacctgctttggtcattacagaaattgcacaagggcgtcctccgggatgaggagatgtaccagctcaagctggagcggcttcgagccaagcaggagcgcggcgcatgacgacctacccacatgcgaagagc

SEQ ID NO: 131 - DNA, 6046 bp, Artificial Sequence
  Description of Artificial Sequence Synthetic polynucleotide
caccggcgcgctgcttcgcgtgccgggtgcagcaatcagatccaagtctgacgacttgcgcgcacgcgccggatccttcaattccaaagtgtcgtccgcgtgcgcttcttcgccttcgtcctcttgaacatccagcgacgcaagcgcagggcgctgggcggctggcgtcccgaaccggcctcggcgcacgcggctgaaattgccgatgtcggcaatgtagtgccgctccgcccacctctcaattaagtttttcagcgcgtggttgggaatgatctgcgctcatggggcgaaagaaggggttcagaggtgctttattgttactcgactgggcgtaccagcattcgtgcatgactgattatacatacaaaagtacagctcgcttcaatgccctgcgattcctactcccgagcgagcactcctctcaccgtcgggttgcttcccacgaccacgccggtaagagggtctgtggcctcgcgcccctcgcgagcgcatctttccagccacgtctgtatgattttgcgctcatacgtctggcccgtcgaccccaaaatgacgggatcctgcataatatcgcccgaaatgggatccaggcattcgtcaggaggcgtcagccccgcgggagatgccggtcccgccgcattggaaaggtgtagagggggtgaatcccccatttcatgaaatgggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgcctcccagctgcgcaagcccgccctggaccccctgcgcgccgtgatctccgccgaccagggctccatctcccccgtgaactcctgcacccccgccgaccgcctgcgcgccggccgcctgatggaggacggctactcctacaaggagaagttcatcgtgcgctcctacgaggtgggcatcaacaagaccgccaccgtggagaccatcgccaacctgctgcaggaggtggcctgcaaccacgtgcagaagtgcggcttctccaccgacggcttcgccaccaccctgaccatgcgcaagctgcacctgatctgggtgaccgcccgcatgcacatcgagatctacaagtaccccgcctggtccgacgtggtggagatcgagacctggtgccagtccgagggccgcatcggcacccgccgcgactggatcctgcgcgactccgccaccaacgaggtgatcggccgcgccacctccaagtgggtgatgatgaaccaggacacccgccgcctgcagcgcgtgaccgacgaggtgcgcgacgagtacctggtgttctgcccccgcgagccccgcctggccttccccgaggagaacaactcctccctgaagaagatccccaagctggaggaccccgcccagtactccatgctggagctgaagccccgccgcgccgacctggacatgaaccagcacgtgaacaacgtgacctacatcggctgggtgctggagtccatcccccaggagatcatcgacacccacgagctgcaggtgatcaccctggactaccgccgcgagtgccagcaggacgacatcgtggactccctgaccacctccgagatccccgacgaccccatctccaagttcaccggcaccaacggctccgccatgtcctccatccagggccacaacgagtcccagttcctgcacatgctgcgcctgtccgagaacggccaggagatcaaccgcggccgcacccagtggcgcaagaagtcctcccgcatggactacaaggaccacgacggcgactacaaggaccacgacatcgactacaaggacgacgacgacaagtgaatcgatggagcgacgagtgtgcgtgcggggctggcgggagtgggacgccctcctcgctcctctctgttctgaacggaacaatcggccaccccgcgctacgcgccacgcatcgagcaacgaagaaaaccccccgatgataggttgcggtggctgccgggatatagatccggccgcacatcaaagggcccctccgccagagaagaagctcctttcccagcagactccttctgctgccaaaacacttctctgtccacagcaacaccaaaggatgaacagatcaacttgcgtctccgcgtagcttcctcggctagcgtgcttgcaacaggtccctgcactattatcttcctgctttcctctgaattatgcggcaggcgagcgctcgctctggcgagcgctccttcgcgccgccctcgctgatcgagtgtacagtcaatgaatggtgagctccgcgcctgcgcgaggacgcagaacaacgctgccgccgtgtcttttgcacgcgcgactccggcgcttcgctggtggcacccccataaagaaaccctcaattctgtttgtggaagacacggtgtacccccacccacccacctgcacctctattattggtattattgacgcgggagtgggcgttgtaccctacaacgtagcttctctagttttcagctggctcccaccattgtaaattcatgctagaatagtgcgtggttatgtgagaggtatagtgtgtctgagcagacggggcgggatgcatgtcgtggtggtgatctttggctcaaggcgtcgtcgacgtgacgtgcccgatcatgagagcaataccgcgctcaaagccgacgcatagcctttactccgcaatccaaacgactgtcgctcgtattttttggatatctattttaaagagcgagcacagcgccgggcatgggcctgaaaggcctcgcggccgtgctcgtggtgggggccgcgagcgcgtggggcatcgcggcagtgcaccaggcgcagacggaggaacgcatggtgcgtgcgcaatataagatacatgtattgttgtcctgcagg

SEQ ID NO: 132 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgcctcccagctgcgcaagcccgccctggaccccctgcgcgccgtgatctccgccgaccagggctccatctcccccgtgaactcctgcacccccgccgaccgcctgcgcgccggccgcctgatggaggacggctactcctacaaggagaagttcatcgtgcgctcctacgaggtgggcatcaacaagaccgccaccgtggagaccatcgccaacctgctgcaggaggtggcctgcaaccacgtgcagaagtgcggcttctccaccgccggcttcgccaccaccctgaccatgcgcaagctgcacctgatctgggtgaccgcccgcatgcacatcgagatctacaagtaccccgcctggtccgacgtggtggagatcgagacctggtgccagtccgagggccgcatcggcacccgccgcgactggatcctgcgcgactccgccaccaacgaggtgatcggccgcgccacctccaagtgggtgatgatgaaccaggacacccgccgcctgcagcgcgtgaccgacgaggtgcgcgacgagtacctggtgttctgcccccgcgagccccgcctggccttccccgaggagaacaactcctccctgaagaagatccccaagctggaggaccccgcccagtactccatgctggagctgaagccccgccgcgccgacctggacatgaaccagcacgtgaacaacgtgacctacatcggctgggtgctggagtccatcccccaggagatcatcgacacccacgagctgcaggtgatcaccctggactaccgccgcgagtgccagcaggacgacatcgtggactccctgaccacctccgagatccccgacgaccccatctccaagttcaccggcaccaacggctccgccatgtcctccatccagggccacaacgagtcccagttcctgcacatgctgcgcctgtccgagaacggccaggagatcaaccgcggccgcacccagtggcgcaagaagtcctcccgcatggactacaaggaccacgacggcgactacaaggaccacgacatcgactacaaggacgacgacgacaagtga

SEQ ID NO: 133 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgcctcccagctgcgcaagcccgccctggaccccctgcgcgccgtgatctccgccgaccagggctccatctcccccgtgaactcctgcacccccgccgaccgcctgcgcgccggccgcctgatggaggacggctactcctacaaggagaagttcatcgtgcgctcctacgaggtgggcatcaacaagaccgccaccgtggagaccatcgccaacctgctgcaggaggtggcctgcaaccacgtgcagaagtgcggcttctccaccgacggcttcgccaccaccctgaccatgcgcaagctgcacctgatctgggtgaccgcccgcatgcacatcgagatctacaagtaccccgcctggtccgacgtggtggagatcgagacctggtgccagtccgagggccgcatcggcacccgccgcgactggatcctgcgcgactccgccaccaacgaggtgatcggccgcgccacctccaagtgggtgatgatgaaccaggacacccgccgcctgcagcgcgtgaccgccgaggtgcgcgacgagtacctggtgttctgcccccgcgagccccgcctggccttccccgaggagaacaactcctccctgaagaagatccccaagctggaggaccccgcccagtactccatgctggagctgaagccccgccgcgccgacctggacatgaaccagcacgtgaacaacgtgacctacatcggctgggtgctggagtccatcccccaggagatcatcgacacccacgagctgcaggtgatcaccctggactaccgccgcgagtgccagcaggacgacatcgtggactccctgaccacctccgagatccccgacgaccccatctccaagttcaccggcaccaacggctccgccatgtcctccatccagggccacaacgagtcccagttcctgcacatgctgcgcctgtccgagaacggccaggagatcaaccgcggccgcacccagtggcgcaagaagtcctcccgcatggactacaaggaccacgacggcgactacaaggaccacgacatcgactacaaggacgacgacgacaagtga

SEQ ID NO: 134 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgcctcccagctgcgcaagcccgccctggaccccctgcgcgccgtgatctccgccgaccagggctccatctcccccgtgaactcctgcacccccgccgaccgcctgcgcgccggccgcctgatggaggacggctactcctacaaggagaagttcatcgtgcgctcctacgaggtgggcatcaacaagaccgccaccgtggagaccatcgccaacctgctgcaggaggtggcctgcaaccacgtgcagaagtgcggcttctccaccgccggcttcgccaccaccctgaccatgcgcaagctgcacctgatctgggtgaccgcccgcatgcacatcgagatctacaagtaccccgcctggtccgacgtggtggagatcgagacctggtgccagtccgagggccgcatcggcacccgccgcgactggatcctgcgcgactccgccaccaacgaggtgatcggccgcgccacctccaagtgggtgatgatgaaccaggacacccgccgcctgcagcgcgtgaccgccgaggtgcgcgacgagtacctggtgttctgcccccgcgagccccgcctggccttccccgaggagaacaactcctccctgaagaagatccccaagctggaggaccccgcccagtactccatgctggagctgaagccccgccgcgccgacctggacatgaaccagcacgtgaacaacgtgacctacatcggctgggtgctggagtccatcccccaggagatcatcgacacccacgagctgcaggtgatcaccctggactaccgccgcgagtgccagcaggacgacatcgtggactccctgaccacctccgagatccccgacgaccccatctccaagttcaccggcaccaacggctccgccatgtcctccatccagggccacaacgagtcccagttcctgcacatgctgcgcctgtccgagaacggccaggagatcaaccgcggccgcacccagtggcgcaagaagtcctcccgcatggactacaaggaccacgacggcgactacaaggaccacgacatcgactacaaggacgacgacgacaagtga

SEQ ID NO: 135 - DNA, 5451 bp, Artificial Sequence
  Description of Artificial Sequence Synthetic polynucleotide
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgatcgagcaggacggcctccacgccggctcccccgccgcctgggtggagcgcctgttcggctacgactgggcccagcagaccatcggctgctccgacgccgccgtgttccgcctgtccgcccagggccgccccgtgctgttcgtgaagaccgacctgtccggcgccctgaacgagctgcaggacgaggccgcccgcctgtcctggctggccaccaccggcgtgccctgcgccgccgtgctggacgtggtgaccgaggccggccgcgactggctgctgctgggcgaggtgcccggccaggacctgctgtcctcccacctggcccccgccgagaaggtgtccatcatggccgacgccatgcgccgcctgcacaccctggaccccgccacctgccccttcgaccaccaggccaagcaccgcatcgagcgcgcccgcacccgcatggaggccggcctggtggaccaggacgacctggacgaggagcaccagggcctggcccccgccgagctgttcgcccgcctgaaggcccgcatgcccgacggcgaggacctggtggtgacccacggcgacgcctgcctgcccaacatcatggtggagaacggccgcttctccggcttcatcgactgcggccgcctgggcgtggccgaccgctaccaggacatcgccctggccacccgcgacatcgccgaggagctgggcggcgagtgggccgaccgcttcctggtgctgtacggcatcgccgcccccgactcccagcgcatcgccttctaccgcctgctggacgagttcttctgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgtgaaaactcgctcgaccgcccgcgtcccgcaggcagcgatgacgtgtgcgtgacctgggtgtttcgtcgaaaggccagcaaccccaaatcgcaggcgatccggagattgggatctgatccgagcttggaccagatcccccacgatgcggcacgggaactgcatcgactcggcgcggaacccagctttcgtaaatgccagattggtgtccgataccttgatttgccatcagcgaaacaagacttcagcagcgagcgtatttggcgggcgtgctaccagggttgcatacattgcccatttctgtctggaccgctttaccggcgcagagggtgagttgatggggttggcaggcatcgaaacgcgcgtgcatggtgtgtgtgtctgttttcggctgcacaatttcaatagtcggatgggcgacggtagaattgggtgttgcgctcgcgtgcatgcctcgccccgtcgggtgtcatgaccgggactggaatcccccctcgcgaccctcctgctaacgctcccgactctcccgcccgcgcgcaggatagactctagttcaaccaatcgacaactagtatggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtgaatcgatgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgagctccagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 136 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 137 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFATTPTMRKLRLIWVTARMHIEIYKYPAWSDVVEIESWGQGEGKIGTRRDWILRDYATGQVIGRATSKWVMMNQDTRRLQKVDADVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 138 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFVTTPTMRKLRLIWVTARMHIEIYKYPAWSDVVEIESWGQGEGKIGTRRDWILRDYATGQVIGRATSKWVMMNQDTRRLQKVDADVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 139 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVACNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 140 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVTCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 141 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVVCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 142 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTAGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 143 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANFLQEVGCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 144 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANKLQEVGCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 145 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANSLQEVGCNHAQSVGYSTGGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 146 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTVGFSTTPTMRKLRLIWVTARMHIEIYKYPAWSDVVEIESWGQGEGKIGTRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 147 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFSTTPTMRKLRLIWVTARMHIEIYKYPAWSDVVEIESWGQGEGKIGFRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 148 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFSTTPTMRKLRLIWVTARMHIEIYKYPAWSDVVEIESWGQGEGKIGARRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 149 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFSTTPTMRKLRLIWVTARMHIEIYKYPAWSDVVEIESWGQGEGKIGKRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 150 - protein, 391 aa, Artificial Sequence
  Description of Artificial Sequence Synthetic polypeptide
MATASTFSAFNARCGDLRRSAGSGPRRPARPLPVRGRAIPPRIIVVSSSSSKVNPLKTEAVVSSGLADRLRLGSLTEDGLSYKEKFIVRCYEVGINKTATVETIANLLQEVGCNHAQSVGYSTGGFSTTPTMRKLRLIWVTARMHIEIYKYPAWSDVVEIESWGQGEGKIGVRRDWILRDYATGQVIGRATSKWVMMNQDTRRLQKVDVDVRDEYLVHCPRELRLAFPEENNSSLKKISKLEDPSQYSKLGLVPRRADLDMNQHVNNVTYIGWVLESMPQEIIDTHELQTITLDYRRECQHDDVVDSLTSPEPSEDAEAVFNHNGTNGSANVSANDHGCRNFLHLLRLSGNGLEINRGRTEWRKKPTRMDYKDHDGDYKDHDIDYKDDDDK

SEQ ID NO: 151 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttcgccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgcggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 152 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttcgtcaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgcggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 153 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtggcgtgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 154 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgacgtgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 155 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtggtgtgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 156 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccgccggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 157 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacttcctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 158 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacaagctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 159 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaactcgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 160 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccgtcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcacccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 161 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcttccgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 162 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcgcgcgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 163 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcaagcgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 164 - DNA, 1176 bp, Artificial Sequence
  Description of Artificial Sequence Synthetic polynucleotide
atggccaccgcatccactttctcggcgttcaatgcccgctgcggcgacctgcgtcgctcggcgggctccgggccccggcgcccagcgaggcccctccccgtgcgcgggcgcgccatccccccccgcatcatcgtggtgtcctcctcctcctccaaggtgaaccccctgaagaccgaggccgtggtgtcctccggcctggccgaccgcctgcgcctgggctccctgaccgaggacggcctgtcctacaaggagaagttcatcgtgcgctgctacgaggtgggcatcaacaagaccgccaccgtggagaccatcgccaacctgctgcaggaggtgggctgcaaccacgcccagtccgtgggctactccaccggcggcttctccaccacccccaccatgcgcaagctgcgcctgatctgggtgaccgcccgcatgcacatcgagatctacaagtaccccgcctggtccgacgtggtggagatcgagtcctggggccagggcgagggcaagatcggcgtgcgccgcgactggatcctgcgcgactacgccaccggccaggtgatcggccgcgccacctccaagtgggtgatgatgaaccaggacacccgccgcctgcagaaggtggacgtggacgtgcgcgacgagtacctggtgcactgcccccgcgagctgcgcctggccttccccgaggagaacaactcctccctgaagaagatctccaagctggaggacccctcccagtactccaagctgggcctggtgccccgccgcgccgacctggacatgaaccagcacgtgaacaacgtgacctacatcggctgggtgctggagtccatgccccaggagatcatcgacacccacgagctgcagaccatcaccctggactaccgccgcgagtgccagcacgacgacgtggtggactccctgacctcccccgagccctccgaggacgccgaggccgtgttcaaccacaacggcaccaacggctccgccaacgtgtccgccaacgaccacggctgccgcaacttcctgcacctgctgcgcctgtccggcaacggcctggagatcaaccgcggccgcaccgagtggcgcaagaagcccacccgcatggactacaaggaccacgacggcgactacaaggaccacgacatcgactacaaggacgacgacgacaagtga

SEQ ID NO: 165 - protein, 391 aa, Artificial Sequence
  Description of Artificial Sequence Synthetic polypeptide
MATASTFSAFNARCGDLRRSAGSGPRRPARPLPVRGRASQLRKPALDPLRAVISADQGSISPVNSCTPADRLRAGRLMEDGYSYKEKFIVRSYEVGINKTATVETIANLLQEVACNHVQKCGFSTDGFATTLTMRKLHLIWVTARMHIEIYKYPAWSDVVEIETWCQSEGRIGTRRDWILRDSATNEVIGRATSKWVMMNQDTRRLQRVTDEVRDEYLVFCPREPRLAFPEENNSSLKKIPKLEDPAQYSMLELKPRRADLDMNQHVNNVTYIGWVLESIPQEIIDTHELQVITLDYRRECQQDDIVDSLTTSEIPDDPISKFTGTNGSAMSSIQGHNESQFLHMLRLSENGQEINRGRTQWRKKSSRMDYKDHDGDYKDHDIDYKDDDDK

SEQ ID NO: 166 - protein, 391 aa, Artificial Sequence
  Description of Artificial Sequence Synthetic polypeptide
MATASTFSAFNARCGDLRRSAGSGPRRPARPLPVRGRASQLRKPALDPLRAVISADQGSISPVNSCTPADRLRAGRLMEDGYSYKEKFIVRSYEVGINKTATVETIANLLQEVACNHVQKCGFSTAGFATTLTMRKLHLIWVTARMHIEIYKYPAWSDVVEIETWCQSEGRIGTRRDWILRDSATNEVIGRATSKWVMMNQDTRRLQRVTDEVRDEYLVFCPREPRLAFPEENNSSLKKIPKLEDPAQYSMLELKPRRADLDMNQHVNNVTYIGWVLESIPQEIIDTHELQVITLDYRRECQQDDIVDSLTTSEIPDDPISKFTGTNGSAMSSIQGHNESQFLHMLRLSENGQEINRGRTQWRKKSSRMDYKDHDGDYKDHDIDYKDDDDK

SEQ ID NO: 167 - protein, 391 aa, Artificial Sequence
  Description of Artificial Sequence Synthetic polypeptide
MATASTFSAFNARCGDLRRSAGSGPRRPARPLPVRGRASQLRKPALDPLRAVISADQGSISPVNSCTPADRLRAGRLMEDGYSYKEKFIVRSYEVGINKTATVETIANLLQEVACNHVQKCGFSTDGFATTLTMRKLHLIWVTARMHIEIYKYPAWSDVVEIETWCQSEGRIGTRRDWILRDSATNEVIGRATSKWVMMNQDTRRLQRVTAEVRDEYLVFCPREPRLAFPEENNSSLKKIPKLEDPAQYSMLELKPRRADLDMNQHVNNVTYIGWVLESIPQEIIDTHELQVITLDYRRECQQDDIVDSLTTSEIPDDPISKFTGTNGSAMSSIQGHNESQFLHMLRLSENGQEINRGRTQWRKKSSRMDYKDHDGDYKDHDIDYKDDDDK

SEQ ID NO: 168 - protein, 391 aa, Artificial Sequence
  Description of Artificial Sequence Synthetic polypeptide
MATASTFSAFNARCGDLRRSAGSGPRRPARPLPVRGRASQLRKPALDPLRAVISADQGSISPVNSCTPADRLRAGRLMEDGYSYKEKFIVRSYEVGINKTATVETIANLLQEVACNHVQKCGFSTAGFATTLTMRKLHLIWVTARMHIEIYKYPAWSDVVEIETWCQSEGRIGTRRDWILRDSATNEVIGRATSKWVMMNQDTRRLQRVTAEVRDEYLVFCPREPRLAFPEENNSSLKKIPKLEDPAQYSMLELKPRRADLDMNQHVNNVTYIGWVLESIPQEIIDTHELQVITLDYRRECQQDDIVDSLTTSEIPDDPISKFTGTNGSAMSSIQGHNESQFLHMLRLSENGQEINRGRTQWRKKSSRMDYKDHDGDYKDHDIDYKDDDDK

SEQ ID NO: 169 - protein, 385 aa, Artificial Sequence; identical to SEQ ID NO: 23
  Description of Artificial Sequence Synthetic polypeptide

SEQ ID NO: 170 - protein, 384 aa, Artificial Sequence; identical to SEQ ID NO: 24
  Description of Artificial Sequence Synthetic polypeptide

SEQ ID NO: 171 - protein, 391 aa, Artificial Sequence; identical to SEQ ID NO: 28
  Description of Artificial Sequence Synthetic polypeptide

SEQ ID NO: 172 - protein, 391 aa, Artificial Sequence; identical to SEQ ID NO: 29
  Description of Artificial Sequence Synthetic polypeptide

SEQ ID NO: 173 - protein, 369 aa, Artificial Sequence; identical to SEQ ID NO: 34
  Description of Artificial Sequence Synthetic polypeptide

SEQ ID NO: 174 - protein, 387 aa, Artificial Sequence; identical to SEQ ID NO: 76
  Description of Artificial Sequence Synthetic polypeptide

SEQ ID NO: 175 - protein, 387 aa, Artificial Sequence; identical to SEQ ID NO: 75
  Description of Artificial Sequence Synthetic polypeptide

SEQ ID NO: 176 - protein, 387 aa, Artificial Sequence
  Description of Artificial Sequence Synthetic polypeptide
MAIAAAAVIVPLGLLFFVSGLIVNLVQAVCFVLIRPLSKNTYRRINRVVAELLWLELVWLIDWWAGVKIKVFTDHETLSLMGKEHALVICNHKSDIDWLVGWVLAQRSGCLGSTLAVMKKSSKFLPVIGWSMWFSEYLPESDDAVAQWCRDIFVEKDALLDKHNAEDTFSGQELQDTGRPIKSLLVVISWAVLVIFGAVKFLQWSSLLSSWKGLAFSGVGLGIITLLMHILILFSQSERSTPAKVAPAKPKKDGESSKTEIEKENVPGALLGQGREHPEVRPEPPEGLPPALLAGPVRGGHPLHPRQAAGRPAVRHLLRPARAPQRADPPHQGLRVLRVPHALLRARHLRRDRGHPQDLPPPHHAAHVQGPVLRAARAPEAPPDEGP

SEQ ID NO: 177 - protein, 384 aa, Artificial Sequence
  Description of Artificial Sequence Synthetic polypeptide
MAIAAAAVIFLFGLIFFASGLIINLFQALCFVLIRPLSKNAYRRINRVFAELLLSELLCLFDWWAGAKLKLFTDPETFRLMGKEHALVIINHMTELDWMVGWVMGQHFGCLGSIISVAKKSTKFLPVLGWSMWFSEYLYLERSWAKDKSTLKSHIERLIDYPLPFWLVIFVEGTRFTRTKLLAAQQYAVSSGLPVPRNVLIPRTKGFVSCVSHMRSFVPAVYDVTVAFPKTSPPPTLLNLFEGQSIMLHVHIKRHAMKDLPESDDAVAEWCRDKFVEKDALLDKHNAEDTFSGQEVCHSGSRQLKSLLVVISWVVVTTFGALKFLQWSSWKGKAFSAIGLGIVTLLMHVLILSSQAERSNPAEVAQAKLKTGLSISKKVTDKEN

SEQ ID NO: 178 - protein, 380 aa, Artificial Sequence
  Description of Artificial Sequence Synthetic polypeptide
MAIPAAVAVIPIGLLFIISGLIVNLIQAVVYVLIRPLSKNLHRKINKPIAELLWLELIWLVDWWAGIKVEVYADSQTLELMGKEHALLICNHRSDIDWLVGWVLAQRARCLGSALAIMKKSAKFLPVIGWSMWFSDYIFLDRTWAKDEKTLKSGFERLADFPMPFWLALFVEGTRFTKAKLLAAQEYAASRGLPVPQNVLIPRTKGFVTAVTHMRSYVPAIYDCTVDISKAHPAPSILRLIRGQSSVVKVQITRHSMQELPETADGISQWCMDLFVTKDGFLEKYHSKDIFGSLPVQNIGRPVKSLIVVLCWYCLMAFGLFKFFMWSSLLSSWEGILSLGLILLAVAIVMQILIQSTESERSTPVKSIQKDPSKETLLQN

SEQ ID NO: 179 - protein, 382 aa, Artificial Sequence; identical to SEQ ID NO: 85
  Description of Artificial Sequence Synthetic polypeptide

SEQ ID NO: 180 - protein, 380 aa, Artificial Sequence
  Description of Artificial Sequence Synthetic polypeptide
MAIPAAVAVIPIGLLFIISGLIVNLIQAVVYVLIRPLSKNLYRKINKPIAELLWLELIWLVDWWAGIKVEVYADSETLESMGKEHALLICNHRSDIDWLVGWVLAQRARCLGSALAIMKKSAKFLPVIGWSMWFSDYIFLDRTWEKDEKTLKSGFERLADFPMPFWLALFVEGTRFTKAKLLAAQEFAASRGLPVPQNVLIPRTKGFVTAVTHMRSYVPAIYDCTVDISKAHPAPSILRLIRGQSSVVKVQITRHSMQELPETPDGISQWCMDLFVTKDAFLEKYHSKDIFGSLPVHDIGRPVKSLIVVLCWYSLMAFGFYKFFMWSSLLSSWEGILSLGLVLIVIAIVMQILIQSSESERSTPVKSVQKDPSKETLLQN

SEQ ID NO: 181 - protein, 374 aa, Artificial Sequence
  Description of Artificial Sequence Synthetic polypeptide
MATGGSLKPSSSDLDLDHPNIEDYLPSGSSINEPAGKLRLRDLLDISPTLTEAAGAIVDDSFTRCFKSIPREPWNWNLYLFPLWCIGVLIRYFILFPGRVIVLTMGWITVISSFIAVRVLLKGHDALQIKLERLIVQLLCSSFVASWTGVVKYHGPRPSIRPKQVYVANHTSMIDFFILDQMTVFSVIMQKHPGWVGLLQSTLLESVGCIWFDRAEAKDRGIVAKKLWDHVHGEGNNPLLIFPEGTCVNNNYSVMFKKGAFELGCTVCPVAIKYNKIFVDAFWNSKKQSFTRHLLQLMTSWAVVCDVWYLEPQTLKPGETPIEFAERVRDIISARAGLKKVPWDGYLKYSRPSPKHRERKQQTFAESVLQRLEE

SEQ ID NO: 182 - protein, 375 aa, Artificial Sequence
  Description of Artificial Sequence Synthetic polypeptide
MATAGSLKPSRSELDFDRPNIEDYLPSGSSIIEPAGKLRLRDLLDISPTLTEAAGAIVDDSFTRCFKSNPPEPWNWNIYLFPLWCFGVLIRYLILFPARVIVLTIGWIIFLSSFIPVHLLLKGHDALRIKLERLLVELICSFFVASWTGVVKYHGPRPSIRPKQVYVANHTSMIDFFILDQMTVFSVIMQKHPGWVGLLQSTLLESVGCIWFDRAEAKDRGIVAKKLWDHVHGEGNNPLLIFPEGTCVNNNYSVMFKKGAFELGCTVCPVAIKYNKIFVDAFWNSKKQSFTRHLLQLMTSWAVVCDVWYLEPQTLKPGETPIEFAERVRDIISVRAGLKKVPWDGYLKYSRPSPKHTERKQQNFAESVLQRLEKK

SEQ ID NO: 183 - protein, 375 aa, Artificial Sequence
  Description of Artificial Sequence Synthetic polypeptide
MATGGRLKPSSSELDLDRANTEDYLPSGSSINEPVGKLRLRDLLDISPTLTEAAGAIVDDSFTRCFKSIPPEPWNWNIYLFPLWCFGVLIRYFILFPARVIVLTIGWITVISSFTAVRFLLKGHNALQIKLERLIVQLLCSSFVASWTGVVKYHGPRPSIRPKQVYVANHTSMIDFLILDQMTVFSVIMQKHPGWVGLLQSTLLESVGCIWFNRAEAKDREIVAKKLWDHVHGEGNNPLLIFPEGTCVNNHYSVMFKKGAFELGCTVCPVAIKYNKIFVDAFWNSRKQSFTMHLLQLMTSWAVVCDVWYLEPQTLKPGETAIEFAERVRDIISVRAGLKKVPWDGYLKYSRPSPKHRESKQQSFAESVLRRLEEK

SEQ ID NO: 184 - protein, 375 aa, Artificial Sequence
  Description of Artificial Sequence Synthetic polypeptide
MATGGRLKPSSSELDLDRANTEDYLPSGSSINEPVGKLRLRDLLDISPTLTEAAGAIVDDSFTRCFKSIPPEPWNWNIYLFPLWCFGVLIRYFILFPARVIVLTIGWITVISSFTAVRFLLKGHNALQIKLERLIVQLLCSSFVASWTGVVKYHGPRPSIRPKQVYVANHTSMIDFLILDQMTVFSVIMQKHPGWVGLLQSTLLESVGCIWFNRAEAKDREIVAKKLWDHVHGEGNNPLLIFPEGTCVNNHYSVMFKKGAFELGCTVCPVAIKYNKIFVDAFWNSKKHSFTRHLLQLMTSWAVVCDVWYLEPQTLKPGETPIEFAERVRDIISVRADLKKVPWDGYLKYSRPSPKHRERKQQKFAESVLRRLEEK

SEQ ID NO: 185 - protein, 377 aa, Artificial Sequence
  Description of Artificial Sequence Synthetic polypeptide
MATAGRLKPSSSELELDLDRPNIEDYLPSGSSINEPAGKLRLRDLLDISPMLTEAAGAIVDDSFTRCFKSIPPEPWNWNIYLFPLWCFGVLIRYLILFPARVIVLTVGWITVISSFITVRFLLKGHDSLRIKLERLIVQLFCSSFVASWTGVVKYHGPRPSIRPQQVYVANHTSMIDFIILNQMTVFSAIMQKHPGWVGLIQSTILESVGCIWFNRAEAKDREIVAKKLLDHVHGEGNNPLLIFPEGTCVNNHYSVMFKKGAFELGCTVCPVAIKYNKIFVDAFWNSKKQSFTMHLLQLMTSWAVVCDVWYLEPQTLKPGETPIEFAERVRDIISVRAGLKKVPWDGYLKYSRPSPKHRERKQQSFAESVLRRLEKR

SEQ ID NO: 186 - protein, 385 aa, Artificial Sequence
  Description of Artificial Sequence Synthetic polypeptide
MATAGRLKPSSSELELDLDRPNIEDYLPSGSSINEPAGKLRLRDLLDISPMLTEAAGAIVDDSFTRCFKSIPPEPWNWNIYLFPLWCFGVLIRYLILFPARVIVLTVGWITVISSFITVRFLLKGHDSLRIKLERLIVQLFCSSFVASWTGVVKYHGPRPSIRPQQVYVANHTSMIDFIILNQMTVFSAIMQKHPGWVGLIQSTILESVGCIWFNRAEAKDREIVAKKLLDHVHGEGNNPLLIFPEGTCVNNHYSVMFKKGAFELGCTVCPVAIKYNKIFVDAFWNSKKLSFTMHLLQLMTSWAVVCDVWYLEPQTLKPGETPIEFAERVRDIISVRAGLKKVPWDGYLKYSRPSPKHRERKQQTFAESVLRRLEEKGNVVPTVN

SEQ ID NO: 187 - protein, 524 aa, Artificial Sequence
  Description of Artificial Sequence Synthetic polypeptide
MAIADGGIIGAAGSISALTADTDPPSLRRRNVPAGQASAVSAFSTESMAKHLCDPSREPSPSPKSSDDGKDPDIGSVDSLNEKPSSPAAGKGRLQHDLRFTYRASSPAHRKVKESPLSSSNIFKQSHAGLFNLCVVVLVAVNSRLIIENLMKYGLLIKTGFWFSSRSLRDWPLFMCCLSLPIFPLAAFLVEKLAQKNRLQEPTVVCCHVLITSVSILYPVLVILRCDSAVLSGVALMLFACIVWLKLVSYAHSNYDMRYVAKSLDKGEPVVDSVIADHPYRVDYKDLVYFMVAPTLCYQLSYPLTPCVRKSWIARQVMKLVLFTGVMGFIVEQYINPIVQNSKHPLKGDLLYAIERVLKLSVPNLYVWLCMFYCFFHLWLNILAELICFGDREFYKDWWNAKTVEEYWRMWNMPVHKWMVRHIYFPCLRNGIPRGVAVLIAFLVSAVFHELCIAVPCHVFKLWAFIGIMFQVPLVLVSNCLQKKFQSSMAGNMFFWFIFCIFGQPMCVLLYYHDLMNRKGSRID

SEQ ID NO: 188 - protein, 528 aa, Artificial Sequence
  Description of Artificial Sequence Synthetic polypeptide
MAIADGGSAGAAGSISGSDPSPSTAPSLRRRNASAGQAFSTESMARDLCDPSREPSLSPKSSDDGKDPADDIGAADSVDSGGVKDEKPSSQAAAKARLEHDLRFTYRASSPAHRKVKESPLSSSNIFKQSHAGLFNLCVVVLVAVNSRLIIENLMKYGLLIKTGFWFSSRSLRDWPLFMCCLSLPIFPLAAFLVEKLAQKNRLQEPTVVCCHVIITSVSILYPVLVILRCDSAVLSGVALMLFACIVWLKLVSYAHANYDMRSVAKSLDKGETVADSVIVDHPYRVDYKDLVYFMVAPTLCYQLSYPLTPYVRKSWVARQVMKLVLFTGVMGFIVEQYINPIVQNSKHPLKGDLLYAIERVLKLSVPNLYVWLCMFYCFFHLWLNILAELTCFGDREFYKDWWNAKTVEEYWRMWNMPVHKWMVRHIYFPCLRNGIPRGVAVLIAFLVSAVFHELCIAVPCHVFKLWAFIGIMFQVPLVLVSNCLQKKFQSSMAGNMFFWFIFCIFGQPMCVLLYYHDLMNRKGSRID

SEQ ID NO: 189 - protein, 463 aa, Artificial Sequence
  Description of Artificial Sequence Synthetic polypeptide
MGLVSVAAAIGVSVPVARFLLCFLATIPVSFLWRLVPGRLPKHLYSAASGAILSYLSFGASSNLHFIVPMTLGYLSMLFFRPFSGLLTFFLGFGYLIGCHVYYMSGDAWKEGGIDATGALMVLTLKVISCSMNYNDGLLKEEGLRESQKKNRLTKMPSLIEYFGYCLCCGSHFAGPVYEMKDYLEWTEGKGIWSRSQKEPKPSPFGGALRAIIQAAVCMAMYLYLVPHHPLTRFTEPVYYEWGFFRRLSYQYMAALTARWKYYFIWSISEASLIISGLGFSGWTESSPPKPRWDRAKNVDIIGVEFAKSSVQLPLVWNIQVSIWLRHYVYDRLVQNGKRPGFFQLLATQTVSAVWHGLYPGYIIFFVQSALMIAGSRVIYRWQQAVPPKMGLVKNIFVFFNFAYTLLVLNYSAVGFMVLSMHETLASYGSVYYIGTILPITLILLSYVIKPGKPARSKAHKEQ

SEQ ID NO: 190 - protein, 463 aa, Artificial Sequence
  Description of Artificial Sequence Synthetic polypeptide
MELGSVAAAIGVSVPVARFLLCFLATIPVSFLWRLVPGRLPKHLYSAASGAILSYLSFGPSSNLHFIVPMTLGYLSMLFFRPFSGLLTFFLGFGYLIGCHVYYMSGDAWKEGGIDATGALMVLTLKVISCSINYNDGLLKEEGLRESQKKNRLTKMPSLIEYIGYCLCCGSHFAGPVYEMKDYLEWTEGKGVWSHSEKEPKPSPFGGALRAIIQAAVCMAMYMYLVPHHPLSRFTEPVYYEWGFFRRLSYQYMAGLTARWKYYFIWSISEASLIISGLGFSGWTESSPPKPRWDRAKNVDIIGVEFAKSSVQLPLVWNIQVSTWLRHYVYDRLVQNGKRPGFFQLLATQTVSAIWHGLYPGYIIFFVQSALMIAGSRVIYRWQQAVPPKMGLVKNIFVFFNFAYTLLVLNYSAVGFMVLSMHETLASYGSVYYIGTILPITLILLSYVIKPGKPARSKAHKEQ

SEQ ID NO: 191 - protein, 465 aa, Artificial Sequence
  Description of Artificial Sequence Synthetic polypeptide
MELEIGSVAAAIGVSVPVARFLLCFLATIPVSFLCRLLPARLPKHLYSAASGAILSYLSFGPSSNLHFIVPMSLGYLSMLFFRPFSGLLTFFLGFGYLIGCHVYYMSGDAWKEGGIDATGALMVLTLKVISCSINYNDGLLKEEGLRESQKKNRLTKMPSLIEYFGYCLCCGSHFAGPVYEMKDYLEWTEGKGIWSRSEKDPKPSPFGGALRAIIQAAVCMAMHMYLVPHHPLTRFTEPVYYEWGFFRRLSYQYMAAQTARWKYYFIWSISEASLIISGLGFSGWTESSPPKPRWDKAKNVDIIGVEFAKSSVQLPLVWNIQVSTWLRHYVYDRLVQNGKRPGFFQLLATQTVSAVWHGLYPGYIIFFVQSALMIAGSRVIYRWQQAVPQKMGLVKNIFVFFNFAYTLLVLNYSAVGFMVLSMHETLASYGSVYYIGTILPITLILLSYVIKPGKPTRSKVHKEQ

SEQ ID NO: 192 - protein, 465 aa, Artificial Sequence
  Description of Artificial Sequence Synthetic polypeptide
MELEMEPLAAAIGVSVAVFRFLVCFIATIPVSFICRLVPGGLPRHLFSAASGAVLSYLSFGFSSNLHFLVPMTLGYLSMILFRRFCGILTFFLGFGYLIGCHVYYMSGDAWKEGGIDATGALMVLTLKVISCSINYNDGLLKEEGLRESQKKNRLIRLPSLIEYFGYCLCCGSHFAGPVYEMKDYLDWTEGKGIWSHSEKGPKPSPLRAALRAIIQAGFCMAMYLYLVPHYPLTRFTDPVYYEWGILRRLSYQYMASFTARWKYYFIWSISEASLIISGLGFSGWTESSPPKPRWDRAKNVDILGVELAKSSVQIPLVWNIQVSTWLRHYVYDRLVQNGKRPGFLQLLATQTVSAIWHGVYPGYLIFFVQSALMIAGSRAIYRWQQAVPPKMSLVKNTLVFFNFAYTLLVLNYSAVGFMVLSMHETLASYGSVYYVGTILPVTLILLGYVIKPGKSPRSKASKEQ

SEQ ID NO: 193 - protein, 245 aa, Artificial Sequence
  Description of Artificial Sequence Synthetic polypeptide
MNFDFLSNIPWFGAKASDNAGSSFGSATIVIQQPPPVSRGFDIRHWGWPWSVLSVLPWGKPGCDELRAPPTTINRRLKRNATSMHSSAVRGNAEAARVRFRPYVSKVPWHTGFRGLLSQLFPRYGHYCGPNWSSGKNGGSPVWDQRPIDWLDYCCYCHDIGYDTHDQAKLLEADLAFLECLERPSYPTKGDAHVAHMYKTMCVTGLRNVLIPYRTQLLRLNSRQPLIDFGWLSNAAWKGWNAQKS

SEQ ID NO: 194 - protein, 236 aa, Artificial Sequence
  Description of Artificial Sequence Synthetic polypeptide
MNLDFLSKIPWFEAKASENPGLNLGSTTIVIKQPRQGFDIRHWGWPWSVLTWGNRVTDEVHAPPTTINRRLKRNATGPAVQGDTEAARLRFRPYVSKVPWHTGFRGLLSQLFPRYGHYCGPNWSSGKNGGSPVWDQRPIDWLDYCCYCHDIGYDTHDQAKLLEADLAFLECLERPSYPTTGDAHVAHMYKTMCVTGLRNVLIPYRTQLLRLNFRQPLIDFGWLSNAAWKGWSAQKT

SEQ ID NO: 195 - protein, 158 aa, Artificial Sequence
  Description of Artificial Sequence Synthetic polypeptide
MVHLPHTLKLGLVIAISISGLCFSSTPARALNVGIQAAGVTVSVGKGCSRKCESDFCKVPPFLRYGKYCGLMYSGCPGEKPCDGLDACCMKHDACVQAKNNDYLSQECSQNLLNCMASFRMSGGKQFKGSTCQVDEVVDVLTVVMEAALLAGRYLHKP

SEQ ID NO: 196 - protein, 158 aa, Artificial Sequence
  Description of Artificial Sequence Synthetic polypeptide
MVHLPHTLKLGLVIAISISGLCLSSTPARALNVGIQAAGVTVSVGKGCSRKCESDFCKVPPFLRYGKYCGLMYSGCPGEKPCDGLDACCMKHDACVQAKNDDYLSQECSQNLLNCMASFRMSGGKQFKGSTCQVDEVVDVLTVVMEAALLAGRYLHKP

SEQ ID NO: 197 - DNA, 6405 bp, Artificial Sequence
  Description of Artificial Sequence Synthetic polynucleotide
agcggaagagcgcccaatgtttaaacccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgcctgctcaagcgggcgctcaacatgcagagcgtcagcgagacgggctgtggcgatcgcgagacggacgaggccgcctctgccctgtttgaactgagcgtcagcgctggctaaggggagggagactcatccccaggctcgcgccagggctctgatcccgtctcgggcggtgatcggcgcgcatgactacgacccaacgacgtacgagactgatgtcggtcccgacgaggagcgccgcgaggcactcccgggccaccgaccatgtttacaccgaccgaaagcactcgctcgtatccattccgtgcgcccgcacatgcatcatcttttggtaccgacttcggtcttgttttacccctacgacctgccttccaaggtgtgagcaactcgcccggacatgaccgagggtgatcatccggatccccaggccccagcagcccctgccagaatggctcgcgctttccagcctgcaggcccgtctcccaggtcgacgcaacctacatgaccaccccaatctgtcccagaccccaaacaccctccttccctgcttctctgtgatcgctgatcagcaacaactagtaacaatggccaccgcctccaccttctccgccttcaacgcccgctgcggcgacctgcgccgctccgccggctccggcccccgccgccccgcccgccccctgcccgtgcgcgccgccatcaacgactccgcccaccccaaggccaacggctccgccgtgagcctgaagagcggcagcctgaacacccaggaggacacctcctccagcccccccccccgcaccttcctgcaccagctgcccgactggagccgcctgctgaccgccatcaccaccgtgttcgtgaagtccaagcgccccgacatgcacgaccgcaagtccaagcgccccgacatgctggtggacagcttcggcctggagtccaccgtgcaggacggcctggtgttccgccagtccttctccatccgctcctacgagatcggcaccgaccgcaccgccagcatcgagaccctgatgaaccacctgcaggagacctccctgaaccactgcaagagcaccggcatcctgctggacggcttcggccgcaccctggagatgtgcaagcgcgacctgatctgggtggtgatcaagatgcagatcaaggtgaaccgctaccccgcctggggcgacaccgtggagatcaacacccgcttcagccgcctgggcaagatcggcatgggccgcgactggctgatctccgactgcaacaccggcgagatcctggtgcgcgccaccagcgcctacgccatgatgaaccagaagacccgccgcctgtccaagctgccctacgaggtgcaccaggagatcgtgcccctgttcgtggacagccccgtgatcgaggactccgacctgaaggtgcacaagttcaaggtgaagaccggcgacagcatccagaagggcctgacccccggctggaacgacctggacgtgaaccagcacgtgtccaacgtgaagtacatcggctggatcctggagagcatgcccaccgaggtgctggagacccaggagctgtgctccctggccctggagtaccgccgcgagtgcggccgcgactccgtgctggagagcgtgaccgccatggaccccagcaaggtgggcgtgcgctcccagtaccagcacctgctgcgcctggaggacggcaccgccatcgtgaacggcgccaccgagtggcgccccaagaacgccggcgccaacggcgccatctccaccggcaagaccagcaacggcaactccgtgtccatggactacaaggaccacgacggcgactacaaggaccacgacatcgactacaaggacgacgacgacaagtgactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatttacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgagctccagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgactgaagagcgttt

SEQ ID NO: 198 - DNA, 6922 bp, Artificial Sequence
  Description of Artificial Sequence Synthetic polynucleotide
agcggaagagcgcccaatgtttaaacccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatatcaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacaattgacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaggatcccgcgtctcgaacagagcgcgcagaggaacgctgaaggtctcgcctctgtcgcacctcagcgcggcatacaccacaataaccacctgacgaatgcgcttggttcttcgtccattagcgaagcgtccggttcacacacgtgccacgttggcgaggtggcaggtgacaatgatcggtggagctgatggtcgaaacgttcacagcctagggatatcgaattcggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccactagtatggccaccgcctccaccttctccgccttcaacgcccgctgcggcgacctgcgccgctccgccggctccggcccccgccgccccgcccgccccctgcccgtgcgcgccgccatcaactcccgcgcccaccccaaggccaacggctccgccgtgtccctgaagtccggctccctgaacacccaggaggacacctcctcctccccccccccccgcaccttcctgcaccagctgcccgactggtcccgcctgctgaccgccatcaccaccgtgttcgtgaagtccaagcgccccgacatgcacgaccgcaagtccaagcgccccgacatgctgatggactccttcggcctggagtccatcgtgcaggagggcctggagttccgccagtccttctccatccgctcctacgagatcggcaccgaccgcaccgcctccatcgagaccctgatgaactacctgcaggagacctccctgaaccactgcaagtccaccggcatcctgctggacggcttcggccgcacccccgagatgtgcaagcgcgacctgatctgggtggtgaccaagatgaagatcaaggtgaaccgctaccccgcctggggcgacaccgtggagatcaacacctggttctcccgcctgggcaagatcggcaagggccgcgactggctgatctccgactgcaacaccggcgagatcctgatccgcgccacctccgcctacgccaccatgaaccagaagacccgccgcctgtccaagctgccctacgaggtgcaccaggagatcgcccccctgttcgtggactccccccccgtgatcgaggacaacgacctgaagctgcacaagttcgaggtgaagaccggcgactccatccacaagggcctgacccccggctggaacgacctggacgtgaaccagcacgtgtccaacgtgaagtacatcggctggatcctggagtccatgcccaccgaggtgctggagacccaggagctgtgctccctggccctggagtaccgccgcgagtgcggccgcgactccgtgctggagtccgtgaccgccatggaccccaccaaggtgggcggccgctcccagtaccagcacctgctgcgcctggaggacggcaccgacatcgtgaagtgccgcaccgagtggcgccccaagaaccccggcgccaacggcgccatctccaccggcaagacctccaacggcaactccgtgtccatggactacaaggaccacgacggcgactacaaggaccacgacatcgactacaaggacgacgacgacaagtgattaattaactcgaggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaaagcttgagctccagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgactgaagagcgtttaaaccgcct

(19) United States
(12) Patent Application Publication
(10) Pub. No.: US 2018/0142218 A1
(43) Pub. Date: May 24, 2018

(54) NOVEL ACYLTRANSFERASES, VARIANT THIOESTERASES, AND USES THEREOF

(71) Applicant: TerraVia Holdings, Inc., South San Francisco, CA (US)

(72) Inventors: Jeffrey Leo Moseley, Redwood City, CA (US); Jason Casolari, Palo Alto, CA (US); Xinhua Zhao, Dublin, CA (US); Aren Ewing, South San Francisco, CA (US); Aravind Somanchi, Redwood City, CA (US); Scott Franklin, La Jolla, CA (US); David Davis, South San Francisco, CA (US)

(21) Appl. No.: 15/725,222

(22) Filed: Oct. 4, 2017

Related U.S. Application Data
(60) Provisional application No. 62/404,667, filed on Oct. 5, 2016.

(51) Int. Cl. C12N 9/10; C12P 7/64; C12N 15/82

(52) U.S. Cl. CPC C12N 9/1029; C12P 7/6463; C12N 2800/22; C12N 15/82; C12Y 203/01051

(57) ABSTRACT

Recombinant nucleic acids and vector constructs encoding acyltransferases and variant thioesterases, and the acyltransferases and variant thioesterases encoded by the nucleic acids are provided. The acyltransferases and variant thioesterases are useful in fatty acid synthesis and triacylglycerol production. Host cells that express the recombinant nucleic acids as well as methods of cultivating the host cells, methods of producing oils from the host cells are provided. The recombinant host cells and the oils produced therefrom have altered fatty acid profiles and/or triacylglycerols with altered regiospecificity.

### CROSS-REFERENCE TO RELATED APPLICATIONS

This application claims the benefit under 35 U.S.C. 119(e) of U.S. Provisional Patent Application No. 62/404,667, filed Oct. 5, 2016, entitled “Novel Acyltransferases, Variant Thioesterases, And Uses Thereof”, which is incorporated herein by reference in its entirety for all purposes.

### REFERENCE TO A SEQUENCE LISTING

This application includes a list of sequences, as shown at the end of the detailed description. The instant application contains a Sequence Listing which has been submitted electronically in ASCII format and is hereby incorporated by reference in its entirety. Said ASCII copy, created on Jan. 9, 2018, is named CORBP072US_SL.txt and is 606,605 bytes in size.

### FIELD OF THE INVENTION

Embodiments of the present invention relate to oils/fats, fuels, foods, and oleochemicals and their production from cultures of genetically engineered cells. Embodiments relate to nucleic acids and proteins that are involved in the fatty acid synthetic pathways; oils with a high content of triglycerides bearing fatty acyl groups upon the glycerol backbone in particular regiospecific patterns, highly stable oils, oils with high levels of oleic or mid-chain fatty acids, and products produced from such oils.

### BACKGROUND OF THE INVENTION

Co-owned patent applications WO2008/151149, WO2010/063031, WO2010/063032, WO2011/150410, WO2011/150411, WO2012/061647, WO2012/061647, WO2012/106560, WO2013/158938, WO2014/120829, WO2014/151904, WO2015/051319, WO2016/007862, WO2016/014968, WO2016/044779, and WO2016/164495 relate to microbial oils and methods for producing those oils in host cells, including microalgae. These publications also describe the use of such oils to make foods, oleochemicals, fuels and other products.

Certain enzymes of the fatty acyl-CoA elongation pathway function to extend the length of fatty acyl-CoA molecules. Elongase-complex enzymes extend fatty acyl-CoA molecules in 2 carbon additions, for example myristoyl-CoA to palmitoyl-CoA, stearoyl-CoA to arachidyl-CoA, or oleoyl-CoA to eicosanoyl-CoA, eicosanoyl-CoA to erucyl-CoA. In addition, elongase enzymes also extend acyl chain length in 2 carbon increments. KCS enzymes condense acyl-CoA molecules with two carbons from malonyl-CoA to form beta-ketoacyl-CoA. KCS and elongases may show specificity for condensing acyl substrates of particular carbon length, modification (such as hydroxylation), or degree of saturation. For example, the jojoba (Simmondsia chinensis) beta-ketoacyl-CoA synthase has been demonstrated to prefer monounsaturated and saturated C18- and C20-CoA substrates to elevate production of erucic acid in transgenic plants (Lassner et al., Plant Cell, 1996, Vol 8(2), pp. 281-292), whereas specific elongase enzymes of Trypanosoma brucei show preference for elongating short and midchain saturated CoA substrates (Lee et al., Cell, 2006, Vol 126(4), pp. 691-9).

The type II fatty acid biosynthetic pathway employs a series of reactions catalyzed by soluble proteins with intermediates shuttled between enzymes as thioesters of acyl carrier protein (ACP). By contrast, the type I fatty acid biosynthetic pathway uses a single, large multifunctional polypeptide.

The oleaginous, non-photosynthetic alga, Prototheca moriformis, stores copious amounts of triacylglyceride oil under conditions when the nutritional carbon supply is in excess, but cell division is inhibited due to limitation of other essential nutrients. Bulk biosynthesis of fatty acids with carbon chain lengths up to C18 occurs in the plastids; fatty acids are then exported to the endoplasmic reticulum where (if it occurs) elongation past C18 and incorporation into triacylglycerides (TAGs) is believed to occur. Lipids are stored in large cytoplasmic organelles called lipid bodies until environmental conditions change to favor growth, whereupon they are mobilized to provide energy and carbon molecules for anabolic metabolism.

### SUMMARY OF THE INVENTION

In various aspects, the inventions disclosed herein include one or more of the following embodiments. The embodiments can be practiced alone or in combination with each other.

### Embodiment 1

This embodiment of the invention provides a recombinant vector construct or a host cell comprising nucleic acids that encode an acyltransferase that optionally is operable to produce an altered fatty acid profile or an altered sn-2 profile in an oil produced by a host cell expressing the nucleic acids. The nucleic acids can be a nucleic acid construct or a vector construct that also includes one or more regulatory elements. The one or more regulatory elements include promoters, targeting sequences, secretion signals and other elements that control or direct the expression of the encoded protein in the host cell. The acyltransferase encoded by the nucleic acids have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196. The acyl transferases of this invention is a lysophosphatidic acid acyltransferase (LPAAT), glycerol phosphate acyltransferase (GPAT), diacyl glycerol acyltransferase (DGAT), lysophosphatidylcholine acyltransferase (LPCAT), or phospholipase A2 (PLA2). The acyltransferases of the invention are shown in Table 5. In one embodiment, the acyltransferases of the invention have acyltransferase activity and the amino acid sequence comprises at least 96.3%, 98%, or 99% identity to an acyltransferase of clade 1 of Table 5. In another embodiment, the acyltransferases of the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. In one embodiment, the recombinant vector construct of host cell comprises nucleic acids that 75%, 80%, 85%, 90%, 95%, 98%, 99%, or 100%, or at least 75%, 80%, 85%, 90%, 95%, 98%, 99%, or 100% identity to an acyltransferase encoded by SEQ ID NOs: 19, 20, 21, 22, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125.

### Embodiment 2

This embodiment of the invention provides nucleic acids that encode an acyltransferase that when expressed produces an altered fatty acid profile or an altered sn-2 profile in an oil produced by a host cell expressing the nucleic acids. The nucleic acids can be a nucleic acid construct or a vector construct that also includes one or more regulatory elements. The one or more regulatory elements include promoters, targeting sequences, secretion signals and other elements that control or direct the expression of the encoded protein in the host cell. The acyltransferase encoded by the nucleic acids have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196. The acyl transferases of this invention is a lysophosphatidic acid acyltransferase (LPAAT), glycerol phosphate acyltransferase (GPAT), diacyl glycerol acyltransferase (DGAT), lysophosphatidylcholine acyltransferase (LPCAT), or phospholipase A2 (PLA2). The acyltransferases of the invention are shown in Table 5. In one embodiment, the acyltransferases of the invention have acyltransferase activity and the amino acid sequence comprises at least 96.3%, 98%, or 99% identity to an acyltransferase of clade 1 of Table 5. In another embodiment, the acyltransferases of the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. In one embodiment, the nucleic acids comprise nucleic acids that are 75%, 80%, 85%, 90%, 95%, 98%, 99%, or 100%, or at least 75%, 80%, 85%, 90%, 95%, 98%, 99%, or 100% identity to an acyltransferase encoded by SEQ ID NOs: 19, 20, 21, 22, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125.

### Embodiment 3

This embodiment of the invention provides codon-optimized nucleic acids that encodes an acyltransferase operable to produce an altered fatty acid profile and/or an altered sn-2 profile in an oil produced by a host cell expressing the nucleic acids. In one aspect, the codons are optimized for expression in the host cell, including host cells derived from plants. In another aspect, the codons are optimized for expression in Prototheca or Chlorella. In a further aspect the codons are optimized for expression in Prototheca moriformis or Chlorella protothecoides. The codon-optimized nucleic acids can be a nucleic acid construct or a vector construct that also includes one or more regulatory elements. The one or more regulatory elements are also codon-optimized for Prototheca or Chlorella. The one or more regulatory elements include promoters, targeting sequences, secretion signals and other elements that control or direct the expression of the encoded protein in the host cell. The acyltransferase encoded by the codon-optimized nucleic acids have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196. When the codons are optimized for expression in a host organism, at least 60%, 65%, 70%, 75%, 80%, 85%, 90%, 95%, or 100% of the codons used is the most preferred codon. Alternately, at least 60%, 65%, 70%, 75%, 80%, 85%, 90%, 95%, or 100% of the codons used is the first or second most preferred codon. The codon-optimized nucleic acids encode acyltransferases that are shown in Table 5. In one embodiment, the acyltransferases of the invention have acyltransferase activity and the amino acid sequence comprises at least 96.3%, 98%, or 99% identity to an acyltransferase of clade 1 of Table 5. In another embodiment, the acyltransferases of the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. The acyltransferase encoded by the codon-optimized nucleic acids have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196. In one embodiment, the codon-optimizes nucleic acids comprise nucleic acids that 75%, 80%, 85%, 90%, 95%, 98%, 99%, or 100%, or at least 75%, 80%, 85%, 90%, 95%, 98%, 99%, or 100% identity to an acyltransferase encoded by SEQ ID NOs: 19, 20, 21, 22, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125.

### Embodiment 4

In this embodiment, the invention provides host cells that are oleaginous microorganism cells or plant cells. The microorganisms of the invention are eukaryotic microorganism. In one aspect, the host cells are microalgae. In one embodiment, the microalgae are of the phylum Chlorophyta, the class Trebouxiophytae, the order Chlorellales, or the family Chlorellacae. In one embodiment, the microalgae are of the genus Prototheca or Chlorella. In one embodiment, the microalgae are of the species Prototheca moriformis, Prototheca zopfii, Prototheca wickerhamii Prototheca blaschkeae, Prototheca chlorelloides, Prototheca crieana, Prototheca dilamenta, Prototheca hydrocarbonea, Prototheca kruegeri, Prototheca portoricensis, Prototheca salmonis, Prototheca segbwema, Prototheca stagnorum, Prototheca trispora Prototheca ulmea, or Prototheca viscosa. Preferably, the microalga is of the species Prototheca moriformis. In one embodiment, the microalgae are of the species Chlorella autotrophica, Chlorella colonials, Chlorella lewinii, Chlorella minutissima, Chlorella pituitam, Chlorella pulchelloides, Chlorella pyrenoidosa, Chlorella rotunda, Chlorella singularis, Chlorella sorokiniana, Chlorella variabilis, or Chlorella volutis. Preferably, the microalga is of the species Chlorella protothecoides or Auxenochlorella protothecoides. The host cells express the nucleic acids for Embodiments relating to acyltransferases of the invention.

### Embodiment 5

In this embodiment, the acyl transferase is lysophosphatidic acid acyltransferase (LPAAT), glycerol phosphate acyltransferase (GPAT), diacyl glycerol acyltransferase (DGAT), lysophosphatidylcholine acyltransferase (LPCAT), or phospholipase A2 (PLA2). In one embodiment, the acyltransferases of the invention are shown in Table 5. In one embodiment, the acyltransferases of the invention have acyltransferase activity and the amino acid sequence comprises at least 96.3%, 98%, or 99% identity to an acyltransferase of clade 1 of Table 5. In another embodiment, the acyltransferases of the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. The acyltransferase have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196.

### Embodiment 6

In this embodiment, nucleic acids encoding acyltransferases increases the production of C8:0 and/or C10:0 fatty acids or alters the sn-2 profile in the host cell. The acyltransferases of the invention have acyltransferase activity and the amino acid sequence comprises at least 96.3%, 98%, or 99% identity to an acyltransferase of clade 1 of Table 5. In another embodiment, the acyltransferases of the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. The C8:0 or the C10:0 content of the oil of the host cell is increased by 5%, 10%, 15%, 20%, 25%, 30%, 35%, 40%, 45%, 50%, 55%, 60%, 70%, 80%, 90%, or higher as compared the C8:0 and/or C10:0 content of a cell oil that does not express the recombinant nucleic acids encoding the LPAATs of the invention. The sn-2 profile of the oil is altered by the expression of the LPAATs of the invention and/or the C8:0 and/or C10:0 fatty acid at the sn-2 position is increased by 5%, 10%, 15%, 20%, 25%, 30%, 35%, 40%, 45%, 50%, 55%, 60%, 70%, 80%, 90%, or higher as compared to the C8:0 and/or C10:0 fatty acid at the sn-2 position of the cell oil that does not express the recombinant nucleic acids encoding the LPAATs of the invention. The acyltransferase encoded by the codon-optimized nucleic acids have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196.

### Embodiment 7

This embodiment comprises nucleic acids encoding LPAATs, shown in Table 5, and disclosed herein. The LPAATs encoded by the nucleic acids have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, or 180.

### Embodiment 8

In this embodiment, nucleic acids encoding GPATs of the invention have 75%, 80%, 85%, 90%, 95%, 98%, 99%, or 100%, or at least 75%, 80%, 85%, 90%, 95%, 98%, 99%, or 100% identity to SEQ ID NOs: 181, 182, 183, 184, 185, or 186.

### Embodiment 9

In this embodiment, nucleic acids encoding DGATs of the invention have 75%, 80%, 85%, 90%, 95%, 98%, 99%, or 100%, or at least 75%, 80%, 85%, 90%, 95%, 98%, 99%, or 100% identity to SEQ ID NOs: 187, or 188.

### Embodiment 10

In this embodiment, nucleic acids encoding LPCATs of the invention have 75%, 80%, 85%, 90%, 95%, 98%, 99%, or 100%, or at least 75%, 80%, 85%, 90%, 95%, 98%, 99%, or 100% identity to SEQ ID NOs: 189, 190, 191, or 192,

### Embodiment 11

This embodiment comprises nucleic acids encoding PLA2s. The PLA2s encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 193, 194, 195, or 196.

### Embodiment 12

This embodiment is a method of cultivating a host cell expressing nucleic acids that encode the one or more acyl transferases of embodiments 1-11

### Embodiment 13

This embodiment is a method of producing an oil by cultivating host cells that express nucleic acids that encode the one or more acyl transferases of Embodiments 1-12 and recovering the oil.

### Embodiment 14

This embodiment is an oil produced by cultivating host cells that express the one or more nucleic acids that encode the acyltransferases of Examples 1-11, and recovering the oil from the host cell. When the host cell is a microalgae, the cell oil produced by the host cell has sterols that are different than the sterols produced by a plant cell. The cell oil has a sterol profile that is different than an oil obtained from a plant.

### Embodiment 15

In this embodiment, a recombinant acyltransferase is provided. The recombinant acyltransferase can be produced by a host cell. The glycosylation of the recombinant acyl transferase is altered from the glycosylation pattern observed in the acyl transferase produced by the non-recombinant, wild-type cell from which the gene encoding the acyl transferase was derived. In one embodiment, the recombinant acyltransferase the invention have acyltransferase activity and the amino acid sequence comprises at least 96.3%, 98%, or 99% identity to an acyltransferase of clade 1 of Table 5. In one embodiment, the recombinant acyltransferase the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. The acyltransferase encoded have 75%, 80%, 85%, 90%, 95%, 98%, 99%, or 100%, or at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196.

### Embodiment 16

This embodiment of the invention provides a recombinant vector construct or a host cell comprising nucleic acids that encode a variant Brassica fatty acyl-ACP thioesterase that optionally is operable to produce an altered fatty acid profile in an oil produced by a host cell expressing the nucleic acids. The nucleic acids can be a nucleic acid construct or a vector construct that also includes one or more regulatory elements. The one or more regulatory elements include promoters, targeting sequences, secretion signals and other elements that control or direct the expression of the encoded protein in the host cell. The thioesterase encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 165, 166, 167, or 168 and comprise one or more of amino acid variants D124A, D209A, D127A or D212A. In one embodiment, the Brassica Rapa, Brassica napus or the Brassica juncea thioesterases of the invention have fatty acyl hydrolysis activity and prefer to hydrolyze long chain fatty acyl groups from the acyl carrier protein. In one embodiment, the thioesterase genes, isolated from higher plants, are altered to create variant thioesterases that have certain amino acids that have been altered from the wild type enzyme. Due to the altered amino acid(s), the substrate specificity of the thioesterase is altered. The variant BnOTE enzymes increased C18:0 content by DCW, decreased C18:1 content by DCW, and decreased C18:2 content by DCW in host cells and the oils recovered from the host cells.

### Embodiment 17

This embodiment of the invention provides a recombinant vector construct or a host cell comprising nucleic acids that encode a Garcinia mangostana variant fatty acyl-ACP thioesterase (GmFATA) that optionally is operable to produce an altered fatty acid profile in an oil produced by a host cell expressing the nucleic acids. The nucleic acids can be a nucleic acid construct or a vector construct that also includes one or more regulatory elements. The one or more regulatory elements include promoters, targeting sequences, secretion signals and other elements that control or direct the expression of the encoded protein in the host cell. The variant Garcinia thioesterase encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 137, 138, 139, 140, 141, 142, 143, 144, 145, 146, 147, 148, 149, 150, comprise one more of amino acid variants D variants L91F, L91K, L91S, G96A, G96T, G96V, G108A, G108V, S111A, S111V T156F, T156A, T156K, T156V, or V193A. In one embodiment, the G mangostana thioesterases of the invention have fatty acyl hydrolysis activity and prefer to hydrolyze long chain fatty acyl groups from the acyl carrier protein. In one embodiment, the thioesterase genes, isolated from higher plants, are altered to create variant thioesterases that have certain amino acids that have been altered from the wild type enzyme. Due to the altered amino acid(s), the substrate specificity of the thioesterase is altered. The variant BnOTE enzymes increased C18:0 content by DCW, decreased C18:1 content by DCW, and decreased C18:2 content by DCW in host cells and the oils recovered from the host cells.

### Embodiment 18

This embodiment of the invention provides nucleic acids that encode variant Brassica thioesterases or variant Garcinia thioestrases that when expressed produce an altered fatty acid profile in an oil produced by a host cell expressing the nucleic acids. The nucleic acids can be a nucleic acid construct or a vector construct that also includes one or more regulatory elements. The one or more regulatory elements include promoters, targeting sequences, secretion signals and other elements that control or direct the expression of the encoded protein in the host cell. The variant Brassica thioesterases encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 165, 166, 167, or 168 and comprise one or more of amino acid variants D124A, D209A, D127A or D212A. The variant variant Garcinia thioestrases encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 137, 138, 139, 140, 141, 142, 143, 144, 145, 146, 147, 148, 149, 150 and comprise one or more of amino acid variants L91F, L91K, L91S, G96A, G96T, G96V, G108A, G108V, S111A, S111V T156F, T156A, T156K, T156V, or V193A.

### Embodiment 19

This embodiment of the invention provides codon-optimized nucleic acids that encodes a variant Brassica thioesterase or a variant Garcinia thioestrase operable to produce an altered fatty acid profile in an oil produced by a host cell expressing the nucleic acids. In one aspect, the codons are optimized for expression in the host cell, including host cells derived from plants. In another aspect, the codons are optimized for expression in Prototheca or Chlorella. In a further aspect the codons are optimized for expression in Prototheca moriformis or Chlorella protothecoides. The codon-optimized nucleic acids can be a nucleic acid construct or a vector construct that also includes one or more regulatory elements. The one or more regulatory elements are also codon-optimized for Prototheca or Chlorella. The one or more regulatory elements include promoters, targeting sequences, secretion signals and other elements that control or direct the expression of the encoded protein in the host cell. The variant Brassica thioesterases encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 165, 166, 167, or 168 and comprise one or more of amino acid variants D124A, D209A, D127A or D212A. The variant variant Garcinia thioestrases encoded by the nucleic acids have 75%, 80%, 85%, 90%, 95%, 98%, 99%, or 100%, or at least 75%, 80%, 85%, 90%, 95%, 98%, 99%, or 100% identity to SEQ ID NOs: 137, 138, 139, 140, 141, 142, 143, 144, 145, 146, 147, 148, 149, or 150 and comprise one or more of amino acid variants L91F, L91K, L91S, G96A, G96T, G96V, G108A, G108V, S111A, S111V T156F, T156A, T156K, T156V, or V193A. When the codons are optimized for expression in a host organism, at least 60%, 65%, 70%, 75%, 80%, 85%, 90%, 95%, or 100% of the codons used is the most preferred codon. Alternately, at least 60%, 65%, 70%, 75%, 80%, 85%, 90%, 95%, or 100% of the codons used is the first or second most preferred codon. The codon-optimized nucleic acids encode variant Brassica thioesterases and variant Garcinia thioestrases. In one embodiment, the variant Brassica thioesterases and variant Garcinia thioestrases of the invention have thioesterase activity.

### Embodiment 20

In this embodiment, the invention provides host cells that are oleaginous microorganism cells or plant cells. The microorganisms of the invention are eukaryotic microorganism. In one aspect, the host cells are microalgae. In one embodiment, the microalgae are of the phylum Chlorophyta, the class Trebouxiophytae, the order Chlorellales, or the family Chlorellacae. In one embodiment, the microalgae are of the genus Prototheca or Chlorella. In one embodiment, the microalgae are of the species Prototheca moriformis, Prototheca zopfii, Prototheca wickerhamii Prototheca blaschkeae, Prototheca chlorelloides, Prototheca crieana, Prototheca dilamenta, Prototheca hydrocarbonea, Prototheca kruegeri, Prototheca portoricensis, Prototheca salmonis, Prototheca segbwema, Prototheca stagnorum, Prototheca trispora Prototheca ulmea, or Prototheca viscosa. Preferably, the microalga is of the species Prototheca moriformis. In one embodiment, the microalgae are of the species Chlorella autotrophica, Chlorella colonials, Chlorella lewinii, Chlorella minutissima, Chlorella pituitam, Chlorella pulchelloides, Chlorella pyrenoidosa, Chlorella rotunda, Chlorella singularis, Chlorella sorokiniana, Chlorella variabilis, or Chlorella volutis. Preferably, the microalga is of the species Chlorella protothecoides or Auxenochlorella protothecoides. The host cells express the nucleic acids for Embodiments relating to acyltransferases of the invention.

### Embodiment 21

In this embodiment, the nucleic acid encoding the variant Brassica thioesterase encodes a variant thioesterase that has 75%, 80%, 85%, 90%, 95%, 98%, 99%, or 100%, or at least 75%, 80%, 85%, 90%, 95%, 98%, 99%, or 100% identity to SEQ ID NOs: 165, 166, 167, or 168 and comprise one or more of amino acid variants D124A, D209A, D127A or D212A. In another aspect, the nucleic acid encoding the variant Garcinia thioesterase encodes a variant thioesterase that has 75%, 80%, 85%, 90%, 95%, 98%, 99%, or 100%, or at least 75%, 80%, 85%, 90%, 95%, 98%, 99%, or 100% identity to SEQ ID NOs: 137, 138, 139, 140, 141, 142, 143, 144, 145, 146, 147, 148, 149, or 150, and comprise one or more of amino acid variants L91F, L91K, L91S, G96A, G96T, G96V, G108A, G108V, S111A, S111V T156F, T156A, T156K, T156V, or V193A.

### Embodiment 22

In this embodiment, nucleic acids encoding a variant Brassica thioesterase or a variant Garcinia thioesetrase that decrease the production of C18:0 and/or decrease the production of C18:1 fatty acids and/or decreases the production of C18:2 fatty acids sn-2 in the host cell.

### Embodiment 23

In this embodiment, nucleic acids encoding a variant Brassica thioesterase of the invention have SEQ ID NOs: 165, 166, 167, or 168 and comprise one or more of amino acid variants D124A, D209A, D127A or D212A.

### Embodiment 24

In this embodiment, nucleic acids encoding a variant Garcinia thioesetrase of the invention have 75%, 80%, 85%, 90%, 95%, 98%, 99%, or 100%, or at least 75%, 80%, 85%, 90%, 95%, 98%, 99%, or 100% identity to SEQ ID NOs: 137, 138, 139, 140, 141, 142, 143, 144, 145, 146, 147, 148, 149, or 150 and comprise one or more of amino acid variants L91F, L91K, L91S, G96A, G96T, G96V, G108A, G108V, S111A, S111V T156F, T156A, T156K, T156V, or V193A.

### Embodiment 25

This embodiment is a method of cultivating a host cell expressing nucleic acids that encode the one or more acyl transferases of embodiments 16-24.

### Embodiment 26

This embodiment is a method of producing an oil by cultivating host cells that express nucleic acids that encode the one or more variant thioesterases of Embodiments 16-25 and recovering the oil.

### Embodiment 27

This embodiment is an oil produced by cultivating host cells that express the one or more nucleic acids that encode the variant transferases of Examples 16-24, and recovering the oil from the host cell. When the host cell is a microalgae, the cell oil produced by the host cell has sterols that are different than the sterols produced by a plant cell. The cell oil has a sterol profile that is different than an oil obtained from a plant.

### Embodiment 28

In this embodiment, a recombinant variant thioesterase is provided. The recombinant variant thioesterase is produce by a host cell. The glycosylation of the recombinant variant thioesterase is altered from the glycosylation pattern observed in the variant thioesterase produced by the non-recombinant, wild-type cell from which the gene encoding the variant thioesterase was derived.

By way of example and not intended to be the only combination, the acyltransferase and/or the variant acyl-ACP thioesterrases of the invention can be expressed in a cell in which an endogenous desaturase, KAS, and/or fatty acyl-ACP thioesterase has been ablated or downregulated as demonstrated in the Examples. The co-expression of an acyltransferase and/or a variant acyl-ACP thioesterase concomitantly with an invertase is an embodiment of the invention, as was demonstrated in the disclosed Examples. Additionally, the expression of an acyltansferase and/or a variant acyl-ACP thioesterase with concomitant expression of a invertase and ablation or downregulation of a desaturase, KAS and/or fatty acyl-ACP thioesterase is an embodiment of the invention, as demonstrated in the disclosed Examples.

### BRIEF DESCRIPTION OF THE DRAWINGS

FIG. 1. TAG profiles of S7815 versus the S6573 parent. TAGs in brackets co-elute with the peak of the main TAG, but are present in trace amounts, and do not contribute significantly to the area. M=myristate (C14:0), P=palmitate (C16:0), Po=palmitoleate (C16:1), Ma=margaric (C17:0), S=stearate (C18:0), 0=oleate (C18:1), L=linoleate (C18:2), Ln=linolenate (C18:3 α), A=arachidate (C20:0), B=behenate (C22:0), Lg=lignocerate (C24:0), Hx=hexacosanoate (C26:0). Sat-Sat-Sat=trisaturates. See Example 5.

FIG. 2. TAG profiles of lipids from fermentations of S7815 versus S6573. TAGs in brackets co-elute with the peak of the main TAG, but are present in trace amounts, and do not contribute significantly to the area. M=myristate (C14:0), P=palmitate (C16:0), S=stearate (C18:0), 0=oleate (C18:1), L=linoleate (C18:2), Ln=linolenate (C18:3 α), A=arachidate (C20:0), B=behenate (C22:0), Lg=lignocerate (C24:0), Hx=hexacosanoate (C26:0). Sat-Sat-Sat=trisaturates. See Example 5.

### DETAILED DESCRIPTION OF THE INVENTION

### I. Definitions

An “allele” refers to a copy of a gene where an organism has multiple similar or identical gene copies, even if on the same chromosome. An allele may encode the same or similar protein.

An “oil,” “cell oil” or “cell fat” shall mean a predominantly triglyceride oil obtained from an organism, where the oil has not undergone blending with another natural or synthetic oil, or fractionation so as to substantially alter the fatty acid profile of the triglyceride. In connection with an oil comprising triglycerides of a particular regiospecificity, the cell oil or cell fat has not been subjected to interesterification or other synthetic process to obtain that regiospecific triglyceride profile, rather the regiospecificity is produced naturally, by a cell or population of cells. For a cell oil produced by a cell, the sterol profile of oil is generally determined by the sterols produced by the cell, not by artificial reconstitution of the oil by adding sterols in order to mimic the cell oil. In connection with a cell oil or cell fat, and as used generally throughout the present disclosure, the terms oil, and fat are used interchangeably, except where otherwise noted. Thus, an “oil” or a “fat” can be liquid, solid, or partially solid at room temperature, depending on the makeup of the substance and other conditions. Here, the term “fractionation” means removing material from the oil in a way that changes its fatty acid profile relative to the profile produced by the organism, however accomplished. The terms “oil,” “cell oil” and “cell fat” encompass such oils obtained from an organism, where the oil has undergone minimal processing, including refining, bleaching, deodorized, and/or degumming, which does not substantially change its triglyceride profile. A cell oil can also be a “noninteresterified cell oil”, which means that the cell oil has not undergone a process in which fatty acids have been redistributed in their acyl linkages to glycerol and remain essentially in the same configuration as when recovered from the organism.

As used herein, an oil is said to be “enriched” in one or more particular fatty acids if there is at least a 10% increase in the mass of that fatty acid in the oil relative to the non-enriched oil. For example, in the case of a cell expressing a heterologous FatB gene described herein, the oil produced by the cell is said to be enriched in, e.g., C8 and C16 fatty acids if the mass of these fatty acids in the oil is at least 10% greater than in oil produced by a cell of the same type that does not express the heterologous FatB gene (e.g., wild type oil).

“Exogenous gene” shall mean a nucleic acid that codes for the expression of an RNA and/or protein that has been introduced into a cell (e.g. by transformation/transfection), and is also referred to as a “transgene”. A cell comprising an exogenous gene may be referred to as a recombinant cell, into which additional exogenous gene(s) may be introduced. The exogenous gene may be from a different species (and so heterologous), or from the same species (and so homologous), relative to the cell being transformed. Thus, an exogenous gene can include a homologous gene that occupies a different location in the genome of the cell or is under different control, relative to the endogenous copy of the gene. An exogenous gene may be present in more than one copy in the cell. An exogenous gene may be maintained in a cell as an insertion into the genome (nuclear or plastid) or as an episomal molecule.

“FADc”, also referred to as “FAD2” or “FAD” is a gene encoding a delta-12 fatty acid desaturase. “SAD” is a gene encoding a stearoyl ACP desaturase, a delta-9 fatty acid desaturase. The desaturases desaturates a fatty acyl chain to create a double bond. SAD converts stearic acid, C18:0 to oleic acid, C18:1 and FAD converts oleic acid, C18:1 to linoleic acid, C18:2.

“Fatty acids” shall mean free fatty acids, fatty acid salts, or fatty acyl moieties in a glycerolipid. It will be understood that fatty acyl groups of glycerolipids can be described in terms of the carboxylic acid or anion of a carboxylic acid that is produced when the triglyceride is hydrolyzed or saponified.

“Fixed carbon source” is a molecule(s) containing carbon, typically an organic molecule that is present at ambient temperature and pressure in solid or liquid form in a culture media that can be utilized by a microorganism cultured therein. Accordingly, carbon dioxide is not a fixed carbon source. Typical fixed carbon source include sucrose, glucose, fructose and other well-known monosaccharides, disaccharides and polysaccharides.

“In operable linkage” is a functional linkage between two nucleic acid sequences, such a control sequence (typically a promoter) and the linked sequence (typically a sequence that encodes a protein, also called a coding sequence). A promoter is in operable linkage with an exogenous gene if it can mediate transcription of the gene.

“Microalgae” are eukaryotic microbial organisms that contain a chloroplast or other plastid, and optionally that is capable of performing photosynthesis, or a prokaryotic microbial organism capable of performing photosynthesis. Microalgae include obligate photoautotrophs, which cannot metabolize a fixed carbon source as energy, as well as heterotrophs, which can live solely off of a fixed carbon source. Microalgae also include mixotrophic organisms that can perform photosynthesis and metabolize one or more fixed carbon source. Microalgae include unicellular organisms that separate from sister cells shortly after cell division, such as Chlamydomonas, as well as microbes such as, for example, Volvox, which is a simple multicellular photosynthetic microbe of two distinct cell types. Microalgae include cells such as Chlorella, Dunaliella, and Prototheca. Microalgae also include other microbial photosynthetic organisms that exhibit cell-cell adhesion, such as Agmenellum, Anabaena, and Pyrobotrys. Microalgae also include obligate heterotrophic microorganisms that have lost the ability to perform photosynthesis, such as certain dinoflagellate algae species and species of the genus Prototheca.

As used with respect to nucleic acids, the term “isolated” refers to a nucleic acid that is free of at least one other component that is typically present with the naturally occurring nucleic acid. Thus, a naturally occurring nucleic acid is isolated if it has been purified away from at least one other component that occurs naturally with the nucleic acid.

In connection with fatty acid length, “mid-chain” shall mean C8 to C16 fatty acids.

In connection with a recombinant cell, the term “knockdown” refers to a gene that has been partially suppressed (e.g., by about 1-95%) in terms of the production or activity of a protein encoded by the gene. Inhibitory RNA technology to down-regulate or knockdown expression of a gene are well known. These techniques include dsRNA, hairpin RNA, antisense RNA, interfering RNA (RNAi) and others.

Also, in connection with a recombinant cell, the term “knockout” refers to a gene that has been completely or nearly completely (e.g., >95%) suppressed in terms of the production or activity of a protein encoded by the gene. Knockouts can be prepared by ablating the gene by homologous recombination of a nucleic acid sequence into a coding sequence, gene deletion, mutation or other method. When homologous recombination is performed, the nucleic acid that is inserted (“knocked-in”) can be a sequence that encodes an exogenous gene of interest or a sequence that does not encode for a gene of interest. The ablation by homologous recombination can be performed in one, two or more alleles of the gene of interest.

An “oleaginous” cell is a cell capable of producing at least 20% lipid by dry cell weight, naturally or through recombinant or classical strain improvement. An “oleaginous microbe” or “oleaginous microorganism” is a microbe, including a microalga that is oleaginous (especially eukaryotic microalgae that store lipid). An oleaginous cell also encompasses a cell that has had some or all of its lipid or other content removed, and both live and dead cells.

An “ordered oil” or “ordered fat” is one that forms crystals that are primarily of a given polymorphic structure. For example, an ordered oil or ordered fat can have crystals that are greater than 50%, 60%, 70%, 80%, or 90% of the 0 or (3′ polymorphic form.

In connection with a cell oil, a “profile” is the distribution of particular species or triglycerides or fatty acyl groups within the oil. A “fatty acid profile” is the distribution of fatty acyl groups in the triglycerides of the oil without reference to attachment to a glycerol backbone. Fatty acid profiles are typically determined by conversion to a fatty acid methyl ester (FAME), followed by gas chromatography (GC) analysis with flame ionization detection (FID), as in Example 1. The fatty acid profile can be expressed as one or more percent of a fatty acid in the total fatty acid signal determined from the area under the curve for that fatty acid. FAME-GC-FID measurement approximate weight percentages of the fatty acids. A “sn-2 profile” is the distribution of fatty acids found at the sn-2 position of the triacylglycerides in the oil. A “regiospecific profile” is the distribution of triglycerides with reference to the positioning of acyl group attachment to the glycerol backbone without reference to stereospecificity. In other words, a regiospecific profile describes acyl group attachment at sn-1/3 vs. sn-2. Thus, in a regiospecific profile, POS (palmitate-oleate-stearate) and SOP (stearate-oleate-palmitate) are treated identically. A “stereospecific profile” describes the attachment of acyl groups at sn-1, sn-2 and sn-3. Unless otherwise indicated, triglycerides such as SOP and POS are to be considered equivalent. A “TAG profile” is the distribution of fatty acids found in the triglycerides with reference to connection to the glycerol backbone, but without reference to the regiospecific nature of the connections. Thus, in a TAG profile, the percent of SSO in the oil is the sum of SSO and SOS, while in a regiospecific profile, the percent of SSO is calculated without inclusion of SOS species in the oil. In contrast to the weight percentages of the FAME-GC-FID analysis, triglyceride percentages are typically given as mole percentages; that is the percent of a given TAG molecule in a TAG mixture.

The term “percent sequence identity,” in the context of two or more amino acid or nucleic acid sequences, refers to two or more sequences or subsequences that are the same or have a specified percentage of amino acid residues or nucleotides that are the same, when compared and aligned for maximum correspondence, as measured using a sequence comparison algorithm or by visual inspection. For sequence comparison to determine percent nucleotide or amino acid identity, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted using the NCBI BLAST software (ncbi.nlm.nih.gov/BLAST/) set to default parameters. For example, to compare two nucleic acid sequences, one may use blastn with the “BLAST 2 Sequences” tool Version 2.0.12 (Apr. 21, 2000) set at the following default parameters: Matrix: BLOSUM62; Reward for match: 1; Penalty for mismatch: −2; Open Gap: 5 and Extension Gap: 2 penalties; Gap x drop-off: 50; Expect: 10; Word Size: 11; Filter: on. For a pairwise comparison of two amino acid sequences, one may use the “BLAST 2 Sequences” tool Version 2.0.12 (Apr. 21, 2000) with blastp set, for example, at the following default parameters: Matrix: BLOSUM62; Open Gap: 11 and Extension Gap: 1 penalties; Gap x drop-off 50; Expect: 10; Word Size: 3; Filter: on.

“Recombinant” is a cell, nucleic acid, protein or vector that has been modified due to the introduction of an exogenous nucleic acid or the alteration of a native nucleic acid. Thus, e.g., recombinant cells can express genes that are not found within the native (non-recombinant) form of the cell or express native genes differently than those genes are expressed by a non-recombinant cell. Recombinant cells can, without limitation, include recombinant nucleic acids that encode for a gene product or for suppression elements such as mutations, knockouts, antisense, interfering RNA (RNAi), hairpin RNA or dsRNA that reduce the levels of active gene product in a cell. A “recombinant nucleic acid” is a nucleic acid originally formed in vitro, in general, by the manipulation of nucleic acid, e.g., using polymerases, ligases, exonucleases, and endonucleases, using chemical synthesis, or otherwise is in a form not normally found in nature. Recombinant nucleic acids may be produced, for example, to place two or more nucleic acids in operable linkage. Thus, an isolated nucleic acid or an expression vector formed in vitro by ligating DNA molecules that are not normally joined in nature, are both considered recombinant for the purposes of this invention. Once a recombinant nucleic acid is made and introduced into a host cell or organism, it may replicate using the in vivo cellular machinery of the host cell; however, such nucleic acids, once produced recombinantly, although subsequently replicated intracellularly, are still considered recombinant for purposes of this invention. Similarly, a “recombinant protein” is a protein made using recombinant techniques, i.e., through the expression of a recombinant nucleic acid. A recombinant protein will have a different pattern of glycosylation than the protein isolated from the wild-type organism.

The genes can be used in a variety of genetic constructs including plasmids or other vectors for expression or recombination in a host cell. The genes can be codon optimized for expression in a target host cell. The proteins produced by the genes can be used in vivo or in purified form.

For example, the gene can be prepared in an expression vector comprising an operably linked promoter and 5′UTR. Where a plastidic cell is used as the host, a suitably active plastid targeting peptide can be fused to the FATB gene, as in the examples below. Generally, for the newly identified FATB genes, there are roughly 50 amino acids at the N-terminal that constitute a plastid transit peptide, which are responsible for transporting the enzyme to the chloroplast. In the examples below, this transit peptide is replaced with a 38 amino acid sequence that is effective in the Prototheca moriformis host cell for transporting the enzyme to the plastids of those cells. Thus, the invention contemplates deletions and fusion proteins in order to optimize enzyme activity in a given host cell. For example, a transit peptide from the host or related species may be used instead of that of the newly discovered plant genes described here.

A selectable marker gene may be included in the vector to assist in isolating a transformed cell. Examples of selectable markers useful in microlagae include sucrose invertase antibiotic resistance genes and other genes useful as selectable markers. The S. carlbergensis MEL1 gene (conferring the ability to grow on melibiose), A. thaliana THIC gene (conferring the ability to grow in media free of thiamine, Saccharomyces sucrose invertase (conferring the ability to grow on sucrose) are disclosed in the Examples. Other known selectable markers are useful and within the ambit of a skilled artisan.

The terms “triglyceride”, “triacylglyceride” and “TAG” are used interchangeably as is known in the art.

### II. Embodiments of the Invention

Illustrative embodiments of the present invention feature oleaginous cells that produce altered fatty acid profiles and/or altered regiospecific distribution of fatty acids in glycerolipids, and products produced from the cells. Examples of oleaginous cells include microbial cells having a type II fatty acid biosynthetic pathway, including plastidic oleaginous cells such as those of oleaginous algae and, where applicable, oil producing cells of higher plants including but not limited to commercial oilseed crops such as soy, corn, rapeseed/canola, cotton, flax, sunflower, safflower and peanut. Other specific examples of cells include heterotrophic or obligate heterotrophic microalgae of the phylum Chlorophtya, the class Trebouxiophytae, the order Chlorellales, or the family Chlorellacae. Examples of oleaginous microalgae and methods of cultivation are also provided in co-owned applications WO2008/151149, WO2010/063031, WO2010/063032, WO2011/150410, WO2011/150411, WO2012/061647, WO2012/061647, WO2012/106560, and WO2013/158938, WO2014/120829, WO2014/151904, WO2015/051319, WO2016/007862, WO2016/014968, WO2016/044779, WO2016/164495, all of which are incorporated by reference, including species of Chlorella and Prototheca, a genus comprising obligate heterotrophs. The oleaginous cells can be, for example, capable of producing 25%, 30%, 40%, 50%, 60%, 70%, 80%, 85%, or about 90% oil by cell weight, ±5%. Optionally, the oils produced can be low in highly unsaturated fatty acids such as DHA or EPA fatty acids. For example, the oils can comprise less than 5%, 2%, or 1% DHA and/or EPA. The above-mentioned publications also disclose methods for cultivating such cells and extracting oil, especially from microalgal cells; such methods are applicable to the cells disclosed herein and incorporated by reference for these teachings. When microalgal cells are used they can be cultivated autotrophically (unless an obligate heterotroph) or in the dark using a sugar (e.g., glucose, fructose and/or sucrose) In any of the embodiments described herein, the cells can be heterotrophic cells comprising an exogenous invertase gene so as to allow the cells to produce oil from a sucrose feedstock. Alternately, or in addition, the cells can metabolize xylose from cellulosic feedstocks. For example, the cells can be genetically engineered to express one or more xylose metabolism genes such as those encoding an active xylose transporter, a xylulose-5-phosphate transporter, a xylose isomerase, a xylulokinase, a xylitol dehydrogenase and a xylose reductase. See WO2012/154626, “GENETICALLY ENGINEERED MICROORGANISMS THAT METABOLIZE XYLOSE”, published Nov. 15, 2012, including disclosure of genetically engineered Prototheca strains that utilize xylose.

The host cells expressing the acyltransferases or the variant B. napus thioesterases or the variant G. mangostana thioesterase may, optionally, be cultivated in a bioreactor/fermenter. For example, heterotrophic oleaginous microalgal cells can be cultivated on a sugar-containing nutrient broth. Optionally, cultivation can proceed in two stages: a seed stage and a lipid-production stage. In the seed stage, the number of cells is increased from a starter culture. Thus, the seed stage(s) typically includes a nutrient rich, nitrogen replete, media designed to encourage rapid cell division. After the seed stage(s), the cells may be fed sugar under nutrient-limiting (e.g. nitrogen sparse) conditions so that the sugar will be converted into triglycerides. As used herein, “standard lipid production conditions” are disclosed here. In one embodiment, the culture conditions are nitrogen limiting. Sugar and other nutrients can be added during the fermentation but no additional nitrogen is added. The cells will consume all or nearly all of the nitrogen present, but no additional nitrogen is provided. For example, the rate of cell division in the lipid-production stage can be decreased by 50%, 80%, or more relative to the seed stage. Additionally, variation in the media between the seed stage and the lipid-production stage can induce the recombinant cell to express different lipid-synthesis genes and thereby alter the triglycerides being produced. For example, as discussed below, nitrogen and/or pH sensitive promoters can be placed in front of endogenous or exogenous genes. This is especially useful when an oil is to be produced in the lipid-production phase that does not support optimal growth of the cells in the seed stage.

The oleaginous cells express one or more exogenous genes encoding fatty acid biosynthesis enzymes. As a result, some embodiments feature cell oils that were not obtainable from a non-plant or non-seed oil, or not obtainable at all.

The oleaginous cells, including microalgal cells, can be improved via classical strain improvement techniques such as UV and/or chemical mutagenesis followed by screening or selection under environmental conditions, including selection on a chemical or biochemical toxin. For example the cells can be selected on a fatty acid synthesis inhibitor, a sugar metabolism inhibitor, or an herbicide. As a result of the selection, strains can be obtained with increased yield on sugar, increased oil production (e.g., as a percent of cell volume, dry weight, or liter of cell culture), or improved fatty acid or TAG profile. Co-owned application PCT/US2016/025023 filed on 31 Mar. 2016, herein incorporated by reference, describes methods for classically mutagenizing oleaginous cells.

The cells can be selected on one or more of 1,2-Cyclohexanedione; 19-Norethindone acetate; 2,2-dichloropropionic acid; 2,4,5-trichlorophenoxyacetic acid; 2,4,5-trichlorophenoxyacetic acid, methyl ester; 2,4-dichlorophenoxyacetic acid; 2,4-dichlorophenoxyacetic acid, butyl ester; 2,4-dichlorophenoxyacetic acid, isooctyl ester; 2,4-dichlorophenoxyacetic acid, methyl ester; 2,4-dichlorophenoxybutyric acid; 2,4-dichlorophenoxybutyric acid, methyl ester; 2,6-dichlorobenzonitrile; 2-deoxyglucose; 5-Tetradecyloxy-w-furoic acid; A-922500; acetochlor; alachlor; ametryn; amphotericin; atrazine; benfluralin; bensulide; bentazon; bromacil; bromoxynil; Cafenstrole; carbonyl cyanide m-chlorophenyl hydrazone (CCCP); carbonyl cyanide-p-trifluoromethoxyphenylhydrazone (FCCP); cerulenin; chlorpropham; chlorsulfuron; clofibric acid; clopyralid; colchicine; cycloate; cyclohexamide; C75; DACTHAL (dimethyl tetrachloroterephthalate); dicamb a; dichloroprop ((R)-2-(2,4-dichlorophenoxy)propanoic acid); Diflufenican; dihyrojasmonic acid, methyl ester; diquat; diuron; dimethylsulfoxide; Epigallocatechin gallate (EGCG); endothall; ethalfluralin; ethanol; ethofumesate; Fenoxaprop-p-ethyl; Fluazifop-p-Butyl; fluometuron; fomasefen; foramsulfuron; gibberellic acid; glufosinate ammonium; glyphosate; haloxyfop; hexazinone; imazaquin; isoxaben; Lipase inhibitor THL ((−)-Tetrahydrolipstatin); malonic acid; MCPA (2-methyl-4-chlorophenoxyacetic acid); MCPB (4-(4-chloro-o-tolyloxy)butyric acid); mesotrione; methyl dihydroj asmonate; metolachlor; metribuzin; Mildronate; molinate; naptalam; norharman; orlistat; oxadiazon; oxyfluorfen; paraquat; pendimethalin; pentachlorophenol; PF-04620110; phenethyl alcohol; phenmedipham; picloram; Platencin; Platensimycin; prometon; prometryn; pronamide; propachlor; propanil; propazine; pyrazon; Quizalofop-p-ethyl; s-ethyl dipropylthiocarbamate (EPTC); s,s,s-tributylphosphorotrithioate; salicylhydroxamic acid; sesamol; siduron; sodium methane arsenate; simazine; T-863 (DGAT inhibitor); tebuthiuron; terbacil; thiobencarb; tralkoxydim; triallate; triclopyr; triclosan; trifluralin; and vulpinic acid and others.

The oleaginous cells produce a storage oil, which is primarily triacylglyceride and may be stored in storage bodies of the cell. A raw oil may be obtained from the cells by disrupting the cells and isolating the oil. The raw oil may comprise sterols produced by the cells. Patent applications WO2008/151149, WO2010/063031, WO2010/063032, WO2011/150410, WO2011/150411, WO2012/061647, WO2012/061647, WO2012/106560, WO2013/158938, WO2014/120829, WO2014/151904, WO2015/051319, WO2016/007862, WO2016/014968, WO2016/044779, and WO2016/164495 disclose heterotrophic cultivation and oil isolation techniques for oleaginous microalgae. For example, oil may be obtained by providing or cultivating, drying and pressing the cells. The oils produced may be refined, bleached and deodorized (RBD) as known in the art or as described in WO2010/120939. The raw or RBD oils may be used in a variety of food, chemical, and industrial products or processes. Even after such processing, the oil may retain a sterol profile characteristic of the source. Sterol profiles of microalga and the microalgal cell oils are disclosed below. After recovery of the oil, a valuable residual biomass remains. Uses for the residual biomass include the production of paper, plastics, absorbents, adsorbents, drilling fluids, as animal feed, for human nutrition, or for fertilizer.

In an embodiment of the invention nucleic acids that encode novel acyl transferases are provided. The novel acyltransferases are useful in altering the fatty acid profile and/or altering the regiospecific profile of an oil produced by a host cell. The nucleic acids of the invention may contain control sequences upstream and downstream in operable linkage with the gene of interest. These control sequences include promoters, targeting sequences, untranslated sequences and other control elements. Nucleic acids of the invention encode acyltransferases that function in type II fatty acid synthesis. The acyltransferase genes are isolated from higher plants and can be expressed in a wide variety of host cells. The acyltransferases include lysophosphatidic acid acyltransferase (LPAAT), glycerol phosphate acyltransferase (GPAT), diacyl glycerol acyltransferase (DGAT), lysophosphatidylcholine acyltransferase (LPCAT), or phospholipase A2 (PLA2). and other lipid biosynthetic pathway genes as discussed herein. The acyltransferases of the invention are shown in Table 5. In one embodiment, the acyltransferases of the invention have acyltransferase activity and the amino acid sequence comprises at least 96.3%, 98%, or 99% identity to an acyltransferase of clade 1 of Table 5. In another embodiment, the acyltransferases of the invention have acyltransferase activity and the amino acid sequence comprises at least 93.9%, 98%, or 99% identity to an acyltransferase of clade 2 of Table 5. In one embodiment, the acyltransferases of the invention have acyltransferase activity and the amino acid sequence comprises at least 86.5%, 90%, 95%, 98%, or 99% identity to an acyltransferase of clade 3 of Table 5. In one embodiment, the acyltransferases of the invention have acyltransferase activity and the amino acid sequence comprises at least 78.5%, 80%, 85%, 90%, 95%, 98%, or 99% identity to an acyltransferase of clade 4 of Table 5. The acyltransferases when expressed increase the SOS, POP, POS, SLS, PLO, and/or PLO content DCW in host cells and the oils recovered from the host cells. The acyltransferases when expressed in host cells decreases the sat-sat-sat content of the oil by DCW. The acyltransferases when expressed in host cells increases the sat-unsat-sat/sat-sat-sat ratio of the oil by DCW.

In an embodiment of the invention nucleic acids that encode variant Brassica napus thiosterases (FATA) are provided. The novel thioesterases are useful in altering the fatty acid profile of an oil produced by a host cell. The variant Brassica napus thiosterases prefer to hydrolyze long chain fatty acyl groups from the acyl carrier protein. The nucleic acids of the invention may contain control sequences upstream and downstream in operable linkage with the gene of interest. These control sequences include promoters, targeting sequences, untranslated sequences and other control elements. Nucleic acids of the invention encode thiosterases that function in type II fatty acid synthesis. The thioesterase genes, isolated from higher plants, are altered to create variant thioesterases that have certain amino acids that have been altered from the wild type enzyme. Due to the altered amino acid(s), the substrate specificity of the thioesterase is altered. The variant thioesterases can be expressed in a wide variety of host cells. The nucleic acids encode the variant thioesterases having amino acid sequences that are 75%, 80%, 85%, 90%, 95%, 98%, 99%, or 100%, or at least 75%, 80%, 85%, 90%, 95%, 98%, 99%, or 100% identical to SEQ ID NOs: 165, 166, 167, or 198 and comprise one or more of amino acid variants D124A, D209A, D127A or D212A. The variant BnOTE enzymes increased C18:0 content by DCW, decreased C18:1 content by DCW, and decreased C18:2 content by DCW in host cells and the oils recovered from the host cells.

In an embodiment of the invention nucleic acids that encode variant Garcinia mangostana thiosterases (FATA) are provided. The novel thioesterases are useful in altering the fatty acid profile of an oil produced by a host cell. The variant Garcinia mangostana thiosterases prefer to hydrolyze long chain fatty acyl groups from the acyl carrier protein. The nucleic acids of the invention may contain control sequences upstream and downstream in operable linkage with the gene of interest. These control sequences include promoters, targeting sequences, untranslated sequences and other control elements. Nucleic acids of the invention encode thiosterases that function in type II fatty acid synthesis. The thioesterase genes, isolated from higher plants, are altered to create variant thioesterases that have certain amino acids that have been altered from the wild type enzyme. Due to the altered amino acid(s), the substrate specificity of the thioesterase is altered. The variant thioesterases can be expressed in a wide variety of host cells. The nucleic acids encode the variant thioesterases having amino acid sequences that are 75%, 80%, 85%, 90%, 95%, 98%, 99%, or 100%, or at least 75%, 80%, 85%, 90%, 95%, 98%, 99%, or 100% identical to SEQ ID NOs: 137, 138, 139, 140, 141, 142, 143, 144, 145, 146, 147, 148, 149, or 150 and comprise one or more of amino acid variants L91F, L91K, L91S, G96A, G96T, G96V, G108A, G108V, S111A, S111V T156F, T156A, T156K, T156V, or V193A. The variant GmFATA enzymes increased C18:0 content by DCW, decreased C18:1 content by DCW, and decreased C18:2 content by DCW in host cells and the oils recovered from the host cells.

The nucleic acids of the invention can be codon optimized for expression in a target host cell (e.g., using the codon usage tables of Tables 1a, 1b, 2a, and 2b. For example, at least 60%, 65%, 70%, 75%, 80%, 85%, 90%, 95%, or 100% of the codons used can be the most preferred codon according to Tables 1a, 1b, 2a, and 2b. Alternately, at least 60%, 65%, 70%, 75%, 80%, 85%, 90%, 95%, or 100% of the codons used can be the first or second most preferred codon according to Tables 1a, 1b, 2a, and 2b. Preferred codons for Prototheca strains and for Chlorella protothecoides are shown below in Tables 1a and 1b, respectively.

TABLE 1a
Preferred codon usage in Prototheca strains.

| | | | | | | | |
|---|---|---|---|---|---|---|---|
| Ala | GCG | 345 | (0.36) | Asn | AAT | 8 | (0.04) |
| | GCA | 66 | (0.07) | | AAC | 201 | (0.96) |
| | GCT | 101 | (0.11) | | | | |
| | GCC | 442 | (0.46) | Pro | CCG | 161 | (0.29) |
| | | | | | CCA | 49 | (0.09) |
| Cys | TGT | 12 | (0.10) | | CCT | 71 | (0.13) |
| | TGC | 105 | (0.90) | | CCC | 267 | (0.49) |
| Asp | GAT | 43 | (0.12) | Gln | CAG | 226 | (0.82) |
| | GAC | 316 | (0.88) | | CAA | 48 | (0.18) |
| Glu | GAG | 377 | (0.96) | Arg | AGG | 33 | (0.06) |
| | GAA | 14 | (0.04) | | AGA | 14 | (0.02) |
| | | | | | CGG | 102 | (0.18) |
| Phe | TTT | 89 | (0.29) | | CGA | 49 | (0.08) |
| | TTC | 216 | (0.71) | | CGT | 51 | (0.09) |
| | | | | | CGC | 331 | (0.57) |
| Gly | GGG | 92 | (0.12) | | | | |
| | GGA | 56 | (0.07) | Ser | AGT | 16 | (0.03) |
| | GGT | 76 | (0.10) | | AGC | 123 | (0.22) |
| | GGC | 559 | (0.71) | | TCG | 152 | (0.28) |
| | | | | | TCA | 31 | (0.06) |
| His | CAT | 42 | (0.21) | | TCT | 55 | (0.10) |
| | CAC | 154 | (0.79) | | TCC | 173 | (0.31) |
| Ile | ATA | 4 | (0.01) | Thr | ACG | 184 | (0.38) |
| | ATT | 30 | (0.08) | | ACA | 24 | (0.05) |
| | ATC | 338 | (0.91) | | ACT | 21 | (0.05) |
| | | | | | ACC | 249 | (0.52) |
| Lys | AAG | 284 | (0.98) | | | | |
| | AAA | 7 | (0.02) | Val | GTG | 308 | (0.50) |
| | | | | | GTA | 9 | (0.01) |
| Leu | TTG | 26 | (0.04) | | GTT | 35 | (0.06) |
| | TTA | 3 | (0.00) | | GTC | 262 | (0.43) |
| | CTG | 447 | (0.61) | | | | |
| | CTA | 20 | (0.03) | Trp | TGG | 107 | (1.00) |
| | CTT | 45 | (0.06) | | | | |
| | CTC | 190 | (0.26) | Tyr | TAT | 10 | (0.05) |
| | | | | | TAC | 180 | (0.95) |
| Met | ATG | 191 | (1.00) | | | | |

| | |
|---|---|
| Stop | TGA/TAG/TAA |

TABLE 1b
Preferred codon usage in Chlorella protothecoides.

| | | | |
|---|---|---|---|
| TTC (Phe) | TAC (Tyr) | TGC (Cys) | TGA (Stop) |
| TGG (Trp) | CCC (Pro) | CAC (His) | CGC (Arg) |
| CTG (Leu) | CAG (Gln) | ATC (Ile) | ACC (Thr) |
| GAC (Asp) | TCC (Ser) | ATG (Met) | AAG (Lys) |
| GCC (Ala) | AAC (Asn) | GGC (Gly) | GTG (Val) |
| GAG (Glu) | | | |

TABLE 2a
Codon usage for Cuphea wrightii

| | | | |
|---|---|---|---|
| UUU F 0.48 19.5 ( 52) | UCU S 0.21 19.5 ( 52) | UAU Y 0.45 6.4 ( 17) | UGU C 0.41 10.5 ( 28) |
| UUC F 0.52 21.3 ( 57) | UCC S 0.26 23.6 ( 63) | UAC Y 0.55 7.9 ( 21) | UGC C 0.59 15.0 ( 40) |
| UUA L 0.07 5.2 ( 14) | UCA S 0.18 16.8 ( 45) | UAA * 0.33 0.7 ( 2) | UGA * 0.33 0.7 ( 2) |
| UUG L 0.19 14.6 ( 39) | UCG S 0.11 9.7 ( 26) | UAG * 0.33 0.7 ( 2) | UGG W 1.00 15.4 ( 41) |
| CUU L 0.27 21.0 ( 56) | CCU P 0.48 21.7 ( 58) | CAU H 0.60 11.2 ( 30) | CGU R 0.09 5.6 ( 15) |
| CUC L 0.22 17.2 ( 46) | CCC P 0.16 7.1 ( 19) | CAC H 0.40 7.5 ( 20) | CGC R 0.13 7.9 ( 21) |
| CUA L 0.13 10.1 ( 27) | CCA P 0.21 9.7 ( 26) | CAA Q 0.31 8.6 ( 23) | CGA R 0.11 6.7 ( 18) |
| CUG L 0.12 9.7 ( 26) | CCG P 0.16 7.1 ( 19) | CAG Q 0.69 19.5 ( 52) | CGG R 0.16 9.4 ( 25) |
| AUU I 0.44 22.8 ( 61) | ACU T 0.33 16.8 ( 45) | AAU N 0.66 31.4 ( 84) | AGU S 0.18 16.1 ( 43) |
| AUC I 0.29 15.4 ( 41) | ACC T 0.27 13.9 ( 37) | AAC N 0.34 16.5 ( 44) | AGC S 0.07 6.0 ( 16) |
| AUA I 0.27 13.9 ( 37) | ACA T 0.26 13.5 ( 36) | AAA K 0.42 21.0 ( 56) | AGA R 0.24 14.2 ( 38) |
| AUG M 1.00 28.1 ( 75) | ACG T 0.14 7.1 ( 19) | AAG K 0.58 29.2 ( 78) | AGG R 0.27 16.1 ( 43) |
| GUU V 0.28 19.8 ( 53) | GCU A 0.35 31.4 ( 84) | GAU D 0.63 35.9 ( 96) | GGU G 0.29 26.6 ( 71) |
| GUC V 0.21 15.0 ( 40) | GCC A 0.20 18.0 ( 48) | GAC D 0.37 21.0 ( 56) | GGC G 0.20 18.0 ( 48) |
| GUA V 0.14 10.1 ( 27) | GCA A 0.33 29.6 ( 79) | GAA E 0.41 18.3 ( 49) | GGA G 0.35 31.4 ( 84) |
| GUG V 0.36 25.1 ( 67) | GCG A 0.11 9.7 ( 26) | GAG E 0.59 26.2 ( 70) | GGG G 0.16 14.2 ( 38) |

TABLE 2b
Codon usage for Arabidopsis

| | | | |
|---|---|---|---|
| UUU F 0.51 21.8 (678320) | UCU S 0.28 25.2 (782818) | UAU Y 0.52 14.6 (455089) | UGU C 0.60 10.5 (327640) |
| UUC F 0.49 20.7 (642407) | UCC S 0.13 11.2 (348173) | UAC Y 0.48 13.7 (427132) | UGC C 0.40 7.2 (222769) |
| UUA L 0.14 12.7 (394867) | UCA S 0.20 18.3 (568570) | UAA * 0.36 0.9 ( 29405) | UGA * 0.44 1.2 ( 36260) |
| UUG L 0.22 20.9 (649150) | UCG S 0.10 9.3 (290158) | UAG * 0.20 0.5 ( 16417) | UGG W 1.00 12.5 (388049) |
| CUU L 0.26 24.1 (750114) | CCU P 0.38 18.7 (580962) | CAU H 0.61 13.8 (428694) | CGU R 0.17 9.0 (280392) |
| CUC L 0.17 16.1 (500524) | CCC P 0.11 5.3 (165252) | CAC H 0.39 8.7 (271155) | CGC R 0.07 3.8 (117543) |
| CUA L 0.11 9.9 (307000) | CCA P 0.33 16.1 (502101) | CAA Q 0.56 19.4 (604800) | CGA R 0.12 6.3 (195736) |
| CUG L 0.11 9.8 (305822) | CCG P 0.18 8.6 (268115) | CAG Q 0.44 15.2 (473809) | CGG R 0.09 4.9 (151572) |
| AUU I 0.41 21.5 (668227) | ACU T 0.34 17.5 (544807) | AAU N 0.52 22.3 (693344) | AGU S 0.16 14.0 (435738) |
| AUC I 0.35 18.5 (576287) | ACC T 0.20 10.3 (321640) | AAC N 0.48 20.9 (650826) | AGC S 0.13 11.3 (352568) |
| AUA I 0.24 12.6 (391867) | ACA T 0.31 15.7 (487161) | AAA K 0.49 30.8 (957374) | AGA R 0.35 19.0 (589788) |
| AUG M 1.00 24.5 (762852) | ACG T 0.15 7.7 (240652) | AAG K 0.51 32.7 (1016176) | AGG R 0.20 11.0 (340922) |
| GUU V 0.40 27.2 (847061) | GCU A 0.43 28.3 (880808) | GAU D 0.68 36.6 (1139637) | GGU G 0.34 22.2 (689891) |
| GUC V 0.19 12.8 (397008) | GCC A 0.16 10.3 (321500) | GAC D 0.32 17.2 (535668) | GGC G 0.14 9.2 (284681) |
| GUA V 0.15 9.9 (308605) | GCA A 0.27 17.5 (543180) | GAA E 0.52 34.3 (1068012) | GGA G 0.37 24.2 (751489) |
| GUG V 0.26 17.4 (539873) | GCG A 0.14 9.0 (280804) | GAG E 0.48 32.2 (1002594) | GGG G 0.16 10.2 (316620) |

The cell oils of this invention can be distinguished from conventional vegetable or animal triacylglycerol sources in that the sterol profile will be indicative of the host organism as distinguishable from the conventional source. Conventional sources of oil include soy, corn, sunflower, safflower, palm, palm kernel, coconut, cottonseed, canola, rape, peanut, olive, flax, tallow, lard, cocoa, shea, mango, sal, illipe, kokum, and allanblackia.

The oils provided herein are not vegetable oils. Vegetable oils are oils extracted from plants and plant seeds. Vegetable oils can be distinguished from the non-plant oils provided herein on the basis of their oil content. A variety of methods for analyzing the oil content can be employed to determine the source of the oil or whether adulteration of an oil provided herein with an oil of a different (e.g. plant) origin has occurred. The determination can be made on the basis of one or a combination of the analytical methods. These tests include but are not limited to analysis of one or more of free fatty acids, fatty acid profile, total triacylglycerol content, diacylglycerol content, peroxide values, spectroscopic properties (e.g. UV absorption), sterol profile, sterol degradation products, antioxidants (e.g. tocopherols), pigments (e.g. chlorophyll), d13C values and sensory analysis (e.g. taste, odor, and mouth feel). Many such tests have been standardized for commercial oils such as the Codex Alimentarius standards for edible fats and oils.

Sterol profile analysis is a particularly well-known method for determining the biological source of organic matter. Campesterol, b-sitosterol, and stigamsterol are common plant sterols, with b-sitosterol being a principle plant sterol. For example, b-sitosterol was found to be in greatest abundance in an analysis of certain seed oils, approximately 64% in corn, 29% in rapeseed, 64% in sunflower, 74% in cottonseed, 26% in soybean, and 79% in olive oil (Gul et al. J. Cell and Molecular Biology 5:71-79, 2006).

The sterol profile of a microalgal oil is distinct from the sterol profile of oils obtained from higher plants or animals. Oil isolated from Prototheca moriformis strain UTEX1435 were separately clarified (CL), refined and bleached (RB), or refined, bleached and deodorized (RBD) and were tested for sterol content according to the procedure described in JAOCS vol. 60, no. 8, Aug. 1983. Results of the analysis are shown Table 3 below (units in mg/100 g):

TABLE 3
(units in mg/100 g)

| | | | | | Refined, |
|---|---|---|---|---|---|
| | | | | Refined & | bleached, & |
| | Sterol | Crude | Clarified | bleached | deodorized |
| 1 | Ergosterol | 384 | 398 | 293 | 302 |
| | | (56%) | (55%) | (50%) | (50%) |
| 2 | 5,22-cholestadien-24- | 14.6 | 18.8 | 14 | 15.2 |
| | methyl-3-ol | (2.1%) | (2.6%) | (2.4%) | (2.5%) |
| | (Brassicasterol) | | | | |
| 3 | 24-methylcholest-5- | 10.7 | 11.9 | 10.9 | 10.8 |
| | en-3-ol (Campesterol or | (1.6%) | (1.6%) | (1.8%) | (1.8%) |
| | 22,23- | | | | |
| | dihydrobrassicasterol) | | | | |
| 4 | 5,22-cholestadien-24- | 57.7 | 59.2 | 46.8 | 49.9 |
| | ethyl-3-ol (Stigmasterol | (8.4%) | (8.2%) | (7.9%) | (8.3%) |
| | or poriferasterol) | | | | |
| 5 | 24-ethylcholest-5-en- | 9.64 | 9.92 | 9.26 | 10.2 |
| | 3-ol (β-Sitosterol or | (1.4%) | (1.4%) | (1.6%) | (1.7%) |
| | clionasterol) | | | | |
| 6 | Other sterols | 209 | 221 | 216 | 213 |
| | Total sterols | 685.64 | 718.82 | 589.96 | 601.1 |

These results show three striking features. First, ergosterol was found to be the most abundant of all the sterols, accounting for about 50% or more of the total sterols. The amount of ergosterol is greater than that of campesterol, β-sitosterol, and stigmasterol combined. Ergosterol is steroid commonly found in fungus and not commonly found in plants, and its presence particularly in significant amounts serves as a useful marker for non-plant oils. Secondly, the oil was found to contain brassicasterol. With the exception of rapeseed oil, brassicasterol is not commonly found in plant based oils. Thirdly, less than 2% β-sitosterol was found to be present. β-sitosterol is a prominent plant sterol not commonly found in microalgae, and its presence particularly in significant amounts serves as a useful marker for oils of plant origin. In summary, Prototheca moriformis strain UTEX1435 has been found to contain both significant amounts of ergosterol and only trace amounts of β-sitosterol as a percentage of total sterol content. Accordingly, the ratio of ergosterol: β-sitosterol or in combination with the presence of brassicasterol can be used to distinguish this oil from plant oils.

In some embodiments, the oil content of an oil provided herein contains, as a percentage of total sterols, less than 20%, 15%, 10%, 5%, 4%, 3%, 2%, or 1% β-sitosterol. In other embodiments the oil is free from β-sitosterol.

In some embodiments, the oil is free from one or more of β-sitosterol, campesterol, or stigmasterol. In some embodiments the oil is free from β-sitosterol, campesterol, and stigmasterol. In some embodiments the oil is free from campesterol. In some embodiments the oil is free from stigmasterol.

In some embodiments, the oil content of an oil provided herein comprises, as a percentage of total sterols, less than 20%, 15%, 10%, 5%, 4%, 3%, 2%, or 1% 24-ethylcholest-5-en-3-ol. In some embodiments, the 24-ethylcholest-5-en-3-ol is clionasterol. In some embodiments, the oil content of an oil provided herein comprises, as a percentage of total sterols, at least 1%, 2%, 3%, 4%, 5%, 6%, 7%, 8%, 9%, or 10% clionasterol.

In some embodiments, the oil content of an oil provided herein contains, as a percentage of total sterols, less than 20%, 15%, 10%, 5%, 4%, 3%, 2%, or 1% 24-methylcholest-5-en-3-ol. In some embodiments, the 24-methylcholest-5-en-3-ol is 22, 23-dihydrobrassicasterol. In some embodiments, the oil content of an oil provided herein comprises, as a percentage of total sterols, at least 1%, 2%, 3%, 4%, 5%, 6%, 7%, 8%, 9%, or 10% 22,23-dihydrobrassicasterol.

In some embodiments, the oil content of an oil provided herein contains, as a percentage of total sterols, less than 20%, 15%, 10%, 5%, 4%, 3%, 2%, or 1% 5,22-cholestadien-24-ethyl-3-ol. In some embodiments, the 5, 22-cholestadien-24-ethyl-3-ol is poriferasterol. In some embodiments, the oil content of an oil provided herein comprises, as a percentage of total sterols, at least 1%, 2%, 3%, 4%, 5%, 6%, 7%, 8%, 9%, or 10% poriferasterol.

In some embodiments, the oil content of an oil provided herein contains ergosterol or brassicasterol or a combination of the two. In some embodiments, the oil content contains, as a percentage of total sterols, at least 5%, 10%, 20%, 25%, 35%, 40%, 45%, 50%, 55%, 60%, or 65% ergosterol. In some embodiments, the oil content contains, as a percentage of total sterols, at least 25% ergosterol. In some embodiments, the oil content contains, as a percentage of total sterols, at least 40% ergosterol. In some embodiments, the oil content contains, as a percentage of total sterols, at least 5%, 10%, 20%, 25%, 35%, 40%, 45%, 50%, 55%, 60%, or 65% of a combination of ergosterol and brassicasterol.

In some embodiments, the oil content contains, as a percentage of total sterols, at least 1%, 2%, 3%, 4%, or 5% brassicasterol. In some embodiments, the oil content contains, as a percentage of total sterols less than 10%, 9%, 8%, 7%, 6%, or 5% brassicasterol.

In some embodiments the ratio of ergosterol to brassicasterol is at least 5:1, 10:1, 15:1, or 20:1.

In some embodiments, the oil content contains, as a percentage of total sterols, at least 5%, 10%, 20%, 25%, 35%, 40%, 45%, 50%, 55%, 60%, or 65% ergosterol and less than 20%, 15%, 10%, 5%, 4%, 3%, 2%, or 1% β-sitosterol. In some embodiments, the oil content contains, as a percentage of total sterols, at least 25% ergosterol and less than 5% β-sitosterol. In some embodiments, the oil content further comprises brassicasterol.

Sterols contain from 27 to 29 carbon atoms (C27 to C29) and are found in all eukaryotes. Animals exclusively make C27 sterols as they lack the ability to further modify the C27 sterols to produce C28 and C29 sterols. Plants however are able to synthesize C28 and C29 sterols, and C28/C29 plant sterols are often referred to as phytosterols. The sterol profile of a given plant is high in C29 sterols, and the primary sterols in plants are typically the C29 sterols b-sitosterol and stigmasterol. In contrast, the sterol profiles of non-plant organisms contain greater percentages of C27 and C28 sterols. For example the sterols in fungi and in many microalgae are principally C28 sterols. The sterol profile and particularly the striking predominance of C29 sterols over C28 sterols in plants has been exploited for determining the proportion of plant and marine matter in soil samples (Huang, Wen-Yen, Meinschein W. G., “Sterols as ecological indicators”; Geochimica et Cosmochimia Acta. Vol 43. pp 739-745).

In some embodiments the primary sterols in the microalgal oils provided herein are sterols other than b-sitosterol and stigmasterol. In some embodiments of the microalgal oils, C29 sterols make up less than 50%, 40%, 30%, 20%, 10%, or 5% by weight of the total sterol content.

In some embodiments the microalgal oils provided herein contain C28 sterols in excess of C29 sterols. In some embodiments of the microalgal oils, C28 sterols make up greater than 50%, 60%, 70%, 80%, 90%, or 95% by weight of the total sterol content. In some embodiments the C28 sterol is ergosterol. In some embodiments the C28 sterol is brassicasterol.

Where a fatty acid profile of a triglyceride (also referred to as a “triacylglyceride” or “TAG”) cell oil is given here, it will be understood that this refers to a nonfractionated sample of the storage oil extracted from the cell analyzed under conditions in which phospholipids have been removed or with an analysis method that is substantially insensitive to the fatty acids of the phospholipids (e.g. using chromatography and mass spectrometry). The oil may be subjected to an RBD process to remove phospholipids, free fatty acids and odors yet have only minor or negligible changes to the fatty acid profile of the triglycerides in the oil. Because the cells are oleaginous, in some cases the storage oil will constitute the bulk of all the TAGs in the cell. Examples 1 and 2 below give analytical methods for determining TAG fatty acid composition and regiospecific structure.

Broadly categorized, certain embodiments of the invention include (i) recombinant oleaginous cells that comprise an ablation of one or two or all alleles of an endogenous polynucleotide, including polynucleotides encoding lysophosphatidic acid acyltransferase (LPAAT) or (ii) cells that produce oils having low concentrations of polyunsaturated fatty acids, including cells that are auxotrophic for unsaturated fatty acids; (iii) cells producing oils having high concentrations of particular fatty acids due to expression of one or more exogenous genes encoding enzymes that transfer fatty acids to glycerol or a glycerol ester; (iv) cells producing regiospecific oils, (v) genetic constructs or cells encoding a an LPAAT, a lysophosphatidylcholine acyltransferase (LPCAT), a phosphatidylcholine diacylglycerol cholinephosphotransferase (PDCT), diacylglycerol cholinephosphotransferase (DAG-CPT) or fatty acyl elongase (FAE), (vi) cells producing low levels of saturated fatty acids and/or high levels of C18:1, C18:2, C18:3, C20:1 or C22:1, (vii) and other inventions related to producing cell oils with altered profiles. The embodiments also encompass the oils made by such cells, the residual biomass from such cells after oil extraction, oleochemicals, fuels and food products made from the oils and methods of cultivating the cells.

In any of the embodiments below, the cells used are optionally cells having a type II fatty acid biosynthetic pathway such as plant cells, yeast cells, microalgal cells including heterotrophic or obligate heterotrophic microalgal cells, including cells classified as Chlorophyta, Trebouxiophyceae, Chlorellales, Chlorellaceae, or Chlorophyceae, or cells engineered to have a type II fatty acid biosynthetic pathway using the tools of synthetic biology (i.e., transplanting the genetic machinery for a type II fatty acid biosynthesis into an organism lacking such a pathway). Use of a host cell with a type II pathway avoids the potential for non-interaction between an exogenous acyl-ACP thioesterase or other ACP-binding enzyme and the multienzyme complex of type I cellular machinery. In specific embodiments, the cell is of the species Prototheca moriformis, Prototheca krugani, Prototheca stagnora or Prototheca zopfii or has a 23 S rRNA sequence with at least 65, 70, 75, 80, 85, 90 or 95% nucleotide identity SEQ ID NO: 25. By cultivating in the dark or using an obligate heterotroph, the cell oil produced can be low in chlorophyll or other colorants. For example, the cell oil can have less than 100, 50, 10, 5, 1, 0.0.5 ppm of chlorophyll without substantial purification.

The stable carbon isotope value 613C is an expression of the ratio of ¹³C/¹²C relative to a standard (e.g. PDB, carbonite of fossil skeleton of Belemnite americana from Peedee formation of South Carolina). The stable carbon isotope value δ13C (⁰/₀₀) of the oils can be related to the δ13C value of the feedstock used. In some embodiments the oils are derived from oleaginous organisms heterotrophically grown on sugar derived from a C4 plant such as corn or sugarcane. In some embodiments the 613C (⁰/₀₀) of the oil is from −10 to −17 ⁰/₀₀ or from −13 to −16⁰/₀₀.

In specific embodiments and examples discussed below, one or more fatty acid synthesis genes (e.g., encoding an acyl-ACP thioesterase, a keto-acyl ACP synthase, an LPAAT, an LPCAT, a PDCT, a DAG-CPT, an FAE a stearoyl ACP desaturase, or others described herein) is incorporated into a microalga. It has been found that for certain microalga, a plant fatty acid synthesis gene product is functional in the absence of the corresponding plant acyl carrier protein (ACP), even when the gene product is an enzyme, such as an acyl-ACP thioesterase, that requires binding of ACP to function. Thus, optionally, the microalgal cells can utilize such genes to make a desired oil without co-expression of the plant ACP gene.

For the various embodiments of recombinant cells comprising exogenous genes or combinations of genes, it is contemplated that substitution of those genes with genes having 60%, 70%, 80%, 85%, 90%, 91%, 92%, 93%, 94%, 95%, 96%, 97%, 98%, or 99% or 100% nucleic acid sequence identity can give similar results, as can substitution of genes encoding proteins having 60%, 70%, 80%, 85%, 90%, 91% 92%, 93%, 94%, 95%, 95.5%, 96%, 96.5%, 97%, 97.5%, 98%, 98.5%, 99% or 100% amino acid sequence identity. Nucleic acids encoding the acyltransferases encode acyltransferases that have 60%, 65%, 70%, 75%, 80%, 85%, 90%, 95%, 98%, 99%, or 100%, or at least 60%, 65%, 70%, 75%, 80%, 85%, 90%, 95%, 98%, 99%, or 100% amino acid sequence identity to the acyltransferase disclosed in clade 1, clade 2, clade 3 or clade 4 of Table 5. Likewise, for novel regulatory elements, it is contemplated that substitution of those nucleic acids with nucleic acids having 60%, 70%, 80%, 85%, 90%, 91%, 92%, 93%, 94%, 95%, 96%, 97%, 98%, 99% or 100% nucleic acid can be efficacious. In the various embodiments, it will be understood that sequences that are not necessary for function (e.g. FLAG® tags or inserted restriction sites) can often be omitted in use or ignored in comparing genes, proteins and variants.

The novel genes and gene combinations reported here can be used in higher plants using techniques that are well known in the art. For example, the use of exogenous lipid metabolism genes in higher plants is described in U.S. Pat. Nos. 6,028,247; 5,850,022; 5,639,790; 5,455,167; 5,512,482; and 5,298,421 disclose higher plants with exogenous acyl-ACP thioesterases. WO2009129582 and WO1995027791 disclose cloning of LPAAT in plants. FAD2 ablation and/or down regulation in higher plants is taught in WO 2013112578, and WO2008/006171. SAD ablation and/or down regulation in higher plants is taught in WO 2013112578, and WO 2008006171.

The expression of the novel acyltransferases is shown in Examples 4, 5, 6 and 7. The expression of Cuphea paucipetala or Cuphea ignea LPATs markedly increased the C8:0 and C10:0 fraction of the cell oil. Additionally, the expression of Cuphea paucipetala or Cuphea ignea LPAATs markedly increased the incorporation of C8:0 and C10:0 fatty acids in the sn-2 position of the TAG. This is disclosed in Example 4.

The expression of LPAT genes in host cells increased C18:2 levels and elevated the sat-unsat-sat/sat-sat-sat, (e.g., SOS/SSS) ratio of the cell oil. For example, the expression of Theobroma cacoa LPAT2 drives the transfer of unsaturated fatty acids toward the sn-2 position and reduces the incorporation of saturated fatty acids at sn-2.

The novel LPAATs, GPATs, DGATs, LPCATs, and PLA2 with specificity for mid-chain fatty acids are disclosed. In Example 7, expression of LPAATs and DGATs are disclosed.

When an acyltransferase of the invention is expressed in a host cell, one or more additional exogenous genes can concomitantly be expressed. An embodiment of this invention provides host cells that express a recombinant acyltransferase and concomitantly express one or more additional recombinant genes. The one or more additional genes include invertase, fatty acyl-ACP thioesterase (FATA, FATB), melibiase, ketoacyl synthase (KASI, KASII, KASIII, KASIV), antibiotic selective markers, tags such as FLAG, and THIC. In Examples 4, 5, 6, and 7, the co-expression of nucleic acids that encode LPAATs co-expressed with one or more exogenous genes that encode invertase, fatty acyl-ACP thioesterase, melibiase, ketoacyl synthase, THIC are disclosed.

When an acyltransferase of the invention is expressed in a host cell, an endogenous gene of the host call can concomitantly be ablated or downregulated, thereby eliminating or decreasing the expression of the gene of the host cell. This can be accomplished by using homologous recombination techniques or other RNA inhibitory technologies. The ablated or downregulated gene can be any gene in the host cell. The ablated or downregulated endogenous gene can be stearoyl ACP desaturase, fatty acyl desaturase, fatty acyl-ACP thioesterase (FATA or FATB), ketoacyl synthase (KASI, KASII, KASIII or KAS IV), or an acyltransferase (LPAAT, DGAT, GPAT, LPCAT). When an endogenous is ablated, one, two or more alleles of the endogenous can be ablated. In Example 5, the expression of a Brassica LPAAT, while concomitantly ablating an endogenous stearoyl ACP desaturase is disclosed. In Example 6, LPAATs, GPATs, DGATs, LPCATs and PLA2s with specificity for mid-chain fatty acids were expressed, while ablating a gene encoding stearoyl ACP desaturase. In Example 7 the down regulation of an endogenous FAD2 and a hairpin RNA is disclosed. In co-owned PCT/US2016/026265, applicants disclosed concomitant ablation of an endogenous LPAAT and expression of an exogenous LPAAT.

In one embodiment, the expression of the acyl transferases alters the fatty acid profile and/or the sn-2 profile of the oil produced by the host organism. The fatty acid profiles and the sn-2 profiles that result from the expression of various acyltransferases are disclosed in Tables 6, 7, 10, 11, 12, 13, 16, 17, 18, 19, 20, 22, 23, and 24. The invention provides host cells with altered fatty acid profiles and altered sn-2 profiles according to Tables 6, 7, 10, 11, 12, 13, 16, 17, 18, 19, 20, 22, 23, and 24.

As described in PCT/US2016/026265, co-owned by applicant, transcript profiling was used to discover promoters that modulate expression in response to low nitrogen conditions. The promoters are useful to selectively express various genes and to alter the fatty acid composition of microbial oils. In accordance with an embodiment, there are non-natural constructs comprising a heterologous promoter and a gene, wherein the promoter comprises at least 60%, 65%, 70%, 75%, 80%, 85%, 90%, 95%, or 100% sequence identity to any of the promoters of SEQ ID NOs: 1-18 and the gene is differentially expressed under low vs. high nitrogen conditions. In particular, the Prototheca moriformis AMT02 (SEQ ID NO: 18) and AMT03 promoter (SEQ ID NO: 18) are useful promoters for controlling the expression of an exogenous gene. For example, the promoters can be placed in front of a FAD2 gene in a linoleic acid auxotroph to produce an oil with less than 5, 4, 3, 2, or 1% linoleic acid after culturing first under high nitrogen conditions, then next culturing under low nitrogen conditions. Additional promoters, in particulare Prototheca and Chlorella promoters are described in the sequences and descriptions in this application. For example, the Prototheca HXT1, SAD, LDH1 and other Prototheca promoters are described in Examples 6, 7, 8, and 9. Additionally, the Chlorella SAD, ACT and other Chlorella promoters are described in Examples 6, 7, 8, and 9.

In embodiments of the present invention, oleaginous cells expressing one or more of the genes encoding acyltransferases and/or variant FATA can produce an oil with at least 20, 40, 60 or 70% of C8, C10, C12, C14, C16, or C18 fatty acids.

The invention also provides host cells expressing one or more of the genes encoding acyltransferases and/or variant FATA can produce an oil enriched is oils that are sat-unsat-sat. Oils of this type include SOS, POP, POS, SLS, PLO, PLO. The sat-unsat-sat oils comprise at least 30%, 40%, 50%, 60%, 65%, 70%, 75%, 80%, 85%, 90%, or 95% of the cell oil by dry cell weight.

The invention also provides host cells expressing one or more of the genes encoding acyltransferases and/or variant FATA can produce an oil that is decreased in tri-saturated oils, sat-sat-sat. Oils of this type include PPP, PSS, PPS, SSS, SPS, and PSP. The sat-sat-sat oils comprise less than 50%, 40%, 30%, 20%, 15%, 10%, 8%, 6%, 5%, 4%, 3%, 2%, or 1% of the cell oil by molar fraction or dry cell weight.

The host cells of the invention can produce 25%, 30%, 40%, 50%, 60%, 70%, 80%, 85%, or about 90% oil by cell weight, ±5%. Optionally, the oils produced can be low in DHA or EPA fatty acids. For example, the oils can comprise less than 5%, 2%, or 1% DHA and/or EPA.

In other embodiments of the invention, there is a process for producing an oil, triglyceride, fatty acid, or derivative of any of these, comprising transforming a cell with any of the nucleic acids discussed herein. In another embodiment, the transformed cell is cultivated to produce an oil and, optionally, the oil is extracted. Oil extracted in this way can be used to produce food, oleochemicals or other products.

The oils discussed above alone or in combination are useful in the production of foods, fuels and chemicals (including plastics, foams, films, etc). The oils, triglycerides, fatty acids from the oils may be subjected to C—H activation, hydroamino methylation, methoxy-carbonation, ozonolysis, enzymatic transformations, epoxidation, methylation, dimerization, thiolation, metathesis, hydro-alkylation, lactonization, or other chemical processes.

After extracting the oil, a residual biomass may be left, which may have use as a fuel, as an animal feed, or as an ingredient in paper, plastic, or other product. For example, residual biomass from heterotrophic algae can be used in such products.

### EXAMPLES

### Example 1: Fatty Acid Analysis by Fatty Acid Methyl Ester Detection

Lipid samples were prepared from dried biomass. 20-40 mg of dried biomass was resuspended in 2 mL of 5% H₂SO₄ in MeOH, and 200 ul of toluene containing an appropriate amount of a suitable internal standard (C19:0) was added. The mixture was sonicated briefly to disperse the biomass, then heated at 70-75° C. for 3.5 hours. 2 mL of heptane was added to extract the fatty acid methyl esters, followed by addition of 2 mL of 6% K₂CO₃ (aq) to neutralize the acid. The mixture was agitated vigorously, and a portion of the upper layer was transferred to a vial containing Na₂SO₄ (anhydrous) for gas chromatography analysis using standard FAME GC/FID (fatty acid methyl ester gas chromatography flame ionization detection) methods. Fatty acid profiles reported below were determined by this method.

### Example 2: Analysis of Regiospecific Profile

LC/MS TAG distribution analyses were carried out using a Shimadzu Nexera ultra high performance liquid chromatography system that included a SIL-30AC autosampler, two LC-30AD pumps, a DGU-20A5 in-line degasser, and a CTO-20A column oven, coupled to a Shimadzu LCMS 8030 triple quadrupole mass spectrometer equipped with an APCI source. Data was acquired using a Q3 scan of m/z 350-1050 at a scan speed of 1428 u/sec in positive ion mode with the CID gas (argon) pressure set to 230 kPa. The APCI, desolvation line, and heat block temperatures were set to 300, 250, and 200° C., respectively, the flow rates of the nebulizing and drying gases were 3.0 L/min and 5.0 L/min, respectively, and the interface voltage was 4500 V. Oil samples were dissolved in dichloromethane-methanol (1:1) to a concentration of 5 mg/mL, and 0.8 μL of sample was injected onto Shimadzu Shim-pack XR-ODS III (2.2 μm, 2.0×200 mm) maintained at 30° C. A linear gradient from 30% dichloromethane-2-propanol (1:1)/acetonitrile to 51% dichloromethane-2-propanol (1:1)/acetonitrile over 27 minutes at 0.48 mL/min was used for chromatographic separations.

### Example 3: Cultivation of Microalgae

### Standard Lipid Production Conditions:

Cells scraped from a source plate with toothpicks were used to inoculate pre-seed cultures of 0.5 mL EB03, 0.5% glucose, 1×DAS2 cultures in 96-well blocks. Pre-seed cultures were grown for 70-75 h at 28° C., 900 rpm in a Multitron shaker. 40 μL of pre-seed cultures were used to inoculate seed cultures of 0.46 mL H29, 4% glucose, 25 mM citrate pH 5 or 100 mM PIPES pH 7.3, 1×DAS2 (8% inoculum), and grown for 24-28 h at 28° C., 900 rpm in a Multitron shaker. 40 μL of seed cultures were used to inoculate lipid production cultures of 0.46 mL H43, 6% glucose, 25 mM citrate pH 5, 1×DAS2 (8% inoculum), and grown for 70-75 h at 28° C., 900 rpm in a Multitron shaker. Fatty acid profiles and lipid titer analyses were performed as disclosed in Examples 1 and 2.

### 50 mL Shake Flask Format

Cells scraped from a source plate with inoculation loops, or cell cultures from cryovials were used to inoculate pre-seed cultures of 10 mL EB03, 0.5% glucose, 1×DAS2 cultures in 50 mL bioreactor tubes. Pre-seed cultures were grown for 70-75 h at 28° C., 200 rpm in a Kuhner shaker. 0.8 mL of pre-seed cultures were used to inoculate seed cultures of 10 mL H29, 4% glucose, 25 mM citrate pH 5 or 100 mM PIPES pH 7.3, 1×DAS2 (8% inoculum), and grown for 24-28 h at 28° C., 200 rpm in a Kuhner shaker. 100 μL of seed cultures were used to inoculate lipid production cultures of 49.9 mL H43, 6% glucose, 25 mM citrate pH 5 or 100 mM PIPES pH 7.3, 1×DAS2 (0.2% inoculum), and grown for 118-122 h at 28° C., 200 rpm in a Kuhner shaker. Fatty acid profiles and lipid titer analyses were performed as disclosed in Examples 1 and 2.

### EB03

| Dry chemicals | |
|---|---|
| Component | Concentration (g/L) |
| K2HPO4 | 3 |
| Sodium Phosphate Dibasic Heptahydrate | 5.66 |
| (Na2HPO4 7H2O) | |
| citric acid monohydrate | 1.2 |
| ammonium sulfate | 1 |
| MgSO4 7H2O | 0.23 |
| CaCl2 2H2O | 0.03 |

| Stock solutions | |
|---|---|
| Component | Concentration (mL/L) |
| 100X C-Trace (3) | 10 |
| Antifoam Sigma 204 | 0.225 |

### H29

| Dry chemicals | Final |
|---|---|
| Component | Concentration (g/L) |
| K₂HPO₄ (Potassium phosphate | 0.25 |
| dibasic anhydrous) | |
| NaH₂PO₄ (Sodium phosphate | 0.18 |
| monobasic) | |
| MgSO₄•7H₂O (Magnesium | 0.24 |
| sulfate heptahydrate) | |
| Citric acid monohydrate | 0.25 |
| Stock solutions | |
| Component | Concentration (mL/L) |
| 0.017M stock CaCl₂•2H₂O | 10 |
| 0.151M (NH₄)₂SO₄ | 52.2 |
| 100X C-Trace (2) | 10 |
| Antifoam Sigma 204 | 0.225 |

### H43

| Dry chemicals | Final |
|---|---|
| Component | Concentration (g/L) |
| K2HPO4 | 0.25 |
| NaH2PO4 | 0.18 |
| MgSO4 7H2O | 0.24 |
| Citric acid H2O | 0.25 |
| Stock solutions | |
| Component | Concentration (mL/L) |
| 0.017M stock CaCl2 2H2O | 10 |
| 100X C-Trace (2) | 10 |
| Antifoam Sigma 204 | 0.225 |
| 0.151M (NH4)2SO4 | 12.5 |

### 1000×DAS2

| Dry chemicals | Final |
|---|---|
| Component | Concentration (g/L) |
| Thiamine-HCl | 0.67 |
| d-Biotin | 0.010 |
| Cyanocobalimin (vit B-12) | 0.008 |
| Calcium Pantothenate | 0.02 |
| PABA (p-aminobenzoic acid) | 0.04 |

### 100×C-Trace(2)

| Dry chemicals | Final |
|---|---|
| Component | Concentration (g/L) |
| CuSO4—5H2O | 0.011 |
| CoC12—6H2O | 0.081 |
| H3BO3 | 0.33 |
| ZnSO4—7H2O | 1.4 |
| MnSO4—H2O | 0.81 |
| Na2MoO4—2H2O | 0.039 |
| FeSO4—7H2O | 0.11 |
| NiCl2—6H2O | 0.013 |
| Citric Acid Monohydrate | 3.0 |

### 100×C-Trace (3)

| Dry chemicals | Final |
|---|---|
| Component | Concentration (g/L) |
| CuSO4—5H2O | 0.011 |
| H3BO3 | 0.33 |
| ZnSO4—7H2O | 1.4 |
| MnSO4—H2O | 0.81 |
| Na2MoO4—2H2O | 0.039 |
| FeSO4—7H2O | 0.11 |
| MCl2—6H2O | 0.013 |
| Citric Acid Monohydrate | 3.0 |

### Example 4: Identification of Novel LPAAT Genes from Sequenced Transcriptomes and Engineering Sn-2 Tag Regiospecificity in Utex1435 by Expression of Heterologous LPAAT Genes from Cuphea Paucipetala, Cuphea Ignea, Cuphea Painteri, and Cuphea Hookeriana

Lysophosphatidic acyltransferase (LPAAT) genes from plant seeds were cloned and expressed in the transgenic strain, S6511, derived from UTEX 1435 (P. moriformis). Expression of the heterologous LPAATs increases C8:0 and C10:0 fatty acid levels and dramatically increases incorporation of C8:0 and C10:0 fatty acids at the sn-2 position of triacylglycerols (TAGs) in transgenic strains.

TAGs are synthesized from various chain length acyl-CoAs and glycerol-3-phosphate by consecutive action of three ER-resident enzymes of the Kennedy pathway—glycerol phosphate acyltransferase (GPAT), LPAAT, and diacylglycerol acyltransferase (DGAT). Substrate specificities of these acyltransferases are known to determine the fatty acid composition of the resulting TAGs. LPAAT acylates the sn-2 hydroxyl group of lysophosphatidic acid (LPA) to form phosphatidic acid (PA), a precursor to TAG. In co-owned applications WO2013/158938, WO2015/051139, and PCT/US2016/026265 we demonstrated expression of LPAAT from Cocos nucifera (CnLPAAT, accession no. AAC49119; Knutzon et al., 1995).

Strain S6511 expresses the acyl-ACP thioesterase (FATB2) gene from Cuphea hookeriana (ChFATB2), leading to C8:0 and C10:0 fatty acid accumulation of ca. 14% and 28%, respectively. Strain S6511 is a strain made according to the methods disclosed in co-owned WO2010/063031 and WO2010/063032, herein incorporated by reference. Briefly, S6511 is a strain that express sucrose invertase and a C. hookeriana FATB2. The construct pSZ3101:6S::CrTUB2-ScSUC2-CvNR_a:PmAMT03-CpSAD1tp_trimmed:ChFATB2-CvNR_d::6S was engineered into S3150, a strain classically mutagenized to increase lipid yield. We identified novel C8:0- and C10:0-specific LPAATs from seeds exhibiting high levels of C8:0 and C10:0 fatty acids. After we identified and cloned LPAATs we expressed the LPAAT genes in S6511.

### Method for Identification of LPAATs

Seeds were obtained from species exhibiting elevated levels of midchain and other specialized fatty acids (Table 4).

TABLE 4
Fatty acid profiles of mature seeds.
The percentage of each fatty acid making up the seed oil is shown;
abundant and unusual fatty acid species are indicated in bold.

| | | | | | | | | | C18:1 |
|---|---|---|---|---|---|---|---|---|---|
| | | C8:0 | C10:0 | C12:0 | C14:0 | C16:0 | C18:0 | C18:1 | (petroselinate) |
| S01_Cc | Cinnamomum | 0.4 | 54.7 | 39.0 | 1.6 | 0.7 | 0.1 | 2.9 | |
| | camphora | | | | | | | | |
| S02_Uc | Umbellularia | 0.9 | 28.8 | 63.0 | 2.3 | 0.4 | 0.1 | 3.4 | |
| | californica | | | | | | | | |
| S03_Ld | Limnanthes | 0.0 | 0.0 | 0.0 | 0.4 | 0.7 | 0.4 | 2.7 | |
| | douglasii | | | | | | | | |
| S04_Chs | Cuphea | 0.2 | 6.5 | 83.7 | 5.1 | 1.1 | 0.1 | 0.0 | |
| | hyssopifolia | | | | | | | | |
| S05_Ccr | Cuphea | 1.6 | 8.1 | 59.2 | 15.2 | 3.9 | 0.6 | 0.0 | |
| | carthagenensis | | | | | | | | |
| S06_Cpr | Cuphea | 2.0 | 11.5 | 61.3 | 10.8 | 2.7 | 0.5 | 0.0 | |
| | parsonsia | | | | | | | | |
| S07_Cg | Cuphia | 7.1 | 85.1 | 1.7 | 0.3 | 1.0 | 0.2 | 0.0 | |
| | glossostoma | | | | | | | | |
| S08_Cht | Cuphea | 3.5 | 44.3 | 40.0 | 4.3 | 1.2 | 0.3 | 2.2 | |
| | heterophylla | | | | | | | | |
| S11_Dc | Daucus | 0.0 | 0.0 | 0.0 | 0.1 | 5.9 | 0.8 | 11.5 | 65.9 |
| | carrota | | | | | | | | |
| S14_Cw | Cuphea | 0.5 | 20.2 | 62.5 | 5.8 | 2.2 | 0.3 | 2.7 | |
| | wrightii | | | | | | | | |
| S15_Bj | Brassica | 0.0 | 0.0 | 0.0 | 0.1 | 3.2 | 0.7 | 12.1 | |
| | juncea | | | | | | | | |
| S16_Br | Brassica | 0.0 | 0.0 | 0.0 | 0.1 | 2.8 | 1.0 | 16.0 | |
| | rapa | | | | | | | | |
| | nipposinica | | | | | | | | |
| S17_Ca | Cuphea | 90.8 | 2.7 | 0.0 | 0.1 | 1.2 | 0.1 | 1.8 | |
| | avigera var. | | | | | | | | |
| | pulcherrima | | | | | | | | |
| S18_Ch | Cuphea | 64.7 | 29.7 | 0.1 | 0.2 | 1.3 | 0.1 | 1.9 | |
| | hookeriana | | | | | | | | |
| S19_Cpal | Cuphea | 28.9 | 0.8 | 1.3 | 55.1 | 6.2 | 0.2 | 3.0 | |
| | palustris | | | | | | | | |
| S20_Cpai | Cuphea | 67.0 | 20.8 | 0.1 | 0.2 | 2.6 | 0.3 | 3.1 | |
| | painteri | | | | | | | | |
| S21_Cpau | Cuphea | 1.5 | 91.0 | 1.2 | 0.7 | 1.5 | 0.2 | 1.1 | |
| | paucipetala | | | | | | | | |
| S22_Chook | Cuphea | 62.8 | 31.9 | 0.2 | 0.2 | 1.0 | 0.1 | 2.1 | |
| | hookeriana | | | | | | | | |
| S23_Cglut | Cuphea | 5.2 | 29.9 | 46.4 | 3.9 | 1.9 | 0.4 | 0.0 | |
| | glutinosa | | | | | | | | |
| S24_Caequ | Cuphea | 27.1 | 0.0 | 1.4 | 57.4 | 6.0 | 0.2 | 3.2 | |
| | aequipetala | | | | | | | | |
| S25_Ccalc | Cuphea | 8.0 | 20.4 | 46.8 | 7.6 | 3.2 | 0.6 | 3.7 | |
| | calcarata | | | | | | | | |
| S26_Chook | Cuphea | 70.4 | 23.1 | 0.1 | 0.2 | 1.5 | 0.2 | 2.5 | |
| | hookeriana | | | | | | | | |
| S27_Cproc | Cuphea | 0.9 | 86.3 | 0.0 | 1.6 | 2.2 | 0.4 | 3.2 | |
| | procumbens | | | | | | | | |
| S28_Cignea | Cuphea | 3.1 | 84.9 | 0.7 | 0.3 | 2.6 | 0.2 | 2.9 | |
| | ignea | | | | | | | | |
| S35_Ccras | Cuphea | 1.3 | 87.7 | 1.3 | 0.4 | 2.0 | 0.5 | 3.3 | |
| | crassiflora | | | | | | | | |
| S36_Ckoe | Cuphea | 0.0 | 87.4 | 1.4 | 0.8 | 2.2 | 0.4 | 2.3 | |
| | koehneana | | | | | | | | |
| S37_Clept | Cuphea | 1.3 | 86.1 | 1.3 | 0.4 | 2.2 | 0.5 | 3.1 | |
| | leptopoda | | | | | | | | |
| S40_Clop | Cuphea | 0.5 | 82.3 | 2.4 | 1.6 | 3.0 | 0.6 | 3.9 | |
| | lophostoma | | | | | | | | |
| S41_Sal | Sassafras | 4.3 | 65.2 | 22.8 | 0.9 | 0.8 | 5.1 | 0.0 | |
| | albidum db | | | | | | | | |

| | | | | | C22: | C22: | C22:2n9, | |
|---|---|---|---|---|---|---|---|---|
| | C18:2 | C20:0 | C20:1 | C22:0 | 1n17 | 1n9 | 17 | C22:2n6 |
| S01_Cc | 0.6 | 0.0 | | | | | | |
| S02_Uc | 0.6 | 0.0 | | | | | | |
| S03_Ld | 1.5 | 1.5 | 59.9 | 0.3 | 2.8 | 17.4 | 9.3 | 0.5 |
| S04_Chs | 1.7 | 0.1 | | | | | | |
| S05_Ccr | 5.4 | 0.2 | | | | | | |
| S06_Cpr | 5.2 | 0.1 | | | | | | |
| S07_Cg | 2.1 | 0.1 | | | | | | |
| S08_Cht | 3.6 | 0.1 | | | | | | |
| S11_Dc | 13.0 | 0.5 | 0.3 | 0.3 | | | | |
| S14_Cw | 4.7 | | | | | | | |
| S15_Bj | 19.2 | 0.5 | 6.3 | 0.8 | | 38.9 | | 1.3 |
| S16_Br | 16.8 | 0.7 | 8.3 | 1.0 | | 40.1 | | 0.8 |
| S17_Ca | 2.8 | | | | | | | |
| S18_Ch | 2.0 | | | | | | | |
| S19_Cpal | 3.4 | | | | | | | |
| S20_Cpai | 4.5 | | | | | | | |
| S21_Cpau | 2.1 | | | | | | | |
| S22_Chook | 1.2 | | | | | | | |
| S23_Cglut | 8.1 | | | | | | | |
| S24_Caequ | 3.8 | | | | | | | |
| S25_Ccalc | 8.5 | | | | | | | |
| S26_Chook | 1.8 | | | | | | | |
| S27_Cproc | 3.3 | | | | | | | |
| S28_Cignea | 4.4 | | | | | | | |
| S35_Ccras | 2.7 | | | | | | | |
| S36_Ckoe | 4.5 | | | | | | | |
| S37_Clept | 4.1 | | | | | | | |
| S40_Clop | 4.9 | | | | | | | |
| S41_Sal | 0.6 | | | | | | | |

Briefly, RNA was extracted from dried plant seeds and submitted for paired-end sequencing using the Illumina Hiseq 2000 platform. RNA sequence reads were assembled into corresponding seed transcriptomes using the Trinity software package. LPAAT-containing cDNA contigs were identified by mining transcriptomes for sequences with homology to a known LPAAT that was previously identified in-house, CuPSR23 LPAAT2-1 (seeWO2013/158938), using BLAST. For some sequences, a high-confidence, full-length transcript was assembled using Trinity. The resulting amino acid sequences of all new LPAATs were subjected to phylogenetic analyses using previously known, full-length LPAAT sequences (available via NCBI) as well as sequences of previously known LPAATs whose sequences were derived at Solazyme. The analysis showed that the amino acid sequences of the newly discovered LPPAATs were not similar to previously known LPAATs. Table 5 shows the clade analysis in which the novel LPAATs were clustered according to a neighbor joining algorithm. These were found to form 4 clades as listed in Table 5.

TABLE 5
Clade Analysis of LPAATs

| | | | | Percent |
|---|---|---|---|---|
| | | | | amino acid |
| | Amino Acid | | | identity |
| Clade | SEQ ID Nos. | | | to members |
| No. | in Clade | Full Genus Species | Function | of clade |
| 1 | S15 BjLPAAT1d | Brassica juncea | | 96.3 |
| | S15 BjLPAAT1c | Brassica juncea | | |
| | S15 BjLPAAT1a | Brassica juncea | | |
| | S15 BjLPAAT1b | Brassica juncea | | |
| 2 | CuPSR23LPAAT2-1 | Cuphea PSR23 | Prefer C8/ | 93.9 |
| | S40 ClopLPAAT1 | Cuphea lophostoma | C10 sn-2 | |
| | S21 CpauLPAAT1 | Cuphea paucipetala | | |
| | S37 CleptLPAAT1 | Cuphea leptopoda | | |
| | S27 CprocLPAAT1b | Cuphea procumbens | | |
| | S27 CprocLPAAT1 | Cuphea procumbens | | |
| | S04 ChsLPAAT2 | Cuphea hyssopifolia | | |
| | S28 CigneaLPAAT1 | Cuphea ignea | | |
| | S05 CcrLPAAT2a | Cuphea carthagenensis | | |
| | S06 CprLPAAT1 | Cuphea parsonsia | | |
| | S05 CcrLPAAT2b | Cuphea carthagenensis | | |
| | S17 CaLPAAT3 | Cuphea avigera var. | | |
| | | pulcherrima | | |
| | S26 ChookLPAAT1 | Cuphea hookeriana | | |
| | S20 CpaiLPAAT1 | Cuphea painteri | | |
| | S04 ChsLPAAT1 | Cuphea hyssopifolia | | |
| | S25 Ccalc1a | Cuphea calcarata | | |
| | S25 Ccalc1b | Cuphea calcarata | | |
| | S14 CwLPAAT1 | Cuphea wrightii | | |
| | S08 ChtLPAAT1a | Cuphea heterophylla | | |
| | S08 ChtLPAAT1b | Cuphea heterophylla | | |
| | S36 CkoeLPAAT2 | Cuphea koehneana | | |
| | S02 UcLPAAT1b | Umbellularia californica | | |
| | S02 UcLPAAT1a | Umbellularia californica | | |
| | S01 CcLPAAT1a | Cinnamomum camphora | | |
| | S01 CcLPAAT1b | Cinnamomum camphora | | |
| | S41 SaILPAAT1 | Sassafras albidum db | | |
| 3 | S14 CwLPAAT2a | Cuphea wrightii | C18:2 | 86.5 |
| | S14 CwLPAAT2b | Cuphea wrightii | | |
| | S25 CcalcLPAAT2 | Cuphea calcarata | | |
| | S19 CpaILPAAT1 | Cuphea palustris | | |
| | S22 ChookLPAAT3b | Cuphea hookeriana | | |
| | S17 CaLPAAT1 | Cuphea avigera var. | | |
| | | pulcherrima | | |
| | S22 ChookLPAAT3a | Cuphea hookeriana | | |
| | CuPSR23LPAAT3-1 | Cuphea PSR23 | | |
| | S27 CprocLPAAT2b | Cuphea procumbens | | |
| | S27 CprocLPAAT2a | Cuphea procumbens | | |
| | S18 ChLPAAT2a | Cuphea hookeriana | | |
| | S24 CaequLPAAT1d | Cuphea aequipetala | | |
| | S24 CaequLPAAT1b | Cuphea aequipetala | | |
| | S24 CaequLPAAT1a | Cuphea aequipetala | | |
| | S24 CaequLPAAT1c | Cuphea aequipetala | | |
| | S23 CglutLPAAT1a | Cuphea glutinosa | | |
| | S23 CglutLPAAT1b | Cuphea glutinosa | | |
| | S26 ChookLPAAT2b | Cuphea hookeriana | | |
| | S07 CgLPAAT1c | Cuphia glossostoma | | |
| | S07 CgLPAAT1b | Cuphia glossostoma | | |
| | S07 CgLPAAT1a | Cuphia glossostoma | | |
| | S28 CigneaLPAAT2 | Cuphea ignea | | |
| | S36 CkoeLPAAT1 | Cuphea koehneana | | |
| | S35 CcrasLPAAT1a | Cuphea crassiflora | | |
| | S35 CcrasLPAAT1c | Cuphea crassiflora | | |
| | S35 CcrasLPAAT1b | Cuphea crassiflora | | |
| | S35 CcrasLPAAT1d | Cuphea crassiflora | | |
| 4 | Gh LPAAT2B | Garcinia hombroriana | Reduced | 78.5 |
| | Gi LPAAT2B-1 | Garcinia indica | trisaturates, | |
| | Gh LPAAT2A | Garcinia hombroriana | increase | |
| | Gi LPAAT2A | Garcinia indica | unsaturates | |
| | Gh LPAAT2C | Garcinia hombroriana | at Sn-2 | |
| | Gi LPAAT2C-2 | Garcinia indica | position | |
| | S03 LdLPAAT1 | Limnanthes douglasii | | |
| | S11 DcLPAAT1 | Daucus carrota | | |
| | | (carrot) | | |
| | S11 DcLPAAT2 | Daucus carrota | | |
| | | (carrot) | | |
| | S11 DcLPAAT2 | Daucus carrota | | |
| | (truncated) | (carrot) | | |

### Functionality of LPAATs in P. Moriformis

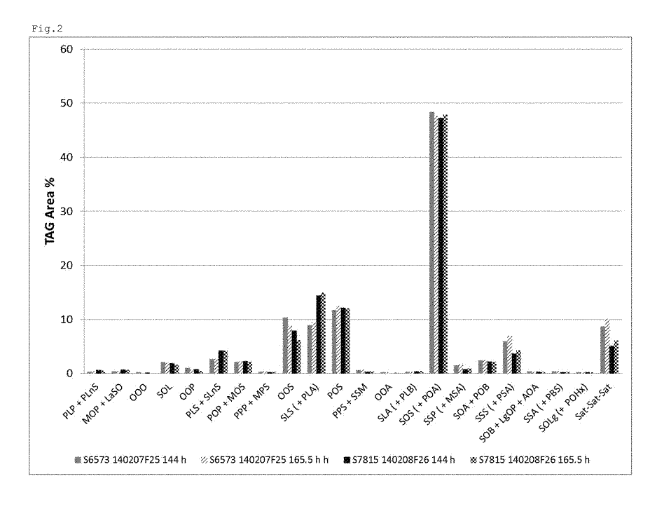

To increase the levels of C8:0 and C10:0 fatty acids in strain S6511, as well as to test the functionality of the newly identified LPAATs, we identified midchain-specific LPAATs from the transcriptomes of species exhibiting high levels of C8:0 and C10:0 fatty acids in their oil seeds and introduced the genes into S56511. LPAATs that co-clustered with CuPSR23 LPAAT2-1, specifically CpauLPAAT1, CigneaLPAAT1, ChookLPAAT1, and CpaiLPAAT1, were selected for synthesis and testing. CpauLPAAT1, CigneaLPAAT1, ChookLPAAT1, and CpaiLPAAT1 were synthesized in a codon-optimized form to reflect UTEX 1435 codon usage. Transgenic strains were generated via transformation of the strain S6511 with a construct encoding one of the four LPAAT genes. The construct pSZ3840 encoding CpauLPAAT1 is shown as an example, but identical methods were used to generate each of the remaining three constructs. Construct pSZ3840 can be written as pLOOP::PmHXT1-ScarMEL1-CvNR:PmAMT3-CpauLPAAT1-CvNR::pLOOP. The sequence of the transforming DNA is provided in FIG. 2 (pSZ3840). The relevant restriction sites in the construct from 5′-3′, BspQI, KpnI, SpeI, XhoI, EcoRI, SpeI, XhoI, SacI, BspQI, respectively, are indicated in lowercase, bold, and underlined. BspQI sites delimit the 5′ and 3′ ends of the transforming DNA. Bold lowercase sequences at the 5′ and 3′ end of the construct represent genomic DNA from UTEX 1435 that target integration to the pLOOP locus via homologous recombination. Proceeding in the 5′ to 3′ direction, the selection cassette has the P. moriformis HXT1 promoter driving expression of the Saccharomyces carlsbergensis MEL1 (conferring the ability to grow on melibiose) and the Chlorella vulgaris Nitrate reductase (NR) gene 3′ UTR. The promoter is indicated by lowercase, boxed text. The initiator ATG and terminator TGA for ScarMEL1 are indicated in bold, uppercase italics, while the coding region is indicated with lowercase italics. The 3′ UTR is indicated by lowercase underlined text. The second cassette containing the codon optimized CpauLPAAT1 gene from Cuphea paucipetala is driven by the P. moriformis AMT3 promoter and has the Chlorella vulgaris Nitrate reductase (NR) gene 3′ UTR. In this cassette, the AMT3 promoter is indicated by lowercase, boxed text. The initiator ATG and terminator TGA for the CpauLPAAT1 gene are indicated in bold, uppercase italics, while the coding region is indicated by lowercase italics. The 3′ UTR is indicated by lowercase underlined text. The final construct was sequenced to ensure correct reading frame and targeting sequences.

SEQ ID NO: 19 pSZ3840/D2554 transforming construct (CpauLPAAT1)
gctcttccgctaacggaggtctgtcaccaaatggaccccgtctattgcgggaaaccacggcgatggcacgtttcaaaacttgatga
aatacaatattcagtatgtcgcgggcggcgacggcggggagctgatgtcgcgctgggtattgcttaatcgccagcttcgcccccgt
cttggcgcgaggcgtgaacaagccgaccgatgtgcacgagcaaatcctgacactagaagggctgactcgcccggcacggctgaa
ttacacaggcttgcaaaaataccagaatttgcacgcaccgtattcgcggtattttgttggacagtgaatagcgatgcggcaatggc
ttgtggcgttagaaggtgcgacgaaggtggtgccaccactgtgccagccagtcctggcggctcccagggccccgatcaagagcca
ggacatccaaactacccacagcatcaacgccccggcctatactcgaaccccacttgcactctgcaatggtatgggaaccacgggg

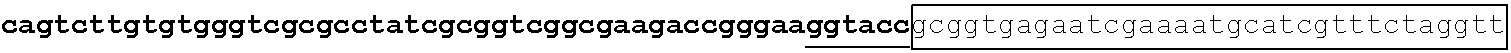

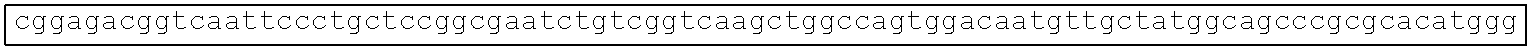

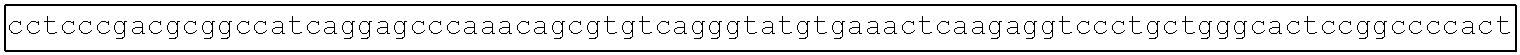

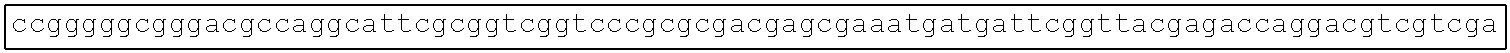

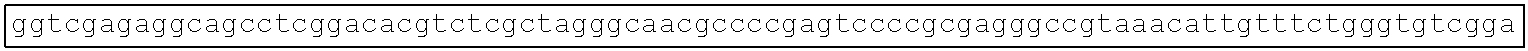

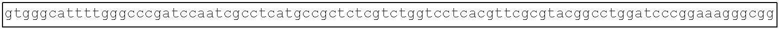

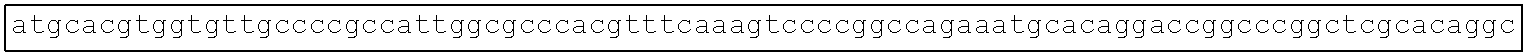

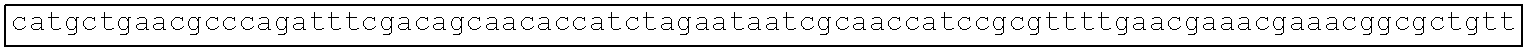

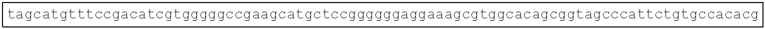

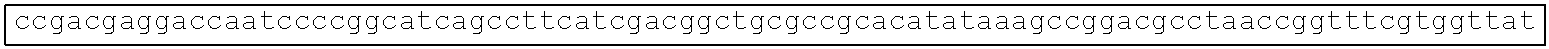

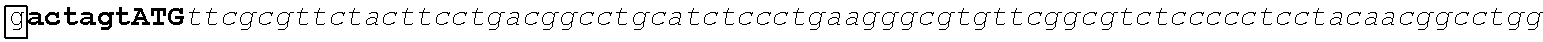

gcctgacgccccagatgggctgggacaactggaacacgttcgcctgcgacgtctccgagcagctgctgctggacacggccga
ccgcatctccgacctgggcctgaaggacatgggctacaagtacatcatcctggacgactgctggtcctccggccgcgactccga
cggcttcctggtcgccgacgagcagaagttccccaacggcatgggccacgtcgccgaccacctgcacaacaactccttcctgtt
cggcatgtactcctccgcgggcgagtacacgtgcgccggctaccccggctccctgggccgcgaggaggaggacgcccagttct
tcgcgaacaaccgcgtggactacctgaagtacgacaactgctacaacaagggccagttcggcacgcccgagatctcctacca
ccgctacaaggccatgtccgacgccctgaacaagacgggccgccccatcttctactccctgtgcaactggggccaggacctga
ccttctactggggctccggcatcgcgaactcctggcgcatgtccggcgacgtcacggcggagttcacgcgccccgactcccgct
gcccctgcgacggcgacgagtacgactgcaagtacgccggcttccactgctccatcatgaacatcctgaacaaggccgccccc
atgggccagaacgcgggcgtcggcggctggaacgacctggacaacctggaggtcggcgtcggcaacctgacggacgacga
ggagaaggcgcacttctccatgtgggccatggtgaagtcccccctgatcatcggcgcgaacgtgaacaacctgaaggcctcct
cctactccatctactcccaggcgtccgtcatcgccatcaaccaggactccaacggcatccccgccacgcgcgtctggcgctacta
cgtgtccgacacggacgagtacggccagggcgagatccagatgtggtccggccccctggacaacggcgaccaggtcgtggc
gctgctgaacggcggctccgtgtcccgccccatgaacacgaccctggaggagatcttcttcgactccaacctgggctccaagaa
gctgacctccacctgggacatctacgacctgtgggcgaaccgcgtcgacaactccacggcgtccgccatcctgggccgcaaca
agaccgccaccggcatcctgtacaacgccaccgagcagtcctacaaggacggcctgtccaagaacgacacccgcctgttcgg
ccagaagatcggctccctgtcccccaacgcgatcctgaacacgaccgtccccgcccacggcatcgcgttctaccgcctgcgcccc
tcctccTGAtacgtactcgaggcagcagcagctcggatagtatcgacacactctggacgctggtcgtgtgatggactgttgccgcc
acacttgctgccttgacctgtgaatatccctgccgcttttatcaaacagcctcagtgtgtttgatcttgtgtgtacgcgcttttgcgagtt
gctagctgcttgtgctatttgcgaataccacccccagcatccccttccctcgtttcatatcgcttgcatcccaaccgcaacttatctacg
ctgtcctgctatccctcagcgctgctcctgctcctgctcactgcccctcgcacagccttggtttgggctccgcctgtattctcctggtact

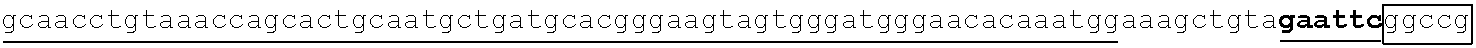

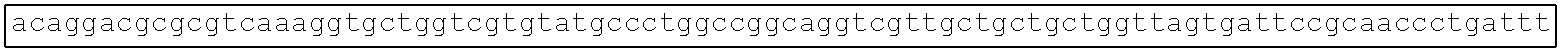

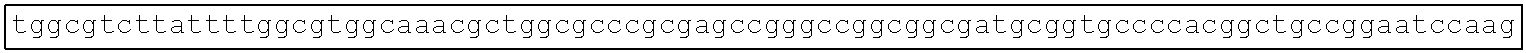

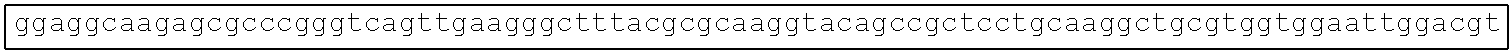

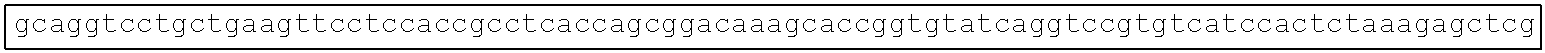

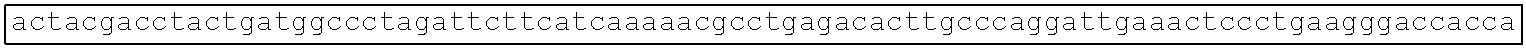

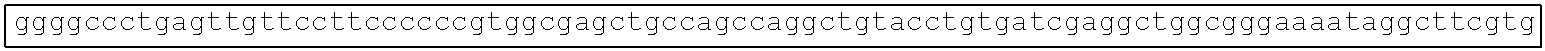

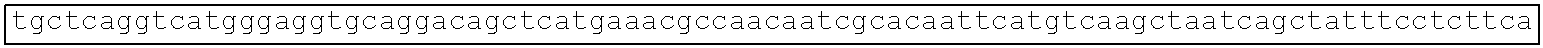

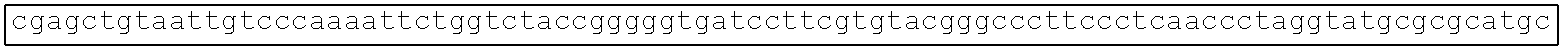

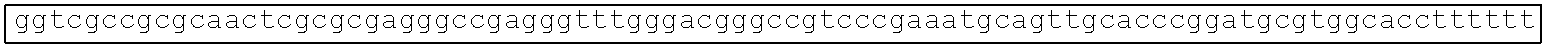

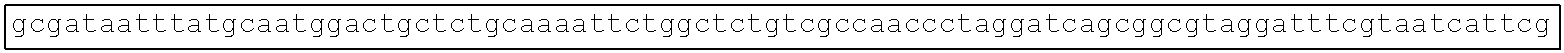

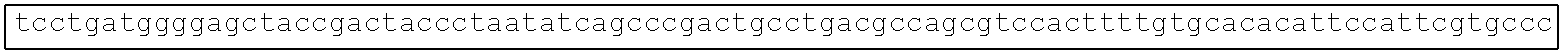

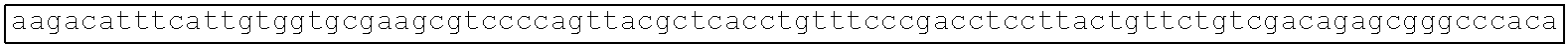

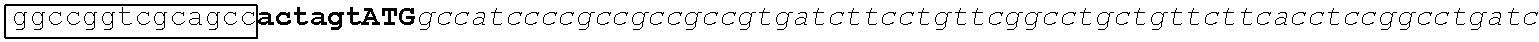

atcaacctgttccaggccagtgatcgtgaggtgtggcccagtccaagaacgcctaccgccgcatcaaccgcgtgttcgccg
agctgctgctgtccgagctgctgtgcctgttcgactggtgggccggcgccaagctgaagctgttcaccgaccccgagaccttcc
gcctgatgggcaaggagcacgccctggtgatcatcaaccacatgaccgagctggactggatgctgggctgggtgatgggcca
gcacctgggctgcctgggctccatcctgtccgtggccaagaagtccaccaagttcctgcccgtgctgggctggtccatgtggttct
ccgagtacctgtacatcgagcgctcctgggccaaggaccgcaccaccctgaagtcccacatcgagcgcctgaccgactacccc
ctgcccttctggatggtgatcttcgtggagggcacccgcttcacccgcaccaagctgctggccgcccagcagtacgccgcctcct
ccggcctgcccgtgccccgcaacgtgctgatcccccgcaccaagggcttcgtgtcctgcgtgtcccacatgcgctccttcgtgccc
gccgtgtacgacgtgaccgtggccttccccaagacctcccccccccccaccctgctgaacctgttcgagggccagtccatcgtgc
tgcacgtgcacatcaagcgccacgccatgaaggacctgcccgagtccgacgacgccgtggcccagtggtgccgcgacaagtt
cgtggagaaggacgccctgctggacaagcacaacgccgaggacaccttctccggccaggaggtgcaccgcaccggctcccg
ccccatcaagtccctgctggtggtgatctcctgggtggtggtgatcaccttcggcgccctgaagttcctgcagtggtcctcctgga
agggcaaggccttctccgtgatcggcctgggcatcgtgaccctgctgatgcacatgctgatcctgtcctcccaggccgagcgctc
ctccaaccccgccaaggtggcccaggccaagctgaagaccgagctgtccatctccaagaaggccaccgacaaggagaacT
GActcgaggcagcagcagctcggatagtatcgacacactctggacgctggtcgtgtgatggactgttgccgccacacttgctgcctt
gacctgtgaatatccctgccgcttttatcaaacagcctcagtgtgtttgatcttgtgtgtacgcgcttttgcgagttgctagctgcttgtg
ctatttgcgaataccacccccagcatccccttccctcgtttcatatcgcttgcatcccaaccgcaacttatctacgctgtcctgctatcc
ctcagcgctgctcctgctcctgctcactgcccctcgcacagccttggtttgggctccgcctgtattctcctggtactgcaacctgtaaac
cagcactgcaatgctgatgcacgggaagtagtgggatgggaacacaaatggaaagcttgagctcagcggcgacggtcctgctacc
gtacgacgttgggcacgcccatgaaagtttgtataccgagcttgttgagcgaactgcaagcgcggctcaaggatacttgaactcct
ggattgatatcggtccaataatggatggaaaatccgaacctcgtgcaagaactgagcaaacctcgttacatggatgcacagtcgc
cagtccaatgaacattgaagtgagcgaactgttcgcttcggtggcagtactactcaaagaatgagctgctgttaaaaatgcactct
cgttctctcaagtgagtggcagatgagtgctcacgccttgcacttcgctgcccgtgtcatgccctgcgccccaaaatttgaaaaaag
ggatgagattattgggcaatggacgacgtcgtcgctccgggagtcaggaccggcggaaaataagaggcaacacactccgcttctt
agctcttc

The sequence for all of the other LPAAT constructs are identical to that of pSZ3840 with the exception of the encoded LPAAT. The LPAAT sequence alone with flanking SpeI and XhoI restriction sites is provided for the remaining LPAAT constructs are shown below. The amino acid sequence of the LPAAT proteins is provided below.

pSZ3841/D2555 (CpaiLPAAT1)
SEQ ID NO: 20
actagt [symbol] gccatcccctccgccgccgtggtgttcctgttcggcctgctgttcttcacctccggcctgatcatcaacctgttccag
gccttctgcttcgtgctgatctcccccctgtccaagaacgcctaccgccgcatcaaccgcgtgttcgccgagctgctgcccctgga
gttcctgtggctgttccactggtgcgccggcgccaagctgaagctgttcaccgaccccgagaccttccgcctgatgggcaagga
gcacgccctggtgatcatcaaccacaagatcgagctggactggatggtgggctgggtgctgggccagcacctgggctgcctg
ggctccatcctgtccgtggccaagaagtccaccaagttcctgcccgtgttcggctggtccctgtggttctccggctacctgttcctg
gagcgctcctgggccaaggacaagatcaccctgaagtcccacatcgagtccctgaaggactaccccctgcccttctggctgatc
atcttcgtggagggcacccgcttcacccgcaccaagctgctggccgcccagcagtacgccgcctcctccggcctgcccgtgcccc
gcaacgtgctgatcccccacaccaagggcttcgtgtcctccgtgtcccacatgcgctccttcgtgcccgccatctacgacgtgacc
gtggccttccccaagacctcccccccccccaccatgctgaagctgttcgagggccagtccgtggagctgcacgtgcacatcaag
cgccacgccatgaaggacctgcccgagtccgacgacgccgtggcccagtggtgccgcgacaagttcgtggagaaggacgcc
ctgctggacaagcacaactccgaggacaccttctccggccaggaggtgcaccacgtgggccgccccatcaaggccctgctggt
ggtgatctcctgggtggtggtgatcatcttcggcgccctgaagttcctgctgtggtcctccctgctgtcctcctggaagggcaagg
ccttctccgtgatcggcctgggcatcgtggccggcatcgtgaccctgctgatgcacatcctgatcctgtcctcccaggccgaggg
ctccaaccccgtgaaggccgcccccgccaagctgaagaccgagctgtcctcctccaagaaggtgaccaacaaggagaac [symbol] [symbol]
ctcgag
pSZ3842/D2556 (CigneaLPAAT1)
SEQ ID NO: 21
actagt [symbol] gccatcgccgccgccgccgtgatcttcctgttcggcctgctgttcttcgcctccggcatcatcatcaacctgttccag
gccctgtgcttcgtgctgatctggcccctgtccaagaacgtgtaccgccgcatcaaccgcgtgttcgccgagctgctgctgatgg
acctgctgtgcctgttccactggtgggccggcgccaagatcaagctgttcaccgaccccgagaccttccgcctgatgggcatgg
agcacgccctggtgatcatgaaccacaagaccgacctggactggatggtgggctggatcctgggccagcacctgggctgcct
gggctccatcctgtccatcgccaagaagtccaccaagttcatccccgtgctgggctggtccgtgtggttctccgagtacctgttcc
tggagcgctcctgggccaaggacaagtccaccctgaagtcccacatggagaagctgaaggactaccccctgcccttctggctg
gtgatcttcgtggagggcacccgcttcacccgcaccaagctgctggccgcccagcagtacgccgcctcctccggcctgcccgtgc
cccgcaacgtgctgatcccccacaccaagggcttcgtgtcctgcgtgtccaacatgcgctccttcgtgcccgccgtgtacgacgt
gaccgtggccttccccaagtcctcccccccccccaccatgctgaagctgttcgagggccagtccatcgtgctgcacgtgcacatc
aagcgccacgccctgaaggacctgcccgagtccgacgacgccgtggcccagtggtgccgcgacaagttcgtggagaaggac
gccctgctggacaagcacaacgccgaggacaccttctccggccaggaggtgcaccacatcggccgccccatcaagtccctgct
ggtggtgatcgcctgggtggtggtgatcatcttcggcgccctgaagttcctgcagtggtcctccctgctgtccacctggaagggc
aaggccttctccgtgatcggcctgggcatcgccaccctgctgatgcacatgctgatcctgtcctcccaggccgagcgctccaacc
ccgccaaggtggccaag [symbol] ctcgag
pSZ3844/D2557 (ChookLPAAT1)
SEQ ID NO: 22
actagt [symbol] gccatcccctccgccgccgtggtgttcctgttcggcctgctgttcttcacctccggcctgatcatcaacctgttccag
gccttctgcttcgtgctgatctcccccctgtccaagaacgcctaccgccgcatcaaccgcgtgttcgccgagctgctgcccctgga
gttcctgtggctgttccactggtgcgccggcgccaagctgaagctgttcaccgaccccgagaccttccgcctgatgggcaagga
gcacgccctggtgatcatcaaccacaagatcgagctggactggatggtgggctgggtgctgggccagcacctgggctgcctg
ggctccatcctgtccgtggccaagaagtccaccaagttcctgcccgtgttcggctggtccctgtggttctccgagtacctgttcctg
gagcgctcctgggccaaggacaagatcaccctgaagtcccacatcgagtccctgaaggactaccccctgcccttctggctgatc
atcttcgtggagggcacccgcttcacccgcaccaagctgctggccgcccagcagtacgccgcctcctccggcctgcccgtgcccc
gcaacgtgctgatcccccacaccaagggcttcgtgtcctccgtgtcccacatgcgctccttcgtgcccgccatctacgacgtgacc
gtggccttccccaagacctcccccccccccaccatgctgaagctgttcgagggccagtccgtggagctgcacgtgcacatcaag
cgccacgccatgaaggacctgcccgagtccgacgacgccgtggcccagtggtgccgcgacaagttcgtggagaaggacgcc
ctgctggacaagcacaactccgaggacaccttctccggccaggaggtgcaccacgtgggccgccccatcaaggccctgctggt
ggtgatctcctgggtggtggtgatcatcttcggcgccctgaagttcctgctgtggtcctccctgctgtcctcctggaagggcaagg
ccttctccgtgatcggcctgggcatcgtggccggcatcgtgaccctgctgatgcacatcctgatcctgtcctcccaggccgaggg
ctccaaccccgtgaaggccgcccccgccaagctgaagaccgagctgtcctcctccaagaaggtgaccaacaaggagaac [symbol] [symbol]
ctcgag

To determine the impact of the CpauLPAAT1, CigneaLPAAT1, ChookLPAAT1, and CpaiLPAAT1 genes on mid-chain fatty acid accumulation, the above constructs containing the codon optimized CpauLPAAT1, CigneaLPAAT1, ChookLPAAT1, and CpaiLPAAT1 genes were transformed into strain S6511. Primary transformants were clonally purified and grown under standard lipid production conditions at pH7.0 (all the strains require growth at pH 7.0 to allow for maximal expression of the LPAAT gene driven by the pH-regulated AMT3 promoter). The resulting profiles from a set of representative clones arising from these transformations are shown in Table 6.

TABLE 6
Transformants of pSZ3840 (CpauLPAAT1), pSZ3841 (CpaiLPAAT1),
pSZ3842 (CigneaLPAAT1), and pSZ3844 (ChookLPAAT1). The fatty acid profiles for
transgenic strains expressing LPAATs derived from C. paucipetala, C. painteri, C. ignea, and C. hookeriana.

| | Sample ID | C8:0 | C10:0 | C12:0 | C14:0 | C16:0 | C18:0 | C18:1 | C18:2 | C18:3a |
|---|---|---|---|---|---|---|---|---|---|---|
| Parent | S6511a | 14.4 | 27.7 | 0.6 | 1.3 | 8.8 | 1.6 | 38.2 | 5.4 | 0.4 |
| | S6511b | 14.5 | 27.7 | 0.6 | 1.3 | 8.6 | 1.6 | 38.4 | 5.3 | 0.4 |
| pSZ3840 CpauLPAAT1 | S6511; T792; D2554-20 | 16.6 | 29.9 | 0.7 | 1.3 | 8.0 | 1.0 | 35.2 | 5.2 | 0.5 |
| | S6511; T792; D2554-17 | 14.6 | 28.7 | 0.6 | 1.3 | 8.4 | 1.7 | 37.1 | 5.7 | 0.5 |
| | S6511; T792; D2554-41 | 15.2 | 28.5 | 0.7 | 1.3 | 8.3 | 1.4 | 37.5 | 5.2 | 0.4 |
| | S6511; T792; D2554-35 | 14.7 | 28.4 | 0.6 | 1.3 | 8.6 | 1.6 | 37.3 | 5.6 | 0.5 |
| | S6511; T792; D2554-27 | 15.2 | 27.6 | 0.7 | 1.3 | 9.5 | 1.5 | 37.1 | 5.1 | 0.4 |
| pSZ3841 CpaiLPAAT1 | S6511; T792; D2555-34 | 17.3 | 29.5 | 0.7 | 1.3 | 7.8 | 1.2 | 35.1 | 5.1 | 0.4 |
| | S6511; T792; D2555-43 | 17.5 | 29.1 | 0.7 | 1.3 | 8.0 | 0.9 | 35.4 | 5.0 | 0.5 |
| | S6511; T792; D2555-10 | 15.7 | 28.3 | 0.7 | 1.3 | 8.6 | 1.6 | 36.2 | 5.7 | 0.5 |
| | S6511; T792; D2555-22 | 16.0 | 27.9 | 0.7 | 1.3 | 8.4 | 0.9 | 37.8 | 5.0 | 0.4 |
| | S6511; T792; D2555-44 | 15.3 | 27.5 | 0.6 | 1.3 | 8.1 | 1.8 | 38.2 | 5.4 | 0.4 |
| pSZ3842 CigneaLPAAT1 | S6511; T792; D2556-38 | 16.2 | 29.2 | 0.7 | 1.3 | 8.1 | 1.3 | 36.1 | 5.2 | 0.5 |
| | S6511; T792; D2556-22 | 14.3 | 28.5 | 0.7 | 1.3 | 8.5 | 1.6 | 37.6 | 5.7 | 0.5 |
| | S6511; T792; D2556-44 | 13.6 | 28.4 | 0.7 | 1.4 | 9.0 | 1.5 | 36.3 | 6.7 | 0.7 |
| | S6511; T792; D2556-14 | 14.1 | 28.0 | 0.6 | 1.3 | 8.6 | 1.7 | 38.0 | 5.6 | 0.5 |
| | S6511; T792; D2556-36 | 14.3 | 28.0 | 0.6 | 1.3 | 8.6 | 1.7 | 37.9 | 5.7 | 0.5 |
| pSZ3844 ChookLPAAT1 | S6511; T792; D2557-47 | 15.8 | 29.3 | 0.7 | 1.3 | 8.2 | 1.2 | 36.5 | 5.0 | 0.5 |
| | S6511; T792; D2557-24 | 16.8 | 28.8 | 0.7 | 1.3 | 8.1 | 1.2 | 35.8 | 5.4 | 0.5 |
| | S6511; T792; D2557-30 | 15.2 | 28.3 | 0.7 | 1.3 | 8.5 | 1.6 | 36.8 | 5.7 | 0.5 |
| | S6511; T792; D2557-39 | 14.7 | 28.2 | 0.7 | 1.3 | 8.7 | 1.5 | 37.3 | 5.7 | 0.5 |
| | S6511; T792; D2557-26 | 15.3 | 27.7 | 0.7 | 1.4 | 8.7 | 0.9 | 37.7 | 5.4 | 0.5 |

The transformants in Table 6 display a marked increase in the production of C8:0 and C10:0 fatty acids upon expression of the heterologous LPAATs. To determine if expression of the heterologous LPAAT genes affected the regiospecificity of fatty acids at the sn-2 position, we analyzed TAGs from representative D2554 (CpauLPAAT1), D2555 (CpaiLPAAT1), D2556 (CigneaLPAAT1), and D2557 (ChookLPAAT1) strains utilizing the porcine pancreatic lipase method. Cells were grown under conditions to maximize midchain fatty acid levels and to generate sufficient biomass for TAG analysis. TAG and sn-2 profiles are shown in Table 7.

Table 7: Inclusion of C8:0 and C10:0 fatty acids at the sn-2 position of TAGs. Selected transformants were subjected to porcine pancreatic lipase determination of fatty acid inclusion at the sn-2 position. The general fatty acid distribution in triacylglycerols (TAG) is shown to indicate fatty acid abundance for each transformant. In addition, the sn-2-specific distribution is shown. Numbers highlighted in bold and italic reflect significantly increased inclusion of the noted fatty acid compared to the parent S6511.

TABLE 7

| | | Strain: | | | | | | | | | |
|---|---|---|---|---|---|---|---|---|---|---|---|
| | | | | S6511; T792; | | S6511; T792; | | S6511; T792; | | S6511; T792; | |
| | | | | D2554-20 | | D2555-34 | | D2556-38 | | D2557-24 | |
| | | S6511 | | (CpauLPAAT1) | | (CpaiLPAAT1) | | (CigneaLPAAT1) | | (ChookLPAAT1) | |
| | | Analysis | | | | | | | | | |
| | | TAG | sn-2 | TAG | sn-2 | TAG | sn-2 | TAG | sn-2 | TAG | sn-2 |
| Fatty Acid | C8:0 | 14.4 | 8.5 | 16.6 | 12.8 | 17.3 | [symbol] | 16.2 | 10.0 | 16.8 | [symbol] |
| (area %) | C10:0 | 27.7 | 26.4 | 29.9 | [symbol] | 29.5 | 22.2 | 29.2 | [symbol] | 28.8 | 19.4 |
| | C12:0 | 0.6 | 0.4 | 0.7 | 0.3 | 0.7 | 0.4 | 0.7 | 0.4 | 0.7 | 0.3 |
| | C14:0 | 1.3 | 1.0 | 1.3 | 1.0 | 1.3 | 0.9 | 1.3 | 1.2 | 1.3 | 0.9 |
| | C16:0 | 8.8 | 0.9 | 8.0 | 1.1 | 7.8 | 1.1 | 8.1 | 1.2 | 8.1 | 0.9 |
| | C18:0 | 1.6 | 0.2 | 1.0 | 0.4 | 1.2 | 0.5 | 1.3 | 0.5 | 1.2 | 0.3 |
| | C18:1 | 38.2 | 52.5 | 35.2 | 37.8 | 35.1 | 43.6 | 36.1 | 42.2 | 35.8 | 40.7 |
| | C18:2 | 5.4 | 8.9 | 5.2 | 6.2 | 5.1 | 7.9 | 5.2 | 7.0 | 5.4 | 7.1 |
| | C18:3 α | 0.4 | 0.8 | 0.5 | 0.7 | 0.4 | 0.9 | 0.5 | 0.8 | 0.5 | 0.7 |
| | C8 + C10 | 42.2 | 34.9 | 46.4 | 51.8 | 46.8 | 44.5 | 45.5 | 46.1 | 45.6 | 48.5 |
| | sum | | | | | | | | | | |

As disclosed in Table 7, the CpauLPAAT1 and CigneaLPAAT1 genes show remarkable specificity towards C10:0 fatty acids. D2554-20 exhibits 39.0% of C10:0 in the sn-2 position versus just 26.4% in the S6511 base strain without the heterologous LPAAT, demonstrating a 1.5 fold increase in C10:0 inclusion at the sn-2 position. D2556-38 exhibits 36.2% of C10:0 in the sn-2 position versus 26.4% in the S6511 base strain, demonstrating a 1.4 fold increase in C10:0 inclusion at the sn-2 position. Although there is a small increase in C8:0 levels in the D2554-20 and D2555-34 strains, the vast majority of sn-2 targeting is C10:0-specific. Similarly, CpaiLPAAT1 and ChookLPAAT1 show remarkable specificity towards C8:0 fatty acids. D2555-34 exhibits 22.3% C8:0 in the sn-2 position versus just 8.5% in the S6511 base strain without the heterologous LPAAT, demonstrating a 2.6 fold increase in C8:0 inclusion at the sn-2 position. D2557-24 exhibits 29.1% C8:0 in the sn-2 position versus 8.5%, demonstrating a 3.4 fold increase in C8:0 inclusion at the sn-2 position. We teach that CpauLPAAT1 and CigneaLPAAT1 are C10:0-specific LPAATs and that CpaiLPAAT1 and ChookLPAAT1 are C8:0-specific LPAATs. Knutzon D S, Lardizabal K D, Nelsen J S, Bleibaum J L, Davies H M, Metz J G (1995) Cloning of a coconut endosperm cDNA encoding a 1-acyl-sn-glycerol-3-phosphate acyltransferase that accepts medium-chain-length substrates. Plant Physiol 109:999-1006

### Amino Acid Sequences for Novel LPAAT Genes

SEQ ID NO: 23 CpauLPAAT1
MAIPAAAVIFLFGLLFFTSGLIINLFQALCFVLVWPLSKNAYRRINRVFA
ELLLSELLCLFDWWAGAKLKLFTDPETFRLMGKEHALVIINHMTELDWML
GWVMGQHLGCLGSILSVAKKSTKFLPVLGWSMWFSEYLYIERSWAKDRTT
LKSHIERLTDYPLPFWMVIFVEGTRFTRTKLLAAQQYAASSGLPVPRNVL
IPRTKGFVSCVSHMRSFVPAVYDVTVAFPKTSPPPTLLNLFEGQSIVLHV
HIKRHAMKDLPESDDAVAQWCRDKFVEKDALLDKHNAEDTFSGQEVHRTG
SRPIKSLLVVISWVVVITFGALKFLQWSSWKGKAFSVIGLGIVTLLMHML
ILSSQAERSSNPAKVAQAKLKTELSISKKATDKEN
SEQ ID NO: 24 CprocLPAAT1
MAIPAAAVIFLFGLIFFASGLIINLFQALCFVLIWPISKNAYRRINRVFA
ELLLSELLCLFDWWAGAKLKLFTDPETFRLMGKEHALVIINHMTELDWMV
GWVMGQHFGCLGSILSVAKKSTKFLPVLGWSMWFTEYLYIERSWNKDKST
LKSHIERLKDYPLPFWLVIFAEGTRFTQTKLLAAQQYAASSGLPVPRNVL
IPRTKGFVSCVSHMRSFVPAVYDLTVAFPKTSPPPTLLNLFEGQSVVLHV
HIKRHAMKDLPESDDEVAQWCRDKFVEKDALLDKHNAEDTFSGQELQHTG
RRPIKSLLVVISWVVVIAFGALKFLQWSSWKGKAFSVIGLGIVTLLMHML
ILSSQAERSKPAKVAQAKLKTELSISKTVTDKEN
SEQ ID NO: 25 CprocLPAAT1b
MAIPAAAVIFLFGLIFFASGLIINLFQALCFVLIWPISKNAYRRINRVFA
ELLLSELLCLFDWWAGAKLKLFTDPETFRLMGKEHALVIINHMTELDWMV
GWVMGQHFGCLGSILSVAKKSTKFLPVLGWSMWFTEYLYIERSWNKDKST
LKSHIERLKDYPLPFWLVIFAEGTRFTQTKLLAAQQYAASSGLPVPRNVL
IPRTKGFVSCVSHMRSFVPAVYDLTVAFPKTSPPPTLLNLFEGQSVVLHV
HIKRHAMKDLPESDDEVAQWCRDKFVEK
SEQ ID NO: 26 CprocLPAAT2a
IVNLVQAVCFVLVRPLSKNTYRRINRVVAELLWLELVWLIDWWAGVKIKV
FTDHETFHLMGKEHALVICNHKSDIDWLVGWVLAQRSGCLGSTLAVMKKS
SKFLPVIGWSMWFSEYLFLERNWAKDESTLKSGLNRLKDYPLPFWLALFV
EGTRFTRAKLLAAQQYAASSGLPVPRNVLIPRTKGFVSSVSHMRSFVPAI
YDVTVAIPKTSPPPTLIRMFKGQSSVLHVHLKRHVMKDLPESDDAVAQWC
RDIFVEKDALLDKHNADDTFSGQELQDTGRPIKSLLVVISWAVLEVFGAV
KFLQWSSLLSSWKGLAFSGIGLGIITLLMHILILFSQSERSTPAKVAPAK
AKIEGESSKTEMEKEK
SEQ ID NO: 27 CprocLPAAT2b
IVNLVQAVCFVLVRPLSKNTYRRINRVVAELLWLELVWLIDWWAGVKIKV
FTDHETFHLMGKEHALVICNHKSDIDWLVGWVLAQRSGCLGSTLAVMKKS
SKFLPVIGWSMWFSEYLFLERNWAKDESTLKSGLNRLKDYPLPFWLALFV
EGTRFTRAKLLAAQQYAASSGLPVPRNVLIPRTKGFVSSVSHMRSFVPAI
YDVTVAIPKTSPPPTLIRMFKGQSSVLHVHLKRHVMKDLPESDDAVAQWC
RDIFVEKDALLDKHNADDTFSGQELQDTGRPIKSLLV
SEQ ID NO: 28 CpaiLPAAT1
MAIPSAAVVFLFGLLFFTSGLIINLFQAFCFVLISPLSKNAYRRINRVFA
ELLPLEFLWLFHWCAGAKLKLFTDPETFRLMGKEHALVIINHKIELDWMV
GWVLGQHLGCLGSILSVAKKSTKFLPVFGWSLWFSGYLFLERSWAKDKIT
LKSHIESLKDYPLPFWLIIFVEGTRFTRTKLLAAQQYAASSGLPVPRNVL
IPHTKGFVSSVSHMRSFVPAIYDVTVAFPKTSPPPTMLKLFEGQSVELHV
HIKRHAMKDLPESDDAVAQWCRDKFVEKDALLDKHNSEDTFSGQEVHHVG
RPIKALLVVISWVVVIIFGALKFLLWSSLLSSWKGKAFSVIGLGIVAGIV
TLLMHILILSSQAEGSNPVKAAPAKLKTELSSSKKVTNKEN
SEQ ID NO: 29 ChookLPAAT1
MAIPSAAVVFLFGLLFFTSGLIINLFQAFCFVLISPLSKNAYRRINRVFA
ELLPLEFLWLFHWCAGAKLKLFTDPETFRLMGKEHALVIINHKIELDWMV
GWVLGQHLGCLGSILSVAKKSTKFLPVFGWSLWFSEYLFLERSWAKDKIT
LKSHIESLKDYPLPFWLIIFVEGTRFTRTKLLAAQQYAASSGLPVPRNVL
IPHTKGFVSSVSHMRSFVPAIYDVTVAFPKTSPPPTMLKLFEGQSVELHV
HIKRHAMKDLPESDDAVAQWCRDKFVEKDALLDKHNSEDTFSGQEVHHVG
RPIKALLVVISWVVVIIFGALKFLLWSSLLSSWKGKAFSVIGLGIVAGIV
TLLMHILILSSQAEGSNPVKAAPAKLKTELSSSKKVTNKEN
SEQ ID NO: 30 ChookLPAAT2a
LSLLFFVSGLIVNLVQAVCFVLIRPLSKNTYRRINRVVAELLWLELVWLI
DWWAGVKIKVFTDHETFNLMGKEHALVVCNHKSDIDWLVGWVLAQRSGCL
GSTLAVMKKSSKFLPVIGWSMWFSEYLFLERSWAKDESTLKSGLKRLKDY
PLPFWLALFVEGTRFTQAKLLAAQQYAASSGLPVPRNVLIPRTKGFVSSV
SHMRSFVPAIYDVTVAIPKTSVPPTMLRIFKGQSSVLHVHLKRHLMKDLP
ESDDAVAQWCRDIFVEKDALLDKHNAEDTFSGQELQDIGRPIKSLLVVIS
WAVLVIFGAVKFLQWSSLLSSWKGLAFSGIGLGIVTLLMHILILFSQSER
STPAKVAPAKPKNEGESSKTEMEKEH
SEQ ID NO: 31 ChookLPAAT2b
QIKVFTDHETFNLMGKEHALVVCNHKSDIDWLVGWVLAQWSGCLGSTLAV
MKKSSKFLPVIGWSMWFSEYLFLERSWAKDESTLKSGLKRLKDYPLPFWL
ALFVEGTRFTQAKLLAAQQYAASSGLPVPRNVLIPRTKGFVSSVSHMRSF
VPAIYDVTVAIPKTSVPPTMLRIFKGQSSVLHVHLKRHLMKDLPESDDAV
AQWCRDIFVEKDALLDKHNAEDTFSGQELQDIGRPIKSLLVVISWAVLVI
FGAVKFLQWSSLLSSWKGLAFSGIGLGIVTLLMHILILFSQSERSTPAKV
APAKLKKEGESSKPETDKQN
SEQ ID NO: 32 ChookLPAAT3a
LSLLFFVSGLIVNLVQAVCFVLIRPLLKNTYRRINRVVAELLWLELVWLI
DWWAGIKIKVFTDHETFHLMGKEHALVICNHKSDIDWLVGWVLAQRSGCL
GSTLAVMKKSSKFLPVIGWSMWFSEYLFLERNWAKDESTLKSGLNRLKDY
PLPFWLALFVEGTRFTRAKLLAAQQYAASSGLPVPRNVLIPRTKGFVSSV
SQMRSFVPAIYDVTVAIPKTSPPPTLLRMFKGQSSVLHVHLKRHLMNDLP
ESDDAVAQWCRDIFVEKDALLDKHNAEDTFSGQELQDTGRPIKSLLVVIS
WATLVVFGAVKFLQWSSLLSSWKGLAFSGIGLGIITLLMHILILFSQSER
STPAKVAPAKPKNEGESSKTEMEKEH
SEQ ID NO: 33 ChookLPAAT3b
LSLLFFVSGLIVNLVQAVCFVLIRPLLKNTYRRINRVVAELLWLELVWLI
DWWAGIKIKVFTDHETFHLMGKEHALVICNHKSDIDWLVGWVLAQRSGCL
GSTLAVMKKSSKFLPVIGWSMWFSEYLFLERNWAKDESTLKSGLNRLKDY
PLPFWLALFVEGTRFTRAKLLAAQQYAASSGLPVPRNVLIPRTKGFVSSV
SQMRSFVPAIYDVTVAIPKTSPPPTLLRMFKGQSSVLHVHLKRHLMNDLP
ESDDAVAQWCRDIFVEKDALLDKHNAEDTFSGQELQDIGRPIKSLLVVIS
WAVLEIFGAVKFLQWSSLLSSWKGLAFSGIGLGIVTLLMHILILFSQSER
STPAKVAPAKPKKEGESSKPETDKEN
SEQ ID NO: 34 CigneaLPAAT1
MAIAAAAVIFLFGLLFFASGIIINLFQALCFVLIWPLSKNVYRRINRVFA
ELLLMDLLCLFHWWAGAKIKLFTDPETFRLMGMEHALVIMNHKTDLDWMV
GWILGQHLGCLGSILSIAKKSTKFIPVLGWSVWFSEYLFLERSWAKDKST
LKSHMEKLKDYPLPFWLVIFVEGTRFTRTKLLAAQQYAASSGLPVPRNVL
IPHTKGFVSCVSNMRSFVPAVYDVTVAFPKSSPPPTMLKLFEGQSIVLHV
HIKRHALKDLPESDDAVAQWCRDKFVEKDALLDKHNAEDTFSGQEVHHIG
RPIKSLLVVIAWVVVIIFGALKFLQWSSLLSTWKGKAFSVIGLGIATLLM
HMLILSSQAERSNPAKVAK
SEQ ID NO: 35 CigneaLPAAT2
MAIAAAAVIFLFGLLFFASGIIINLFQALCFVLIWPLSKNVYRRINRVFA
ELLLMDLLCLFHWWAGAKIKLFTDPETFRLMGMEHALVIMNHKTDLDWMV
GWILGQHLGCLGSILSIAKKSTKFIPVLGWSVWFSEYLFLERSWAKDEST
LKSGLNRLKDYPLPFWLALFVEGTRFTRAKLLAAQQYAASSGLPVPKNVL
IPRTKGFVSSVSHMRSFVPAIYDVTVAIPKTSAPPTLLRMFKGQSSVLHV
HLKRHLMKDLPESDDAVAQWCRDIFVEKDALLDKHNAEDTFSGQELHDIG
RPVKSLLVVISWAMLVVFGAVKFLQWSSLLSSWKGLAFSGIGLGIITLLM
HILILFSQSERSTPAKVAPAKQKNNEGESSKTEMEKEH
SEQ ID NO: 36 DcLPAAT1
SGLVVNLIQAFFFVLVRPFSKNAYRKINRVVAELLWLELIWLIDWWAGVK
IQLYTDPETFKLMGKEHALVICNHKSDIDWLVGWILAQRSGCLGSALAVM
KKSSKFLPVIGWSMWFSEYLFLERSWAKDENTLKSGFQRLRDFPHAFWLA
LFVEGTRFTQAKLLAAQEYASSMGLPAPRNVLIPRTKGFVTAVTHMRPFV
PAVYDVTLAIPKTSPPPTMLRLFKGQSSVVHIHLKRHLMSDLPKSDDSVA
QWCKDAFVVKDNLLDKHKENDSFGDGVLQDTGRPLNSLVVVISWACLLIF
GALKFFQWSSILSSWKGLAFSAVGLGIVTVLMQILIQFSQSERSNRPMPS
KHAK
SEQ ID NO: 37 DcLPAAT2
MAIPTAAYVVPLGAIFFFSGLLVNLIQAFFFITVWPLSKKTYIRINKVIV
ELLWLEFVWLADWWAGLKIEVYADAETFQLMGKEHALVICNHKSDIDWLV
GWILAQRAGCLGSSFAVTKKSARYLPVVGWSIWFSGAIFLERSWEKDENT
LKAGFQRLREFPCAFWLGLFVEGTRFTQAKLLAAQEYASTMGLPFPRNVL
IPRTKGFIAAVNHMREFVPAIYDLTFAFPKDSPPPTMLRLLKGQPSVVHV
HIKRHLMKDLPEKNEAVAQWCKDVFLVKDKLLDKHKDDGSFGDGELHEIG
RPLKSLVVVTTWACLLILGTLKFLLWSSLLSSWKGLIFSATGLAVLTVLM
QFLIQSTQSERSNPASLSK
SEQ ID NO: 38 CcrLPAAT1a
LGLLFFISGLAVNLIQAVCFVFLRPLSKNTYRKINRVLAELLWLQLVWLV
DWWAGVKIKVFADRESFNLMGKEHALVICNHKSDIDWLVGWVLAQRSGCL
GSSLAVMKKSSKFLPVIGWSMWFSEYLFLERSWAKDESTLKEGLRRLKDF
PRPFWLALFVEGTRFTQAKLLAAQEYATSQGLPVPRNVLIPRTKVHVHVK
RHLMKELPETDEAVAQWCKDLFVEKDKLLDKHVAEDTFSDQPLQDIGRPV
KPLLVVSSWACLVAYGALKFLQWSSLLSSWKGIAVSAVALAIVTILMQIM
ILFSQSERSIPAKVA
SEQ ID NO: 39 CcrLPAAT1b
LGLLFFISGLAVNLIQAVCFVFLRPLSKNTYRKINRVLAELLWLQLVWLV
DWWAGVKIKVFADRESFNLMGKEHALVICNHKSDIDWLVGWVLAQRSGCL
GSSLAVMKKSSKFLPVIGWSMWFSEYLFLERSWAKDESTLKEGLRRLKDF
PRPFWLALFVEGTRFTQAKLLAAQEYATSQGLPVPRNVLIPRTKGFVSAV
SHMRSFVPAVYDMTVAIPKSSPSPTMLRLFKGQSSVVHVHVKRHLMKELP
ETDEAVAQWCKDLFVEKDKLLDKHVAEDTFSDQPLQDIGRPVKPLLVVSS
WACLVAYGALKFLQWSSLLSSWKGIAVSAVALAIVTILMQIMILFSQSER
SIPTKVA
SEQ ID NO: 40 CcrLPAAT2a
MAIAAAAVVFLFGLLFFTSGLIINLAQAVCFVLIWPLSKNAYRRINRVFA
ELLLLELLWLFHWRAGAKLKLFADPETFRLFGKEHALVICNHRTDLDWMV
GWVLGQHFGCLGSILSVAKKSTKFLPVLGWSMWFSEYLFLERSWAKDKST
LKSHTERLKDYPLPFWLGIFVEGTRFTRAKLLAAQQYAASSGLPVPRNVL
IPHTKLHVHIKRYAMKDLPESDDAVAQWCRDIYVEKDAFLDKHNAEDTFS
GQEVHHIGRPIKSLLVVISWVVVIIFGALKFLRWSSLLSSWKGKAFSVIG
LGIVTLLVNILILSSQAERSNPAKVAPAKLKTELSPSKKVTNKEN
SEQ ID NO: 41 CcrLPAAT2b
MAIAAAAVVFLFGLLFFTSGLIINLAQAVCFVLIWPLSKNAYRRINRVFA
ELLLLELLWLFHWRAGAKLKLFADPETFRLFGKEHALVICNHRTDLDWMV
GWVLGQHFGCLGSILSVAKKSTKFLPVLGWSMWFSEYLFLERSWAKDKST
LKSHTERLKDYPLPFWLGIFVEGTRFTRAKLLAAQQYAASSGLPVPRNVL
IPHTKGFVSSMSHMRSFVPAVYDLTVAFPKTSPPPTLLKLFEGQSVVLHV
HIKRYAMKDLPESDDAVAQWCRDIYVEKDAFLDKHNAEDTFSGQEVHHIG
RPIKSLLVVISWVVVIIFGALKFLRWSSLLSSWKGKAFSVIGLGIVTLLV
NILILSSQAERSNPAKVAPAKLKTELSPSKKVTNKEN
SEQ ID NO: 42 BrLPAAT1a
AAAVIVPLGILFFISGLVVNLLQAICYVLIRPLSKNTYRKINRVVAETLW
LELVWIVDWWAGVKIQVFADNETFNRMGKEHALVVCNHRSDIDWLVGWIL
AQRSGCLGSALAVMKKSSKFLPVIGWSMWFSEYLFLERNWAKDESTLKSG
LQRLNDFPRPFWLALFVEGTRFTEAKLKAAQEYAASSELPVPRNVLIPRT
KGFVSAVSNMRSFVPAIYDMTVAIPKTSPPPTMLRLFKGQPSVVHVHIKC
HSMKDLPESDDAIAQWCRDQFVAKDALLDKHIAADTFPGQQEQNIGRPIK
SLAVVLSWSCLLILGAMKFLHWSNLFSSWKGIAFSALGLGIITLCMQILI
RSSQSERSTPAKVVPAKPKDNHNDSGSSSQTE
SEQ ID NO: 43 BrLPAAT1b
AAAVIVPLGILFFISGLVVNLLQAVCYVLVRPMSKNTYRKINRVVAETLW
LELVWIVDWWAGVKIQVFADDETFNRMGKEHALVVCNHRSDIDWLVGWIL
AQRSGCLGSALAVMKKSSKFLPVIGWSMWFSEYLFLERNWAKDESTLKSG
LQRLNDFPRPFWLALFVEGTRFTEAKLKAAQEYAASSELPVPRNVLIPRT
KGFVSAVSNMRSFVPAIYDMTVAIPKTSPPPTMLRLFKGQPSVVHVHIKC
HSMKDLPESDDAIAQWCRDQFVAKDALLDKHIAADTFPGQQEQNIGRPIK
SLAVVLSWSCLLILGAMKFLHWSNLFSSWKGIAFSALGLGIITLCMQILI
RSSQSERSTPAKVVPAKPKDNHNDSGSSSQTE
SEQ ID NO: 44 BrLPAAT1c
MAIAAAVIVPLGLLFFISGLLMNLLQAICYVLVRPLSKNTYRKINRVVAE
TLWLELVWIVDWWAGVKIKVFADNETFSRMGKEHALVVCNHRSDIDWLVG
WILAQRSGCLGSALAVMKKSSKFLPVIGWSMWFSEYLFLERNWAKDESTL
KSGLQRLNDFPRPFWLALFVEGTRFTEAKLKAAQEYAASSELPVPRNVLI
PRTKGFVSAVSNMRSFVPAIYDMTVAIPKTSPPPTMLRLFKGQPSVVHVH
IKCHSMKDLPESDDAIAQWCRDQFVAKDALLDKHIAADTFPGQQEQNIGR
PIKSLAVVLSWSCLLILGAMKFLHWSNLFSSWKGIAFSALGLGIITLCMQ
ILIRSSQSERSTPAKVVPAKPKDNHNDSGSSSQTE
SEQ ID NO: 45 BjLPAAT1a
INLVVAETLWLELVWIVDWWAGVKIQVFADDETFNRIVIGKEHALVVCNH
RSDIDWLVGWILAQRSGCLGSALAVMKKSSKFLPVIGWSMWFSEYLFLER
NWAKDESTLKSGLQRLNDFPRPFWLALFVEGTRFTEAKLKAAQEYAASSE
LPVPRNVLIPRTKGFVSAVSNMRSFVPAIYDMTVAIPKTSPPPTMLRLFK
GQPSVVHVHIKCHSMKDLPESDDAIAQWCRDQFVAKDALLDKHIAADTFP
GQKEQNIGRPIKSLAVSLIKTFPWLHPHQLTNIFVLFQVVVSWACLLTLG
AMKFLHWSNLFSSWKGIALSAFGLGIITLCMQILIRSSQSERSTPAKVAP
AKPK
SEQ ID NO: 46 BjLPAAT1b
INLVVAETLWLELVWIVDWWAGVKIQVFADDETFNRIVIGKEHALVVCNH
RSDIDWLVGWILAQRSGCLGSALAVMKKSSKFLPVIGWSMWFSEYLFLER
NWAKDESTLKSGLQRLNDFPRPFWLALFVEGTRFTEAKLKAAQEYAASSE
LPVPRNVLIPRTKGFVSAVSNMRSFVPAIYDMTVAIPKTSPPPTMLRLFK
GQPSVVHVHIKCHSMKDLPEPEDEIAQWCRDQFVAKDALLDKHIAADTFP
GQKEQNIGRPIKSLAVVVSWACLLTLGAMKFLHWSNLFSSWKGIALSAFG
LGIITLCMQILIRSSQSERSTPAKVAPAKPK
SEQ ID NO: 47 BjLPAAT1c
INLVVAETLWLELVWIVDWWAGVKIQVFADDETFNRIVIGKEHALVVCNH
RSDIDWLVGWILAQRSGCLGSALAVMKKSSKFLPVIGWSMWFSEYLFLER
NWAKDESTLKSGLQRLNDFPRPFWLALFVEGTRFTEAKLKAAQEYAASSE
LPVPRNVLIPRTKGFVSAVSNMRSFVPAIYDMTVAIPKTSPPPTMLRLFK
GQPSVVHVHIKCHSMKDLPESDDAIAQWCRDQFVAKDALLDKHIAADTFP
GQQEQNIGRPIKSLAVVLSWSCLLILGAMKFLHWSNLFSSWKGIAFSALG
LGIITLCMQILIRSSQSERSTPAKVVPAKPKDNHNDSGS5SQTE
SEQ ID NO: 48 BjLPAAT1d
INLVVAETLWLELVWIVDWWAGVKIQVFADDETFNRIVIGKEHALVVCNH
RSDIDWLVGWILAQRSGCLGSALAVMKKSSKFLPVIGWSMWFSEYLFLER
NWAKDESTLKSGLQRLNDFPRPFWLALFVEGTRFTEAKLKAAQEYAASSE
LPVPRNVLIPRTKGFVSAVSNMRSFVPAIYDMTVAIPKTSPPPTMLRLFK
GQPSVVHVHIKCHSMKDLPESDDAIAQWCRDQFVAKDALLDKHIAADTFP
GQQEQNIGRPIKSLAVSLS
SEQ ID NO: 49 CcLPAAT1a
MAIGVAAIVVPLGLLFILSGLMVNLIQAICFILVRPLSKNMYRRVNRVVV
ELLWLELIWLIDWWGGVKVDVYADSETFQSLGKEHALVVSNHRSDIDWLV
GWVLAQRSGCLGSTLAVMKKSSKFLPVIGWSMWFSEYVFLERSWAKDEST
LKSGLRRLKDFPRPFWLALFVEGTRFTQAKLLAAREYAASTGLPIPRNVL
IPRTKGFVSAVSNMRSFVPAIYDVTVAIPKTQPSPTMLRIFNRQPSVVHV
HIKRHSMNQLPQTDEGVGQWCKDIFVAKDALLDRHLAE
SEQ ID NO: 50 CcLPAAT1b
MAIGVAAIVVPLGLLFILSGLMVNLIQAICFILVRPLSKNMYRRVNRVVV
ELLWLELIWLIDWWGGVKVDVYADSETFQSLGKEHALVVSNHRSDIDWLV
GWVLAQRSGCLGSTLAVMKKSSKFLPVIGWSMWFSEYVFLERSWAKDEST
LKSGLRRLKDFPRPFWLALFVEGTRFTQAKLLAAREYAASTGLPIPRNVL
IPRTKGFVSAVSNMRSFVPAIYDVTVAIPKTQPSPTMLRIFNRQPSVVHV
HIKRHSMNQLPQTDEGVAQWCKDIFVAKDALLDRHLAEGKFDEKEFKRIR
RPIKSLLVISSWSFLLMFGVFKFLKWSALLSTWKGVAVSTTVLLLVTVVM
YMFILFSQSERSSPRKVAPSGPENG
SEQ ID NO: 51 UcLPAAT1a
MAIGVAAIVVPLGLLFILSGLIINLIQAICFILVRPLSKNMYRKVNRVVV
ELLWLELIWLIDWWGGVKVDVYADSETFQSLGKEHALVVSNHRSDIDWLV
GWVLAQRSGCLGSTLAVMKKSSKFLPVIGWSMWFSEYVFLERSWAKDEST
LKSGLQRLKDFPRPFWLALFVEGTRFTQAKLLAAQEYAASTGLPIPRNVL
IPRTKGFVSAVSNMRSFVPAIYDVTVAIPKTQPSPTMLRIFNRQPSVVHV
HIKRHSMNQLPQTDEGVAQWCKDIFVAKDALLDRHLAEGKFDEKEFKLIR
RPIKSLLVISSWSFLLMFGVFKFLKWSALLSTWKGVAVSTAVLLLVTVVM
YMFILFSQSERSSPRKVAPIGPENG
SEQ ID NO: 52 UcLPAAT1b
MAIGVAAIVVPLGLLFILSGLIINLIQAICFILVRPLSKNMYRKVNRVVV
ELLWLELIWLIDWWGGVKVDVYADSETFQSLGKEHALVVSNHRSDIDWLV
GWVLAQRSGCLGSTLAVMKKSSKFLPVIGWSMWFSEYVFLERSWAKDEST
LKSGLQRLKDFPRPFWLALFVEGTRFTQAKLLAAQEYAASTGLPIPRNVL
IPRTKGFVSAVSNMRSFVPAIYDVTVAIPKTQPSPTMLRIFNRQPSVVHV
HIKRHSMNQLPQTDEGVAQWCKDIFVAKDALLDRHLAE
SEQ ID NO: 53 LdLPAAT1
SLLFFMSGLVVNFIQAVFYVLVRPISKNTYRRINTLVAELLWLELVWVID
WWAGVKVQLYTDTESFRLMGKEHALLICNHRSDIDWLIGWVLAQRCGCLS
SSIAVMKKSSKFLPVIGWSMWFSEYLFLERNWAKDENTLKSGLQRLNDFP
KPFWLALFVEGTRFTKAKLLAAQEYAASAGLPVPRNVLIPRTKGFVSAVS
NMRSFVPAIYDLTVAIPKTTEQPTMLRLFRGKSSVVHVHLKRHLMKDLPK
TDDGVAQWCKDQFISKDALLDKHVAEDTFSGLEVQDIGRPMKSLVVVVSW
MCLLCLGLVKFLQWSALLSSWKGMMITTFVLGIVTVLMHILIRSSQSEHS
TPAK
SEQ ID NO: 54 CaequLPAAT1a
QRSGCLGSTLAVMKKSSKFLPVIGWSMWFSEYLFLERSWAKDESTLKSGL
KRLKDYPLPFWLALFVEGTRFTQAKLLAAQQYAASSGLPVPRNVLIPRTK
GFVSSVSHMRSFVPAIYDVTVAIPKMSTPPTMLRIFKGQSSVLHVHLKRH
LMKDLPESDDAVAQWCRDIFVEKDALLDKHNAEDTFSGQELQDIGRPVKS
LLVVISWAVLVIFGAVKFLQWSSLLSSWKGLAFSGIGLGIVTLLMHILIL
FSQSERSTPAKVAPAKPKKEGESSKTETEKEN
SEQ ID NO: 55 CaequLPAAT1b
DWWAGVKIKVFTDHETLSLMGKEHALVISNHKSDIDWLVGWVLAQRSGCL
GSTLAVMKKSSKFLPVIGWSMWFSEYLFLERSWAKDESTLKSGLKRLKDY
PLPFWLALFVEGTRFTQAKLLAAQQYAASSGLPVPRNVLIPRTKGFVSSV
SHMRSFVPAIYDVTVAIPKMSTPPTMLRIFKGQSSVLHVHLKRHLMKDLP
ESDDAVAQWCRDIFVEKDALLDKHNAEDTFSGQELQDIGRPVKSLLV
SEQ ID NO: 56 CaequLPAAT1c
DWWAGVKIKVFTDHETLSLMGKEHALVISNHKSDIDWLVGWVLAQRSGCL
GSTLAVMKKSSKFLPVIGWSMWFSEYLFLERSWAKDESTLKSGLKRLKDY
PLPFWLALFVEGTRFTQAKLLAAQQYAASSGLPVPRNVLIPRTKGFVSSV
SHMRSFVPAIYDVTVAIPKMSTPPTMLRIFKGQSSVLHVHLKRHLMKDLP
ESDDAVAQWCRDIFVEKDALLDKHNAEDTFSGQELQDIGRPVKSLLVVIS
WAVLVIFGAVKFLQWSSLLSSWKGLAFSGIGLGIVTLLMHILILFSQSER
STPAKVAPAKPKKEGESSKTETEKEN
SEQ ID NO: 57 CaequLPAAT1d
QRSGCLGSTLAVMKKSSKFLPVIGWSMWFSEYLFLERSWAKDESTLKSGL
KRLKDYPLPFWLALFVEGTRFTQAKLLAAQQYAASSGLPVPRNVLIPRTK
GFVSSVSHMRSFVPAIYDVTVAIPKMSTPPTMLRIFKGQSSVLHVHLKRH
LMKDLPESDDAVAQWCRDIFVEKDALLDKHNAEDTFSGQELQDIGRPVKS
LLV
SEQ ID NO: 58 CglutLPAAT1a
LSLLFFVSGLFVNLVQAVCFVLIRPFSKNTYRRINRVVAELLWLELVWLI
DWWAGVKIKVFTDHETLSLMGKEHALVISNHKSDIDWLVGWVLAQRSGCL
GSTLAVIVIKKSSKFLPVIGWSMWFSEYLFLERSWAKDESTLKSGLKRLK
DYPLPFWLALFVEGTRFTQAKLLAAQQYAASSGLPVPRNVLIPRTKGFVS
SVSHMRSFVPAIYDVTVAIPKMSTPPTMLRIFKGQSSVLHVHLKRHLMKD
LPESDDAVAQWCRDIFVEKDALLDKHNAEDTFSGQELQDIGRPVKSLLVV
ISWAVLVIFGAVKFLQWSSLLSSWKGLAFSGIGLGIVTLLMHILILFSQS
ERSTPAKVAPAKPKKEGESSKTETEKEN
SEQ ID NO: 59 CglutLPAAT1b
QAVCFVLIRPFSKNTYRRINRVVAELLWLELVWLIDWWAGVKIKVFTDHE
TLSLMGKEHALVISNHKSDIDWLVGWVLAQRSGCLGSTLAVMKKSSKFLP
VIGWSMWFSEYLFLERSWAKDESTLKSGLKRLKDYPLPFWLALFVEGTRF
TQAKLLAAQQYAASSGLPVPRNVLIPRTKGFVSSVSHMRSFVPAIYDVTV
AIPKMSTPPTMLRIFKGQSSVLHVHLKRHLMKDLPESDDAVAQWCRDIFV
EKDALLDKHNAEDTFSGQELQDIGRPVKSLLVVISWAVLVIFGAVKFLQW
SSLLSSWKGLAFSGIGLGIVTLLMHILILFSQSERSTPAKVAPAKPKKEG
ESSKTETEKEN
SEQ ID NO: 60 CprLPAAT1
MAIAAAAVVFLFGLLFFTSGLIINLAQAVCFVLIWPLSKNAYRRINRVFA
ELLLLELLWLFHWRAGAKLKLFADPETFRLFGKEHALVICNHRTDLDWMV
GWVLGQHFGCLGSILSVAKKSTKFLPVLGWSMWFSEYLFLERSWAKDKST
LKSHTERLKDYPLPFWLGIFVEGTRFTRAKLLAAQQYAASSGLPVPRNVL
IPHTKGFVSSMSHMRSFVPAVYDLTVAFPKTSPPPTLLKLFEGQSVVLHV
HIKRYAMKDLPESDDAVAQWCRDIYVEKDAFLDKHNAEDTFSGQEVHHIG
RPIKSLLVVISWVVVIIFGALKFLRWSSLLSSWKGKAFSVIGLGIVTLLV
NILILSSQAERSNPAKVVPAKLKTELSPSKKVTNKEN
SEQ ID NO: 61 ChsLPAAT1
MAIPSAAVVFLFGLLFFASGLIINLVQAVCFVLIWPLSKNTCRRINIVFQ
DMLLSELLWLFHWRAGAKLKFFTDPETYRHMGKEHALVITNHRTDLDWMI
GWVLGEHLGCLGSILSVVKKSTKFLPVLGWSMWFSEYLFLERNWAKDKST
FKSHIERLEDFPQPFWFGIFVEGTRFTRAKLLAAQQYAASSGLPVPRNVL
IPHTKGFVSSVSHMRSFVPAVYETTMTFPKTSPPPTLLKLFEGQPLVLHI
HMKRHAMKDIPESDDAVAQWCRDKFVEKDALLDKHNAEDTFGGLEVHIGR
SIKSLMVVICWVVVIIFGALKFLQWSSLLSSWKGIAFIGIGLGIVNLLVH
VLILSSQAERSAPTKVAPAKLKTKLLSSKKITNKEN
SEQ ID NO: 62 ChsLPAAT2
MAIPSAAVVFLFGLLFFASGLIINLVQAVCFVLIWPLSKNTCRRINIVFQ
DMLLSELLWLFHWRAGAKLKFFTDPETYRHMGKEHALVITNHRTDLDWMI
GWVLGEHLGCLGSILSVVKKSTKFLPVLGWSMWFSEYLFLERNWAKDKST
FKSHIERLEDFPQPFWFGIFVEGTRFTRAKLLAAQQYAASSGLPVPRNVL
IPRTKGFVSSVSHMRSFVPAIYDVTVAIPKTSPPPTMLRMFKGQSSVLHV
HLKRHLMKDLPESDDAVAQWCRDIFVEKDALLDKHNAEDTFSGQELQDIG
RPIKSLVVVISWAALVVFGAVKFLQWSSLLSSWKGLAFSGIGLGIITLLM
HILILFSQSERSTPAKVAPAKPKREGESSKTEMDKEN
SEQ ID NO: 63 CcalcLPAAT1a
MAIPAAAVVFLFGLLFFPSGLIINLFQAVCFVLIWPFSRNTCRRINIVFQ
EMLLSELLWLFHWRAGAKLKLFADPETYRHMGKEHALLITNHRTDLDWMI
GWALGQHLGCLGSILSVVKKSTKFLPSHIERLEDFPQPFWMAIFVEGTRF
TRAKLLAAQQYAASSGLPVPRNVLIPRTKGFVSCVSHMRSFVPAVYETTM
TFPKTSPPPTLLKLFEGQPIVLHVHMKRHAMKDIPESDEAVAQWCRDKFV
EKDSLLDKHNAGDTFSCQEIHIGRPIKSLMVVISWVVVIIFGALKFLQWS
SLLSSWKGIAFSGIGLGIVTLLVHILILSSQAERSTPAKVAPAKLKTELS
SSTKVTNKEN
SEQ ID NO: 64 CcalcLPAAT1b
MAIPAAAVVFLFGLLFFPSGLIINLFQAVCFVLIWPFSRNTCRRINIVFQ
EMLLSELLWLFHWRAGAKLKLFADPETYRHMGKEHALLITNHRTDLDWMI
GWALGQHLGCLGSILSVVKKSTKFLPVLGWSMWFSEYLFLERNWAKDKST
FKSHIERLEDFPQPFWMAIFVEGTRFTRAKLLAAQQYAASSGLPVPRNVL
IPRTKGFVSCVSHMRSFVPAVYETTMTFPKTSPPPTLLKLFEGQPIVLHV
HMKRHAMKDIPESDEAVAQWCRDKFVEKDSLLDKHNAGDTFSCQEIHIGR
PIKSLMVVISWVVVIIFGALKFLQWSSLLSSWKGIAFSGIGLGIVTLLVH
ILILSSQAERSTPAKVAPAKLKTELSSSTKVTNKEN
SEQ ID NO: 65 CcalcLPAAT2
LSLLFFVSGLIVNLVQAVCFVLIRPLSKNTYRRINRVVAELLWLELVWLI
DWWAGVKIKVFTDHETFRLMGTEHALVISNHKSDIDWLVGWVLAQRSGCL
GSTLAVIVIKKSSKFLPVIGWSMWFSEYLFLERSWAKDESTLKSGLNRLK
DYPLPFWLALFVEGTRFTRAKLLAAQQYAASSGLPVPRNVLIPRTKGFVS
SVSHMRSFVPAIYDVTVAIPKTSPPPTMLRMFKGQSSVLHVHLKRHLMKD
LPESDDAVAQWCRDIFVEKDALLDKHNAEDTFSGQELQDIGRPIKSLVVV
ISWAALVVFGAVKFLQWSSLLSSWKGLAFSGIALGIITLLMHILILFSQS
ERSTPAKVAPAKPKKEGESSKTETDKEN
SEQ ID NO: 66 ChtLPAAT1a
MAIPAAAVIFLFSILFFASGLIINLVQAVCFVLIWPLSKNTCRRINLVFQ
EMLLSELLGLFHWRAGAKLKLYTDPETYPLLGKEHALLMINHRTDLDWMI
GWVLGQHLGCLGSILSVVKKSTKFLPVLGWSMWFSEYLFLERNWAKDKST
FKSHIERLEDFPQPFWMAIFVEGTRFTRAKLLAAQQYAASSGLPVPRNVL
IPHTKGFVSTVSHMRSFVPAVYDTTLTFPKTSPPPTLLNLFAGQPIVLHI
HIKRHAMKDIPESDDAVAQWCRDKFVEKDALLDKHNAEDAFSDQEFPISR
SIKSLMVVISWVMVIIFGALKFLQWSSLLSSWKGKAFSVIAVGIVTLLMH
MSILSSQAERSNPAKVALPKLKTELPSSKKVLNKEN
SEQ ID NO: 67 ChtLPAAT1b
MAIPAAAVIFLFSILFFASGLIINLVQAVCFVLIWPLSKNTCRRINLVFQ
EMLLSELLGLFHWRAGAKLKLYTDPETYPLLGKEHALLMINHRTDLDWMI
GWVLGQHLGCLGSILSVVKKSTKFLPVLGWSMWFSEYLFLERNWAKDKST
FKSHIERLEDFPQPFWMAIFVEGTRFTRAKLLAAQQYAASSGLPVPRNVL
IPHTKGFVSTVSHMRSFVPAVYDTTLTFPKTSPPPTLLNLFAGQPIVLHI
HIKRHAMKDIPESDDAVAQWCRDKFVEKDALLDKHNAEDAFSDQEFPISR
SIKSLMVVISWVMVIIFGALKFLQWSSLLSSWKGIAFSGIGLGIVTLLMH
ILILSSQAERSTPAKVAQAKVKTELPSSTKVTNKGN
SEQ ID NO: 68 CwLPAAT1
MAIPAAAVIFLFGILFFASGLIINLVQAVCFVLIWPLSKNTCRRINLVFQ
EMLLSELLWLFHWRAGAELKLFTDPETYRLLGKEHALVMTNHRTDLDWMI
GWVTGQHLGCLGSILSIAKKSTKFLPVLGWSMWFSEYLFLERNWAKDKST
FKSHIERLEDFPQPFWMAIFVEGTRFTRAKLLAAQQYAASSGLPVPRNVL
IPHTKGFVSSVCHMRSFVPAVYDTTLTFPKNSPPPTLLNLFAGQPIVLHI
HIKRHAMKDMPKSDDAVAQWCRDKFVKKDALLDKHNTEDTFSDQEFPIGR
PIKSLMVVISWVVVIIFGTLKFLQWSSLLSSWKGIAFSGIGLGIVTLLVH
ILILSSQAERSTPPKVAPAKLKTELSSTTKVINKGN
SEQ ID NO: 69 CwLPAAT2b
LGLLFFVSGLIVNLVQAVCFVLIRPLSKNTYRRLNRVVAELLWLELVWLI
DWWAGVKIKVFTDHETFHLMGKEHALVICNHKSDIDWLVGWVLAQRSGCL
GSTLAVMKKSSKFLPVIGWSMWFSEYLFLERSWAKDESTLKSGLNRLKDY
PLPFWLALFVEGTRFTRAKLLAAQQYAASSGLPVPRNVLIPRTKGFVSSV
SHMIRSFVPAIYDVTVAIPKTSPPPTMLRMFKGQSSVDALLDKHNADDTF
SGQELHDIGRPIKSLLVVISWAVLVVFGAVKFLQWSSLLSSWKGIAFSGI
GLGIVTLLVHILILSSQAERSTSAKVAQAKVKTELSSSKKVKNKGN
SEQ ID NO: 70 CwLPAAT2a
LGLLFFVSGLIVNLVQAVCFVLIRPLSKNTYRRLNRVVAELLWLELVWLI
DWWAGVKIKVFTDHETFHLMGKEHALVICNHKSDIDWLVGWVLAQRSGCL
GSTLAVMKKSSKFLPVIGWSMWFSEYLFLERSWAKDESTLKSGLNRLKDY
PLPFWLALFVEGTRFTRAKLLAAQQYAASSGLPVPRNVLIPRTKGFVSSV
SHMIRSFVPAIYDVTVAIPKTSPPPTMLRMFKGQSSVLHVHLKRHLMKDL
PESDDAVAQWCRDIFVEKDVLLDKHNAEDTFSGQELQDIGRPVKSLLVVI
SWTLLVIFGAVKFLQWSSLLSSWKGLAFSGIGLGIVTLLMHILILFSQSE
RSTPAKVAPAKPKKEGESSKMETDKEN
SEQ ID NO: 71 CgLPAAT1a
LAGWMGSSSGCLGSTLAVMKKSSKFLPVIGWSMWFSEYLFLERSWAKDES
TLKSGLNRLKDYPLPFWLALFVEGTRFTRAKLLAAQQYAASLGLPVPRNV
LIPRTKGFVSSVSHMIRSFVPAIYDVTVAIPKTSPPPTMIRMFKGQSSVL
HVHLKRHVMKDLPESDDAVAQWCRDIFVEKDALLDKHNAEDTFSGQELQD
TGRPIKSLLVVISWAVLEVFGAVKFLQWSSLLSSWKGLAFSGIGLGIITL
LMHILILFSQSERSTPAKVAPAKPKNEGESSKAEMEKEK
SEQ ID NO: 72 CgLPAAT1b
LAGWMGSSSGCLGSTLAVMKKSSKFLPVIGWSMWFSEYLFLERSWAKDES
TLKSGLNRLKDYPLPFWLALFVEGTRFTRAKLLAAQQYAASLGLPVPRNV
LIPRTKGFVSSVSHMIRSFVPAIYDVTVAIPKTSPPPTMIRMFKGQSSVL
HVHLKRHVMKDLPESDDAVAQWCRDIFVEKDALLDKHNAEDTFSGQELQD
TGRPIKSLLVRCFLVLSLIYLNGIMLKLRGPCLQVVISWAVLEVFGAVKF
LQWSSLLSSWKGLAFSGIGLGIITLLMHILILFSQSERSTPAKVAPAKPK
NEGESSKAEMEKEK
SEQ ID NO: 73 CgLPAAT1c
LAGWMGSSSGCLGSTLAVMKKSSKFLPVIGWSMWFSEYLFLERSWAKDES
TLKSGLNRLKDYPLPFWLALFVEGTRFTRAKLLAAQQYAASLGLPVPRNV
LIPRTKGFVSSVSHMIRSFVPAIYDVTVAIPKTSPPPTMIRMFKGQSSVL
HVHLKRHVMKDLPESDDAVAQWCRDIFVEKDALLDKHNAEDTFSGQELQD
TGRPIKSLLVVTSWAVLVISGAVKFLQWSSLLSSWKGLAFSGIGLGIVTL
LMHILILFSQSERSTPAKVAPAKPKKEGESSKTEKDKEN
SEQ ID NO: 74 CpalLPAAT1
LGLLFFVSGLIVNLVQAVCFVLIRPLSKNTYRRINRVVAELLWLELVWLI
DWWAGVKIKVFTDHETLSLMGKEHALVICNHKSDIDWLVGWVLAQRSGCL
GSTLAVMKKSSKFLPVIGWSMWFSEYLFLERSWAKDENTLKSGLNRLKDY
PLPFWLALFVEGTRFTRAKLLAAQQYATSSGLPVPRNVLIPRTKGFVSSV
SHMIRSFVPAIYDVTVAIPKTSPPPTMLRMFKGQSSVLHVHLKRHLMKDL
PESDDAVAQWCRDIFVEKDALLDKHNAEDTFSGQELQDTGRPIKSLLVVI
SWAVLVIFGAVKFLQWSSLLSSWKGLAFSGVGLGIITLLMHILILFSQSE
RSTPAKVAPAKPKKDGESSKTEIEKEN
SEQ ID NO: 75 CaLPAAT1
MAIAAAAVIVPVSLLFFVSGLIVNLVQAVCFVLIRPLFKNTYRRINRVVA
ELLWLELVWLIDWWAGVKIKVFTDHETFHLMGKEHALVICNHKSDIDWLV
GWVLAQRSGCLGSTLAVMKKSSKFLPVIGWSMWFSEYLFLERNWAKDEST
LKSGLNRLKDYPLPFWLALFVEGTRFTRAKLLAAQQYAASSGLPVPRNVL
IPRTKGFVSSVSHMRSFVPAIYDVTVAIPKTSPPPTLLRMFKGQSSVLHV
HLKRHQMNDLPESDDAVAQWCRDIFVEKDALLDKHNAEDTFSGQELQDTG
RPIKSLLIVISWAVLVVFGAVKFLQWSSLLSSWKGLAFSGIGLGVITLLM
HILILFSQSERSTPAKVAPAKPKIEGESSKTEMEKEH
SEQ ID NO: 76 CaLPAAT3
MTIASAAVVFLFGILLFTSGLIINLFQAFCSVLVWPLSKNAYRRINRVFA
EFLPLEFLWLFHWWAGAKLKLFTDPETFRLMGKEHALVIINHKIELDWMV
GWVLGQHLGCLGSILSVAKKSTKFLPVFGWSLWFSEYLFLERNWAKDKKT
LKSHIERLKDYPLPFWLIIFVEGTRFTRTKLLAAQQYAASAGLPVPRNVL
IPHTKGFVSSVSHMRSFVPAIYDVTVAFPKTSPPPTMLKLFEGHFVELHV
HIKRHAMKDLPESEDAVAQWCRDKFVEKDALLDKHNAEDTFSGQEVHHVG
RPIKSLLVVISWVVVIIFGALKFLQWSSLLSSWKGIAFSVIGLGTVALLM
QILILSSQAERSIPAKETPANLKTELSSSKKVTNKEN
SEQ ID NO: 77 SalLPAAT1
MAIGAAAIVVPLGLLFMLSGLMVNLIQAICFILVRPLSKNMYRRVNRVVV
ELLWLELIWLIDWWGGVKVDVYADSETFQSLGKEHALVVSNHKSDIDWLV
GWVLAQRSGCLGSTLAVMKKSSKFLPVIGWSMWFSEYVFLERSWAKDEST
LKSGLQRLKDFPRPFWLALFVEGTRFTQAKLLAAQEYAASTGLPIPRNVL
IPRTKGFVSAVSNMRSFVPAIYDVTVAIPKTQPSPTMLRIFNRQPSVVHV
RIKRHSMNQLPPTDEGVAQWCKDIFVAKDALLDRHLAEGKFDEKEFKRIR
RPIKSLLVISSWSFLLLFGVFKFLKWSALLSTWKGVAVSTAVLLLVTVVM
YMFILFSQSERSSPRKVAPSGPENG
SEQ ID NO: 78 CleptLPAAT1
MAIPAAVVIFLFGLLFFSSGLIINLFQALCFVLIWPLSKNAYRRINRVFA
ELLLSELLCLFDWWAGAKLKLFTDPETFRLMGKEHALVIINHMTELDWMV
GWVMGQHFGCLGSILSVAKKSTKFLPVLGWSMWFTEYLYIERSWDKDKST
LKSHIERLKDYPLPFWLVIFAEGTRFTRTKLLAAQQYAASSGLPVPRNVL
IPRTKGFVSCVNHMRSFVPAVYDLTVAFPKTSPPPTLLNLFEGQSVVLHV
HIKRHAMKDLPESDDAVAQWCRDKFVEKDALLDKHNAEDTFSSQEVHHTG
SRPIKSLLVVISWVVVITFGALKFLQWSSWKGKAFSVIGLGIVTLLMHML
ILSSQAERSKPAKVTQAKLKTELSISKKVTDKEN
SEQ ID NO: 79 ClopLPAAT1
MAIAAAAVIFLFGLLFFASGLIINLFQALCFVLIRPLSKNAYRRINRVFA
ELLLSELLCLFDWWAGAKLKLFTDPETLRLMGKEHALIIINHMTELDWMV
GWVMGQHFGCLGSIISVAKKSTKFLPVLGWSMWFSEYLYLERSWAKDKST
LKSHIERLKDYPLPFWLVIFVEGTRFTRTKLLAAQEYAASSGLPVPRNVL
IPRTKGFVSCVNHMRSFVPAVYDVTVAFPKTSPQPTLLNLFEGRSIVLHV
HIKRHAMKDLPESDDAVAQWCRDKFVEKDALLDKHNAEDTFSGQEVHHTG
RRPIKSLLVVMSWVVVTTFGALKFLQWSSWKGKAFSVIGLGIVTLLMHVL
ILSSQAERSNPAKVVQAELNTELSISKKVTNKGN
SEQ ID NO: 80 CcrasLPAAT1a
MAIPAAAVIFLFGLIFFASGLIINLFQALCFVLIWPLWKNAYRRINRVFA
ELLLSELLCLFDWWAGAKLKLFTDPETFRLMGKEHALVIINHMTELDWMV
GWVMGQHFGCLGSILSVAKKSTKFLPVLGWSMWFTEYLYIERSWDKDKST
LKSHIERLKDYPLPFWLVIFAEGTRFTRTKLLAAQQYAASSGLPVPRNVL
IPRTKGFVSSVSHMRSFVPAIYDVTVAIPKTSPPPTLIRMFKGQSSVLHV
HLKRHVMKDLPESDDAVAQWCRDIFVEKDALLDKHNAEDTFSGQELQDTG
RPIKSLLVVISWAVLEVFGAVKFLQWSSLLSSWKGLAFSGIGLGIITLLM
HILILFSQSERSTPAKVAPAKAK
SEQ ID NO: 81 CcrasLPAAT1b
MAIPAAAVIFLFGLIFFASGLIINLFQALCFVLIWPLWKNAYRRINRVFA
ELLLSELLCLFDWWAGAKLKLFTDPETFRLMGKEHALVIINHMTELDWMV
GWVMGQHFGCLGSILSVAKKSTKFLPVLGWSMWFTEYLYIERSWDKDKST
LKSHIERLKDYPLPFWLVIFAEGTRFTRTKLLAAQQYAASSGLPVPRNVL
IPRTKGFVSSVSHMRSFVPAIYDVTVAIPKTSPPPTLIRMFKGQSSVLHV
HLKRHVMKDLPESDDAVAQWCRDIFVEKDALLDKHNAEDTFSGQELQDTG
RPIKSLLVRCFLVLSLIYLNGIILKLCGLCLQVVISWAVLEVFGAVKFLQ
WSSLLSSWKGLAFSGIGLGIITLLMHILILFSQSERSTPAKVAPAKAK
SEQ ID NO: 82 CcrasLPAAT1c
MAIPAAAVIFLFGLIFFASGLIINLFQALCFVLIWPLWKNAYRRINRVFA
ELLLSELLCLFDWWAGAKLKLFTDPETFRLMGKEHALVIINHMTELDWMV
GWVMGQHFGCLGSILSVAKKSTKFLPVLGWSMWFTEYLYIERSWDKDKST
LKSHIERLKDYPLPFWLVIFAEGTRFTRTKLLAAQQYAASSGLPVPRNVL
IPRTKGFVSSVSHMRSFVPAIYDVTVAIPKTSPPPTLIRMFKGQSSVLHV
HLKRHVMKDLPESDDAVAQWCRDIFVEKDALLDKHNAEDTFSGQELQDTG
RPIKSLLVVISWAVLEVFGAVKFLQWSSLLSSWKGLAFSGIGLGIITLLM
HILILFSQSERSTPAKVAPAKAKMEGESSKTEMEMEK
SEQ ID NO: 83 CcrasLPAAT1d
MAIPAAAVIFLFGLIFFASGLIINLFQALCFVLIWPLWKNAYRRINRVFA
ELLLSELLCLFDWWAGAKLKLFTDPETFRLMGKEHALVIINHMTELDWMV
GWVMGQHFGCLGSILSVAKKSTKFLPVLGWSMWFTEYLYIERSWDKDKST
LKSHIERLKDYPLPFWLVIFAEGTRFTRTKLLAAQQYAASSGLPVPRNVL
IPRTKGFVSSVSHMRSFVPAIYDVTVAIPKTSPPPTLIRMFKGQSSVLHV
HLKRHVMKDLPESDDAVAQWCRDIFVEKDALLDKHNAEDTFSGQELQDTG
RPIKSLLVRCFLVLSLIYLNGIILKLCGLCLQVVISWAVLEVFGAVKFLQ
WSSLLSSWKGLAFSGIGLGIITLLMHILILFSQSERSTPAKVAPAKAKME
GESSKTEMEMEK
SEQ ID NO: 84 CkoeLPAAT1
MAIAAAPVIFLFGLLFFASGLIINLFQAICFVLIWPLSKNAYRRINRVFA
ELLLSELLCLFDWWAGAKLKLFTDPETFRLMGKEHALVITNHKIDLDWMI
GWILGQHFGCLGSVISIAKKSTKFLPIFGWSLWFSEYLFLERNWAKDKRT
LKSHIERMKDYPLPLWLILFVEGTRFTRTKLLAAQQYAASSGLPVPRNVL
IPHTKGFVSSVSHMRSFVPAIYDVTVAIPKTSPPPTLIRMFKGQSSVLHV
HLKRHLMKDLPESDDAVAQWCRDIFVEKDALLDKHNAEDTFSGQELQETG
RPIKSLLVVISWAVLEVYGAVKFLQWSSLLSSWKGLAFSGIGLGLITLLM
HILILFSQSERSTPAKVAPAKPKKEGESSKTEMEKEK
SEQ ID NO: 85 CkoeLPAAT2
MHVLLEMVTFRFSSFFVFDNVQALCFVLIWPLSKSAYRKINRVFAELLLS
ELLCLFDWWAGAKLKLFTDPETFRLMGKEHALVITNHKIDLDWMIGWILG
QHFGCLGSVISIAKKSTKFLPIFGWSLWFSEYLFLERNWAKDKRTLKSHI
ERMKDYPLPLWLILFVEGTRFTRTKLLAAQQYAASSGLPVPRNVLIPHTK
GFVSSVSHMRSFVPAVYDVTVAFPKTSPPPTMLSLFEGQSVVLHVHIKRH
AMKDLPDSDDAVAQWCRDKFVEKDALLDKHNAEDTFSGQEVHHVGRPIKS
LLVVISWMVVIIFGALKFLQWSSLLSSWKGKAFSAIGLGIATLLMHVLVV
FSQADRSNPAKVPPAKLNTELSSSKKVTNKEN

### Example 5: Expression of LPAATs to Improve Sn-2 Selectivity in Prototheca Moriformis

In the example we disclose genetically engineered Prototheca moriformis strains in which we have modified fatty acid and triacylglycerol biosynthesis to maximize the accumulation of Stearoyl-Oleoyl-Stearoyl (SOS) TAGs, and minimize the production of trisaturated TAGs. Oils from these strains resemble plant seed oils known as “structuring fats”, which have high proportions of Saturated-Oleate-Saturated TAGs and low levels of trisaturates. These structuring fats (often called “butters”) are generally solid at room temperature but melt sharply between 35-40° C.

Strains with high SOS and low trisaturates were obtained by three successive transformations, beginning with S5100, a classically improved derivative of S376 (improved to increase lipid titer), a wild type isolate of Prototheca moriformis. S5100 was transformed with a construct to which increased expression of PmKASII-1 and ablated the SAD2-1 allele. The resultant strain, S5780, produced oil with increased C18:0 and lower C16:0 content relative to S5100. S5780 was prepared according to the methods disclosed in co-owned application WO2013/158938 and as described below. C18:0 levels were increased further by transformation of S5780 with a construct overexpressing the C18:0-specific FATA1 thioesterase gene from Garcinia mangostana (GarmFATA1), generating strain S6573. S6573 was disclosed in co-owned application WO2015/051319. Finally, accumulation of trisaturated TAGs was reduced by expression of genes encoding LPAATs from Brassica napus, Theobroma cacao, Garcinia hombororiana or Garcinia indica in S6573 as described below.

### Construct Used for SAD2 Knockout and PmKASII-1 Overexpression in S5100 to Produce S5780

The sequence of the transforming DNA from the SAD2-1 ablation, PmKASII over-expression construct, pSZ2624, is shown below. The construct is written as: pSZ2624:SAD2-1vD::PmKASII-1tp_PmKASII-1_FLAG-CvNR:CpACT-AtTHIC-CpEF1a::SAD2-1vE Relevant restriction sites are indicated in lowercase, bold, and are from 5′-3′ PmeI, SpeI, AscI, ClaI, SacI, AvrII, EcoRV, AflII, KpnI, XbaI, MfeI, BamHI, BspQI and PmeI. Underlined sequences at the 5′ and 3′ flanks of the construct represent genomic DNA from P. moriformis that enable targeted integration of the transforming DNA via homologous recombination at the SAD2-1 locus. The SAD2-1 5′ integration flank contained the endogeneous SAD2-1 promoter, enabling the in situ activation of the PmKASII gene. Proceeding in the 5′ to 3′ direction, the region encoding the PmKASII plastid targeting sequence is indicated by lowercase, underlined italics. The sequence that encodes the mature PmKASII polypeptide is indicated with lowercase italics, while a 3×FLAG epitope encoding sequence is in bold italics. The initiator ATG and terminator TGA for PmKASII-FLAG are indicated by uppercase italics. The 3′ UTR of the Chlorella vulgaris nitrate reductase (CvNR) gene is indicated by small capitals. Two spacer regions are represented by lowercase text. The CpACT promoter driving the expression of the AtTHIC gene (encoding 4-amino-5-hydroxymethyl-2-methylpyrimidine synthase activity, thereby permitting the strain to grow in the absence of exogeneous thiamine) is indicated by lowercase, boxed text. The initiator ATG and terminator TGA for AtTHIC are indicated by uppercase italics, while the coding region is indicated with lowercase italics. The 3′ UTR of the Chlorella protothecoides EF1a (CpEF1a) gene is indicated by small capitals. The use of THIC as a selection marker was described in co-owned applications WO2011/150410 and WO2013/150411.

pSZ2624 Nucleotide sequence of the transforming DNA
SEQ ID NO: 86
gtttaaacGCCGGTCACCACCCGCATGCTCGTACTACAGCGCACGCACCGCTTCGTGA
TCCACCGGGTGAACGTAGTCCTCGACGGAAACATCTGGTTCGGGCCTCCTGCTTG
CACTCCCGCCCATGCCGACAACCTTTCTGCTGTTACCACGACCCACAATGCAACG
CGACACGACCGTGTGGGACTGATCGGTTCACTGCACCTGCATGCAATTGTCACAA
GCGCTTACTCCAATTGTATTCGTTTGTTTTCTGGGAGCAGTTGCTCGACCGCCCGC
GTCCCGCAGGCAGCGATGACGTGTGCGTGGCCTGGGTGTTTCGTCGAAAGGCCA
GCAACCCTAAATCGCAGGCGATCCGGAGATTGGGATCTGATCCGAGTTTGGACC
AGATCCGCCCCGATGCGGCACGGGAACTGCATCGACTCGGCGCGGAACCCAGCT
TTCGTAAATGCCAGATTGGTGTCCGATACCTGGATTTGCCATCAGCGAAACAAGA
CTTCAGCAGCGAGCGTATTTGGCGGGCGTGCTACCAGGGTTGCATACATTGCCCA
TTTCTGTCTGGACCGCTTTACTGGCGCAGAGGGTGAGTTGATGGGGTTGGCAGGC
ATCGAAACGCGCGTGCATGGTGTGCGTGTCTGTTTTCGGCTGCACGAATTCAATA
GTCGGATGGGCGACGGTAGAATTGGGTGTGGCGCTCGCGTGCATGCCTCGCCCC
GTCGGGTGTCATGACCGGGACTGGAATCCCCCCTCGCGACCATCTTGCTAACGCT
CCCGACTCTCCCGACCGCGCGCAGGATAGACTCTTGTTCAACCAATCGACAactagt
ATGcagaccgcccaccagcgcccccccaccgagggccactgcttcggcgcccgcctgcccaccgcctcccgccgcgccgtgc
gccgcgcctggtcccgcatcgcccgcgggcgcgccgccgccgccgccgacgccaaccccgcccgccccgagcgccgcgtggt
gatcaccggccagggcgtggtgacctccctgggccagaccatcgagcagttctactcctccctgctggagggcgtgtccggcatct
cccagatccagaagttcgacaccaccggctacaccaccaccatcgccggcgagatcaagtccctgcagctggacccctacgtgc
ccaagcgctgggccaagcgcgtggacgacgtgatcaagtacgtgtacatcgccggcaagcaggccctggagtccgccggcctg
cccatcgaggccgccggcctggccggcgccggcctggaccccgccctgtgcggcgtgctgatcggcaccgccatggccggcat
gacctccttcgccgccggcgtggaggccctgacccgcggcggcgtgcgcaagatgaaccccttctgcatccccttctccatctcca
acatgggcggcgccatgctggccatggacatcggcttcatgggccccaactactccatctccaccgcctgcgccaccggcaacta
ctgcatcctgggcgccgccgaccacatccgccgcggcgacgccaacgtgatgctggccggcggcgccgacgccgccatcatcc
cctccggcatcggcggcttcatcgcctgcaaggccctgtccaagcgcaacgacgagcccgagcgcgcctcccgcccctgggac
gccgaccgcgacggcttcgtgatgggcgagggcgccggcgtgctggtgctggaggagctggagcacgccaagcgccgcggcg
ccaccatcctggccgagctggtgggcggcgccgccacctccgacgcccaccacatgaccgagcccgacccccagggccgcgg
cgtgcgcctgtgcctggagcgcgccctggagcgcgcccgcctggcccccgagcgcgtgggctacgtgaacgcccacggcacct
ccacccccgccggcgacgtggccgagtaccgcgccatccgcgccgtgatcccccaggactccctgcgcatcaactccaccaagt
ccatgatcggccacctgctgggcggcgccggcgccgtggaggccgtggccgccatccaggccctgcgcaccggctggctgcac
cccaacctgaacctggagaaccccgcccccggcgtggaccccgtggtgctggtgggcccccgcaaggagcgcgccgaggacc
tggacgtggtgctgtccaactccttcggcttcggcggccacaactcctgcgtgatatccgcaagtacgacgag [symbol] [symbol] [symbol] [symbol] [symbol] [symbol]
TGAatcgatAGATCTCTT
AAGGCAGCAGCAGCTCGGATAGTATCGACACACTCTGGACGCTGGTCGTGTGAT
GGACTGTTGCCGCCACACTTGCTGCCTTGACCTGTGAATATCCCTGCCGCTTTTAT
CAAACAGCCTCAGTGTGTTTGATCTTGTGTGTACGCGCTTTTGCGAGTTGCTAGCT
GCTTGTGCTATTTGCGAATACCACCCCCAGCATCCCCTTCCCTCGTTTCATATCGC
TTGCATCCCAACCGCAACTTATCTACGCTGTCCTGCTATCCCTCAGCGCTGCTCCT
GCTCCTGCTCACTGCCCCTCGCACAGCCTTGGTTTGGGCTCCGCCTGTATTCTCCT
GGTACTGCAACCTGTAAACCAGCACTGCAATGCTGATGCACGGGAAGTAGTGGG
ATGGGAACACAAATGGAAAGCTTAATTAAgagctccgcgtctcgaacagagcgcgcagaggaacgct
gaaggtctcgcctctgtcgcacctcagcgcggcatacaccacaataaccacctgacgaatgcgcttggttcttcgtccattagcgaagc
gtccggttcacacacgtgccacgttggcgaggtggcaggtgacaatgatcggtggagctgatggtcgaaacgttcacagcctaggtg
atatccatcttaaggatctaagtaagattcgaagcgctcgaccgtgccggacggactgcagccccatgtcgtagtgaccgccaatgta
agtgggctggcgtttccctgtacgtgagtcaacgtcactgcacgcgcaccaccctctcgaccggcaggaccaggcatcgcgagatac
agcgcgagccagacacggagtgccgagctatgcgcacgctccaactaggtaccagtttaggtccagcgtccgtggggggggacg
ggctgggagcttgggccgggaagggcaagacgatgcagtccctctggggagtcacagccgactgtgtgtgttgcactgtgcggccc
gcagcactcacacgcaaaatgcctggccgacaggcaggccctgtccagtgcaacatccacggtccctctcatcaggctcaccttgct
cattgacataacggaatgcgtaccgctattcagatctgtccatccagagaggggagcaggctccccaccgacgctgtcaaacttgctt
cctgcccaaccgaaaacattattgtttgagggggggggggggggggcagattgcatggcgggatatctcgtgaggaacatcactgg
gacactgtggaacacagtgagtgcagtatgcagagcatgtatgctaggggtcagcgcaggaagggggcctttcccagtctcccatgc
cactgcaccgtatccacgactcaccaggaccagcttcttgatcggcttccgctcccgtggacaccagtgtgtagcctctggactccagg
tatgcgtgcaccgcaaaggccagccgatcgtgccgattcctgggtggaggatatgagtcagccaacttggggctcagagtgcacact
ggggcacgatacgaaacaacatctacaccgtgtcctccatgctgacacaccacagcttcgctccacctgaatgtgggcgcatgggcc
cgaatcacagccaatgtcgctgctgccataatgtgatccagaccctctccgcccagatgccgagcggatcgtgggcgctgaatagatt
cctgtttcgatcactgtttgggtcctttccttttcgtctcggatgcgcgtctcgaaacaggctgcgtcgggctttcggatcccttttgctccct
ccgtcaccatcctgcgcgcgggcaagttgcttgaccctgggctgataccagggttggagggtattaccgcgtcaggccattcccagcc
cggattcaattcaaagtctgggccaccaccctccgccgctctgtctgatcactccacattcgtgcatacactacgttcaagtcctgatcca
ggcgtgtctegggacaaggtgtgatgagtttgaatctcaaggacccactccagcacagctgctggttgaccccgccctcgcaatcta
gaATGgccgcgtccgtccactgcaccctgatgtccgtggtctgcaacaacaagaaccactccgcccgccccaagctgcccaac
tcctccctgctgcccggcttcgacgtggtggtccaggccgcggccacccgcttcaagaaggagacgacgaccacccgcgccacg
ctgacgttcgacccccccacgaccaactccgagcgcgccaagcagcgcaagcacaccatcgacccctcctcccccgacttcca
gcccatcccctccttcgaggagtgcttccccaagtccacgaaggagcacaaggaggtggtgcacgaggagtccggccacgtcct
gaaggtgcccttccgccgcgtgcacctgtccggcggcgagcccgccttcgacaactacgacacgtccggcccccagaacgtcaa
cgcccacatcggcctggcgaagctgcgcaaggagtggatcgaccgccgcgagaagctgggcacgccccgctacacgcagatg
tactacgcgaagcagggcatcatcacggaggagatgctgtactgcgcgacgcgcgagaagctggaccccgagttcgtccgctc
cgaggtcgcgcggggccgcgccatcatcccctccaacaagaagcacctggagctggagcccatgatcgtgggccgcaagttcc
tggtgaaggtgaacgcgaacatcggcaactccgccgtggcctcctccatcgaggaggaggtctacaaggtgcagtgggccacc
atgtggggcgccgacaccatcatggacctgtccacgggccgccacatccacgagacgcgcgagtggatcctgcgcaactccgc
ggtccccgtgggcaccgtccccatctaccaggcgctggagaaggtggacggcatcgcggagaacctgaactgggaggtgttcc
gcgagacgctgatcgagcaggccgagcagggcgtggactacttcacgatccacgcgggcgtgctgctgcgctacatccccctga
ccgccaagcgcctgacgggcatcgtgtcccgcggcggctccatccacgcgaagtggtgcctggcctaccacaaggagaacttcg
cctacgagcactgggacgacatcctggacatctgcaaccagtacgacgtcgccctgtccatcggcgacggcctgcgccccggct
ccatctacgacgccaacgacacggcccagttcgccgagctgctgacccagggcgagctgacgcgccgcgcgtgggagaagga
cgtgcaggtgatgaacgagggccccggccacgtgcccatgcacaagatccccgagaacatgcagaagcagctggagtggtgc
aacgaggcgcccttctacaccctgggccccctgacgaccgacatcgcgcccggctacgaccacatcacctccgccatcggcgcg
gccaacatcggcgccctgggcaccgccctgctgtgctacgtgacgcccaaggagcacctgggcctgcccaaccgcgacgacgt
gaaggcgggcgtcatcgcctacaagatcgccgcccacgcggccgacctggccaagcagcacccccacgcccaggcgtggga
cgacgcgctgtccaaggcgcgcttcgagttccgctggatggaccagttcgcgctgtccctggaccccatgacggcgatgtccttcc
acgacgagacgctgcccgcggacggcgcgaaggtcgcccacttctgctccatgtgcggccccaagttctgctccatgaagatca
cggaggacatccgcaagtacgccgaggagaacggctacggctccgccgaggaggccatccgccagggcatggacgccatgt
ccgaggagttcaacatcgccaagaagacgatctccggcgagcagcacggcgaggtcggcggcgagatctacctgcccgagtc
ctacgtcaaggccgcgcagaagTGAcaattgACGGAGCGTCGTGCGGGAGGGAGTGTGCCGAG
CGGGGAGTCCCGGTCTGTGCGAGGCCCGGCAGCTGACGCTGGCGAGCCGTACGC
CCCGAGGGTCCCCCTCCCCTGCACCCTCTTCCCCTTCCCTCTGACGGCCGCGCCTG
TTCTTGCATGTTCAGCGACggatccTAGGGAGCGACGAGTGTGCGTGCGGGGCTGGC
GGGAGTGGGACGCCCTCCTCGCTCCTCTCTGTTCTGAACGGAACAATCGGCCACC
CCGCGCTACGCGCCACGCATCGAGCAACGAAGAAAACCCCCCGATGATAGGTTG
CGGTGGCTGCCGGGATATAGATCCGGCCGCACATCAAAGGGCCCCTCCGCCAGA
GAAGAAGCTCCTTTCCCAGCAGACTCCTTCTGCTGCCAAAACACTTCTCTGTCCA
CAGCAACACCAAAGGATGAACAGATCAACTTGCGTCTCCGCGTAGCTTCCTCGG
CTAGCGTGCTTGCAACAGGTCCCTGCACTATTATCTTCCTGCTTTCCTCTGAATTA
TGCGGCAGGCGAGCGCTCGCTCTGGCGAGCGCTCCTTCGCGCCGCCCTCGCTGAT
CGAGTGTACAGTCAATGAATGGTCCTGGGCGAAGAACGAGGGAATTTGTGGGTA
AAACAAGCATCGTCTCTCAGGCCCCGGCGCAGTGGCCGTTAAAGTCCAAGACCG
TGACCAGGCAGCGCAGCGCGTCCGTGTGCGGGCCCTGCCTGGCGGCTCGGCGTG
CCAGGCTCGAGAGCAGCTCCCTCAGGTCGCCTTGGACGGCCTCTGCGAGGCCGG
TGAGGGCCTGCAGGAGCGCCTCGAGCGTGGCAGTGGCGGTCGTATCCGGGTCGC
CGGTCACCGCCTGCGACTCGCCATCCgaagagcgtttaaac

Construct D1683 (pSZ2624), was transformed into S5100. Primary transformants were clonally purified and grown under standard lipid production conditions at pH 5. Integration of pSZ2624 at the SAD2-1 locus was verified by DNA blot analysis. The fatty acid profiles and lipid titers of lead strains were assayed in 50-mL shake flasks (Table 8). Simultaneous ablation of SAD2-1 and over-expression of PmKASII (driven in situ by the SAD2-1 promoter) resulted in C18:0 levels up to 26.1%. C16:0 accumulation was reduced from 15.3% in S5100 to ≤6% the strains derived from D1683, demonstrating that PmKASII-1 over-expression promoted the elongation of C16:0 to C18:0. S5780 was chosen for further development as it had the highest lipid titer relative to the S5100 parent.

TABLE 8
Fatty acid profiles of SAD2-1 ablation, PmKASII-1 overexpression
strains derived from D1683-1, compared to the S5100 parent.

| | | | Primary | | | | |
|---|---|---|---|---|---|---|---|
| | | | S5100; T531; D1683.1 | | | | |
| | | Strain | | | | | |
| | | S5100 | S5780 | S5781 | S5782 | S5783 | S5784 |
| Fatty Acid | C14:0 | 0.7 | 0.7 | 0.8 | 0.7 | 0.7 | 0.7 |
| Area % | C16:0 | 15.3 | 5.9 | 6.0 | 6.0 | 5.8 | 5.8 |
| | C16:1 | 0.5 | 0.1 | 0.0 | 0.1 | 0.0 | 0.0 |
| | C18:0 | 4.0 | 25.6 | 26.1 | 26.0 | 25.0 | 25.3 |
| | C18:1 | [symbol] | 55.7 | 54.5 | 54.6 | 56.3 | 55.6 |
| | C18:2 | 7.3 | 8.0 | 8.5 | 8.5 | 8.1 | 8.4 |
| | C18:3 α | 0.5 | 0.7 | 0.8 | 0.8 | 0.7 | 0.7 |
| | C20:0 | 0.3 | 1.8 | 1.9 | 1.8 | 1.8 | 1.8 |
| | C20:1 | 0.2 | 0.6 | 0.6 | 0.6 | 0.7 | 0.7 |
| | C22:0 | 0.1 | 0.2 | 0.3 | 0.3 | 0.3 | 0.2 |
| | C24:0 | 0.1 | 0.4 | 0.4 | 0.4 | 0.4 | 0.4 |
| | saturates | 20.6 | 34.7 | 35.6 | 35.4 | 34.1 | 34.5 |

We disclose additional methods of elevating C18:0 levels that can be used in conjunction with SAD2 knockout and KASII over-expression. Previously we described acyl-ACP thioesterases from Brassica napus (BnFATA) (Co-owned application WO2012/106560), Garcinia mangostana (GarmFATA1) (Co-owned application WO2015/051319) and Theobroma cacao (TcFATA) (Co-owned application WO2013/158938) with specificity towards cleavage of C18:0-ACP, and we observed that average C18:0 levels were higher in strains in which we replaced the native BnFATA transit peptide with the Chlorella protothecoides SAD1 transit peptide (CpSAD1tp). A DNA construct was made for expression of a chimeric gene encoding CpSAD1tp fused to the predicted GarmFATA1 mature polypeptide and a FLAG tag sequence.

The sequence of the transforming DNA from the GarmFATA1 expression construct pSZ3204 is shown below. The construct is written as pSZ3204:6SA::CrTUB2-ScSUC2-CvNR:PmSAD2-2-CpSAD1_tp_GarmFATA1_FLAG-CvNR::6SB. Relevant restriction sites are indicated in lowercase, bold, and are from 5′-3′ BspQI, KpnI, XbaI, MfeI, BamHI, AvrII, EcoRV, SpeI, AscI, ClaI, AflII, SacI and BspQI. Underlined sequences at the 5′ and 3′ flanks of the construct represent genomic DNA from P. moriformis that enable targeted integration of the transforming DNA via homologous recombination at the 6S locus. Proceeding in the 5′ to 3′ direction, the CrTUB2 promoter driving the expression of Saccharomyces cerevisiae SUC2 (ScSUC2) gene, enabling strains to utilize exogeneous sucrose, is indicated by lowercase, boxed text. The initiator ATG and terminator TGA of ScSUC2 are indicated by uppercase italics, while the coding region is represented by lowercase italics. The 3′ UTR of the CvNR gene is indicated by small capitals. A spacer region is represented by lowercase text. The P. moriformis SAD2-2 (PmSAD2-2) promoter driving the expression of the chimeric CpSAD1tp_GarmFATA1_FLAG gene is indicated by lowercase, boxed text. The initiator ATG and terminator TGA are indicated by uppercase italics; the sequence encoding CpSAD1tp is represented by lowercase, underlined italics; the sequence encoding the GarmFATA1 mature polypeptide is indicated by lowercase italics; and the 3× FLAG epitope tag is represented by uppercase, bold italics. A second CvNR 3′ UTR is indicated by small capitals.

pSZ3204
SEQ ID NO: 87
gctcttcGCCGCCGCCACTCCTGCTCGAGCGCGCCCGCGCGTGCGCCGCCAGCGCCTT
GGCCTTTTCGCCGCGCTCGTGCGCGTCGCTGATGTCCATCACCAGGTCCATGAGG
TCTGCCTTGCGCCGGCTGAGCCACTGCTTCGTCCGGGCGGCCAAGAGGAGCATG
AGGGAGGACTCCTGGTCCAGGGTCCTGACGTGGTCGCGGCTCTGGGAGCGGGCC
AGCATCATCTGGCTCTGCCGCACCGAGGCCGCCTCCAACTGGTCCTCCAGCAGCC
GCAGTCGCCGCCGACCCTGGCAGAGGAAGACAGGTGAGGGGGGTATGAATTGTA
CAGAACAACCACGAGCCTTGTCTAGGCAGAATCCCTACCAGTCATGGCTTTACCT
GGATGACGGCCTGCGAACAGCTGTCCAGCGACCCTCGCTGCCGCCGCTTCTCCCG
CACGCTTCTTTCCAGCACCGTGATGGCGCGAGCCAGCGCCGCACGCTGGCGCTGC
GCTTCGCCGATCTGAGGACAGTCGGGGAACTCTGATCAGTCTAAACCCCCTTGCG
CGTTAGTGTTGCCATCCTTTGCAGACCGGTGAGAGCCGACTTGTTGTGCGCCACC
CCCCACACCACCTCCTCCCAGACCAATTCTGTCACCTTTTTGGCGAAGGCATCGG
CCTCGGCCTGCAGAGAGGACAGCAGTGCCCAGCCGCTGGGGGTTGGCGGATGCA
CGCTCAggtaccctttcttgcgctatgacacttccagcaaaaggtagggegggctgcgagacggcttcceggcgctgcatgcaa
caccgatgatgcttcgaccccccgaagctccttcggggctgcatgggcgctccgatgccgctccagggcgagcgctgtttaaatagc
caggcccccgattgcaaagacattatagcgagctaccaaagccatattcaaacacctagatcactaccacttctacacaggccactcga
gettgtgatcgcactccgctaagggggcgcctatcctcttcgtttcagtcacaacccgcaaactctagaatatcaATGctgctgcag
gccttcctgttcctgctggccggcttcgccgccaagatcagcgcctccatgacgaacgagacgtccgaccgccccctggtgcactt
cacccccaacaagggctggatgaacgaccccaacggcctgtggtacgacgagaaggacgccaagtggcacctgtacttccagt
acaacccgaacgacaccgtctgggggacgcccttgttctggggccacgccacgtccgacgacctgaccaactgggaggaccag
cccatcgccatcgccccgaagcgcaacgactccggcgccttctccggctccatggtggtggactacaacaacacctccggcttctt
caacgacaccatcgacccgcgccagcgctgcgtggccatctggacctacaacaccccggagtccgaggagcagtacatctcct
acagcctggacggcggctacaccttcaccgagtaccagaagaaccccgtgctggccgccaactccacccagttccgcgacccg
aaggtcttctggtacgagccctcccagaagtggatcatgaccgcggccaagtcccaggactacaagatcgagatctactcctccg
acgacctgaagtcctggaagctggagtccgcgttcgccaacgagggcttcctcggctaccagtacgagtgccccggcctgatcga
ggtccccaccgagcaggaccccagcaagtcctactgggtgatgttcatctccatcaaccccggcgccccggccggcggctccttc
aaccagtacttcgtcggcagcttcaacggcacccacttcgaggccttcgacaaccagtcccgcgtggtggacttcggcaaggact
actacgccctgcagaccttcttcaacaccgacccgacctacgggagcgccctgggcatcgcgtgggcctccaactgggagtactc
cgccttcgtgcccaccaacccctggcgctcctccatgtccctcgtgcgcaagttctccctcaacaccgagtaccaggccaacccgg
agacggagctgatcaacctgaaggccgagccgatcctgaacatcagcaacgccggcccctggagccggttcgccaccaacac
cacgttgacgaaggccaacagctacaacgtcgacctgtccaacagcaccggcaccctggagttcgagctggtgtacgccgtcaa
caccacccagacgatctccaagtccgtgttcgcggacctctccctctggttcaagggcctggaggaccccgaggagtacctccgc
atgggcttcgaggtgtccgcgtcctccttcttcctggaccgcgggaacagcaaggtgaagttcgtgaaggagaacccctacttcac
caaccgcatgagcgtgaacaaccagcccttcaagagcgagaacgacctgtcctactacaaggtgtacggcttgctggaccaga
acatcctggagctgtacttcaacgacggcgacgtcgtgtccaccaacacctacttcatgaccaccgggaacgccctgggctccgt
gaacatgacgacgggggtggacaacctgttctacatcgacaagttccaggtgcgcgaggtcaagTGAcaattgGCAGCA
GCAGCTCGGATAGTATCGACACACTCTGGACGCTGGTCGTGTGATGGACTGTTGC
CGCCACACTTGCTGCCTTGACCTGTGAATATCCCTGCCGCTTTTATCAAACAGCCT
CAGTGTGTTTGATCTTGTGTGTACGCGCTTTTGCGAGTTGCTAGCTGCTTGTGCTA
TTTGCGAATACCACCCCCAGCATCCCCTTCCCTCGTTTCATATCGCTTGCATCCCA
ACCGCAACTTATCTACGCTGTCCTGCTATCCCTCAGCGCTGCTCCTGCTCCTGCTC
ACTGCCCCTCGCACAGCCTTGGTTTGGGCTCCGCCTGTATTCTCCTGGTACTGCAA
CCTGTAAACCAGCACTGCAATGCTGATGCACGGGAAGTAGTGGGATGGGAACAC
AAATGGAggatcccgcgtctcgaacagagcgcgcagaggaacgctgaaggtctcgcctctgtcgcacctcagcgcggcata
caccacaataaccacctgacgaatgcgcttggttcttcgtccattagcgaagcgtccggttcacacacgtgccacgttggcgaggtgg
caggtgacaatgatcggtggagctgatggtcgaaacgttcacagcctagggatatcctgaagaatgggaggcaggtgttgttgattat
gagtgtgtaaaagaaaggggtagagagccgtcctcagatccgactactatgcaggtagccgctcgcccatgcccgcctggctgaata
ttgatgcatgcccatcaaggcaggcaggcatttctgtgcacgcaccaagcccacaatcttccacaacacacagcatgtaccaacgcac
gcgtaaaagttggggtgctgccagtgcgtcatgccaggcatgatgtgctcctgcacatccgccatgatctcctccatcgtctcgggtgtt
tccggcgcctggtccgggagccgttccgccagatacccagacgccacctccgacctcacggggtacttttcgagcgtctgccggtag
tcgacgatcgcgtccaccatggagtagccgaggcgccggaactggcgtgacggagggaggagagggaggagagagagggggg
ggggggggggggatgattacacgccagtctcacaacgcatgcaagacccgtttgattatgagtacaatcatgcactactagatggatg
agcgccaggcataaggcacaccgacgttgatggcatgagcaactcccgcatcatatttcctattgtcctcacgccaagccggtcaccat
ccgcatgctcatattacagcgcacgcaccgcttcgtgatccaccgggtgaacgtagtcctcgacggaaacatctggctcgggcctcgt
gctggcactccctcccatgccgacaacctttctgctgtcaccacgacccacgatgcaacgcgacacgacccggtgggactgatcggtt
cactgcacctgcatgcaattgtcacaagcgcatactccaatcgtatccgtttgatttctgtgaaaactcgctcgaccgcccgcgtcccgc
aggcagcgatgacgtgtgcgtgacctgggtgtttcgtcgaaaggccagcaaccccaaatcgcaggcgatccggagattgggatctg
atccgagcttggaccagatcccccacgatgcggcacgggaactgcatcgactcggcgcggaacccagctttcgtaaatgccagattg
gtgtccgataccttgatttgccatcagcgaaacaagacttcagcagcgagcgtatttggcgggcgtgctaccagggttgcatacattgc
ccatttctgtctggaccgctttaccggcgcagagggtgagttgatggggttggcaggcatcgaaacgcgcgtgcatggtgtgtgtgtct
gttttcggctgcacaatttcaatagtcggatgggcgacggtagaattgggtgttgcgctcgcgtgcatgcctcgccccgtcgggtgtcat
gaccgggactggaatcccccctcgcgaccctcctgctaacgctcccgactctcccgcccgcgcgcaggatagactctagttcaacca
atcgacaactagtATGgccaccgcatccactttctcggcgttcaatgcccgctgcggcgacctgcgtcgctcggcgggctccggg
ccccggcgcccagcgaggcccctccccgtgcgcgggcgcgccatccccccccgcatcatcgtggtgtcctcctcctcctccaagg
tgaaccccctgaagaccgaggccgtggtgtcctccggcctggccgaccgcctgcgcctgggctccctgaccgaggacggcctgt
cctacaaggagaagttcatcgtgcgctgctacgaggtgggcatcaacaagaccgccaccgtggagaccatcgccaacctgctgc
aggaggtgggctgcaaccacgcccagtccgtgggctactccaccggcggcttctccaccacccccaccatgcgcaagctgcgcc
tgatctgggtgaccgcccgcatgcacatcgagatctacaagtaccccgcctggtccgacgtggtggagatcgagtcctggggcca
gggcgagggcaagatcggcacccgccgcgactggatcctgcgcgactacgccaccggccaggtgatcggccgcgccacctcc
aagtgggtgatgatgaaccaggacacccgccgcctgcagaaggtggacgtggacgtgcgcgacgagtacctggtgcactgccc
ccgcgagctgcgcctggccttccccgaggagaacaactcctccctgaagaagatctccaagctggaggacccctcccagtactc
caagctgggcctggtgccccgccgcgccgacctggacatgaaccagcacgtgaacaacgtgacctacatcggctgggtgctgg
agtccatgccccaggagatcatcgacacccacgagctgcagaccatcaccctggactaccgccgcgagtgccagcacgacga
cgtggtggactccctgacctcccccgagccctccgaggacgccgaggccgtgttcaaccacaacggcaccaacggctccgcca
acgtgtccgccaacgaccacggctgccgcaacttcctgcacctgctgcgcctgtccggcaacggcctggagatcaaccgcggcc
gcaccgagtggcgcaagaagcccacccgc [symbol] [symbol] [symbol] [symbol] [symbol] [symbol]
TGAatcgatagatctcttaagGCAGCAG
CAGCTCGGATAGTATCGACACACTCTGGACGCTGGTCGTGTGATGGACTGTTGCC
GCCACACTTGCTGCCTTGACCTGTGAATATCCCTGCCGCTTTTATCAAACAGCCTC
AGTGTGTTTGATCTTGTGTGTACGCGCTTTTGCGAGTTGCTAGCTGCTTGTGCTAT
TTGCGAATACCACCCCCAGCATCCCCTTCCCTCGTTTCATATCGCTTGCATCCCAA
CCGCAACTTATCTACGCTGTCCTGCTATCCCTCAGCGCTGCTCCTGCTCCTGCTCA
CTGCCCCTCGCACAGCCTTGGTTTGGGCTCCGCCTGTATTCTCCTGGTACTGCAAC
CTGTAAACCAGCACTGCAATGCTGATGCACGGGAAGTAGTGGGATGGGAACACA
AATGGAaagcttaattaagagctcTTGTTTTCCAGAAGGAGTTGCTCCTTGAGCCTTTCATTC
TCAGCCTCGATAACCTCCAAAGCCGCTCTAATTGTGGAGGGGGTTCGAATTTAAA
AGCTTGGAATGTTGGTTCGTGCGTCTGGAACAAGCCCAGACTTGTTGCTCACTGG
GAAAAGGACCATCAGCTCCAAAAAACTTGCCGCTCAAACCGCGTACCTCTGCTTT
CGCGCAATCTGCCCTGTTGAAATCGCCACCACATTCATATTGTGACGCTTGAGCA
GTCTGTAATTGCCTCAGAATGTGGAATCATCTGCCCCCTGTGCGAGCCCATGCCA
GGCATGTCGCGGGCGAGGACACCCGCCACTCGTACAGCAGACCATTATGCTACC
TCACAATAGTTCATAACAGTGACCATATTTCTCGAAGCTCCCCAACGAGCACCTC
CATGCTCTGAGTGGCCACCCCCCGGCCCTGGTGCTTGCGGAGGGCAGGTCAACC
GGCATGGGGCTACCGAAATCCCCGACCGGATCCCACCACCCCCGCGATGGGAAG
AATCTCTCCCCGGGATGTGGGCCCACCACCAGCACAACCTGCTGGCCCAGGCGA
GCGTCAAACCATACCACACAAATATCCTTGGCATCGGCCCTGAATTCCTTCTGCC
GCTCTGCTACCCGGTGCTTCTGTCCGAAGCAGGGGTTGCTAGGGATCGCTCCGAG
TCCGCAAACCCTTGTCGCGTGGCGGGGCTTGTTCGAGCTTgaagagc

Construct D1940 (pSZ3204), was transformed into the S5780 parent strain. Primary transformants were clonally purified and grown under standard lipid production conditions at pH 5. Integration of pSZ3204 at the 6S locus was verified by DNA blot analysis. The fatty acid profiles and lipid titers of lead strains were assayed in 50-mL shake flasks (Table 9). Over-expression of GarmFATA1 (driven by the SAD2-2 promoter) resulted in C18:0 levels up to 54.3%. C16:0 levels were comparable in strains derived from D1940 and the S5780 parent. S6573 was chosen for further development as it had the highest lipid titer of the strains with >50% C18:0.

TABLE 9
Fatty acid profiles of GarmFATA1 overexpressing stable strains
derived from D1940 primary transformants.

| | | | Primary | | | | | | | |
|---|---|---|---|---|---|---|---|---|---|---|
| | | | D1683.1 | D1940.19 | | | D1940.20 | D1940.23 | D1940.46 | D1940.5 |
| | | Strain | | | | | | | | |
| | | S5100 | S5780 | S6571 | S6572 | S6573 | S6574 | S6575 | S6578 | S6580 |
| Fatty Acid | C14:0 | 0.7 | 0.0 | 0.8 | 0.0 | 0.8 | 0.7 | 0.7 | 0.0 | 0.0 |
| Area % | C16:0 | 18.0 | 5.9 | 6.3 | 6.6 | 6.3 | 5.0 | 5.1 | 5.0 | 5.3 |
| | C16:1 | 0.5 | 0.0 | 0.1 | 0.1 | 0.1 | 0.0 | 0.1 | 0.1 | 0.1 |
| | C18:0 | 3.9 | 29.0 | 52.7 | 54.3 | 53.7 | 43.1 | 46.0 | 45.4 | 47.9 |
| | C18:1 | 69.8 | 54.3 | 31.4 | 30.1 | 30.5 | 41.5 | 38.5 | 40.0 | 37.2 |
| | C18:2 | 5.9 | 6.4 | 5.7 | 5.8 | 5.6 | 6.3 | 6.2 | 6.1 | 6.2 |
| | C18:3 α | 0.5 | 0.7 | 0.6 | 0.6 | 0.6 | 0.6 | 0.5 | 0.6 | 0.5 |
| | C20:0 | 0.3 | 2.4 | 1.8 | 1.6 | 1.7 | 2.1 | 2.0 | 2.0 | 2.0 |
| | C20:1 | 0.1 | 0.6 | 0.1 | 0.1 | 0.1 | 0.2 | 0.1 | 0.1 | 0.1 |
| | C22:0 | 0.1 | 0.3 | 0.2 | 0.2 | 0.2 | 0.3 | 0.3 | 0.2 | 0.2 |
| | C24:0 | 0.0 | 0.0 | 0.0 | 0.0 | 0.0 | 0.0 | 0.0 | 0.0 | 0.0 |
| | saturates | 23.1 | 37.7 | 61.9 | 62.8 | 62.8 | 51.2 | 54.2 | 52.7 | 55.5 |

Lysophosphatidic acid acetyltransferase (LPAAT) enzymes are responsible for the transfer of acyl groups to the sn-2 position on the glycerol backbone. We disclose here that we can reduce the accumulation of excessive amounts of trisaturates in our high SOS strains by expressing heterologous LPAAT genes which were better than the endogenous acyltransferases at discriminating against saturated fatty acids. Expression of LPAT2 homologs from B. napus, T cacao, Garcinia hombroriana and Garcinia indica and their effect on the formation of trisaturated TAGs in the high-C18:0 S6573 strain is disclosed below.

The sequence of the transforming DNA from the BnLPAT2(Bn1.13) expression construct pSZ4198 is shown below The construct is written as pSZ4198:PLOOP::PmHXT1-ScarMEL1-CvNR:PmSAD2-2v2-BnLPAT2(Bn1.13)-CvNR::PLOOP. Relevant restriction sites are indicated in lowercase, bold, and are from 5′-3′ BspQI, KpnI, SpeI, SnaBI, EcoRI, SpeI, ClaI, BglII, AflII, HindIII, SacI and BspQI. Underlined sequences at the 5′ and 3′ flanks of the construct represent genomic DNA from P. moriformis that enable targeted integration of the transforming DNA via homologous recombination at the PLOOP locus. Proceeding in the 5′ to 3′ direction, the PmHXT1 promoter driving the expression of S. carlbergensis MEL1 (ScarMEL1) gene, enabling strains to utilize exogeneous melibiose, is indicated by lowercase, boxed text. The initiator ATG and terminator TGA of ScarMEL1 are indicated by uppercase italics, while the coding region is represented by lowercase italics. The 3′ UTR of the CvNR gene is indicated by small capitals. The P. moriformis SAD2-2v2 promoter driving the expression of the BnLPAT2(Bn1.13) gene is indicated by lowercase, boxed text. The initiator ATG and terminator TGA are indicated by uppercase italics; the sequence encoding BnLPAT2(Bn1.13) is represented by lowercase, underlined italics. A second CvNR 3′ UTR is indicated by small capitals. The Brassica napus LPAAT2(BN1.13) sequence is from Genbank accession GU045434.

SEQ ID NO: 88: Nucleotide sequence of the transforming DNA from pSZ4198
gctcttccgctAACGGAGGTCTGTCACCAAATGGACCCCGTCTATTGCGGGAAACCACG
GCGATGGCACGTTTCAAAACTTGATGAAATACAATATTCAGTATGTCGCGGGCGG
CGACGGCGGGGAGCTGATGTCGCGCTGGGTATTGCTTAATCGCCAGCTTCGCCCC
CGTCTTGGCGCGAGGCGTGAACAAGCCGACCGATGTGCACGAGCAAATCCTGAC
ACTAGAAGGGCTGACTCGCCCGGCACGGCTGAATTACACAGGCTTGCAAAAATA
CCAGAATTTGCACGCACCGTATTCGCGGTATTTTGTTGGACAGTGAATAGCGATG
CGGCAATGGCTTGTGGCGTTAGAAGGTGCGACGAAGGTGGTGCCACCACTGTGC
CAGCCAGTCCTGGCGGCTCCCAGGGCCCCGATCAAGAGCCAGGACATCCAAACT
ACCCACAGCATCAACGCCCCGGCCTATACTCGAACCCCACTTGCACTCTGCAATG
GTATGGGAACCACGGGGCAGTCTTGTGTGGGTCGCGCCTATCGCGGTCGGCGAA
GACCGGGAAggtaccgcggtgagaatcgaaaatgcatcgtttctaggttcggagacggtcaattccctgctccggcgaatctg
tcggtcaagctggccagtggacaatgttgctatggcagcccgcgcacatgggcctcccgacgcggccatcaggagcccaaacagc
gtgtcagggtatgtgaaactcaagaggtccctgctgggcactccggccccactccgggggcgggacgccaggcattcgcggtcggt
cccgcgcgacgagcgaaatgatgattcggttacgagaccaggacgtcgtcgaggtcgagaggcagcctcggacacgtctcgctag
ggcaacgccccgagtccccgcgagggccgtaaacattgtttctgggtgtcggagtgggcattttgggcccgatccaatcgcctcatgc
cgctctcgtctggtcctcacgttcgcgtacggcctggatcccggaaagggcggatgcacgtggtgttgccccgccattggcgcccac
gtttcaaagtccccggccagaaatgcacaggaccggcccggctcgcacaggccatgctgaacgcccagatttcgacagcaacacca
tctagaataatcgcaaccatccgcgttttgaacgaaacgaaacggcgctgtttagcatgtttccgacatcgtgggggccgaagcatgct
ccggggggaggaaagcgtggcacagcggtagcccattctgtgccacacgccgacgaggaccaatccccggcatcagccttcatcg
acggctgcgccgcacatataaagccggacgcctaaccggtttcgtggttatgactagtATGttcgcgttctacttcctgacggcctgc
atctccctgaagggcgtgttcggcgtctccccctcctacaacggcctgggcctgacgccccagatgggctgggacaactggaaca
cgttcgcctgcgacgtctccgagcagctgctgctggacacggccgaccgcatctccgacctgggcctgaaggacatgggctaca
agtacatcatcctggacgactgctggtcctccggccgcgactccgacggcttcctggtcgccgacgagcagaagttccccaacgg
catgggccacgtcgccgaccacctgcacaacaactccttcctgttcggcatgtactcctccgcgggcgagtacacgtgcgccggct
accccggctccctgggccgcgaggaggaggacgcccagttcttcgcgaacaaccgcgtggactacctgaagtacgacaactgc
tacaacaagggccagttcggcacgcccgagatctcctaccaccgctacaaggccatgtccgacgccctgaacaagacgggccg
ccccatcttctactccctgtgcaactggggccaggacctgaccttctactggggctccggcatcgcgaactcctggcgcatgtccgg
cgacgtcacggcggagttcacgcgccccgactcccgctgcccctgcgacggcgacgagtacgactgcaagtacgccggcttcc
actgctccatcatgaacatcctgaacaaggccgcccccatgggccagaacgcgggcgtcggcggctggaacgacctggacaa
cctggaggtcggcgtcggcaacctgacggacgacgaggagaaggcgcacttctccatgtgggccatggtgaagtcccccctgat
catcggcgcgaacgtgaacaacctgaaggcctcctcctactccatctactcccaggcgtccgtcatcgccatcaaccaggactcc
aacggcatccccgccacgcgcgtctggcgctactacgtgtccgacacggacgagtacggccagggcgagatccagatgtggtc
cggccccctggacaacggcgaccaggtcgtggcgctgctgaacggcggctccgtgtcccgccccatgaacacgaccctggagg
agatcttcttcgactccaacctgggctccaagaagctgacctccacctgggacatctacgacctgtgggcgaaccgcgtcgacaa
ctccacggcgtccgccatcctgggccgcaacaagaccgccaccggcatcctgtacaacgccaccgagcagtcctacaaggacg
gcctgtccaagaacgacacccgcctgttcggccagaagatcggctccctgtcccccaacgcgatcctgaacacgaccgtccccg
cccacggcatcgcgttctaccgcctgcgcccctcctccTGAtacgtactcgagGCAGCAGCAGCTCGGATAGT
ATCGACACACTCTGGACGCTGGTCGTGTGATGGACTGTTGCCGCCACACTTGCTG
CCTTGACCTGTGAATATCCCTGCCGCTTTTATCAAACAGCCTCAGTGTGTTTGATC
TTGTGTGTACGCGCTTTTGCGAGTTGCTAGCTGCTTGTGCTATTTGCGAATACCAC
CCCCAGCATCCCCTTCCCTCGTTTCATATCGCTTGCATCCCAACCGCAACTTATCT
ACGCTGTCCTGCTATCCCTCAGCGCTGCTCCTGCTCCTGCTCACTGCCCCTCGCAC
AGCCTTGGTTTGGGCTCCGCCTGTATTCTCCTGGTACTGCAACCTGTAAACCAGC
ACTGCAATGCTGATGCACGGGAAGTAGTGGGATGGGAACACAAATGGAAagctgtag
aattcctggctcgggcctcgtgctggcactccctcccatgccgacaacattctgctgtcaccacgacccacgatgcaacgcgacacg
acccggtgggactgatcggttcactgcacctgcatgcaattgtcacaagcgcatactccaatcgtatccgtttgatttctgtgaaaactcg
ctcgaccgcccgcgtcccgcaggcagcgatgacgtgtgcgtgacctgggtgtttcgtcgaaaggccagcaaccccaaatcgcaggc
gatccggagattgggatctgatccgagcttggaccagatcccccacgatgcggcacgggaactgcatcgactcggcgcggaaccca
gctttcgtaaatgccagattggtgtccgataccttgatttgccatcagcgaaacaagacttcagcagcgagcgtatttggcgggcgtgct
accagggttgcatacattgcccatttctgtctggaccgctttaccggcgcagagggtgagttgatggggttggcaggcatcgaaacgc
gcgtgcatggtgtgtgtgtctgttttcggctgcacaatttcaatagtcggatgggcgacggtagaattgggtgttgcgctcgcgtgcatgc
ctcgccccgtcgggtgtcatgaccgggactggaatcccccctcgcgaccctcctgctaacgctcccgactctcccgcccgcgcgcag
gatagactctagttcaaccaatcgacaactagtATGgccatggccgccgccgtgatcgtgcccctgggcatcctgttcttcatctcc
ggcctggtggtgaacctgctgcaggccatctgctacgtgctgatccgccccctgtccaagaacacctaccgcaagatcaaccgcg
tggtggccgagaccctgtggctggagctggtgtggatcgtggactggtgggccggcgtgaagatccaggtgttcgccgacaacg
agaccttcaaccgcatgggcaaggagcacgccctggtggtgtgcaaccaccgctccgacatcgactggctggtgggctggatcc
tggcccagcgctccggctgcctgggctccgccctggccgtgatgaagaagtcctccaagttcctgcccgtgatcggctggtccatgt
ggttctccgagtacctgttcctggagcgcaactgggccaaggacgagtccaccctgaagtccggcctgcagcgcctgaacgactt
cccccgccccttctggctggccctgttcgtggagggcacccgcttcaccgaggccaagctgaaggccgcccaggagtacgccgc
ctcctccgagctgcccgtgccccgcaacgtgctgatcccccgcaccaagggcttcgtgtccgccgtgtccaacatgcgctccttcgt
gcccgccatctacgacatgaccgtggccatccccaagacctcccccccccccaccatgctgcgcctgttcaagggccagccctcc
gtggtgcacgtgcacatcaagtgccactccatgaaggacctgcccgagtccgacgacgccatcgcccagtggtgccgcgacca
gttcgtggccaaggacgccctgctggacaagcacatcgccgccgacaccttccccggccagcaggagcagaacatcggccgc
cccatcaagtccctggccgtggtgctgtcctggtcctgcctgctgatcctgggcgccatgaagttcctgcactggtccaacctgttctc
ctcctggaagggcatcgccttctccgccctgggcctgggcatcatcaccctgtgcatgcagatcctgatccgctcctcccagtccga
gcgctccacccccgccaaggtggtgcccgccaagcccaaggacaaccacaacgactccggctcctcctcccagaccgaggtg
gagaagcagaagTGAatcgatagatctcttaagGCAGCAGCAGCTCGGATAGTATCGACACACT
CTGGACGCTGGTCGTGTGATGGACTGTTGCCGCCACACTTGCTGCCTTGACCTGT
GAATATCCCTGCCGCTTTTATCAAACAGCCTCAGTGTGTTTGATCTTGTGTGTACG
CGCTTTTGCGAGTTGCTAGCTGCTTGTGCTATTTGCGAATACCACCCCCAGCATCC
CCTTCCCTCGTTTCATATCGCTTGCATCCCAACCGCAACTTATCTACGCTGTCCTG
CTATCCCTCAGCGCTGCTCCTGCTCCTGCTCACTGCCCCTCGCACAGCCTTGGTTT
GGGCTCCGCCTGTATTCTCCTGGTACTGCAACCTGTAAACCAGCACTGCAATGCT
GATGCACGGGAAGTAGTGGGATGGGAACACAAATGGAaagcttaattaagagctcAGCGG
CGACGGTCCTGCTACCGTACGACGTTGGGCACGCCCATGAAAGTTTGTATACCGA
GCTTGTTGAGCGAACTGCAAGCGCGGCTCAAGGATACTTGAACTCCTGGATTGAT
ATCGGTCCAATAATGGATGGAAAATCCGAACCTCGTGCAAGAACTGAGCAAACC
TCGTTACATGGATGCACAGTCGCCAGTCCAATGAACATTGAAGTGAGCGAACTGT
TCGCTTCGGTGGCAGTACTACTCAAAGAATGAGCTGCTGTTAAAAATGCACTCTC
GTTCTCTCAAGTGAGTGGCAGATGAGTGCTCACGCCTTGCACTTCGCTGCCCGTG
TCATGCCCTGCGCCCCAAAATTTGAAAAAAGGGATGAGATTATTGGGCAATGGA
CGACGTCGTCGCTCCGGGAGTCAGGACCGGCGGAAAATAAGAGGCAACACACTC
CGCTTCTTAgctcttc

Additional transforming constructs to test the activity of LPAATs from B. napus, T. cacao, G. hombroriana and G. indica contained the same selectable marker, restriction sites, promoters and 3′ UTR elements as pSZ4198. The coding sequences of BnLPAT2(Bn1.5), TcLPAT2, GhomLPAT2A, GhomLPAT2B, GhomLPAT2C, GindLPAT2A, GindLPAT2B and GindLPAT2C are shown in below. In each case the initiator ATG and terminator TGA are indicated by uppercase italics; the sequence encoding the LPAT2 homolog is represented by lowercase italics. The Brassica napus LPAAT2(BN1.13) sequence is from Genbank accession GU045435. The Theobroma cacao LPAAT2 sequence is from the cocoaGenDB database.

Nucleotide sequence of the BnLPAT2(1.5) coding sequence,
used in the transforming DNA from pSZ4202
SEQ ID NO: 89
ATGgccatggccgccgccgccgtgatcgtgcccctgggcatcctgttcttcatctccggcctggtggtgaacctgctgcaggccgt
gtgctacgtgctgatccgccccctgtccaagaacacctaccgcaagatcaaccgcgtggtggccgagaccctgtggctggagctg
gtgtggatcgtggactggtgggccggcgtgaagatccaggtgttcgccgacgacgagaccttcaaccgcatgggcaaggagca
cgccctggtggtgtgcaaccaccgctccgacatcgactggctggtgggctggatcctggcccagcgctccggctgcctgggctcc
gccctggccgtgatgaagaagtcctccaagttcctgcccgtgatcggctggtccatgtggttctccgagtacctgttcctggagcgca
actgggccaaggacgagtccaccctgaagtccggcctgcagcgcctgaacgacttcccccgccccttctggctggccctgttcgtg
gagggcacccgcttcaccgaggccaagctgaaggccgcccaggagtacgccgcctcctcccagctgcccgtgccccgcaacgt
gctgatcccccgcaccaagggcttcgtgtccgccgtgtccaacatgcgctccttcgtgcccgccatctacgacatgaccgtggccat
ccccaagacctcccccccccccaccatgctgcgcctgttcaagggccagccctccgtggtgcacgtgcacatcaagtgccactcc
atgaaggacctgcccgagtccgacgacgccatcgcccagtggtgccgcgaccagttcgtggccaaggacgccctgctggacaa
gcacatcgccgccgacaccttccccggccagaaggagcacaacatcggccgccccatcaagtccctggccgtggtggtgtcctg
ggcctgcctgctgaccctgggcgccatgaagttcctgcactggtccaacctgttctcctccctgaagggcatcgccctgtccgccctg
ggcctgggcatcatcaccctgtgcatgcagatcctgatccgctcctcccagtccgagcgctccacccccgccaaggtggcccccg
ccaagcccaaggacaagcaccagtccggctcctcctcccagaccgaggtggaggagaagcagaagTGA
Nucleotide sequence of the TcLPAT2 coding sequence, used
in the transforming DNA from pSZ4206
SEQ ID NO: 90
ATGgccatcgccgccgccgccgtgatcgtgcccctgggcctgctgttcttcatctccggcctggtggtgaacctgatccaggccctgtgcttc
gtgctgatccgccccctgtccaagaacacctaccgcaagatcaaccgcgtggtggccgagctgctgtggctggagctgatctggctggtgg
actggtgggccggcgtgaagatcaaggtgttcatggaccccgagtccttcaacctgatgggcaaggagcacgccctggtggtggccaacc
accgctccgacatcgactggctggtgggctggctgctggcccagcgctccggctgcctgggctccgccctggccgtgatgaagaagtcctcc
aagttcctgcccgtgatcggctggtccatgtggttctccgagtacctgttcctggagcgctcctgggccaaggacgagaacaccctgaaggc
cggcctgcagcgcctgaaggacttcccccgccccttctggctggccttcttcgtggagggcacccgcttcacccaggccaagttcctggccgc
ccaggagtacgccgcctcccagggcctgcccatcccccgcaacgtgctgatcccccgcaccaagggcttcgtgtccgccgtgtcccacatgc
gctccttcgtgcccgccatctacgacatgaccgtggccatccccaagtcctccccctcccccaccatgctgcgcctgttcaagggccagccctc
cgtggtgcacgtgcacatcaagcgctgcctgatgaaggagctgcccgagaccgacgaggccgtggcccagtggtgcaaggacatgttcg
tggagaaggacaagctgctggacaagcacatcgccgaggacaccttctccgaccagcccatgcaggacctgggccgccccatcaagtcc
ctgctggtggtggcctcctgggcctgcctgatggcctacggcgccctgaagttcctgcagtgctcctccctgctgtcctcctggaagggcatcg
ccttcttcctggtgggcctggccatcgtgaccatcctgatgcacatcctgatcctgttctcccagtccgagcgctccacccccgccaaggtggc
ccccggcaagcccaagaacgacggcgagacctccgaggcccgccgcgacaagcagcagTGA
Nucleotide sequence of the GhomLPAT2A coding sequence,
used in the transforming DNA from pSZ4412.
SEQ ID NO: 91
ATGgccatccccgccgccatcgtgatcgtgcccgtgggcctgctgttcttcatctccggcctgatcgtgaacctgctgcaggccctgtgcttcg
tgctgatccgccccctgtccaagtccgcctaccgcaccatcaaccgccagctggtggagctgctgtggctggagctggtgtgcatcgtggac
tggtgggcccgcgtgaagatccagctgttcaccgacaaggagaccctgaactccatgggcaaggagcacgccctggtgatgtgcaacca
ccgctccgacatcgactggctggtgggctggatcctggcccagcgctccggctgcctgggctccaccgtggccgtgatgaagaagtcctcca
aggtgctgcccgtgatcggctggtccatgtggttctccgagtacctgttcctggagcgcaactgggccaaggacgagtccaccctgaagtcc
ggcctgcagcgcctgcgcgacttcccccgccccttctggctggccctgttcgtggagggcacccgcttcacccagcccaagctgctggccgcc
caggagtacgccgcctccaccggcctgcccatcccccgcaacgtgctgatcccccgcaccaagggcttcgtgtccgccgtgtccatcacccgc
tccttcgtgcccgtgatctacgacatcaccgtggccatccccaagtcctccccccagcccaccatgctgcgcctgttcaagggccagtcctccg
tggtgcacgtgcacctgaagcgccacctgatgaaggacctgcccgagtccgacgacgacgtggcccagtggtgccgcgaccagttcgtgg
tgaaggactccctgctggacaagcacatcgccgaggacaccttctccgaccaggagctgcaggacatcggccgccccatcaagtccctgg
tggtgttcacctcctgggtgtgcatcatcaccttcggcgccctgaagttcctgcagtggtcctccctgctgcactcctggaagggcatcgccat
ctccgcctccggcctggccatcgtgaccgtgctgatgcacatcctgatccgcttctcccagtccgagcactccacctccgccaagatcgccgcc
gagaagcacaagaacggcggcgtgtcccaggagatgggccgcgagaagcagcacTGA
Nucleotide sequence of the GhomLPAT2B coding sequence,
used in the transforming DNA from pSZ4413.
SEQ ID NO: 92
ATGgagatccccgccgtggccgtgatcgtgcccatcggcatcctgttcttcatctccggcctgatcgtgaacctgatgcaggccatctgcttc
ttcctgatccgccccctgtccaagaacacccaccgcatcgtgaaccgccagctggccgagctgctgtggctggagctgatctggatcgtgga
ctggtgggccggcgtgaagatccagctgttcaccgacaaggagaccctgcacctgatgggcaaggagcacgccctggtgatctgcaacc
actcctccgacatcgactggctggtgggctggctgctgtgccagcgctccggctgcctgggctccgccctggccgtgatgaagtcctcctcca
aggtgctgcccgtgatcggctggtccatgtggttctccgagtacctgttcctggagcgctcctgggccaaggacgagtccaccctgaagtcc
ggcctgcagcgcctgaaggacttcccccgccccttctggctggccctgttcgtggagggcacccgcttcacccaggccaagctgctggccgc
ccaggagtacgccatgtccgccggcctgcccgtgccccgcaacgtgctgatcccccgcaccaagggcttcgtgtccgccgtgtccaacatgc
gctccttcgtgcccgccatctacgacgtgaccgtggccatccccaagtcctccgtgcagcccaccatgctgcgcctgttcaagggccagtcctc
cgtggtgcaggtgcacctgaagcgccactccatgaaggacctgcccgagtccgaggacgacgtggcccagtggtgccgcgaccgcttcgt
ggtgaaggactccctgctggacaagcacaaggtggaggacaccttcaccgaccaggagctgcaggacctgggccgccccatcaagtccc
tggtggtggtgacctgctgggcctgcatcatcatcttcggcatcctgaagttcctgcagtggtcctccctgctgtactcctggaagggcatggc
catctccgcctccggcctggccgtggtgaccttcctgatgcagatcctgatccgcttctcccagtccgagcgctccacccccgccaagatcgcc
cccgccaagcccaacaaggccggcaactcctccgagaccgtgcgcgacaagcaccagTGA
Nucleotide sequence of the GhomLPAT2C coding sequence,
used in the transforming DNA from pSZ4414.
SEQ ID NO: 93
ATGgccatccccgccgccatcatcatcgtgcccctgggcctgatcttcttcacctccggcctgatcatcaacctgatccaggccgtgtgctacg
tgctgatccgccccctgtccaagtccaccttccgccgcatcaaccgcgagctggccgagctgctgtggctggagctggtgtgggtggtggac
tggtgggccggcgtgaagatccagctgttcaccgacaaggagaccctgcactccatgggcaaggagcacgccctggtgatctgcaaccac
cgctccgacatcgactggctggtgggctggatcctggcccagcgctccggctgcctgggctccgccctggccgtgatgaagaagtcctccaa
ggtgctgcccgtgatcggctggtccatgtggttctccgagtacttcttcctggagcgcaactgggccatggacgagtccaccctgaagtccg
gcctgcagcgcctgaaggacttcccccagcccttctggctggccctgttcgtggagggcacccgcttcacccagcccaagctgctggccgccc
aggagtacgccgcctccgccggcctgcccatcccccgcaacgtgctgatcccccgcaccaagggcttcgtgtccgccgtgaacatcatgcgc
tccttcgtgcccgccatctacgacgtgaccgtggccatccccaagtcctccccccagcccaccatgctgcgcctgttcaagggccagtcctccg
tggtgcacgtgcacctgaagcgccacctgatggaggacctgcccgagaccgacgacgacgtggcccagtggtgccgcgaccgcttcgtgg
tgaaggactccctgctggacaagtacgtggccgaggacaccttctccgaccaggagctgcaggacctgggccgccccatcaagtccctgg
tggtggtgacctcctgggtgtgcatcatcgccttcggctccctgaagttcctgcagtggtcctccctgctgtactcctggaagggcatcgtgat
ctccgccgcctccctggccgtggtgaccgtgctgatgcagatcctgatccgcttctcccagtccgagcgctccacctccgccaagatcgccgcc
gccaagcgcaagaacgtgggcgagcacTGA
Nucleotide sequence of the GindPAT2A coding sequence,
used in the transforming DNA from pSZ4415.
SEQ ID NO: 94
ATGgccatccccgtggtggtggtgatcgtgcccgtgggcctgctgttcttcatctccggcctgatcgtgaacctgctgcaggccctgtgcttc
gtgctgatccgccccctgtccaagtccgcctaccgcaccatcaaccgccagctggtggagctgctgtggctggagctggtgtgcatcgtgga
ctggtgggcccgcgtgaagatccagctgttcatcgacaaggagaccctgaactccatgggcaaggagcacgccctggtgatgtgcaacc
accgctcctacatcgactggctggtgggctggatcctggcccagcgctccggctgcctgggctccaccgtggccgtgatgaagaagtcctcc
aaggtgctgcccgtgatcggctggtccatgtggttctccgagtacctgttcctggagcgcaactgggccaaggacgagtccaccctgaagt
ccggcctgcagcgcctgcgcgacttcccccgccccttctggctggccctgttcgtggagggcacccgcttcacccagcccaagctgctggccg
cccaggagtacgccgcctccaccggcctgcccatcccccgcaacgtgctgatcccccgcaccaagggcttcgtgtccgccgtgtccatcaccc
gctccttcgtgcccgtgatctacgacatcaccgtggccatccccaagtcctcctcccagcccaccatgctgaagctgttcaagggccagtcctc
cgtggtgcacgtgcacctgaagcgccacctgatgaaggacctgcccgagtccgacgacgacgtggcccagtggtgccgcgcccagttcgt
ggtgaaggactccctgctggacaagcacatcgccgaggacaccttctccgaccaggagctgcaggacatcggccgccccatcaagtccct
ggtggtgttcacctcctgggtgtgcatcatcaccttcggcgccctgaagttcctgcagtggtcctccctgctgcactcctggaagggcatcgcc
atctccgcctccggcctggccatcgtgaccgtgctgatgcacatcctgatccgcttctcccagtccgagcactccacctccgccaagatcgccg
ccgagaagcacaagaacggcggcgtgtcccaggagatgggccgcgagaagcagcacTGA
Nucleotide sequence of the GindPAT2B coding sequence,
used in the transforming DNA from pSZ4416.
SEQ ID NO: 95
ATGggcatccccgccgtggccgtgatcgtgcccatcggcatcctgttcttcatctccggcttcatcgtgaacctgatgcaggccatctgcttcg
tgctgatccgccccctgtccaagaacacctaccgcatcgtgaaccgccagctggccgagttcctgtggctggagctgatctgggtggtggac
tggtgggccggcgtgaagatccagctgttcaccgacaaggagaccctgcacctgatgggcaaggagcacgccctggtgatctgcaacca
ccgctccgacatcgactggctggtgggctggctgctgtgccagcgctccggctgcctgggctccgccctggccgtgatgaagtcctcctccaa
ggtgctgcccgtgatcggctggtccatgtggttctccgagtacctgttcctggagcgctcctgggccaaggacgagtccaccctgaagctgg
gcctgcagcgcctgaaggacttcccccgccccttctggctggccctgttcgtggagggcacccgcttcacccaggccaagctgctggccgccc
aggagtacgccatgtccgccggcctgcccgtgccccgcaacgtgctgatcccccgcaccaagggcttcgtgtccgccgtgtccaacatgcgc
tccttcgtgcccgccatctacgacgtgaccgtggccatccccaagtcctccgtgcagcccaccatgctgggcctgttcaagggccagtcctgc
gtggtgcaggtgcacctgaagcgccacctgatgaaggacctgcccgagtccgaggacgacgtggcccagtggtgccgcgagcgcttcgt
ggtgaaggactccctgctggacaagcacaaggtggaggacaccttctccgaccaggagctgcaggacctgggccgccccatcaagtccct
ggtggtggtgatctcctgggcctgcatcctgatcttctggatcctgaagttcctgcagtggtcctccctgctgtactcctggaagggcatcgcc
atctccgcctgcgccatggccgtgatcgccttcctgatgcagatcctgctgcgcttctcccagtccgagcgctccacccccgccaagatcgccc
ccgccaagcccaacaacgcccgcaactcctccgagaccgtgcgcgacaagcaccagTGA
SEQ ID NO: 96 Nucleotide sequence of the GindPAT2C coding sequence,
used in the transforming DNA from pSZ4417.
ATGgccatccccgccgccatcatcatcgtgcccctgggcctgatcttcttcacctccggcttcatcatcaacctgatccaggccgtgtgctacg
tgctgatccgccccctgtccaagtccaccttccgccgcatcaaccgccagctggccgagctgctgtggctggagctggtgtgggtggtggac
tggtgggccggcgtgaagatccagctgttcaccaacaaggagaccctgcactccatcggcaaggagcacgccctggtgatctgcaaccag
cgctccgacatcgactggctggtgggctggatcctggcccagcgctccggctgcctgggctccgccctggccgtgatgaagaagtcctccaa
ggtgctgcccgtgatcggctggtccatgtggttctccgagtacctgttcctggagcgcaactgggccatggacgagtccaccctgaagtccg
gcctgcagtggctgaaggacttcccccagcccttctggctggccctgttcgtggagggcacccgcttcacccagcccaagctgctggccgcc
caggagtacgccgcctccgccggcctgcccatcccccgcaacgtgctgatcccccgcaccaagggcttcgtgtccgccgtgaacatcatgcg
ctccttcgtgcccgccgtgtacgacgtgaccgtggccatccccaagtcctccccccagcccaccatgctgcgcctgttcaagggccagtcctcc
gtggtgcacgtgcacctgaagcgccacctgatggaggacctgcccgagaccgacgacgacgtggcccagtggtgccgcgaccgcttcgtg
gtgaaggactccctgctggacaagcacctggccgaggacaccttctccgaccaggagctgcaggacctgggccgccccatcaagtccctg
gtggtggtgacctcctgggtgtgcatcatcgccttcggcgccctgaagttcctgcagtggtcctccctgctgtactcctggaagggcatcgtg
atctccgccgcctccctggccgtggtgaccgtgctgatgcagatcctgatccgcttctcccagtccgagcgctccacctccgccaaggtggtg
gccgagaagcgcaagaacgtgggcgagcacTGA

Constructs D2971, D2973, D2975, D3219, D3221, D3223, D3225, D3227 and D3229, derived from pSZ4198, pSZ4202, pSZ4206, pSZ4412, pSZ4413, pSZ4414, pSZ4415, pSZ4416 and pSZ4417, respectively, were transformed into the S6573 parent strain. The fatty acid profiles of primary transformants are shown in Table 10. Also shown are the SOS/SSS ratios determined by LC/MS multiple response measurements. Expression of LPAT2 genes had no discernable effect on C16:0 or C18:0 accumulation, but C18:2 levels increased by 1-2% compared to the S6573 parent in strains when expressing the D2971, D2973, D2975, D3221, D3223, and D3227 constructs. Expression of LPAT2 genes increased C18:2 and also elevated ratios of SOS/SSS, showing reduced accumulation of trisaturated TAGs.

TABLE 10
Fatty acid profiles and SOS/SSS ratios of D2971, D2973, D2975,
D3219, D3221, D3223, D3225, D3227 and D3229 primary transformants.

| Strain | LPAAT gene | SOS/SSS | C14:0 | C16:0 | C18:0 | C18:1 | C18:2 | C18:3 α | C20:0 | saturates |
|---|---|---|---|---|---|---|---|---|---|---|
| S5100 | | | 0.7 | 17.7 | 4.1 | 68.5 | 6.8 | 0.6 | 0.4 | 23.3 |
| S6573.1 | | 15 | 0.8 | 6.2 | 50.7 | 33.7 | 5.6 | 0.7 | 1.5 | 59.8 |
| D2971.1 | BnLPAT2(1.13) | 23 | 0.8 | 6.1 | 51.4 | 30.5 | 8.6 | 0.6 | 1.4 | 60.2 |
| D2971.2 | | 16 | 0.8 | 6.1 | 54.3 | 28.9 | 7.0 | 0.6 | 1.5 | 63.3 |
| D2971.4 | | 16 | 0.8 | 6.4 | 53.3 | 29.5 | 7.3 | 0.6 | 1.4 | 62.6 |
| S6573.2 | | 14 | 0.8 | 6.6 | 52.8 | 31.7 | 5.2 | 0.6 | 1.5 | 62.3 |
| D2973.2 | BnLPAT2(1.5) | 22 | 0.8 | 6.2 | 53.4 | 28.3 | 6.4 | 0.6 | 1.7 | 62.7 |
| D2973.38 | | 23 | 0.9 | 7.5 | 51.2 | 29.1 | 6.5 | 0.5 | 1.4 | 61.7 |
| D2973.24 | | 24 | 0.9 | 6.8 | 51.7 | 29.2 | 6.3 | 0.5 | 1.6 | 61.5 |
| S6573.3 | | 14 | 0.8 | 6.6 | 52.8 | 31.7 | 5.2 | 0.6 | 1.5 | 62.3 |
| D2975.33 | TcLPAT2 | 27 | 0.8 | 6.6 | 52.7 | 29.7 | 7.1 | 0.6 | 1.5 | 62.3 |
| D2975.13 | | 32 | 0.8 | 6.5 | 52.4 | 30.2 | 7.3 | 0.6 | 1.4 | 61.7 |
| D2975.35 | | 27 | 0.8 | 6.5 | 52.8 | 29.6 | 7.3 | 0.6 | 1.5 | 62.2 |
| S6573.4 | | 12 | 0.9 | 6.4 | 54.9 | 28.9 | 5.7 | 0.6 | 1.7 | 64.5 |
| D3219.19 | GhomLPAT2A | 12 | 0.9 | 7.1 | 52.4 | 31.2 | 4.8 | 0.5 | 2.0 | 63.1 |
| D3219.20 | | 14 | 0.9 | 6.6 | 53.2 | 30.6 | 5.5 | 0.6 | 1.7 | 63.0 |
| D3219.32 | | 15 | 0.8 | 6.4 | 53.1 | 29.8 | 6.5 | 0.6 | 1.5 | 62.6 |
| S6573.5 | | 12 | 0.9 | 6.4 | 53.7 | 30.3 | 5.5 | 0.6 | 1.6 | 63.3 |
| D3220.1 | GhomLPAT2B | 27 | 0.9 | 6.6 | 52.2 | 30.0 | 7.0 | 0.7 | 1.4 | 61.9 |
| D3221.39 | | 20 | 0.9 | 6.7 | 53.9 | 28.7 | 6.7 | 0.6 | 1.5 | 63.7 |
| D3221.40 | | 22 | 0.8 | 6.5 | 53.7 | 29.1 | 6.8 | 0.6 | 1.4 | 63.2 |
| S6573.6 | | 14 | 0.8 | 6.3 | 54.0 | 30.2 | 5.5 | 0.6 | 1.6 | 63.4 |
| D3223.2 | GhomLPAT2C | 20 | 0.8 | 6.5 | 53.0 | 29.3 | 7.3 | 0.6 | 1.5 | 62.4 |
| D3223.6 | | 21 | 0.8 | 6.5 | 53.5 | 29.3 | 7.0 | 0.6 | 1.4 | 62.7 |
| D3223.7 | | 21 | 0.8 | 6.4 | 52.5 | 30.7 | 6.6 | 0.5 | 1.5 | 61.8 |
| D3225.5 | GindLPAT2A | 13 | 0.9 | 6.6 | 53.5 | 30.2 | 5.6 | 0.6 | 1.6 | 63.2 |
| S6573.7 | | 12 | 0.9 | 6.5 | 53.5 | 29.9 | 5.7 | 0.6 | 1.8 | 63.3 |
| D3227.6 | GindLPAT2B | 23 | 0.8 | 6.4 | 54.1 | 28.8 | 6.8 | 0.6 | 1.6 | 63.5 |
| D3227.3 | | 21 | 0.8 | 6.5 | 53.9 | 29.0 | 6.7 | 0.6 | 1.5 | 63.4 |
| D3227.17 | | 22 | 0.8 | 6.6 | 53.8 | 28.8 | 7.0 | 0.6 | 1.4 | 63.3 |
| S6573.8 | | 11 | 0.8 | 6.4 | 54.3 | 30.1 | 5.4 | 0.6 | 1.7 | 63.8 |
| D3229.41 | GindLPAT2C | 11 | 0.9 | 6.6 | 54.2 | 29.7 | 5.6 | 0.6 | 1.7 | 63.9 |
| D3229.27 | | 13 | 0.8 | 6.4 | 54.1 | 30.0 | 5.6 | 0.6 | 1.7 | 63.6 |
| D3229.33 | | 12 | 0.8 | 6.4 | 54.0 | 30.2 | 5.5 | 0.6 | 1.7 | 63.5 |

Table 11 presents the TAG composition of the lipids produced by D2971, D2973, D2975, D3221, D3223, and D3227 primary transformants relative to the S6573 parent. SOS levels in the LPAT2-expressing strains were equivalent or slightly higher than in the S6573 controls. Trisaturates declined by up to 53%, and total Sat-Unsat-Sat levels improved in all of the strains expressing heterologous LPAT2 genes. Among the LPAT2 genes, the strains expressing the T. cacao LPAT2 homolog showed the greatest improvements in their TAG profiles).

TABLE 11
TAG composition of D2971, D2973, D2975, D3221, D3223, and D3227
primary transformants relative to the S6573 parent.

| | | LPAAT gene | | | | | | | | |
|---|---|---|---|---|---|---|---|---|---|---|
| | | BnLPAT2 | BnLPAT2 | | | Ghom | | Ghom | Gind | |
| | | (1.13) | (1.5) | TcLPAT2 | | LPAT2B | | LPAT2C | LPAT2B | |
| | | Strain | | | | | | | | |
| | | D2971.1 | D2973.38 | D2975.33 | D2975.13 | D3221.39 | D3221.40 | D3223.6 | D3227.3 | D3227.6 |
| % S6573 TAG | SOS | 100 | 100 | 110 | 104 | 107 | 107 | 108 | 103 | 105 |
| | Sat-Sat-Sat | 57 | 63 | 48 | 47 | 74 | 62 | 68 | 62 | 70 |
| | Sat-U-Sat | 109 | 107 | 113 | 110 | 112 | 112 | 109 | 108 | 107 |
| | Sat-O-Sat | 97 | 100 | 105 | 102 | 106 | 105 | 102 | 104 | 104 |
| | Sat-L-Sat | 174 | 147 | 155 | 155 | 139 | 143 | 141 | 130 | 125 |
| | U-U-U/Sat | 85 | 86 | 72 | 83 | 64 | 69 | 78 | 82 | 79 |

We analyzed the fatty acid profiles, TAG profiles and lipid titers from 50 mL shake flask cultures of stable lines generated from D2975-33. C18:0 and C16:0 levels were comparable between the strains and the S6573 control, and lipid titers ranged from 75-105% of the parent strain titer (Table 12). C18:2 levels increased by more than 2% in the TcLPAT2-expressing strains.

TABLE 12
Fatty acid profiles of TcLPAT2-expressing stable lines made from
D2975-33.

| | | Primary | | | | | |
|---|---|---|---|---|---|---|---|
| | | D1940.19 | D2975.33 | | | | |
| | | Strain | | | | | |
| | | S6573 | S7813 | S7815 | S7816 | S7817 | S7819 |
| Fatty | C12:0 | 0.2 | 0.2 | 0.2 | 0.2 | 0.2 | 0.2 |
| Acid | C14.0 | 0.9 | 0.7 | 0.8 | 0.8 | 0.7 | 0.7 |
| Area % | C16:0 | 6.5 | 5.9 | 6.1 | 5.9 | 6.1 | 6.0 |
| | C16:1 cis-9 | 0.1 | 0.1 | 0.1 | 0.1 | 0.1 | 0.1 |
| | C17:0 | 0.2 | 0.2 | 0.2 | 0.2 | 0.2 | 0.2 |
| | C18:0 | 56.1 | 55.6 | 55.9 | 56.2 | 53.9 | 53.9 |
| | C18:1 | 28.1 | 26.8 | 26.6 | 26.5 | 28.8 | 28.4 |
| | C18:2 | 5.5 | 8.1 | 7.7 | 7.9 | 7.7 | 7.8 |
| | C18:3 α | 0.6 | 0.5 | 0.6 | 0.5 | 0.6 | 0.7 |
| | C20:0 | 1.5 | 1.5 | 1.4 | 1.3 | 1.3 | 1.5 |
| | C22:0 | 0.2 | 0.2 | 0.1 | 0.1 | 0.1 | 0.2 |
| | C24:0 | 0.1 | 0.1 | 0.1 | 0.1 | 0.1 | 0.1 |
| | saturates | 65.7 | 64.4 | 65.0 | 64.9 | 62.8 | 62.9 |

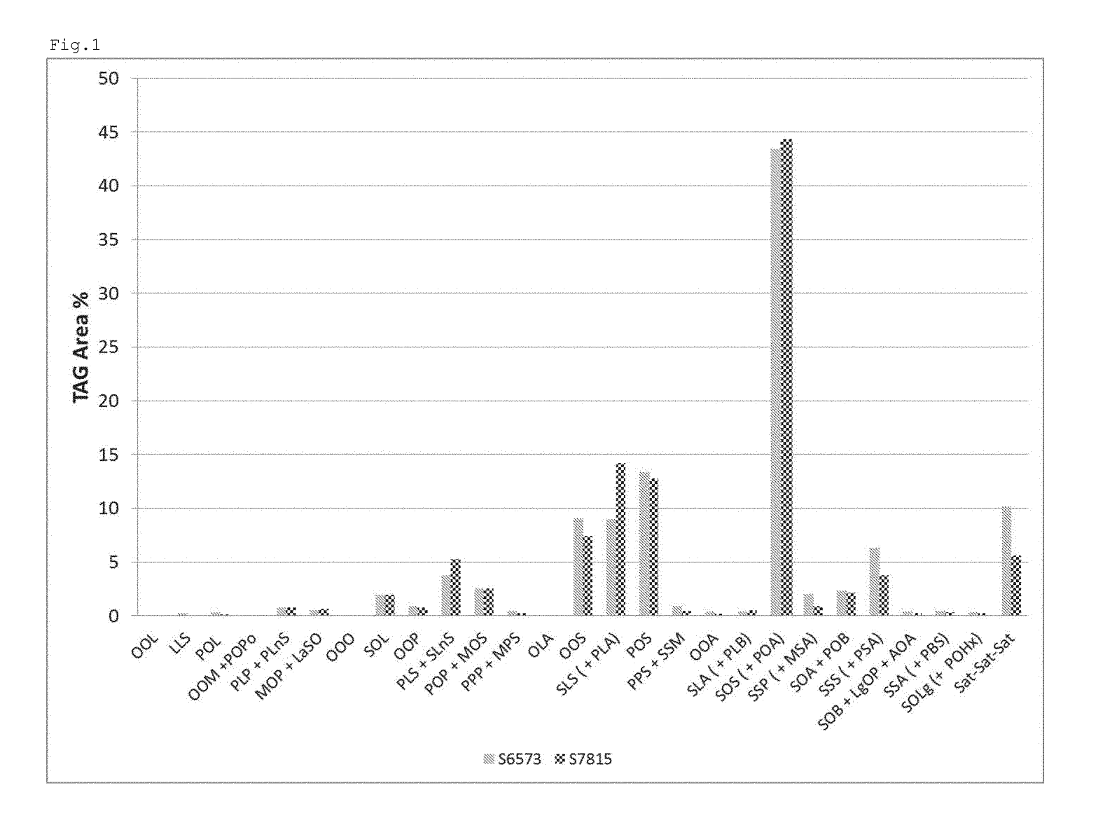

The TAG profiles of S6573 and S57815 are compared in FIG. 1. SOS levels in the LPAT2-expressing strains were higher than in the S6573 control. Trisaturates were reduced from 10.2% in S6573 to 5.6% in S7815. Much of the improvement in total sat-unsat-sat levels in S7815 came from a 4% increase in stearate-linoleate-stearate (SLS) and a 1.5% increase in palmitate-linoleate-stearate (PLS), consistent with the enhanced C18:2 content of that strain. These results indicate that the T. cacoa LPAT2 reduces the incorporation of saturated fatty acids at the sn-2 position.

The performance of S7815 versus the S6573 parent strain was compared in high-density fermentations. The fatty acid profile of each strain at the two time points of the fermentations are shown in Table 13. The strains had very similar composition, with 5.5-5.7% C16:0, 56.4-56.8% C18:0, and 27.2-28.6% C18:1 as the major fatty acids. As was observed in the shake flask assays, (see Table 12), C18:2 levels increased from 5.5% in S6573 to 7.7% in S7815 (Table 13). Normalized lipid titers and yields were comparable between the two strains, indicating that expression of the TcLPAT2 gene in S7815 did not have deleterious effects on growth or lipid accumulation.

TABLE 13
Fatty acid profiles of S7815 versus S6573 fermentations.

| | | |
|---|---|---|
| Strain | S6573 | S7815 |
| Fermentation | 140207F25 | 140208F26 |

| | | | | | |
|---|---|---|---|---|---|
| Fatty Acid | C12:0 | 0.19 | 0.20 | 0.20 | 0.21 |
| Area % | C14:0 | 0.71 | 0.72 | 0.66 | 0.66 |
| | C16:0 | 5.69 | 5.73 | 5.57 | 5.54 |
| | C16:1 cis-7 | 0.05 | 0.05 | 0.05 | 0.06 |
| | C16:1 cis-9 | 0.07 | 0.06 | 0.05 | 0.05 |
| | C17:0 | 0.11 | 0.11 | 0.12 | 0.11 |
| | C18:0 | 56.01 | 56.78 | 55.50 | 56.37 |
| | C18:1 | 29.31 | 28.58 | 27.92 | 27.19 |
| | C18:2 | 5.56 | 5.51 | 7.75 | 7.70 |
| | C18:3 α | 0.34 | 0.32 | 0.40 | 0.37 |
| | C20:0 | 1.51 | 1.50 | 1.35 | 1.34 |
| | C22:0 | 0.16 | 0.16 | 0.14 | 0.14 |
| | C24:0 | 0.10 | 0.09 | 0.09 | 0.08 |
| | sum C18 | 91.22 | 91.19 | 91.57 | 91.63 |
| | saturates | 64.54 | 65.34 | 63.69 | 64.51 |
| | unsaturates | 35.46 | 34.64 | 36.30 | 35.49 |

Table 13 compares the TAG profiles of the lipids produced during high-density fermentation of S7815 versus S6573. SOS and Sat-Oleate-Sat levels were almost identical between S7815 and the S6573 control. However, Sat-Linoleate-Sat levels increased by more than 7%, and di-unsaturated and tri-unsaturated TAGs (U-U-U/Sat) declined by more than 3% in S7815 compared to S6573. Trisaturates at the end points of the fermentations were reduced from 10.1% in S6573 to 6.1% in S7815. These results indicate that the activity of T. cacoa LPAT2 drives the transfer of unsaturated fatty acids towards the sn-2 position and discriminates against the incorporation of saturated fatty acids at sn-2.

### Example 6: Identification and Expression of Novel LPAAT, GPAT, DGAT, LPCAT and PLA2 with Specificity for Mid-Chain Fatty Acids

In this example, we demonstrate the effect of expression of LPAAT, GPAT, DGAT, LPCAT and PLA2 enzymes involved in triacylglycerol biosynthesis (in previously described P. moriformis (UTEX 1435) transgenic strains, S7858 and S8174. S7858 and S8174 were prepared according to co-owned WO2015/051319, herein incorporated by reference. In addition co-owned WO2010/063031 and WO2010/063032 teach the expression Cuphea hookerianas FATB2. Briefly, strain S7858 is a strain that express sucrose invertase and a Cuphea. hookeriana FATB2. To make S7858, the construct pSZ4329 (SEQ ID NO: 197) was engineered into S3150, a strain classically mutagenized to increase lipid yield. The plasmid, pSZ4329 is written as THI4a::CrTUB2-ScSUC2-PmPGH:PmAcp-Plp-CpSAD1_tp_trimmed_ChFATB2_FLAG-CvNR::THI4a The annotation of the coding portions of pSZ4329 is shown in the Table A below.

TABLE A

| | | Nucleotide | Nucleotide | Nucleotide |
|---|---|---|---|---|
| pSZ4329 | Identity | Number | Number | Length |
| THI4a 3′ flank | 3′ flanking | 5,692 | 6,394 | 703 |
| | sequences of | | | |
| | endogenous | | | |
| | THI4 | | | |
| CvNR | 3′UTR | 5,278 | 5,679 | 402 |
| ChFATB2 | CDS | 4,105 | 5,271 | 1,167 |
| CpSAD1tp-trimmed | CDS | 3,991 | 4,104 | 114 |
| PmACP-P1 promoter | promoter | 3,411 | 3,981 | 571 |
| Buffer DNA | | 3,199 | 3,404 | 206 |
| UTR04424 = | | | | |
| PmPGH | | | | |
| UTR | 3′UTR | 2,749 | 3,192 | 444 |
| ScSUC2(o) | CDS | 1,144 | 2,742 | 1,599 |
| CrTUB2 promoter | promoter | 820 | 1,131 | 312 |
| THI4a 5′ flank | 5′ flanking | 27 | 813 | 787 |
| | sequences of | | | |
| | endogenous | | | |
| | THI4 | | | |

Strain S7858, accumulates C8:0 fatty acids to about 12% and C10:0 fatty acids to about 22-24%. Briefly, strain S8174 is a strain that express sucrose invertase and a Cuphea. Avigera var. pulcherrima FATB2. To make S8174, the construct pSZ5078 (SEQ ID NO: 198) was engineered into S3150, a strain classically mutagenized to increase lipid yield. pSZ5078 is written as THI4a5′::CrTUB2_ScSUC2_PmPGH:PmAMT3_CpSAD1_tp_trimmed-CaFATB1_Flag_CvNR::THI4a3′. Strain S8174 accumulates C8:0 fatty acids to about 24% and C10:0 fatty acids to about 10%. The annotation of the coding portions of pSZ5078 is shown in the Table B below.

TABLE B

| | | Nucleotide | Nucleotide | Nucleotide |
|---|---|---|---|---|
| pSZ5078 | Identity | Number | Number | Length |
| THI4a 3′ flank | 3′ flanking | 6,200 | 6,902 | 703 |
| | sequences of | | | |
| | endogenous THI4 | | | |
| CvNR | 3′UTR | 5,786 | 6,187 | 402 |
| CaFATB1 | CDS | 4,602 | 5,771 | 1,170 |
| wild-type | | | | |
| CpSAD1tp | CDS | 4,488 | 4,601 | 114 |
| AMT3 | promoter eukaryotic | 3,411 | 4,481 | 1,071 |
| Buffer DNA | misc_feature | 3,199 | 3,404 | 206 |
| PmPGH | 3′UTR | 2,749 | 3,192 | 444 |
| ScSUC2(o) | CDS | 1,144 | 2,742 | 1,599 |
| CrTUB2 | promoter | 820 | 1,131 | 312 |
| promoter | | | | |
| THI4a 5′ flank | 5′ flanking | 27 | 813 | 787 |
| | sequences of | | | |
| | endogenous THI4 | | | |

The pool of acyl-CoAs in the ER can be utilized for the synthesis of TAGs as well as phospholipids and long chain fatty acids. The enzymes involved in the synthesis of TAGS and phospholids actively compete against each other for the same substrates. Acyl-CoAs can associate with lysophosphatidate to form phosphatidate which is converted to phosphatidylcholine (PC) and other phospholipid species. PC can be desaturated by FAD2 and FAD3 enzymes to generate polyunsaturated fatty acids, which can be cleaved by phosphotransferases and reenter the acyl-CoA pool. Acyl-CoAs can also be generated from PC directly by acyl-CoA:lysophosphatidylcholine acyltransferase (LPCAT). LPCAT can also catalyze the reverse reaction to consume acyl-CoA. Removal of fatty acids from PC to form acyl-CoAs can also be catalyzed by phospholipase A₂ (PLA2). TAG formation in the ER from acyl-CoAs requires action of glycerol phosphate acyltransferase (GPAT), lysophosphatidic acid acyltransferase (LPAAT) and diacyl glycerol acyltransferase (DGAT).

The endogenous P. moriformis TAG biosynthesis machinery has evolved to function with the longer chain fatty acids that the strain normally makes. We introduced heterologous acyltransferases and phospholipases from species that naturally accumulate high levels of short chain fatty acids into Prototheca to increase accumulation of C8:0 fatty acids. We identified the following plant enzymes in NCBI as shown in Table 14 below.

TABLE 14
Genes representing target enzymes identified from
higher plants that produce high amounts of C8:0 and
C10:0. All these genes were synthesized with codon
usage optimized for expression in Prototheca.

| Species | Gene | Enzyme |
|---|---|---|
| Cocos nucifera | CnLPAAT1 | LPAAT |
| Cuphea paucipetala | CpauLPAAT1 | |
| Cuphea procumbens | CprocLPAAT1 | |
| Cuphea painteri | CpaiLPAAT1 | |
| Cuphea hookeriana | ChookLPAAT1 | |
| Cuphea ignea | CigneaLPAAT1 | |
| Cuphea avigera var. pulcherrima | CavigLPAAT1 | |
| Cuphea avigera var. pulcherrima | CavigLPAAT2 | |
| Cuphea palustris | CpalLPAAT1 | |
| Cuphea koehneana | CkoeLPAAT1 | |
| Cuphea koehneana | CkoeLPAAT2 | |
| Cuphea procumbens | CprocLPAAT2 | |
| Cuphea PSR23 | CuPSRLPAAT2 | |
| Cuphea avigera var. pulcherrima | CavigGPAT9 | GPAT |
| Cuphea hookeriana | ChookGPAT9-1 | |
| Cuphea ignea | CignGPAT9-1 | |
| Cuphea ignea | CignGPAT9-2 | |
| Cuphea palustris | CpalGPAT9-1 | |
| Cuphea palustris | CpalGPAT9-2 | |
| Cuphea avigera var. pulcherrima | CavigDGAT1 | DGAT |
| Cuphea hookeriana | ChookDGAT1-1 | |
| Cuphea avigera var. pulcherrima | CavigLPCAT | LPCAT |
| Cuphea palustris | CpalLPCAT | |
| Cuphca paucipetala | CpauLPCAT | |
| Cuphea schumanii | CschuLPCAT1 | |
| Cuphea avigera var. pulcherrima | CavigPLA2-1 | PLA2 |
| Cuphea ignea | CignPLA2-1 | |
| Cuphea procumbens | CprocPLA2-2 | |
| Cuphea PSR23 | CuPSR23PLA2-2 | |

We made a set of constructs expressing heterologous short chain specific acyltransferases and PLA2s as shown in Table 15. The genes were codon optimized to reflect UTEX 1435 codon usage.

TABLE 15
List of constructs transformed into S7858 or S8174

| D# | Strain | Construct |
|---|---|---|
| D4289 | S7858 | SAD2-1vD::CpauLPAAT1-PmATP:PmHXT1-ScarMEL-PmPGK::SAD2Bex |
| D4290 | S7858 | SAD2-1vD::CpaiLPAAT1-PmATP:PmHXT1-ScarMEL-PmPGK::SAD2Bex |
| D4291 | S7858 | SAD2-1vD::CigneaLPAAT1-PmATP:PmHXT1-ScarMEL-PmPGK::SAD2Bex |
| D4292 | S7858 | SAD2-1vD::CprocLPAAT1-PmATP:PmHXT1-ScarMEL-PmPGK::SAD2Bex |
| D4293 | S7858 | SAD2-1vD::ChookLPAAT1-PmATP:PmHXT1-ScarMEL-PmPGK::SAD2Bex |
| D4404 | S7858 | SAD2-1vD::CnLPAAT-PmATP:PmHXT1-ScarMEL1-PmPGK::SAD2Bex |
| D4517 | S8174 | SAD2-1vD::CavigLPAAT1-PmATP:PmHXT1-ScarMEL-PmPGK::SAD2Bex |
| D4518 | S8174 | SAD2-1vD::CavigLPAAT2-PmATP:PmHXT1-ScarMEL-PmPGK::SAD2Bex |
| D4519 | S8174 | SAD2-1vD::CpalLPAAT1-PmATP:PmHXT-ScarMEL-PmPGK::SAD2Bex |
| D4690 | S8174 | SAD2-1vD::CuPSR23 LPAAT2-1-PmATP:PmHXT1-ScarMEL-PmPGK::SAD2Bex |
| D4728 | S8174 | SAD2-1vD::CkoeLPAAT-PmATP:PmHXT1-ScarMEL-PmPGK::SAD2Bex |
| D4729 | S8174 | SAD2-1vD::CkoeLPAAT2-PmATP:PmHXT1-ScarMEL-PmPGK::SAD2Bex |
| D4730 | S8174 | SAD2-1vD::CprocLPAAT2-PmATP:PmHXT1-ScarMEL-PmPGK::SAD2Bex |
| D4551/D4683 | S8174 | SAD2-1vD::CavigGPAT9-PmATP:PmHXT1-ScarMEL-PmPGK::SAD2Bex |
| D4552/D4684 | S8174 | SAD2-1vD::ChookGPAT9-1-PmATP:PmHXT1-ScarMEL-PmPGK::SAD2Bex |
| D4553/D4685 | S8174 | SAD2-1vD::CignGPAT9-1-PmATP:PmHXT1-ScarMEL-PmPGK::SAD2Bex |
| D4554/D4686 | S8174 | SAD2-1vD::CignGPAT9-2-PmATP:PmHXT1-ScarMEL-PmPGK::SAD2Bex |
| D4724 | S8174 | SAD2-1vD::CpalGPAT9-1-PmATP:PmHXT1-ScarMEL-PmPGK::SAD2Bex |
| D4725 | S8174 | SAD2-1vD::CpalGPAT9-2-PmATP:PmHXT1-ScarMEL-PmPGK::SAD2Bex |
| D4549 | S8174 | SAD2-1vD::CavigDGAT1-PmATP:PmHXT1-ScarMEL-PmPGK::SAD2Bex |
| D4681 | S8174 | SAD2-1vD::CavigDGAT1-PmATP:PmHXT1-ScarMEL-PmPGK::SAD2Bex |
| D4555/D4688 | S8174 | SAD2-1vD::CavigLPCAT-PmATP:PmHXT1-ScarMEL-PmPGK::SAD2Bex |
| D4726 | S8174 | SAD2-1vD::CpalLPCAT-PmATP:PmHXT1-ScarMEL-PmPGK::SAD2Bex |
| D4556/D4689 | S8174 | SAD2-1vD::CpauLPCAT-PmATP:PmHXT1-ScarMEL-PmPGK::SAD2Bex |
| D4727 | S8174 | SAD2-1vD::CschuLPCAT1-PmATP:PmHXT1-ScarMEL-PmPGK::SAD2Bex |
| D4732 | S8174 | SAD2-1vD::CavigPLA2-1-PmATP:PmHXT1-ScarMEL-PmPGK::SAD2Bex |
| D4734 | S8174 | SAD2-1vD::CignPLA2-1-PmATP:PmHXT1-ScarMEL-PmPGK::SAD2Bex |
| D4735 | S8174 | SAD2-1vD::CuPSR23PLA2-2-PmATP:PmHXT1-ScarMEL-PmPGK::SAD2Bex |
| D4736 | S8174 | SAD2-1vD::CprocPLA2-2-PmATP:PmHXT1-ScarMEL-PmPGK::SAD2Bex |

All the constructs shown in Table 15 can be written as SAD2-1vD::gene of interest-PmATP-PmHXT1-ScarMEL-PmPGK::SAD2B, and were made to target the transforming DNA to the SAD2 locus on the genome, thereby disrupting the expression of at least one allele of the endogenous stearoyl ACP desaturase. Sequences of all the transforming DNAs are provided below. The relevant restriction sites in the construct from 5′-3′ are Pme I, BspQ I, Kpn I, Xho I, Avr II, Spe I, SnaB I, EcoR V, Sac I, BspQ I, Pme I respectively are indicated in lowercase, bold, and underlined. Pme I sites delimit the 5′ and 3′ ends of the transforming DNA. Bold, lowercase sequences at the 5′ and 3′ end of the construct represent genomic DNA from UTEX 1435 that target integration to the SAD2 locus via homologous recombination, wherein the SAD2 5′ flank provides the promoter for the gene of interest downstream. The primary construct was made with the previously characterized CnLPAAT gene as shown below and all other constructs were made by replacing the CnLPAAT gene with other genes of interest using the restriction sites, Kpn I and Xho I that span the gene on either side. Proceeding in the 5′ to 3′ direction, the first cassette has the codon optimized Cocos nucifera LPAAT and the Prototheca moriformis ATP synthase (PmATP) gene 3′ UTR. The initiator ATG and terminator TGA for cDNAs are indicated by uppercase italics, while the coding region is indicated with lowercase italics. The 3′ UTR is indicated by lowercase underlined text. The second cassette containing the selection gene melibiose from Saccharomyces carlsbergensis (ScarMEL1) is driven by the endogenous HXT1 promoter, and has the endogenous phosphoglycerate kinase (PmPGK) gene 3′ UTR. In this cassette, the PmHXT1 promoter is indicated by lowercase, boxed text. The initiator ATG and terminator TGA for the ScarMEL1 gene are indicated in uppercase italics, while the coding region is indicated by lowercase italics. The 3′ UTR is indicated by lowercase underlined text. All the final constructs were sequenced to ensure correct reading frames and targeting sequences.

SEQ ID NO: 97 pSZX61 Sequence of the transforming DNA expressing
CnLPAAT downstream of the SAD2 promoter in the cassette followed by the ScarMEL1
gene for selection downstream of the PmHXT1 promoter in the second cassette.
gtttaaacgccggtcaccacccgcatgctcgtactacagcgcacgcaccgcttcgtgatccaccgggtgaacgtagtcctcgacgg
aaacatctggttcgggcctcctgcttgcactcccgcccatgccgacaacctttctgctgttaccacgacccacaatgcaacgcgaca
cgaccgtgtgggactgatcggttcactgcacctgcatgcaattgtcacaagcgcttactccaattgtattcgtttgttttctgggagc
agttgctcgaccgcccgcgtcccgcaggcagcgatgacgtgtgcgtggcctgggtgtttcgtcgaaaggccagcaaccctaaatcg
caggcgatccggagattgggatctgatccgagtttggaccagatccgccccgatgcggcacgggaactgcatcgactcggcgcgg
aacccagctttcgtaaatgccagattggtgtccgatacctggatttgccatcagcgaaacaagacttcagcagcgagcgtatttgg
cgggcgtgctaccagggttgcatacattgcccatttctgtctggaccgctttactggcgcagagggtgagttgatggggttggcagg
catcgaaacgcgcgtgcatggtgtgcgtgtctgttttcggctgcacgaattcaatagtcggatgggcgacggtagaattgggtgtg
gcgctcgcgtgcatgcctcgccccgtcgggtgtcatgaccgggactggaatcccccctcgcgaccatcttgctaacgctcccgactc
tcccgaccgcgcgcaggatagactcttgttcaaccaatcgacaggtaccATGgacgcctccggcgcctcctccttcctgcgcggccgct
gcctggagtcctgcttcaaggcctccttcggctacgtaatgtcccagcccaaggacgccgccggccagccctcccgccgccccgccgacgcc
gacgacttcgtggacgacgaccgctggatcaccgtgatcctgtccgtggtgcgcatcgccgcctgcttcctgtccatgatggtgaccaccatc
gtgtggaacatgatcatgctgatcctgctgccctggccctacgcccgcatccgccagggcaacctgtacggccacgtgaccggccgcatgct
gatgtggattctgggcaaccccatcaccatcgagggctccgagttctccaacacccgcgccatctacatctgcaaccacgcctccctggtgg
acatcttcctgatcatgtggctgatccccaagggcaccgtgaccatcgccaagaaggagatcatctggtatcccctgttcggccagctgtac
gtgctggccaaccaccagcgcatcgaccgctccaacccctccgccgccatcgagtccatcaaggaggtggcccgcgccgtggtgaagaag
aacctgtccctgatcatcttccccgagggcacccgctccaagaccggccgcctgctgcccttcaagaagggcttcatccacatcgccctccag
acccgcctgcccatcgtgccgatggtgctgaccggcacccacctggcctggcgcaagaactccctgcgcgtgcgccccgcccccatcaccgt
gaagtacttctcccccatcaagaccgacgactgggaggaggagaagatcaaccactacgtggagatgatccacgccctgtacgtggacc
acctgcccgagtcccagaagcccctggtgtccaagggccgcgacgcctccggccgctccaactccTGAttaattaactcgagatgtggaga
tgtagggtggtcgactcgttggaggtgggtgtttttttttatcgagtgcgcggcgcggcaaacgggtccctttttatcgaggtgttccca
acgccgcaccgccctcttaaaacaacccccaccaccacttgtcgaccttctcgtttgttatccgccacggcgccccggaggggcgtcg
tctggccgcgcgggcagctgtatcgccgcgctcgctccaatggtgtgtaatcttggaaagataataatcgatggatgaggaggaga
gcgtgggagatcagagcaaggaatatacagttggcacgaagcagcagcgtactaagctgtagcgtgttaagaaagaaaaactcg

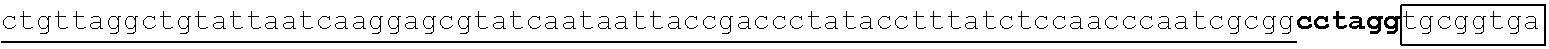

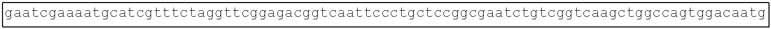

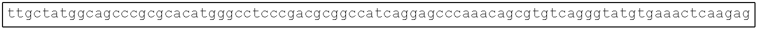

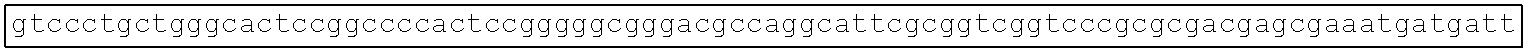

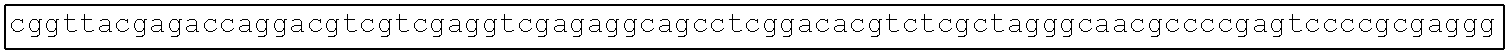

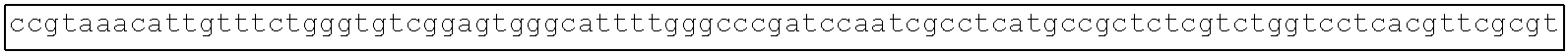

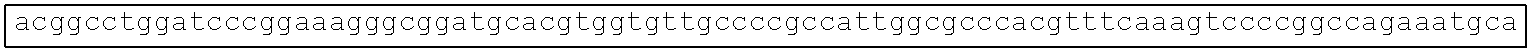

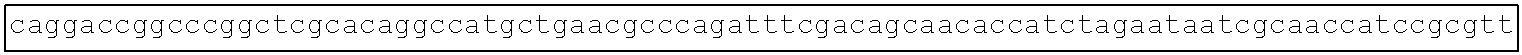

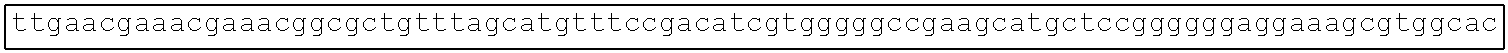

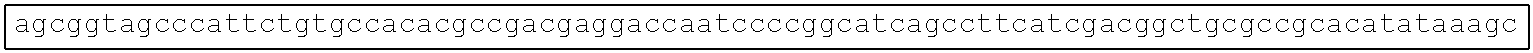

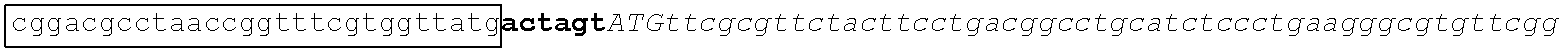

cgtctccccctcctacaacggcctgggcctgacgccccagatgggctgggacaactggaacacgttcgcctgcgacgtctccga
gcagctgctgctggacacggccgaccgcatctccgacctgggcctgaaggacatgggctacaagtacatcatcctggacgact
gctggtcctccggccgcgactccgacggcttcctggtcgccgacgagcagaagttccccaacggcatgggccacgtcgccgac
cacctgcacaacaactccttcctgttcggcatgtactcctccgcgggcgagtacacgtgcgccggctaccccggctccctgggcc
gcgaggaggaggacgcccagttcttcgcgaacaaccgcgtggactacctgaagtacgacaactgctacaacaagggccagt
tcggcacgcccgagatctcctaccaccgctacaaggccatgtccgacgccctgaacaagacgggccgccccatcttctactccct
gtgcaactggggccaggacctgaccttctactggggctccggcatcgcgaactcctggcgcatgtccggcgacgtcacggcgg
agttcacgcgccccgactcccgctgcccctgcgacggcgacgagtacgactgcaagtacgccggcttccactgctccatcatga
acatcctgaacaaggccgcccccatgggccagaacgcgggcgtcggcggctggaacgacctggacaacctggaggtcggcg
tcggcaacctgacggacgacgaggagaaggcgcacttctccatgtgggccatggtgaagtcccccctgatcatcggcgcgaa
cgtgaacaacctgaaggcctcctcctactccatctactcccaggcgtccgtcatcgccatcaaccaggactccaacggcatcccc
gccacgcgcgtctggcgctactacgtgtccgacacggacgagtacggccagggcgagatccagatgtggtccggccccctgg
acaacggcgaccaggtcgtggcgctgctgaacggcggctccgtgtcccgccccatgaacacgaccctggaggagatcttcttc
gactccaacctgggctccaagaagctgacctccacctgggacatctacgacctgtgggcgaaccgcgtcgacaactccacggc
gtccgccatcctgggccgcaacaagaccgccaccggcatcctgtacaacgccaccgagcagtcctacaaggacggcctgtcca
agaacgacacccgcctgttcggccagaagatcggctccctgtcccccaacgcgatcctgaacacgaccgtccccgcccacggc
atcgcgttctaccgcctgcgcccctcctccTGAtacaacttattacgtattctgaccggcgctgatgtggcgcggacgccgtcgtac
tctttcagactttactcttgaggaattgaacctttctcgcttgctggcatgtaaacattggcgcaattaattgtgtgatgaagaaaggg
tggcacaagatggatcgcgaatgtacgagatcgacaacgatggtgattgttatgaggggccaaacctggctcaatcttgtcgcatgt
ccggcgcaatgtgatccagcggcgtgactctcgcaacctggtagtgtgtgcgcaccgggtcgctttgattaaaactgatcgcattgcc
atcccgtcaactcacaagcctactctagctcccattgcgcactcgggcgcccggctcgatcaatgttctgagcggagggcgaagcgt
caggaaatcgtctcggcagctggaagcgcatggaatgcggagcggagatcgaatcagatatcAAGCTCCATCgagctccagc
cacggcaacaccgcgcgccttgcggccgagcacggcgacaagaacctgagcaagatctgcgggctgatcgccagcgacgaggg
ccggcacgagatcgcctacacgcgcatcgtggacgagttcttccgcctcgaccccgagggcgccgtcgccgcctacgccaacatga
tgcgcaagcagatcaccatgcccgcgcacctcatggacgacatgggccacggcgaggccaacccgggccgcaacctcttcgccga
cttctccgcggtcgccgagaagatcgacgtctacgacgccgaggactactgccgcatcctggagcacctcaacgcgcgctggaag
gtggacgagcgccaggtcagcggccaggccgccgcggaccaggagtacgtcctgggcctgccccagcgcttccggaaactcgcc
gagaagaccgccgccaagcgcaagcgcgtcgcgcgcaggcccgtcgccttctcctggatctccgggcgcgagatcatggtctagg
gagcgacgagtgtgcgtgcggggctggcgggagtgggacgccctcctcgctcctctctgttctgaacggaacaatcggccaccccg
cgctacgcgccacgcatcgagcaacgaagaaaaccccccgatgataggttgcggtggctgccgggatatagatccggccgcaca
tcaaagggcccctccgccagagaagaagctcctttcccagcagactcctgaagagcgtttaaac.

The sequence for all of the other acyltransferase constructs are identical to that of pSZEX61 with the exception of the encoded acyltransferase. The acyltransferase sequence alone is provided below for the remaining acyltransferase constructs.

CpauLPAAT1
SEQ ID NO: 98
ggtaccATGgccatccccgccgccgccgtgatcttcctgttcggcctgctgttcttcacctccggcctgatcatcaacctgttccagg
ccctgtgcttcgtgctggtgtggcccctgtccaagaacgcctaccgccgcatcaaccgcgtgttcgccgagctgctgctgtccgagc
tgctgtgcctgttcgactggtgggccggcgccaagctgaagctgttcaccgaccccgagaccttccgcctgatgggcaaggagca
cgccctggtgatcatcaaccacatgaccgagctggactggatgctgggctgggtgatgggccagcacctgggctgcctgggctcc
atcctgtccgtggccaagaagtccaccaagttcctgcccgtgctgggctggtccatgtggttctccgagtacctgtacatcgagcgct
cctgggccaaggaccgcaccaccctgaagtcccacatcgagcgcctgaccgactaccccctgcccttctggatggtgatcttcgtg
gagggcacccgcttcacccgcaccaagctgctggccgcccagcagtacgccgcctcctccggcctgcccgtgccccgcaacgtg
ctgatcccccgcaccaagggcttcgtgtcctgcgtgtcccacatgcgctccttcgtgcccgccgtgtacgacgtgaccgtggccttcc
ccaagacctcccccccccccaccctgctgaacctgttcgagggccagtccatcgtgctgcacgtgcacatcaagcgccacgccat
gaaggacctgcccgagtccgacgacgccgtggcccagtggtgccgcgacaagttcgtggagaaggacgccctgctggacaag
cacaacgccgaggacaccttctccggccaggaggtgcaccgcaccggctcccgccccatcaagtccctgctggtggtgatctcct
gggtggtggtgatcaccttcggcgccctgaagttcctgcagtggtcctcctggaagggcaaggccttctccgtgatcggcctgggc
atcgtgaccctgctgatgcacatgctgatcctgtcctcccaggccgagcgctcctccaaccccgccaaggtggcccaggccaagc
tgaagaccgagctgtccatctccaagaaggccaccgacaaggagaacTGActcgag
CprocLPAAT1
SEQ ID NO: 99
ggtacc

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ctcgag
CpaiLPAAT1
SEQ ID NO: 100
ggtaccATGgccatcccctccgccgccgtggtgttcctgttcggcctgctgttcttcacctccggcctgatcatcaacctgttccagg
ccttctgcttcgtgctgatctcccccctgtccaagaacgcctaccgccgcatcaaccgcgtgttcgccgagctgctgcccctggagtt
cctgtggctgttccactggtgcgccggcgccaagctgaagctgttcaccgaccccgagaccttccgcctgatgggcaaggagcac
gccctggtgatcatcaaccacaagatcgagctggactggatggtgggctgggtgctgggccagcacctgggctgcctgggctcca
tcctgtccgtggccaagaagtccaccaagttcctgcccgtgttcggctggtccctgtggttctccggctacctgttcctggagcgctcc
tgggccaaggacaagatcaccctgaagtcccacatcgagtccctgaaggactaccccctgcccttctggctgatcatcttcgtgga
gggcacccgcttcacccgcaccaagctgctggccgcccagcagtacgccgcctcctccggcctgcccgtgccccgcaacgtgct
gatcccccacaccaagggcttcgtgtcctccgtgtcccacatgcgctccttcgtgcccgccatctacgacgtgaccgtggccttcccc
aagacctcccccccccccaccatgctgaagctgttcgagggccagtccgtggagctgcacgtgcacatcaagcgccacgccatg
aaggacctgcccgagtccgacgacgccgtggcccagtggtgccgcgacaagttcgtggagaaggacgccctgctggacaagc
acaactccgaggacaccttctccggccaggaggtgcaccacgtgggccgccccatcaaggccctgctggtggtgatctcctgggt
ggtggtgatcatcttcggcgccctgaagttcctgctgtggtcctccctgctgtcctcctggaagggcaaggccttctccgtgatcggcc
tgggcatcgtggccggcatcgtgaccctgctgatgcacatcctgatcctgtcctcccaggccgagggctccaaccccgtgaaggc
cgcccccgccaagctgaagaccgagctgtcctcctccaagaaggtgaccaacaaggagaacTGActcgag
ChookLPAAT1
SEQ ID NO: 101
ggtaccATGgccatcccctccgccgccgtggtgttcctgttcggcctgctgttcttcacctccggcctgatcatcaacctgttccagg
ccttctgcttcgtgctgatctcccccctgtccaagaacgcctaccgccgcatcaaccgcgtgttcgccgagctgctgcccctggagtt
cctgtggctgttccactggtgcgccggcgccaagctgaagctgttcaccgaccccgagaccttccgcctgatgggcaaggagcac
gccctggtgatcatcaaccacaagatcgagctggactggatggtgggctgggtgctgggccagcacctgggctgcctgggctcca
tcctgtccgtggccaagaagtccaccaagttcctgcccgtgttcggctggtccctgtggttctccgagtacctgttcctggagcgctcc
tgggccaaggacaagatcaccctgaagtcccacatcgagtccctgaaggactaccccctgcccttctggctgatcatcttcgtgga
gggcacccgcttcacccgcaccaagctgctggccgcccagcagtacgccgcctcctccggcctgcccgtgccccgcaacgtgct
gatcccccacaccaagggcttcgtgtcctccgtgtcccacatgcgctccttcgtgcccgccatctacgacgtgaccgtggccttcccc
aagacctcccccccccccaccatgctgaagctgttcgagggccagtccgtggagctgcacgtgcacatcaagcgccacgccatg
aaggacctgcccgagtccgacgacgccgtggcccagtggtgccgcgacaagttcgtggagaaggacgccctgctggacaagc
acaactccgaggacaccttctccggccaggaggtgcaccacgtgggccgccccatcaaggccctgctggtggtgatctcctgggt
ggtggtgatcatcttcggcgccctgaagttcctgctgtggtcctccctgctgtcctcctggaagggcaaggccttctccgtgatcggcc
tgggcatcgtggccggcatcgtgaccctgctgatgcacatcctgatcctgtcctcccaggccgagggctccaaccccgtgaaggc
cgcccccgccaagctgaagaccgagctgtcctcctccaagaaggtgaccaacaaggagaacTGActcgag
SEQ ID NO: 102 CignLPAAT1
ggtaccATGgccatcgccgccgccgccgtgatcttcctgttcggcctgctgttcttcgcctccggcatcatcatcaacctgttccag
gccctgtgcttcgtgctgatctggcccctgtccaagaacgtgtaccgccgcatcaaccgcgtgttcgccgagctgctgctgatggac
ctgctgtgcctgttccactggtgggccggcgccaagatcaagctgttcaccgaccccgagaccttccgcctgatgggcatggagca
cgccctggtgatcatgaaccacaagaccgacctggactggatggtgggctggatcctgggccagcacctgggctgcctgggctc
catcctgtccatcgccaagaagtccaccaagttcatccccgtgctgggctggtccgtgtggttctccgagtacctgttcctggagcgc
tcctgggccaaggacaagtccaccctgaagtcccacatggagaagctgaaggactaccccctgcccttctggctggtgatcttcgt
ggagggcacccgcttcacccgcaccaagctgctggccgcccagcagtacgccgcctcctccggcctgcccgtgccccgcaacgt
gctgatcccccacaccaagggcttcgtgtcctgcgtgtccaacatgcgctccttcgtgcccgccgtgtacgacgtgaccgtggcctt
ccccaagtcctcccccccccccaccatgctgaagctgttcgagggccagtccatcgtgctgcacgtgcacatcaagcgccacgcc
ctgaaggacctgcccgagtccgacgacgccgtggcccagtggtgccgcgacaagttcgtggagaaggacgccctgctggacaa
gcacaacgccgaggacaccttctccggccaggaggtgcaccacatcggccgccccatcaagtccctgctggtggtgatcgcctg
ggtggtggtgatcatcttcggcgccctgaagttcctgcagtggtcctccctgctgtccacctggaagggcaaggccttctccgtgatc
ggcctgggcatcgccaccctgctgatgcacatgctgatcctgtcctcccaggccgagcgctccaaccccgccaaggtggccaag
TGActcgag
SEQ ID NO: 103 CavigLPAAT1
ggtaccATGaccatcgcctccgccgccgtggtgttcctgttcggcatcctgctgttcacctccggcctgatcatcaacctgttccag
gccttctgctccgtgctggtgtggcccctgtccaagaacgcctaccgccgcatcaaccgcgtgttcgccgagttcctgcccctggag
ttcctgtggctgttccactggtgggccggcgccaagctgaagctgttcaccgaccccgagaccttccgcctgatgggcaaggagc
acgccctggtgatcatcaaccacaagatcgagctggactggatggtgggctgggtgctgggccagcacctgggctgcctgggctc
catcctgtccgtggccaagaagtccaccaagttcctgcccgtgttcggctggtccctgtggttctccgagtacctgttcctggagcgc
aactgggccaaggacaagaagaccctgaagtcccacatcgagcgcctgaaggactaccccctgcccttctggctgatcatcttcg
tggagggcacccgcttcacccgcaccaagctgctggccgcccagcagtacgccgcctccgccggcctgcccgtgccccgcaac
gtgctgatcccccacaccaagggcttcgtgtcctccgtgtcccacatgcgctccttcgtgcccgccatctacgacgtgaccgtggcct
tccccaagacctcccccccccccaccatgctgaagctgttcgagggccacttcgtggagctgcacgtgcacatcaagcgccacgc
catgaaggacctgcccgagtccgaggacgccgtggcccagtggtgccgcgacaagttcgtggagaaggacgccctgctggac
aagcacaacgccgaggacaccttctccggccaggaggtgcaccacgtgggccgccccatcaagtccctgctggtggtgatctcc
tgggtggtggtgatcatcttcggcgccctgaagttcctgcagtggtcctccctgctgtcctcctggaagggcatcgccttctccgtgat
cggcctgggcaccgtggccctgctgatgcagatcctgatcctgtcctcccaggccgagcgctccatccccgccaaggagaccccc
gccaacctgaagaccgagctgtcctcctccaagaaggtgaccaacaaggagaacTGActcgag
SEQ ID NO: 104 CavigLPAAT2
ggtaccATGgccatcgccgccgccgccgtgatcgtgcccgtgtccctgctgttcttcgtgtccggcctgatcgtgaacctggtgca
ggccgtgtgcttcgtgctgatccgccccctgttcaagaacacctaccgccgcatcaaccgcgtggtggccgagctgctgtggctgg
agctggtgtggctgatcgactggtgggccggcgtgaagatcaaggtgttcaccgaccacgagaccttccacctgatgggcaagg
agcacgccctggtgatctgcaaccacaagtccgacatcgactggctggtgggctgggtgctggcccagcgctccggctgcctggg
ctccaccctggccgtgatgaagaagtcctccaagttcctgcccgtgatcggctggtccatgtggttctccgagtacctgttcctggag
cgcaactgggccaaggacgagtccaccctgaagtccggcctgaaccgcctgaaggactaccccctgcccttctggctggccctgt
tcgtggagggcacccgcttcacccgcgccaagctgctggccgcccagcagtacgccgcctcctccggcctgcccgtgccccgca
acgtgctgatcccccgcaccaagggcttcgtgtcctccgtgtcccacatgcgctccttcgtgcccgccatctacgacgtgaccgtgg
ccatccccaagacctcccccccccccaccctgctgcgcatgttcaagggccagtcctccgtgctgcacgtgcacctgaagcgcca
ccagatgaacgacctgcccgagtccgacgacgccgtggcccagtggtgccgcgacatcttcgtggagaaggacgccctgctgg
acaagcacaacgccgaggacaccttctccggccaggagctgcaggacaccggccgccccatcaagtccctgctgatcgtgatct
cctgggccgtgctggtggtgttcggcgccgtgaagttcctgcagtggtcctccctgctgtcctcctggaagggcctggccttctccgg
catcggcctgggcgtgatcaccctgctgatgcacatcctgatcctgttctcccagtccgagcgctccacccccgccaaggtggccc
ccgccaagcccaagatcgagggcgagtcctccaagaccgagatggagaaggagcacTGActcgag
SEQ ID NO: 105 CpalLPAAT1
ggtaccATGgccatcgccgccgccgccgtgatcgtgcccctgggcctgctgttcttcgtgtccggcctgatcgtgaacctggtgca
ggccgtgtgcttcgtgctgatccgccccctgtccaagaacacctaccgccgcatcaaccgcgtggtggccgagctgctgtggctgg
agctggtgtggctgatcgactggtgggccggcgtgaagatcaaggtgttcaccgaccacgagaccctgtccctgatgggcaagg
agcacgccctggtgatctgcaaccacaagtccgacatcgactggctggtgggctgggtgctggcccagcgctccggctgcctggg
ctccaccctggccgtgatgaagaagtcctccaagttcctgcccgtgatcggctggtccatgtggttctccgagtacctgcccgagtcc
gacgacgccgtggcccagtggtgccgcgacatcttcgtggagaaggacgccctgctggacaagcacaacgccgaggacacctt
ctccggccaggagctgcaggacaccggccgccccatcaagtccctgctggtggtgatctcctgggccgtgctggtgatcttcggcg
ccgtgaagttcctgcagtggtcctccctgctgtcctcctggaagggcctggccttctccggcgtgggcctgggcatcatcaccctgct
gatgcacatcctgatcctgttctcccagtccgagcgctccacccccgccaaggtggcccccgccaagcccaagaaggacggcga
gtcctccaagaccgagatcgagaaggagaacgttcctggagcgctcctgggccaaggacgagaacaccctgaagtccggcct
gaaccgcctgaaggactaccccctgcccttctggctggccctgttcgtggagggcacccgcttcacccgcgccaagctgctggcc
gcccagcagtacgccacctcctccggcctgcccgtgccccgcaacgtgctgatcccccgcaccaagggcttcgtgtcctccgtgtc
ccacatgcgctccttcgtgcccgccatctacgacgtgaccgtggccatccccaagacctcccccccccccaccatgctgcgcatgtt
caagggccagtcctccgtgctgcacgtgcacctgaagcgccacctgatgaaggacctTGActcgag
SEQ ID NO: 106 CuPSR23 LPAAT2
ggtaccATGgccatcgccgccgccgccgtgatcttcctgttcggcctgatcttcttcgcctccggcctgatcatcaacctgttccag
gccctgtgcttcgtgctgatccgccccctgtccaagaacgcctaccgccgcatcaaccgcgtgttcgccgagctgctgctgtccgag
ctgctgtgcctgttcgactggtgggccggcgccaagctgaagctgttcaccgaccccgagaccttccgcctgatgggcaaggagc
acgccctggtgatcatcaaccacatgaccgagctggactggatggtgggctgggtgatgggccagcacttcggctgcctgggctc
catcatctccgtggccaagaagtccaccaagttcctgcccgtgctgggctggtccatgtggttctccgagtacctgtacctggagcg
ctcctgggccaaggacaagtccaccctgaagtcccacatcgagcgcctgatcgactaccccctgcccttctggctggtgatcttcgt
ggagggcacccgcttcacccgcaccaagctgctggccgcccagcagtacgccgtgtcctccggcctgcccgtgccccgcaacgt
gctgatcccccgcaccaagggcttcgtgtcctgcgtgtcccacatgcgctccttcgtgcccgccgtgtacgacgtgaccgtggccttc
cccaagacctcccccccccccaccctgctgaacctgttcgagggccagtccatcatgctgcacgtgcacatcaagcgccacgcca
tgaaggacctgcccgagtccgacgacgccgtggccgagtggtgccgcgacaagttcgtggagaaggacgccctgctggacaa
gcacaacgccgaggacaccttctccggccaggaggtgtgccactccggctcccgccagctgaagtccctgctggtggtgatctcc
tgggtggtggtgaccaccttcggcgccctgaagttcctgcagtggtcctcctggaagggcaaggccttctccgccatcggcctggg
catcgtgaccctgctgatgcacgtgctgatcctgtcctcccaggccgagcgctccaaccccgccgaggtggcccaggccaagctg
aagaccggcctgtccatctccaagaaggtgaccgacaaggagaacTGActcgag
SEQ ID NO: 107 CkoeLPAAT1
ggtaccATGgccatccccgccgccgtggccgtgatccccatcggcctgctgttcatcatctccggcctgatcgtgaacctgatcca
ggccgtggtgtacgtgctgatccgccccctgtccaagaacctgcaccgcaagatcaacaagcccatcgccgagctgctgtggctg
gagctgatctggctggtggactggtgggccggcatcaaggtggaggtgtacgccgactcccagaccctggagctgatgggcaag
gagcacgccctgctgatctgcaaccaccgctccgacatcgactggctggtgggctgggtgctggcccagcgcgcccgctgcctgg
gctccgccctggccatcatgaagaagtccgccaagttcctgcccgtgatcggctggtccatgtggttctccgactacatcttcctgga
ccgcacctgggccaaggacgagaagaccctgaagtccggcttcgagcgcctggccgacttccccatgcccttctggctggccctg
ttcgtggagggcacccgcttcaccaaggccaagctgctggccgcccaggagtacgccgcctcccgcggcctgcccgtgccccag
aacgtgctgatcccccgcaccaagggcttcgtgaccgccgtgacccacatgcgctcctacgtgcccgccatctacgactgcaccg
tggacatctccaaggcccaccccgccccctccatcctgcgcctgatccgcggccagtcctccgtggtgaaggtgcagatcacccg
ccactccatgcaggagctgcccgagaccgccgacggcatctcccagtggtgcatggacctgttcgtgaccaaggacggcttcctg
gagaagtaccactccaaggacatcttcggctccctgcccgtgcagaacatcggccgccccgtgaagtccctgatcgtggtgctgtg
ctggtactgcctgatggccttcggcctgttcaagttcttcatgtggtcctccctgctgtcctcctgggagggcatcctgtccctgggcctg
atcctgctggccgtggccatcgtgatgcagatcctgatccagtccaccgagtccgagcgctccacccccgtgaagtccatccaga
aggacccctccaaggagaccctgctgcagaacTGActcgag
SEQ ID NO: 108 CkoeLP AAT2
ggtaccATGcacgtgctgctggagatggtgaccttccgcttctcctccttcttcgtgttcgacaacgtgcaggccctgtgcttcgtgct
gatctggcccctgtccaagtccgcctaccgcaagatcaaccgcgtgttcgccgagctgctgctgtccgagctgctgtgcctgttcga
ctggtgggccggcgccaagctgaagctgttcaccgaccccgagaccttccgcctgatgggcaaggagcacgccctggtgatcac
caaccacaagatcgacctggactggatgatcggctggatcctgggccagcacttcggctgcctgggctccgtgatctccatcgcca
agaagtccaccaagttcctgcccatcttcggctggtccctgtggttctccgagtacctgttcctggagcgcaactgggccaaggaca
agcgcaccctgaagtcccacatcgagcgcatgaaggactaccccctgcccctgtggctgatcctgttcgtggagggcacccgctt
cacccgcaccaagctgctggccgcccagcagtacgccgcctcctccggcctgcccgtgccccgcaacgtgctgatcccccacac
caagggcttcgtgtcctccgtgtcccacatgcgctccttcgtgcccgccgtgtacgacgtgaccgtggccttccccaagacctcccc
cccccccaccatgctgtccctgttcgagggccagtccgtggtgctgcacgtgcacatcaagcgccacgccatgaaggacctgccc
gactccgacgacgccgtggcccagtggtgccgcgacaagttcgtggagaaggacgccctgctggacaagcacaacgccgagg
acaccttctccggccaggaggtgcaccacgtgggccgccccatcaagtccctgctggtggtgatctcctggatggtggtgatcatct
tcggcgccctgaagttcctgcagtggtcctccctgctgtcctcctggaagggcaaggccttctccgccatcggcctgggcatcgcca
ccctgctgatgcacgtgctggtggtgttctcccaggccgaccgctccaaccccgccaaggtgccccccgccaagctgaacaccga
gctgtcctcctccaagaaggtgaccaacaaggagaacTGActcgag
SEQ ID NO: 109 CprocLPAAT2
ggtaccATGgccatccccgccgccgtggccgtgatccccatcggcctgctgttcatcatctccggcctgatcgtgaacctgatcca
ggccgtggtgtacgtgctgatccgccccctgtccaagaacctgtaccgcaagatcaacaagcccatcgccgagctgctgtggctg
gagctgatctggctggtggactggtgggccggcatcaaggtggaggtgtacgccgactccgagaccctggagtccatgggcaag
gagcacgccctgctgatctgcaaccaccgctccgacatcgactggctggtgggctgggtgctggcccagcgcgcccgctgcctgg
gctccgccctggccatcatgaagaagtccgccaagttcctgcccgtgatcggctggtccatgtggttctccgactacatcttcctgga
ccgcacctgggagaaggacgagaagaccctgaagtccggcttcgagcgcctggccgacttccccatgcccttctggctggccct
gttcgtggagggcacccgcttcaccaaggccaagctgctggccgcccaggagttcgccgcctcccgcggcctgcccgtgcccca
gaacgtgctgatcccccgcaccaagggcttcgtgaccgccgtgacccacatgcgctcctacgtgcccgccatctacgactgcacc
gtggacatctccaaggcccaccccgccccctccatcctgcgcctgatccgcggccagtcctccgtggtgaaggtgcagatcaccc
gccactccatgcaggagctgcccgagacccccgacggcatctcccagtggtgcatggacctgttcgtgaccaaggacgccttcct
ggagaagtaccactccaaggacatcttcggctccctgcccgtgcacgacatcggccgccccgtgaagtccctgatcgtggtgctgt
gctggtactccctgatggccttcggcttctacaagttcttcatgtggtcctccctgctgtcctcctgggagggcatcctgtccctgggcct
ggtgctgatcgtgatcgccatcgtgatgcagatcctgatccagtcctccgagtccgagcgctccacccccgtgaagtccgtgcaga
aggacccctccaaggagaccctgctgcagaacTGActcgag
SEQ ID NO: 110 CavigGPAT9
ggtaccATGgccaccggcggctccctgaagccctcctcctccgacctggacctggaccaccccaacatcgaggactacctgcc
ctccggctcctccatcaacgagcccgccggcaagctgcgcctgcgcgacctgctggacatctcccccaccctgaccgaggccgc
cggcgccatcgtggacgactccttcacccgctgcttcaagtccatcccccgcgagccctggaactggaacctgtacctgttccccct
gtggtgcatcggcgtgctgatccgctacttcatcctgttccccggccgcgtgatcgtgctgaccatgggctggatcaccgtgatctcct
ccttcatcgccgtgcgcgtgctgctgaagggccacgacgccctgcagatcaagctggagcgcctgatcgtgcagctgctgtgctcc
tccttcgtggcctcctggaccggcgtggtgaagtaccacggcccccgcccctccatccgccccaagcaggtgtacgtggccaacc
acacctccatgatcgacttcttcatcctggaccagatgaccgtgttctccgtgatcatgcagaagcaccccggctgggtgggcctgc
tgcagtccaccctgctggagtccgtgggctgcatctggttcgaccgcgccgaggccaaggaccgcggcatcgtggccaagaagc
tgtgggaccacgtgcacggcgagggcaacaaccccctgctgatcttccccgagggcacctgcgtgaacaacaactactccgtga
tgttcaagaagggcgccttcgagctgggctgcaccgtgtgccccgtggccatcaagtacaacaagatcttcgtggacgccttctgg
aactccaagaagcagtccttcacccgccacctgctgcagctgatgacctcctgggccgtggtgtgcgacgtgtggtacttggagcc
ccagaccctgaagcccggcgagacccccatcgagttcgccgagcgcgtgcgcgacatcatctccgcccgcgccggcctgaaga
aggtgccctgggacggctacctgaagtactcccgcccctcccccaagcaccgcgagcgcaagcagcagaccttcgccgagtcc
gtgctgcagcgcctggaggagTGActcgag
SEQ ID NO: 111 ChookGPAT9-1
ggtaccATGgccaccgccggctccctgaagccctcccgctccgagctggacttcgaccgccccaacatcgaggactacctgcc
ctccggctcctccatcatcgagcccgccggcaagctgcgcctgcgcgacctgctggacatctcccccaccctgaccgaggccgcc
ggcgccatcgtggacgactccttcacccgctgcttcaagtccaacccccccgagccctggaactggaacatctacctgttccccct
gtggtgcttcggcgtgctgatccgctacctgatcctgttccccgcccgcgtgatcgtgctgaccatcggctggatcatcttcctgtcctc
cttcatccccgtgcacctgctgctgaagggccacgacgccctgcgcatcaagctggagcgcctgctggtggagctgatctgctcctt
cttcgtggcctcctggaccggcgtggtgaagtaccacggcccccgcccctccatccgccccaagcaggtgtacgtggccaaccac
acctccatgatcgacttcttcatcctggaccagatgaccgtgttctccgtgatcatgcagaagcaccccggctgggtgggcctgctg
cagtccaccctgctggagtccgtgggctgcatctggttcgaccgcgccgaggccaaggaccgcggcatcgtggccaagaagctg
tgggaccacgtgcacggcgagggcaacaaccccctgctgatcttccccgagggcacctgcgtgaacaacaactactccgtgatg
ttcaagaagggcgccttcgagctgggctgcaccgtgtgccccgtggccatcaagtacaacaagatcttcgtggacgccttctggaa
ctccaagaagcagtccttcacccgccacctgctgcagctgatgacctcctgggccgtggtgtgcgacgtgtggtacttggagcccc
agaccctgaagcccggcgagacccccatcgagttcgccgagcgcgtgcgcgacatcatctccgtgcgcgccggcctgaagaag
gtgccctgggacggctacctgaagtactcccgcccctcccccaagcacaccgagcgcaagcagcagaacttcgccgagtccgt
gctgcagcgcctggagaagaagTGActcgag
SEQ ID NO: 112 CignGPAT9-1
ggtaccATGgccaccggcggccgcctgaagccctcctcctccgagctggacctggaccgcgccaacaccgaggactacctgc
cctccggctcctccatcaacgagcccgtgggcaagctgcgcctgcgcgacctgctggacatctcccccaccctgaccgaggccg
ccggcgccatcgtggacgactccttcacccgctgcttcaagtccatcccccccgagccctggaactggaacatctacctgttccccc
tgtggtgcttcggcgtgctgatccgctacttcatcctgttccccgcccgcgtgatcgtgctgaccatcggctggatcaccgtgatctcct
ccttcaccgccgtgcgcttcctgctgaagggccacaacgccctgcagatcaagctggagcgcctgatcgtgcagctgctgtgctcc
tccttcgtggcctcctggaccggcgtggtgaagtaccacggcccccgcccctccatccgccccaagcaggtgtacgtggccaacc
acacctccatgatcgacttcctgatcctggaccagatgaccgtgttctccgtgatcatgcagaagcaccccggctgggtgggcctg
ctgcagtccaccctgctggagtccgtgggctgcatctggttcaaccgcgccgaggccaaggaccgcgagatcgtggccaagaag
ctgtgggaccacgtgcacggcgagggcaacaaccccctgctgatcttccccgagggcacctgcgtgaacaaccactactccgtg
atgttcaagaagggcgccttcgagctgggctgcaccgtgtgccccgtggccatcaagtacaacaagatcttcgtggacgccttctg
gaactcccgcaagcagtccttcaccatgcacctgctgcagctgatgacctcctgggccgtggtgtgcgacgtgtggtacttggagc
cccagaccctgaagcccggcgagaccgccatcgagttcgccgagcgcgtgcgcgacatcatctccgtgcgcgccggcctgaag
aaggtgccctgggacggctacctgaagtactcccgcccctcccccaagcaccgcgagtccaagcagcagtccttcgccgagtcc
gtgctgcgccgcctggaggagaagTGActcgag
SEQ ID NO: 113 CignGPAT9-2
ggtaccATGgccaccggcggccgcctgaagccctcctcctccgagctggacctggaccgcgccaacaccgaggactacctgc
cctccggctcctccatcaacgagcccgtgggcaagctgcgcctgcgcgacctgctggacatctcccccaccctgaccgaggccg
ccggcgccatcgtggacgactccttcacccgctgcttcaagtccatcccccccgagccctggaactggaacatctacctgttccccc
tgtggtgcttcggcgtgctgatccgctacttcatcctgttccccgcccgcgtgatcgtgctgaccatcggctggatcaccgtgatctcct
ccttcaccgccgtgcgcttcctgctgaagggccacaacgccctgcagatcaagctggagcgcctgatcgtgcagctgctgtgctcc
tccttcgtggcctcctggaccggcgtggtgaagtaccacggcccccgcccctccatccgccccaagcaggtgtacgtggccaacc
acacctccatgatcgacttcctgatcctggaccagatgaccgtgttctccgtgatcatgcagaagcaccccggctgggtgggcctg
ctgcagtccaccctgctggagtccgtgggctgcatctggttcaaccgcgccgaggccaaggaccgcgagatcgtggccaagaag
ctgtgggaccacgtgcacggcgagggcaacaaccccctgctgatcttccccgagggcacctgcgtgaacaaccactactccgtg
atgttcaagaagggcgccttcgagctgggctgcaccgtgtgccccgtggccatcaagtacaacaagatcttcgtggacgccttctg
gaactccaagaagcactccttcacccgccacctgctgcagctgatgacctcctgggccgtggtgtgcgacgtgtggtacttggagc
cccagaccctgaagcccggcgagacccccatcgagttcgccgagcgcgtgcgcgacatcatctccgtgcgcgccgacctgaag
aaggtgccctgggacggctacctgaagtactcccgcccctcccccaagcaccgcgagcgcaagcagcagaagttcgccgagtc
cgtgctgcgccgcctggaggagaagTGActcgag
SEQ ID NO: 114 CpalGPAT9-1
ggtaccATGgccaccgccggccgcctgaagccctcctcctccgagctggagctggacctggaccgccccaacatcgaggact
acctgccctccggctcctccatcaacgagcccgccggcaagctgcgcctgcgcgacctgctggacatctcccccatgctgaccga
ggccgccggcgccatcgtggacgactccttcacccgctgcttcaagtccatcccccccgagccctggaactggaacatctacctgt
tccccctgtggtgcttcggcgtgctgatccgctacctgatcctgttccccgcccgcgtgatcgtgctgaccgtgggctggatcaccgtg
atctcctccttcatcaccgtgcgcttcctgctgaagggccacgactccctgcgcatcaagctggagcgcctgatcgtgcagctgttct
gctcctccttcgtggcctcctggaccggcgtggtgaagtaccacggcccccgcccctccatccgcccccagcaggtgtacgtggcc
aaccacacctccatgatcgacttcatcatcctgaaccagatgaccgtgttctccgccatcatgcagaagcaccccggctgggtggg
cctgatccagtccaccatcctggagtccgtgggctgcatctggttcaaccgcgccgaggccaaggaccgcgagatcgtggccaa
gaagctgctggaccacgtgcacggcgagggcaacaaccccctgctgatcttccccgagggcacctgcgtgaacaaccactactc
cgtgatgttcaagaagggcgccttcgagctgggctgcaccgtgtgccccgtggccatcaagtacaacaagatcttcgtggacgcct
tctggaactccaagaagcagtccttcaccatgcacctgctgcagctgatgacctcctgggccgtggtgtgcgacgtgtggtacttgg
agccccagaccctgaagcccggcgagacccccatcgagttcgccgagcgcgtgcgcgacatcatctccgtgcgcgccggcctg
aagaaggtgccctgggacggctacctgaagtactcccgcccctcccccaagcaccgcgagcgcaagcagcagtccttcgccga
gtccgtgctgcgccgcctggagaagcgcTGActcgag
SEQ ID NO: 115 CpalGPATt9-2
ggtaccATGgccaccgccggccgcctgaagccctcctcctccgagctggagctggacctggaccgccccaacatcgaggact
acctgccctccggctcctccatcaacgagcccgccggcaagctgcgcctgcgcgacctgctggacatctcccccatgctgaccga
ggccgccggcgccatcgtggacgactccttcacccgctgcttcaagtccatcccccccgagccctggaactggaacatctacctgt
tccccctgtggtgcttcggcgtgctgatccgctacctgatcctgttccccgcccgcgtgatcgtgctgaccgtgggctggatcaccgtg
atctcctccttcatcaccgtgcgcttcctgctgaagggccacgactccctgcgcatcaagctggagcgcctgatcgtgcagctgttct
gctcctccttcgtggcctcctggaccggcgtggtgaagtaccacggcccccgcccctccatccgcccccagcaggtgtacgtggcc
aaccacacctccatgatcgacttcatcatcctgaaccagatgaccgtgttctccgccatcatgcagaagcaccccggctgggtggg
cctgatccagtccaccatcctggagtccgtgggctgcatctggttcaaccgcgccgaggccaaggaccgcgagatcgtggccaa
gaagctgctggaccacgtgcacggcgagggcaacaaccccctgctgatcttccccgagggcacctgcgtgaacaaccactactc
cgtgatgttcaagaagggcgccttcgagctgggctgcaccgtgtgccccgtggccatcaagtacaacaagatcttcgtggacgcct
tctggaactccaagaagctgtccttcaccatgcacctgctgcagctgatgacctcctgggccgtggtgtgcgacgtgtggtacttgg
agccccagaccctgaagcccggcgagacccccatcgagttcgccgagcgcgtgcgcgacatcatctccgtgcgcgccggcctg
aagaaggtgccctgggacggctacctgaagtactcccgcccctcccccaagcaccgcgagcgcaagcagcagaccttcgccg
agtccgtgctgcgccgcctggaggagaagggcaacgtggtgcccaccgtgaacTGActcgag
SEQ ID NO: 116 CavigDGAT1
ggtaccATGgccatcgccgacggcggcatcatcggcgccgccggctccatctccgccctgaccgccgacaccgaccccccct
ccctgcgccgccgcaacgtgcccgccggccaggcctccgccgtgtccgccttctccaccgagtccatggccaagcacctgtgcga
cccctcccgcgagccctccccctcccccaagtcctccgacgacggcaaggaccccgacatcggctccgtggactccctgaacga
gaagccctcctcccccgccgccggcaagggccgcctgcagcacgacctgcgcttcacctaccgcgcctcctcccccgcccaccg
caaggtgaaggagtcccccctgtcctcctccaacatcttcaagcagtcccacgccggcctgttcaacctgtgcgtggtggtgctggt
ggccgtgaactcccgcctgatcatcgagaacctgatgaagtacggcctgctgatcaagaccggcttctggttctcctcccgctccct
gcgcgactggcccctgttcatgtgctgcctgtccctgcccatcttccccctggccgccttcctggtggagaagctggcccagaagaa
ccgcctgcaggagcccaccgtggtgtgctgccacgtgctgatcacctccgtgtccatcctgtaccccgtgctggtgatcctgcgctg
cgactccgccgtgctgtccggcgtggccctgatgctgttcgcctgcatcgtgtggctgaagctggtgtcctacgcccactccaactac
gacatgcgctacgtggccaagtccctggacaagggcgagcccgtggtggactccgtgatcgccgaccacccctaccgcgtgga
ctacaaggacctggtgtacttcatggtggcccccaccctgtgctaccagctgtcctaccccctgaccccctgcgtgcgcaagtcctg
gatcgcccgccaggtgatgaagctggtgctgttcaccggcgtgatgggcttcatcgtggagcagtacatcaaccccatcgtgcag
aactccaagcaccccctgaagggcgacctgctgtacgccatcgagcgcgtgctgaagctgtccgtgcccaacctgtacgtgtggc
tgtgcatgttctactgcttcttccacctgtggctgaacatcctggccgagctgatctgcttcggcgaccgcgagttctacaaggactgg
tggaacgccaagaccgtggaggagtactggcgcatgtggaacatgcccgtgcacaagtggatggtgcgccacatctacttcccct
gcctgcgcaacggcatcccccgcggcgtggccgtgctgatcgccttcctggtgtccgccgtgttccacgagctgtgcatcgccgtgc
cctgccacgtgttcaagctgtgggccttcatcggcatcatgttccaggtgcccctggtgctggtgtccaactgcctgcagaagaagtt
ccagtcctccatggccggcaacatgttcttctggttcatcttctgcatcttcggccagcccatgtgcgtgctgctgtactaccacgacct
gatgaaccgcaagggctcccgcatcgacTGActcgag
SEQ ID NO: 117 ChookDGAT1-1
ggtaccATGgccatcgccgacggcggctccgccggcgccgccggctccatctccggctccgacccctccccctccaccgcccc
ctccctgcgccgccgcaacgcctccgccggccaggccttctccaccgagtccatggcccgcgacctgtgcgacccctcccgcga
gccctccctgtcccccaagtcctccgacgacggcaaggaccccgccgacgacatcggcgccgccgactccgtggactccggcg
gcgtgaaggacgagaagccctcctcccaggccgccgccaaggcccgcctggagcacgacctgcgcttcacctaccgcgcctcc
tcccccgcccaccgcaaggtgaaggagtcccccctgtcctcctccaacatcttcaagcagtcccacgccggcctgttcaacctgtg
cgtggtggtgctggtggccgtgaactcccgcctgatcatcgagaacctgatgaagtacggcctgctgatcaagaccggcttctggtt
ctcctcccgctccctgcgcgactggcccctgttcatgtgctgcctgtccctgcccatcttccccctggccgccttcctggtggagaagc
tggcccagaagaaccgcctgcaggagcccaccgtggtgtgctgccacgtgatcatcacctccgtgtccatcctgtaccccgtgctg
gtgatcctgcgctgcgactccgccgtgctgtccggcgtggccctgatgctgttcgcctgcatcgtgtggctgaagctggtgtcctacg
cccacgccaactacgacatgcgctccgtggccaagtccctggacaagggcgagaccgtggccgactccgtgatcgtggaccac
ccctaccgcgtggactacaaggacctggtgtacttcatggtggcccccaccctgtgctaccagctgtcctaccccctgaccccctac
gtgcgcaagtcctgggtggcccgccaggtgatgaagctggtgctgttcaccggcgtgatgggcttcatcgtggagcagtacatcaa
ccccatcgtgcagaactccaagcaccccctgaagggcgacctgctgtacgccatcgagcgcgtgctgaagctgtccgtgcccaa
cctgtacgtgtggctgtgcatgttctactgcttcttccacctgtggctgaacatcctggccgagctgacctgcttcggcgaccgcgagt
tctacaaggactggtggaacgccaagaccgtggaggagtactggcgcatgtggaacatgcccgtgcacaagtggatggtgcgc
cacatctacttcccctgcctgcgcaacggcatcccccgcggcgtggccgtgctgatcgccttcctggtgtccgccgtgttccacgag
ctgtgcatcgccgtgccctgccacgtgttcaagctgtgggccttcatcggcatcatgttccaggtgcccctggtgctggtgtccaactg
cctgcagaagaagttccagtcctccatggccggcaacatgttcttctggttcatcttctgcatcttcggccagcccatgtgcgtgctgct
gtactaccacgacctgatgaaccgcaagggctcccgcatcgacTGActcgag
SEQ ID NO: 118 CavigLPCAT
ggtaccATGggcctggtgtccgtggccgccgccatcggcgtgtccgtgcccgtggcccgcttcctgctgtgcttcctggccaccat
ccccgtgtccttcctgtggcgcctggtgcccggccgcctgcccaagcacctgtactccgccgcctccggcgccatcctgtcctacct
gtccttcggcgcctcctccaacctgcacttcatcgtgcccatgaccctgggctacctgtccatgctgttcttccgccccttctccggcct
gctgaccttcttcctgggcttcggctacctgatcggctgccacgtgtactacatgtccggcgacgcctggaaggagggcggcatcg
acgccaccggcgccctgatggtgctgaccctgaaggtgatctcctgctccatgaactacaacgacggcctgctgaaggaggagg
gcctgcgcgagtcccagaagaagaaccgcctgaccaagatgccctccctgatcgagtacttcggctactgcctgtgctgcggctc
ccacttcgccggccccgtgtacgagatgaaggactacctggagtggaccgagggcaagggcatctggtcccgctcccagaagg
agcccaagccctcccccttcggcggcgccctgcgcgccatcatccaggccgccgtgtgcatggccatgtacctgtacctggtgccc
caccaccccctgacccgcttcaccgagcccgtgtactacgagtggggcttcttccgccgcctgtcctaccagtacatggccgccctg
accgcccgctggaagtactacttcatctggtccatctccgaggcctccctgatcatctccggcctgggcttctccggctggaccgagt
cctccccccccaagccccgctgggaccgcgccaagaacgtggacatcatcggcgtggagttcgccaagtcctccgtgcagctgc
ccctggtgtggaacatccaggtgtccatctggctgcgccactacgtgtacgaccgcctggtgcagaacggcaagcgccccggctt
cttccagctgctggccacccagaccgtgtccgccgtgtggcacggcctgtaccccggctacatcatcttcttcgtgcagtccgccctg
atgatcgccggctcccgcgtgatctaccgctggcagcaggccgtgccccccaagatgggcctggtgaagaacatcttcgtgttctt
caacttcgcctacaccctgctggtgctgaactactccgccgtgggcttcatggtgctgtccatgcacgagaccctggcctcctacgg
ctccgtgtactacatcggcaccatcctgcccatcaccctgatcctgctgtcctacgtgatcaagcccggcaagcccgcccgctccaa
ggcccacaaggagcagTGActcgag
SEQ ID NO: 119 CpalLPCAT
ggtaccATGgagctgggctccgtggccgccgccatcggcgtgtccgtgcccgtggcccgcttcctgctgtgcttcctggccaccat
ccccgtgtccttcctgtggcgcctggtgcccggccgcctgcccaagcacctgtactccgccgcctccggcgccatcctgtcctacct
gtccttcggcccctcctccaacctgcacttcatcgtgcccatgaccctgggctacctgtccatgctgttcttccgccccttctccggcct
gctgaccttcttcctgggcttcggctacctgatcggctgccacgtgtactacatgtccggcgacgcctggaaggagggcggcatcg
acgccaccggcgccctgatggtgctgaccctgaaggtgatctcctgctccatcaactacaacgacggcctgctgaaggaggagg
gcctgcgcgagtcccagaagaagaaccgcctgaccaagatgccctccctgatcgagtacatcggctactgcctgtgctgcggctc
ccacttcgccggccccgtgtacgagatgaaggactacctggagtggaccgagggcaagggcgtgtggtcccactccgagaagg
agcccaagccctcccccttcggcggcgccctgcgcgccatcatccaggccgccgtgtgcatggccatgtacatgtacctggtgccc
caccaccccctgtcccgcttcaccgagcccgtgtactacgagtggggcttcttccgccgcctgtcctaccagtacatggccggcctg
accgcccgctggaagtactacttcatctggtccatctccgaggcctccctgatcatctccggcctgggcttctccggctggaccgagt
cctccccccccaagccccgctgggaccgcgccaagaacgtggacatcatcggcgtggagttcgccaagtcctccgtgcagctgc
ccctggtgtggaacatccaggtgtccacctggctgcgccactacgtgtacgaccgcctggtgcagaacggcaagcgccccggctt
cttccagctgctggccacccagaccgtgtccgccatctggcacggcctgtaccccggctacatcatcttcttcgtgcagtccgccctg
atgatcgccggctcccgcgtgatctaccgctggcagcaggccgtgccccccaagatgggcctggtgaagaacatcttcgtgttctt
caacttcgcctacaccctgctggtgctgaactactccgccgtgggcttcatggtgctgtccatgcacgagaccctggcctcctacgg
ctccgtgtactacatcggcaccatcctgcccatcaccctgatcctgctgtcctacgtgatcaagcccggcaagcccgcccgctccaa
ggcccacaaggagcagTGActcgag
SEQ ID NO: 120 CpauLPCAT
ggtaccATGgagctggagatcggctccgtggccgccgccatcggcgtgtccgtgcccgtggcccgcttcctgctgtgcttcctgg
ccaccatccccgtgtccttcctgtgccgcctgctgcccgcccgcctgcccaagcacctgtactccgccgcctccggcgccatcctgt
cctacctgtccttcggcccctcctccaacctgcacttcatcgtgcccatgtccctgggctacctgtccatgctgttcttccgccccttctcc
ggcctgctgaccttcttcctgggcttcggctacctgatcggctgccacgtgtactacatgtccggcgacgcctggaaggagggcgg
catcgacgccaccggcgccctgatggtgctgaccctgaaggtgatctcctgctccatcaactacaacgacggcctgctgaaggag
gagggcctgcgcgagtcccagaagaagaaccgcctgaccaagatgccctccctgatcgagtacttcggctactgcctgtgctgcg
gctcccacttcgccggccccgtgtacgagatgaaggactacctggagtggaccgagggcaagggcatctggtcccgctccgaga
aggaccccaagccctcccccttcggcggcgccctgcgcgccatcatccaggccgccgtgtgcatggccatgcacatgtacctggt
gccccaccaccccctgacccgcttcaccgagcccgtgtactacgagtggggcttcttccgccgcctgtcctaccagtacatggccg
cccagaccgcccgctggaagtactacttcatctggtccatctccgaggcctccctgatcatctccggcctgggcttctccggctggac
cgagtcctccccccccaagccccgctgggacaaggccaagaacgtggacatcatcggcgtggagttcgccaagtcctccgtgca
gctgcccctggtgtggaacatccaggtgtccacctggctgcgccactacgtgtacgaccgcctggtgcagaacggcaagcgccc
cggcttcttccagctgctggccacccagaccgtgtccgccgtgtggcacggcctgtaccccggctacatcatcttcttcgtgcagtcc
gccctgatgatcgccggctcccgcgtgatctaccgctggcagcaggccgtgccccagaagatgggcctggtgaagaacatcttcg
tgttcttcaacttcgcctacaccctgctggtgctgaactactccgccgtgggcttcatggtgctgtccatgcacgagaccctggcctcc
tacggctccgtgtactacatcggcaccatcctgcccatcaccctgatcctgctgtcctacgtgatcaagcccggcaagcccacccg
ctccaaggtgcacaaggagcagTGActcgag
SEQ ID NO: 121 CschuLPCAT
ggtaccATGgagctggagatggagcccctggccgccgccatcggcgtgtccgtggccgtgttccgcttcctggtgtgcttcatcg
ccaccatccccgtgtccttcatctgccgcctggtgcccggcggcctgccccgccacctgttctccgccgcctccggcgccgtgctgtc
ctacctgtccttcggcttctcctccaacctgcacttcctggtgcccatgaccctgggctacctgtccatgatcctgttccgccgcttctgc
ggcatcctgaccttcttcctgggcttcggctacctgatcggctgccacgtgtactacatgtccggcgacgcctggaaggagggcgg
catcgacgccaccggcgccctgatggtgctgaccctgaaggtgatctcctgctccatcaactacaacgacggcctgctgaaggag
gagggcctgcgcgagtcccagaagaagaaccgcctgatccgcctgccctccctgatcgagtacttcggctactgcctgtgctgcg
gctcccacttcgccggccccgtgtacgagatgaaggactacctggactggaccgagggcaagggcatctggtcccactccgaga
agggccccaagccctcccccctgcgcgccgccctgcgcgccatcatccaggccggcttctgcatggccatgtacctgtacctggtg
ccccactaccccctgacccgcttcaccgaccccgtgtactacgagtggggcatcctgcgccgcctgtcctaccagtacatggcctc
cttcaccgcccgctggaagtactacttcatctggtccatctccgaggcctccctgatcatctccggcctgggcttctccggctggacc
gagtcctccccccccaagccccgctgggaccgcgccaagaacgtggacatcctgggcgtggagctggccaagtcctccgtgca
gatccccctggtgtggaacatccaggtgtccacctggctgcgccactacgtgtacgaccgcctggtgcagaacggcaagcgccc
cggcttcctgcagctgctggccacccagaccgtgtccgccatctggcacggcgtgtaccccggctacctgatcttcttcgtgcagtcc
gccctgatgatcgccggctcccgcgccatctaccgctggcagcaggccgtgccccccaagatgtccctggtgaagaacaccctg
gtgttcttcaacttcgcctacaccctgctggtgctgaactactccgccgtgggcttcatggtgctgtccatgcacgagaccctggcctc
ctacggctccgtgtactacgtgggcaccatcctgcccgtgaccctgatcctgctgggctacgtgatcaagcccggcaagtcccccc
gctccaaggcctccaaggagcagTGActcgag
SEQ ID NO: 122 CavigPLA2-1
ggtaccATGaacttcgacttcctgtccaacatcccctggttcggcgccaaggcctccgacaacgccggctcctccttcggctccg
ccaccatcgtgatccagcagcccccccccgtgtcccgcggcttcgacatccgccactggggctggccctggtccgtgctgtccgtg
ctgccctggggcaagcccggctgcgacgagctgcgcgccccccccaccaccatcaaccgccgcctgaagcgcaacgccacct
ccatgcactcctccgccgtgcgcggcaacgccgaggccgcccgcgtgcgcttccgcccctacgtgtccaaggtgccctggcaca
ccggcttccgcggcctgctgtcccagctgttcccccgctacggccactactgcggccccaactggtcctccggcaagaacggcgg
ctcccccgtgtgggaccagcgccccatcgactggctggactactgctgctactgccacgacatcggctacgacacccacgacca
ggccaagctgctggaggccgacctggccttcctggagtgcctggagcgcccctcctaccccaccaagggcgacgcccacgtgg
cccacatgtacaagaccatgtgcgtgaccggcctgcgcaacgtgctgatcccctaccgcacccagctgctgcgcctgaactcccg
ccagcccctgatcgacttcggctggctgtccaacgccgcctggaagggctggaacgcccagaagtccTGActcgag
SEQ ID NO: 123 CiPLA2-1
ggtaccATGaacctggacttcctgtccaagatcccctggttcgaggccaaggcctccgagaaccccggcctgaacctgggctcc
accaccatcgtgatcaagcagccccgccagggcttcgacatccgccactggggctggccctggtccgtgctgacctggggcaac
cgcgtgaccgacgaggtgcacgccccccccaccaccatcaaccgccgcctgaagcgcaacgccaccggccccgccgtgcag
ggcgacaccgaggccgcccgcctgcgcttccgcccctacgtgtccaaggtgccctggcacaccggcttccgcggcctgctgtccc
agctgttcccccgctacggccactactgcggccccaactggtcctccggcaagaacggcggctcccccgtgtgggaccagcgcc
ccatcgactggctggactactgctgctactgccacgacatcggctacgacacccacgaccaggccaagctgctggaggccgacc
tggccttcctggagtgcctggagcgcccctcctaccccaccaccggcgacgcccacgtggcccacatgtacaagaccatgtgcgt
gaccggcctgcgcaacgtgctgatcccctaccgcacccagctgctgcgcctgaacttccgccagcccctgatcgacttcggctggc
tgtccaacgccgcctggaagggctggtccgcccagaagaccTGActcgag
SEQ ID NO: 124 CuPSR23PLA2-2
ggtaccATGgtgcacctgccccacaccctgaagctgggcctggtgatcgccatctccatctccggcctgtgcttctcctccacccc
cgcccgcgccctgaacgtgggcatccaggccgccggcgtgaccgtgtccgtgggcaagggctgctcccgcaagtgcgagtccg
acttctgcaaggtgccccccttcctgcgctacggcaagtactgcggcctgatgtactccggctgccccggcgagaagccctgcgac
ggcctggacgcctgctgcatgaagcacgacgcctgcgtgcaggccaagaacaacgactacctgtcccaggagtgctcccagaa
cctgctgaactgcatggcctccttccgcatgtccggcggcaagcagttcaagggctccacctgccaggtggacgaggtggtggac
gtgctgaccgtggtgatggaggccgccctgctggccggccgctacctgcacaagcccTGActcgag
SEQ ID NO: 125 CprocPLA2-2
ggtaccATGgtgcacctgccccacaccctgaagctgggcctggtgatcgccatctccatctccggcctgtgcctgtcctccacccc
cgcccgcgccctgaacgtgggcatccaggccgccggcgtgaccgtgtccgtgggcaagggctgctcccgcaagtgcgagtccg
acttctgcaaggtgccccccttcctgcgctacggcaagtactgcggcctgatgtactccggctgccccggcgagaagccctgcgac
ggcctggacgcctgctgcatgaagcacgacgcctgcgtgcaggccaagaacgacgactacctgtcccaggagtgctcccagaa
cctgctgaactgcatggcctccttccgcatgtccggcggcaagcagttcaagggctccacctgccaggtggacgaggtggtggac
gtgctgaccgtggtgatggaggccgccctgctggccggccgctacctgcacaagcccTGActcgag

The constructs containing the codon optimized genes described above driven by the UTEX 1453 SAD2 promoter, were transformed into strain S57858 or S8714. Transformations, cell culture, lipid production and fatty acid analysis were all carried out as described herein. The transgenic strains were selected for their ability to grow on melibiose. Stable transformants were grown under standard lipid production conditions at pH5 (for transgenic strains generated in the strain S7858) or at pH7 (for the transgenic strains generated in the strain S8174) for fatty acid analysis.

### Expression of LPAATs

In WO2013/158938 we disclosed that Cocos nucifera LPAAT enzymes exhibit chain length specificity for the fatty acid acyl-CoA that it attach to the glycerol backbone. We disclosed the impact of expressing CnLPAAT in a transgenic strain also expressing a laurate specific thioesterase. In this example we transformed 5 LPAAT enzymes derived from C8-C10 rich Cuphea species and the CnLPAAT into S7858, and the remaining 8 LPAAT enzymes were transformed into S8174. The resulting fatty acid profiles from a set of representative transgenic lines arising from these transformations are shown in Tables 16 and 17. Expression of these genes as shown in Table 16 resulted in increases in C8:0 and/or -C10:0 fatty acid accumulation.

TABLE 16
Fatty acid profiles of representative transgenic strains of S7858
expressing optimized versions of the CpauLPAAT1, CpalLPAAT1,
CignLPAAT1, CprocLPAAT1, ChookLPAAT1 and CnLPAAT1.

| Sample ID | C8:0 | C10:0 | C12:0 | C8-C10 |
|---|---|---|---|---|
| S6165 | 0.00 | 0.00 | 0.05 | 0.00 |
| S7858 | 11.70 | 23.36 | 0.48 | 35.06 |

CpauLPAAT1 @ SAD2-1vD locus

| | | | | |
|---|---|---|---|---|
| S7858; D4289-7 | 12.69 | 25.06 | 0.51 | 37.75 |
| S7858; D4289-12 | 11.98 | 24.54 | 0.48 | 36.52 |
| S7858; D4289-2 | 11.68 | 24.14 | 0.49 | 35.82 |
| S7858; D4289-13 | 11.53 | 24.18 | 0.49 | 35.71 |
| S7858; D4289-11 | 11.47 | 23.85 | 0.46 | 35.32 |

CpaiLPAAT1 @ SAD2-1vD locus

| | | | | |
|---|---|---|---|---|
| S7858; D4290-3 | 13.43 | 25.04 | 0.52 | 38.47 |
| S7858; D4290-25 | 12.98 | 24.75 | 0.51 | 37.73 |
| S7858; D4290-5 | 12.27 | 25.00 | 0.52 | 37.27 |
| S7858; D4290-12 | 11.98 | 24.21 | 0.48 | 36.19 |
| S7858; D4290-22 | 11.91 | 23.86 | 0.49 | 35.77 |

CignLPAAT1 @ SAD2-1vD locu

| | | | | |
|---|---|---|---|---|
| S7858; D4291-13 | 12.95 | 24.78 | 0.52 | 37.73 |
| S7858; D4291-20 | 12.13 | 24.63 | 0.49 | 36.76 |
| S7858; D4291-15 | 12.12 | 24.35 | 0.47 | 36.47 |
| S7858; D4291-22 | 11.94 | 24.50 | 0.47 | 36.44 |
| S7858; D4291-7 | 12.11 | 23.14 | 0.50 | 35.25 |

CprocLPAAT1 @ SAD2-1vD locus

| | | | | |
|---|---|---|---|---|
| S7858; D4292-15 | 11.86 | 24.05 | 0.46 | 35.91 |
| S7858; D4292-11 | 11.49 | 24.01 | 0.48 | 35.50 |
| S7858; D4292-22 | 11.49 | 23.81 | 0.47 | 35.30 |
| S7858; D4292-3 | 11.46 | 23.76 | 0.46 | 35.22 |
| S7858; D4292-24 | 11.38 | 23.64 | 0.46 | 35.02 |

ChookLPAAT1 @ SAD2-1vD locus

| | | | | |
|---|---|---|---|---|
| S7858; D4293-4 | 11.09 | 24.48 | 0.51 | 35.57 |
| S7858; D4293-16 | 12.03 | 24.24 | 0.48 | 36.27 |
| S7858; D4293-6 | 11.83 | 23.79 | 0.48 | 35.62 |
| S7858; D4293-2 | 11.81 | 23.69 | 0.47 | 35.50 |
| S7858; D4293-12 | 11.65 | 23.11 | 0.49 | 34.76 |

CnLPAAT1 @ SAD2-1vD locus

| | | | | |
|---|---|---|---|---|
| S7858; D4404-11 | 12.30 | 24.31 | 0.47 | 36.61 |
| S7858; D4404-6 | 12.03 | 24.02 | 0.46 | 36.05 |
| S7858; D4404-13 | 11.48 | 23.98 | 0.46 | 35.46 |
| S7858; D4404-2 | 11.54 | 23.71 | 0.46 | 35.25 |
| S7858; D4404-1 | 11.76 | 23.36 | 0.48 | 35.12 |

TABLE 17
Fatty acid profiles of representative transgenic strains of S8174
expressing CavigLPAAT1, CavigLPAAT2, CpalLPAAT1,
CuPSR23LPAAT1, CkoeLPAAT1, CkoeLPAAT2, CprocLPAAT1
and CprocLPAAT2 before lipase treatment.

| Sample ID | C8:0 | C10:0 | C12:0 | C8-C10 |
|---|---|---|---|---|
| S7485 | 0.00 | 0.00 | 0.07 | 0.00 |
| S8174 | 24.32 | 9.24 | 0.37 | 33.56 |

CavigLPAAT1 @ SAD2-1vD locus

| | | | | |
|---|---|---|---|---|
| S8174: D4517-23 | 25.42 | 9.63 | 0.39 | 35.05 |
| S8174: D4517-9 | 25.44 | 9.61 | 0.39 | 35.05 |
| S8174: D4517-8 | 25.09 | 9.84 | 0.39 | 34.93 |
| S8174: D4517-18 | 25.20 | 9.65 | 0.39 | 34.85 |
| S8174: D4517-2 | 25.20 | 9.57 | 0.37 | 34.77 |

CavigLPAAT2 @ SAD2-1vD locus

| | | | | |
|---|---|---|---|---|
| S8174: D4518-2 | 24.25 | 9.97 | 0.42 | 34.22 |
| S8174: D4518-45 | 24.09 | 9.65 | 0.39 | 33.74 |
| S8174: D4518-34 | 23.94 | 9.71 | 0.38 | 33.65 |
| S8174: D4518-10 | 24.11 | 9.50 | 0.37 | 33.61 |
| S8174: D4518-4 | 23.93 | 9.59 | 0.39 | 33.52 |

CpalLPAAT1 @ SAD2-1vD locus

| | | | | |
|---|---|---|---|---|
| S8174: D4519-27 | 25.06 | 9.75 | 0.37 | 34.81 |
| S8174: D4519-4 | 23.05 | 10.74 | 0.47 | 33.79 |
| S8174: D4519-28 | 24.11 | 9.54 | 0.37 | 33.65 |
| S8174: D4519-10 | 23.57 | 9.51 | 0.38 | 33.08 |
| S8174: D4519-12 | 23.55 | 9.49 | 0.38 | 33.04 |

CuPSR23LPAAT2-1 @ SAD2-1vD locus

| | | | | |
|---|---|---|---|---|
| S8174; D4690-2 | 25.88 | 10.62 | 0.43 | 36.50 |
| S8174; D4690-1 | 24.60 | 9.82 | 0.44 | 34.42 |
| S8174; D4690-3 | 24.13 | 9.62 | 0.47 | 33.75 |
| S8174; D4690-4 | 23.38 | 9.97 | 0.41 | 33.35 |

CkoeLPAAT1 @ SAD2-1vD locus

| | | | | |
|---|---|---|---|---|
| S8174; D4728-8 | 25.44 | 10.31 | 0.46 | 35.75 |
| S8174; D4728-10 | 24.15 | 9.51 | 0.43 | 33.66 |
| S8174; D4728-5 | 23.88 | 9.56 | 0.45 | 33.44 |
| S8174; D4728-6 | 23.58 | 9.28 | 0.40 | 32.86 |
| S8174; D4728-9 | 23.47 | 9.25 | 0.40 | 32.72 |

CkoeLPAAT2-1 @ SAD2-1vD locus

| | | | | |
|---|---|---|---|---|
| S8174; D4729-2 | 25.20 | 9.81 | 0.43 | 35.01 |
| S8174; D4729-1 | 23.49 | 10.60 | 0.46 | 34.09 |
| S8174; D4729-4 | 22.25 | 9.45 | 0.40 | 31.70 |
| S8174; D4729-5 | 18.24 | 8.22 | 0.35 | 26.46 |

CprocLPAAT2 @ SAD2-1vD locus

| | | | | |
|---|---|---|---|---|
| S8174: D4730-14 | 24.97 | 9.92 | 0.41 | 34.89 |
| S8174: D4730-13 | 23.26 | 10.72 | 0.49 | 33.98 |
| S8174: D4730-1 | 23.79 | 10.15 | 0.49 | 33.94 |
| S8174: D4730-7 | 23.42 | 10.13 | 0.36 | 33.55 |
| S8174: D4730-5 | 23.69 | 9.49 | 0.42 | 33.18 |

CuPSR23LPAAT4 @ SAD2-1vD locus

| | | | | |
|---|---|---|---|---|
| S8174; D4731-1 | 25.94 | 10.87 | 0.56 | 36.81 |
| S8174; D4731-3 | 22.79 | 11.52 | 0.59 | 34.31 |
| S8174; D4731-5 | 22.89 | 11.22 | 0.53 | 34.11 |
| S8174; D4731-2 | 22.99 | 11.07 | 0.45 | 34.06 |
| S8174; D4731-4 | 21.15 | 9.63 | 0.43 | 30.78 |

To assess the regiospecific activity of novel LPAAT enzymes, oil extracted from some of these transformants were treated with porcine pancreatic lipase, which selectively hydrolyzes the fatty acids at the sn-1 and sn-3 positions from the glycerol unit of the triacylglycerol, leaving monoacylglycerols (MAGs) with fatty acids located only at the sn-2 position. The resulting mixture of monoacylglycrols (2-MAGs), were isolated by solid phase extraction on an amino propyl cartridge followed by transesterifcation to generate fatty acid methyl esters (FAMEs). The fatty acid profiles of these FAMEs, which represent the profile of fatty acids at the sn-2 position of the various TAGs, were determined by GC-FID. When compared to the fatty acid profiles from transesterification of the oil without lipase treatment, the sn-2 fatty acid profiles show that the expressed LPAAT are selective for the sn-2 position.

The sn-2 analyses after lipase treatment disclosed in Table 18 show that CavigLPAAT1, CpaiLPAAT exhibit selectivity for either C8:0 fatty acids and CpauLPAAT, CignLPAAT are selective for C10:0 fatty acids, demonstrating that the heterologous LPAATs expressed in these transgenic strains have activities that acylate at the sn-2 position with preference for C8:0 or C10:0.

TABLE 18
Fatty acid profiles & sn-2 analysis of representative transgenic strains
of S7858 & S8174 expressing codon optimized versions of the CnLPAAT1,
CpauLPAAT1, CpaiLPAAT1, CignLPAAT1, ChookLPAAT1 and CavigLPAAT1,
CavigLPAAT2, CpalLPAAT1

| | | | pH 5; S7858; | | pH 5; S7858; | | pH 5; S7858; | | pH 5; S7858; | |
|---|---|---|---|---|---|---|---|---|---|---|
| | pH 5; S7858 | | D4404-2; | | D4289-2 | | D4290-5 | | D4291-7 | |
| Fatty Acid | FA profile | sn-2 | FA profile | sn-2 | FA profile | sn-2 | FA profile | sn-2 | FA profile | sn-2 |
| C8:0 | 11.08 | 8.6 | 13.54 | 6.8 | 11.68 | 8.1 | 12.27 | 10.5 | 12.11 | 7.4 |
| C10:0 | 23.58 | 20.3 | 25.04 | 20.5 | 24.14 | 28.2 | 25.00 | 13.7 | 23.14 | 31.9 |
| C12:0 | 0.47 | 0.2 | 0.49 | 0.2 | 0.49 | 0.2 | 0.52 | 0.2 | 0.50 | 0.2 |
| C14:0 | 1.19 | 0.7 | 1.19 | 0.7 | 1.29 | 0.7 | 1.39 | 0.8 | 1.38 | 0.6 |
| C16:0 | 11.63 | 1.2 | 10.28 | 1.0 | 12.57 | 1.2 | 12.72 | 1.5 | 12.63 | 1.2 |
| C18:0 | 1.56 | 0.3 | 1.52 | 0.2 | 3.61 | 0.7 | 5.41 | 0.7 | 4.15 | 0.6 |
| C18:1 | 44.49 | 63.1 | 42.25 | 63.1 | 39.69 | 52.9 | 38.50 | 63.2 | 39.50 | 50.2 |
| C18:2 | 4.78 | 6.4 | 4.54 | 8.4 | 5.01 | 6.5 | 4.85 | 7.9 | 5.23 | 6.4 |
| C18:3 α | 0.31 | 0.7 | 0.25 | 0.7 | 0.50 | 1.0 | 0.54 | 1.2 | 0.49 | 1.2 |

| | | | |
|---|---|---|---|
| CnLPAAT | CpauLPAAT | CpaiLPAAT | CignLPAAT |

| | | | pH 7; S8174; | | pH 7; S8174; | | pH 7; S8174; | |
|---|---|---|---|---|---|---|---|---|
| | pH 7; S8174 | | D4517-23; | | D4518-45; | | D4519-28; | |
| Fatty Acid | FA profile | sn-2 | FA profile | sn-2 | FA profile | sn-2 | FA profile | sn-2 |
| C8:0 | 25.24 | 15.9 | 26.04 | 25.1 | 25.04 | 17.8 | 24.75 | 16.0 |
| C10:0 | 9.33 | 8.8 | 9.02 | 7.2 | 9.01 | 9.0 | 8.94 | 8.7 |
| C12:0 | 0.44 | 0.2 | 0.41 | 0.2 | 0.40 | 0.2 | 0.39 | 0.2 |
| C14:0 | 2.48 | 1.4 | 2.45 | 1.2 | 2.45 | 1.4 | 2.45 | 1.4 |
| C16:0 | 13.88 | 1.1 | 13.91 | 1.1 | 14.19 | 1.2 | 14.38 | 1.1 |
| C18:0 | 1.33 | 0.3 | 3.43 | 0.4 | 3.35 | 0.4 | 3.52 | 0.4 |
| C18:1 | 37.50 | 62.0 | 35.36 | 55.1 | 38.86 | 59.7 | 38.94 | 81.2 |
| C18:2 | 8.52 | 8.4 | 5.87 | 8.0 | 6.08 | 8.4 | 6.14 | 9.1 |
| C18:3 α | 0.65 | 1.3 | 0.53 | 1.3 | 0.58 | 1.3 | 0.58 | 1.5 |

| | | |
|---|---|---|
| CavigLPAAT1 | CavigLPAAT2 | CpalLPAAT1 |

### Expression of GPATs, DGATs, LPCATs and PLA2s:

The constructs expressing the other acyltransferases (GPAT, DGAT, LPCAT, and PLA2) were transformed into S8174. Stable transformants were grown under standard lipid production conditions at pH7 and analyzed for fatty acid profiles. Similar to the transgenic lines expressing LPAATs, expression of these genes (GPAT, DGAT, LPCAT, and PLA2) also resulted in increases in C8:0-C10:0 fatty acid accumulation (Tables 19a, 19b, and 20). The data presented shows that we have identified novel GPATs, DGATs, LPCATs and PLA2s that show high specificity for C8-C10 fatty acids. To determine the regiospecificity of the novel GPAT, DGAT, LPCAT, and PLA2 enzymes, sn-2 analysis is performed as disclosed in this example and elsewhere herein.

TABLE 19a
Fatty acid profiles of representative transgenic strains
of S8174 expressing GPATs and DGATs

| Sample ID | C8:0 | C10:0 | C12:0 | C8-C10 |
|---|---|---|---|---|
| S7485 | 0.00 | 0.00 | 0.07 | 0.00 |
| S8174 | 24.61 | 9.10 | 0.42 | 33.71 |

CavigGPAT9 @ SAD2-1vD locus

| | | | | |
|---|---|---|---|---|
| S8174; D4551-8 | 24.52 | 9.05 | 0.36 | 33.57 |
| S8174; D4551-7 | 24.24 | 9.04 | 0.36 | 33.28 |
| S8174; D4551-2 | 23.93 | 8.92 | 0.37 | 32.85 |
| S8174; D4551-6 | 23.63 | 8.92 | 0.41 | 32.55 |
| S8174; D4551-3 | 23.35 | 8.90 | 0.43 | 32.25 |

ChookGPAT9-1 @ SAD2-1vD locus

| | | | | |
|---|---|---|---|---|
| S8174; D4552-6 | 23.57 | 9.00 | 0.36 | 32.57 |
| S8174; D4552-4 | 23.62 | 8.87 | 0.37 | 32.49 |
| S8174; D4552-9 | 23.39 | 8.97 | 0.40 | 32.36 |
| S8174; D4552-8 | 23.28 | 8.80 | 0.40 | 32.08 |
| S8174; D4552-11 | 23.18 | 8.80 | 0.44 | 31.98 |

CignGPAT9-1 @ SAD2-1vD locus

| | | | | |
|---|---|---|---|---|
| S8174; D4553-12 | 25.19 | 9.42 | 0.40 | 34.61 |
| S8174; D4685-1 | 24.33 | 10.24 | 0.46 | 34.57 |
| S8174; D4553-15 | 25.11 | 9.33 | 0.41 | 34.44 |
| S8174; D4553-1 | 24.56 | 9.50 | 0.44 | 34.06 |
| S8174; D4553-6 | 24.74 | 9.16 | 0.40 | 33.90 |

CignGPAT9-2 @ SAD2-1vD locus

| | | | | |
|---|---|---|---|---|
| S8174; D4554-9 | 24.49 | 9.13 | 0.45 | 33.62 |
| S8174; D4554-3 | 24.28 | 8.90 | 0.42 | 33.18 |
| S8174; D4554-7 | 23.86 | 8.96 | 0.43 | 32.82 |
| S8174; D4554-8 | 23.99 | 8.81 | 0.39 | 32.80 |
| S8174; D4554-4 | 23.87 | 8.78 | 0.4 | 32.65 |

CpalGPAT9-1 @ SAD2-1vD locus

| | | | | |
|---|---|---|---|---|
| S8174; D4724-6 | 25.61 | 9.52 | 0.39 | 35.13 |
| S8174; D4724-7 | 24.91 | 9.36 | 0.41 | 34.27 |
| S8174; D4724-2 | 24.43 | 9.46 | 0.39 | 33.89 |
| S8174; D4724-5 | 24.01 | 9.25 | 0.39 | 33.26 |
| S8174; D4724-4 | 24.30 | 8.93 | 0.39 | 33.23 |

CpalGPAT9-2 @ SAD2-1vD locus

| | | | | |
|---|---|---|---|---|
| S8174; D4725-5 | 24.24 | 10.30 | 0.48 | 34.54 |
| S8174; D4725-6 | 24.81 | 9.29 | 0.41 | 34.10 |
| S8174; D4725-7 | 24.35 | 9.51 | 0.42 | 33.86 |
| S8174; D4725-8 | 24.37 | 9.39 | 0.40 | 33.76 |
| S8174; D4725-9 | 24.28 | 9.29 | 0.41 | 33.57 |

TABLE 19b
Fatty acid profiles of representative transgenic
strains of S8174 expressing DGATs

| Sample ID | C8:0 | C10:0 | C12:0 | C8-C10 |
|---|---|---|---|---|
| S7485 | 0.00 | 0.00 | 0.07 | 0.00 |
| S8174 | 24.61 | 9.10 | 0.42 | 33.71 |
| Cavig DGAT1 @ SAD2-1vD locus | | | | |
| S8174; D4549-7 | 24.89 | 9.28 | 0.36 | 34.17 |
| S8174; D4549-6 | 24.53 | 9.04 | 0.47 | 33.57 |
| S8174; D4549-4 | 23.93 | 8.99 | 0.41 | 32.92 |
| S8174; D4549-1 | 23.93 | 8.97 | 0.38 | 32.90 |
| S8174; D4549-3 | 23.76 | 8.9 | 0.36 | 32.66 |
| Chook DGAT1 @ SAD2-1vD locus | | | | |
| S8174; D4550-1 | 24.67 | 9.12 | 0.41 | 33.79 |
| S8174; D4550-3 | 24.64 | 9.06 | 0.42 | 33.70 |
| S8174; D4682-1 | 23.72 | 9.68 | 0.5 | 33.40 |
| S8174; D4682-2 | 23.49 | 9.66 | 0.41 | 33.15 |
| S8174; D4550-2 | 22.42 | 8.81 | 0.41 | 31.23 |

TABLE 20
Fatty acid profiles of representative transgenic strains
of S8174 expressing LPCATs and PLA2s

| Sample ID | C8:0 | C10:0 | C12:0 | C8-C10 |
|---|---|---|---|---|
| S7485 | 0.00 | 0.00 | 0.07 | 0.00 |
| S8174 | 24.61 | 9.10 | 0.42 | 33.71 |

Cavig LPCAT @ SAD2-1vD locus

| | | | | |
|---|---|---|---|---|
| S8174; D4555-1 | 26.6 | 9.38 | 0.47 | 35.98 |
| S8174; D4555-3 | 26.4 | 9.47 | 0.39 | 35.87 |
| S8174; D4688-1 | 25.95 | 9.67 | 0.44 | 35.62 |
| S8174; D4688-3 | 25.47 | 9.89 | 0.44 | 35.36 |
| S8174; D4555-2 | 25.52 | 9.55 | 0.36 | 35.07 |

Cpau LPCAT @ SAD2-1vD locus

| | | | | |
|---|---|---|---|---|
| S8174; D4556-3 | 25.55 | 9.21 | 0.43 | 34.76 |
| S8174; D4556-4 | 25.24 | 9.46 | 0.41 | 34.70 |
| S8174; D4689-7 | 24.63 | 9.86 | 0.43 | 34.49 |
| S8174; D4556-1 | 25.18 | 9.13 | 0.42 | 34.31 |
| S8174; D4689-6 | 24.05 | 9.89 | 0.48 | 33.94 |

Cpal LPCAT @ SAD2-1vD locus

| | | | | |
|---|---|---|---|---|
| S8174; D4726-4 | 26.34 | 9.76 | 0.41 | 36.10 |
| S8174; D4726-2 | 25.92 | 9.9 | 0.44 | 35.82 |
| S8174; D4726-3 | 26.15 | 9.62 | 0.41 | 35.77 |
| S8174; D4726-5 | 26.09 | 9.55 | 0.41 | 35.64 |
| S8174; D4726-1 | 25.64 | 9.57 | 0.39 | 35.21 |

Cschu LPCAT @ SAD2-1vD locus

| | | | | |
|---|---|---|---|---|
| S8174; D4727-1 | 26.24 | 9.95 | 0.45 | 36.19 |
| S8174; D4727-7 | 26.26 | 9.84 | 0.42 | 36.10 |
| S8174; D4727-9 | 26.13 | 9.87 | 0.42 | 36.00 |
| S8174; D4727-11 | 25.99 | 9.97 | 0.44 | 35.96 |
| S8174; D4727-16 | 26.28 | 9.68 | 0.44 | 35.96 |

Cavig PLA2-1 @ SAD2-1vD locus

| | | | | |
|---|---|---|---|---|
| S8174; D4732-1 | 26.31 | 11.24 | 0.60 | 37.55 |
| S8174; D4732-2 | 25.30 | 11.88 | 0.50 | 37.18 |
| S8174; D4732-3 | 25.29 | 11.01 | 0.48 | 36.30 |
| S8174; D4732-4 | 25.30 | 11.00 | 0.47 | 36.30 |
| S8174; D4732-5 | 25.07 | 11.20 | 0.44 | 36.27 |

CignPLA2-1 @ SAD2-1vD locus

| | | | | |
|---|---|---|---|---|
| S8174; D4734-6 | 26.39 | 11.34 | 0.47 | 37.73 |
| S8174; D4734-1 | 26.17 | 10.90 | 0.46 | 37.07 |
| S8174; D4734-5 | 25.58 | 11.12 | 0.57 | 36.70 |
| S8174; D4734-4 | 25.48 | 11.17 | 0.57 | 36.65 |
| S8174; D4734-2 | 24.75 | 11.32 | 0.46 | 36.07 |

CuPSR23PLA2-2 @ SAD2-1vD locus

| | | | | |
|---|---|---|---|---|
| S8174; D4735-5 | 25.81 | 11.16 | 0.44 | 36.97 |
| S8174; D4735-1 | 25.95 | 10.92 | 0.47 | 36.87 |
| S8174; D4735-8 | 25.54 | 10.91 | 0.42 | 36.45 |
| S8174; D4735-7 | 25.45 | 10.95 | 0.44 | 36.40 |
| S8174; D4735-6 | 25.51 | 10.88 | 0.41 | 36.39 |

Cproc PLA2-2 @ SAD2-1vD locus

| | | | | |
|---|---|---|---|---|
| S8174; D4736-2 | 25.60 | 10.87 | 0.42 | 36.47 |
| S8174; D4736-4 | 25.55 | 10.76 | 0.40 | 36.31 |
| S8174; D4736-3 | 25.40 | 10.87 | 0.36 | 36.27 |
| S8174; D4736-5 | 25.45 | 10.46 | 0.39 | 35.91 |
| S8174; D4736-1 | 24.34 | 11.06 | 0.48 | 35.40 |

### Example 7: Expression of LPAAT and/or DGAT in Prototheca to Produce High SOS and Low Trisaturated Tags

In this example we describe genetically engineered Prototheca moriformis strains in which we have modified fatty acid and triacylglycerol biosynthesis to maximize the accumulation of Stearoyl-Oleoyl-Stearoyl (SOS) TAGs, and minimize the production of trisaturated TAGs. Tailored oils from these strains resemble plant seed oils known as “structuring fats”, which have high proportions of Saturated-Oleate-Saturated TAGs and low levels of trisaturates. These structuring fats (often called “butters”) are generally solid at room temperature but melt sharply between 35-40° C.

High-SOS strains were obtained by three successive transformations beginning with strain S5100, a classically improved derivative, of a wild type isolate of Prototheca moriformis, S376. Strain S5100 was transformed with plasmid pSZ5654 to generate strain S8754, which produces an oil with increased stearic acid (C18:0) content, lower palmitic acid (C16:0) and reduced linoleic acid (C18:2 cisΔ9,12) content relative to S5100. In turn, strain S8754 was transformed with plasmid pSZ5868 to generate strain S8813, which produces oil with higher C18:0, lower C16:0 and improved sn-2 selectivity compared to S8754. Finally, strain S8813 was transformed with plasmids pSZ6383 or pSZ6384 to generate strains S9119, S9120 and S9121, producing oils rich in C18:0 with reduced levels of C18:2 cisΔ9,12 and improved sn-3 selectivity.

Construct used for SAD2 knockout in S5100

The first intermediate strains were prepared by transformation of strain S5100 with integrative plasmid pSZ5654 (SAD2-1vD::PmKASII-1tp_PmKASII-1_FLAG-CvNR:CrTUB2-PmFAD2hpA-CvNR:PmHXT1-2v2-ScarMEL1-PmPGK::SAD2-1vE). The construct targeted ablation of allele 1 of the endogenous stearoyl-ACP desaturase 2 gene (SAD2), concomitant with expression of the PmKASII gene encoding P. moriformis β-keto-acyl-ACP synthase, and a RNAi hairpin sequence to down-regulate fatty acid desaturase (FAD2) gene expression. Deletion of one allele of SAD2 reduced SAD activity, resulting in elevated levels of C18:0. Overexpression of PmKASII stimulated elongation of C16:0 to C18:0, further increasing C18:0. FAD2 is responsible for the conversion of C18:1 cisΔ9 (oleic) to C18:2 cisΔ9,12 (linoleic) fatty acids, and RNAi of FAD2 resulted in decreased C18:2. Thus, the first intermediate strains had higher levels of C18:0 and decreased C16:0 and C18:2 fatty acid levels relative to the S5100 parent. The Saccharomyces carlsbergensis MEL1 gene, encoding a secreted melibiase served as a selectable marker as part of plasmid pSZ5654, enabling the strain to grow on melibiose.

The sequence of the pSZ5654 transforming DNA is provided below. Relevant restriction sites in the construct are indicated in lowercase, bold and underlining and are 5′-3′ PmeI, SpeI, AscI, ClaI, SacI, AvrII, EcoRV, EcoRI, SpeI, BsiWI, XhoI, SacI, KpnI, SnaBI, BspQI and PmeI, respectively. PmeI sites delimit the 5′ and 3′ ends of the transforming DNA. Bold, lowercase sequences represent SAD2-1 5′ genomic DNA that permit targeted integration at the SAD2-1 locus via homologous recombination. Proceeding in the 5′ to 3′ direction, bold, lowercase sequences represent SAD2-1 5′ genomic DNA sequences that permit targeted integration at the FATA-1 locus via homologous recombination. The initiator ATG of the sequence encoding the P. moriformis KASII-1 transit peptide (PmKASII-1tp) is indicated by uppercase, bold italics, and the PmKASII-1tp sequence located between the ATG and the AscI site is indicated with lowercase, underlined italics. The PmKASII-1 coding region is indicated by lowercase italics. A sequence encoding a 3× FLAG tag fused to the C-terminus of PmKASII-1 is represented by uppercase italics, and the TGA terminator codon is indicated with uppercase, bold italics. The Chlorella vulgaris nitrate reductase (NR) gene 3′ UTR is indicated by lowercase underlined text. A spacer sequence is represented by lowercase text. The C. reinhardtii TUB2 promoter, driving expression of the PmFAD2hpA sequence is indicated by boxed text. Bold italics denote the PmFAD2hpA sequence followed by lowercase underlined text representing C. vulgaris nitrate reductase 3′ UTR. A second spacer sequence is represented by lowercase text. The P. moriformis HXT1 promoter driving the expression of the S. carlbergensis MEL1 gene is indicated by boxed text. The initiator ATG and terminator TGA for MEL1 gene are indicated by uppercase, bold italics while the coding region is indicated in lowercase italics. The P. moriformis PGK 3′ UTR is indicated by lowercase underlined text. The SAD2-1 3′ genomic region indicated by bold, lowercase text.

SEQ ID NO: 126 Nucleotide sequence of transforming DNA contained in
pSZ5654
gtttaaacgccggtcaccacccgcatgctcgtactacagcgcacgcaccgcttcgtgatccaccgggtgaacgtagtcctcgacgg
aaacatctggttcgggcctcctgcttgcactcccgcccatgccgacaacctttctgctgttaccacgacccacaatgcaacgcgaca
cgaccgtgtgggactgatcggttcactgcacctgcatgcaattgtcacaagcgcttactccaattgtattcgtttgttttctgggagc
agttgctcgaccgcccgcgtcccgcaggcagcgatgacgtgtgcgtggcctgggtgtttcgtcgaaaggccagcaaccctaaatcg
caggcgatccggagattgggatctgatccgagtttggaccagatccgccccgatgcggcacgggaactgcatcgactcggcgcgg
aacccagctttcgtaaatgccagattggtgtccgatacctggatttgccatcagcgaaacaagacttcagcagcgagcgtatttgg
cgggcgtgctaccagggttgcatacattgcccatttctgtctggaccgctttactggcgcagagggtgagttgatggggttggcagg
catcgaaacgcgcgtgcatggtgtgcgtgtctgttttcggctgcacgaattcaatagtcggatgggcgacggtagaattgggtgtg
gcgctcgcgtgcatgcctcgccccgtcgggtgtcatgaccgggactggaatcccccctcgcgaccatcttgctaacgctcccgactc
tcccgaccgcgcgcaggatagactcttgttcaaccaatcgacaactagtATGcagaccgcccaccagcgcccccccaccgagg
gccactgcttcggcgcccgcctgcccaccgcctcccgccgcgccgtgcgccgcgcctggtcccgcatcgcccgcgggcgcgccgc
cgccgccgccgacgccaaccccgcccgccccgagcgccgcgtggtgatcaccggccagggcgtggtgacctccctgggccag
accatcgagcagttctactcctccctgctggagggcgtgtccggcatctcccagatccagaagttcgacaccaccggctacacc
accaccatcgccggcgagatcaagtccctgcagctggacccctacgtgcccaagcgctgggccaagcgcgtggacgacgtga
tcaagtacgtgtacatcgccggcaagcaggccctggagtccgccggcctgcccatcgaggccgccggcctggccggcgccgg
cctggaccccgccctgtgcggcgtgctgatcggcaccgccatggccggcatgacctccttcgccgccggcgtggaggccctgac
ccgcggcggcgtgcgcaagatgaaccccttctgcatccccttctccatctccaacatgggcggcgccatgctggccatggacatc
ggcttcatgggccccaactactccatctccaccgcctgcgccaccggcaactactgcatcctgggcgccgccgaccacatccgcc
gcggcgacgccaacgtgatgctggccggcggcgccgacgccgccatcatcccctccggcatcggcggcttcatcgcctgcaag
gccctgtccaagcgcaacgacgagcccgagcgcgcctcccgcccctgggacgccgaccgcgacggcttcgtgatgggcgagg
gcgccggcgtgctggtgctggaggagctggagcacgccaagcgccgcggcgccaccatcctggccgagctggtgggcggcg
ccgccacctccgacgcccaccacatgaccgagcccgacccccagggccgcggcgtgcgcctgtgcctggagcgcgccctggag
cgcgcccgcctggcccccgagcgcgtgggctacgtgaacgcccacggcacctccacccccgccggcgacgtggccgagtaccg
cgccatccgcgccgtgatcccccaggactccctgcgcatcaactccaccaagtccatgatcggccacctgctgggcggcgccgg
cgccgtggaggccgtggccgccatccaggccctgcgcaccggctggctgcaccccaacctgaacctggagaaccccgcccccg
gcgtggaccccgtggtgctggtgggcccccgcaaggagcgcgccgaggacctggacgtggtgctgtccaactccttcggcttc
ggcggccacaactcctgcgtgatcttccgcaagtacgacgagATGGACTACAAGGACCACGACGGCGACTACAA
GGACCACGACATCGACTACAAGGACGACGACGACAAGTGAatcgatgcagcagcagctcggatagtatcgaca
cactctggacgctggtcgtgtgatggactgttgccgccacacttgctgccttgacctgtgaatatccctgccgcttttatcaaacagcc
tcagtgtgtttgatcttgtgtgtacgcgcttttgcgagttgctagctgcttgtgctatttgcgaataccacccccagcatccccttccctc
gtttcatatcgcttgcatcccaaccgcaacttatctacgctgtcctgctatccctcagcgctgctcctgctcctgctcactgcccctcgca
cagccttggtttgggctccgcctgtattctcctggtactgcaacctgtaaaccagcactgcaatgctgatgcacgggaagtagtggga
tgggaacacaaatggagagctccgcgtctcgaacagagcgcgcagaggaacgctgaaggtctcgcctctgtcgcacctcagcgcg
gcatacaccacaataaccacctgacgaatgcgcttggttcttcgtccattagcgaagcgtccggttcacacacgtgccacgttggcg

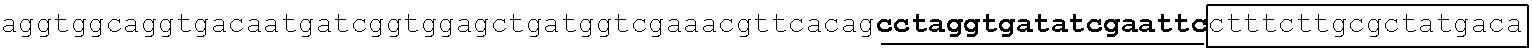

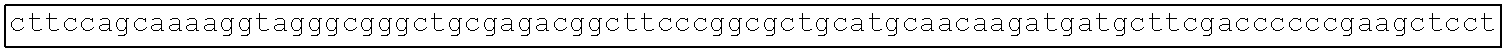

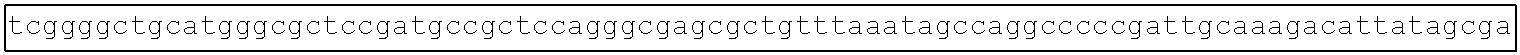

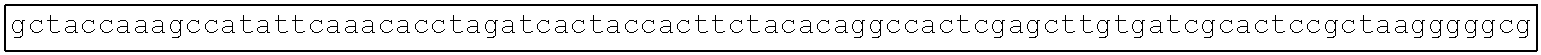

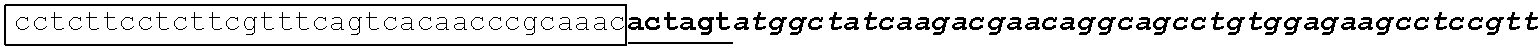

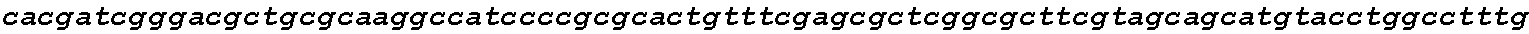

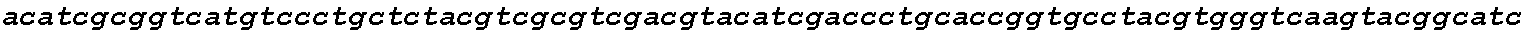

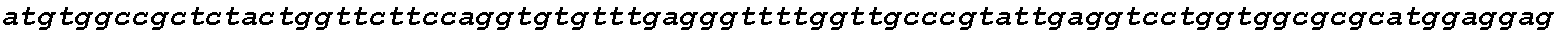

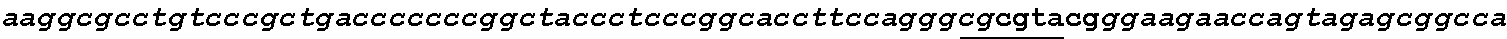

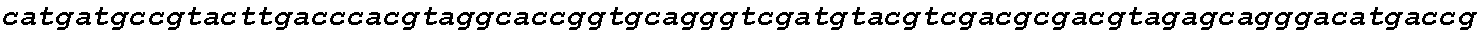

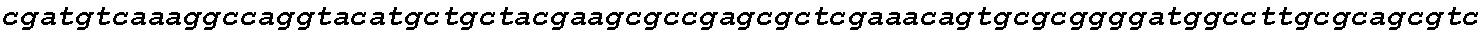

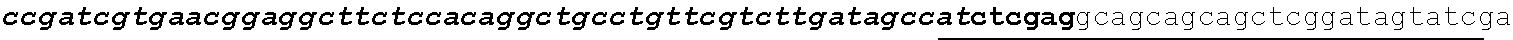

cacactctggacgctggtcgtgtgatggactgttgccgccacacttgctgccttgacctgtgaatatccctgccgcttttatcaaacag
cctcagtgtgtttgatcttgtgtgtacgcgcttttgcgagttgctagctgcttgtgctatttgcgaataccacccccagcatccccttccc
tcgtttcatatcgcttgcatcccaaccgcaacttatctacgctgtcctgctatccctcagcgctgctcctgctcctgctcactgcccctcg
cacagccttggtttgggctccgcctgtattctcctggtactgcaacctgtaaaccagcactgcaatgctgatgcacgggaagtagtgg
gatgggaacacaaatggaaagctgtagagctcgatctaagtaagattcgaagcgctcgaccgtgccggacggactgcagccccat
gtcgtagtgaccgccaatgtaagtgggctggcgtttccctgtacgtgagtcaacgtcactgcacgcgcaccaccctctcgaccggca

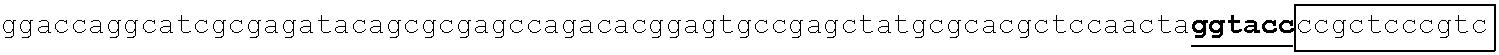

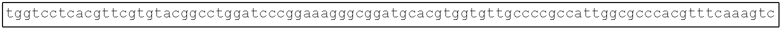

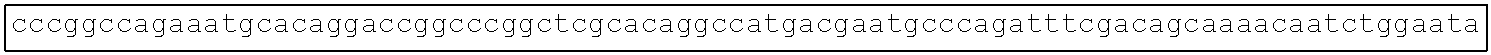

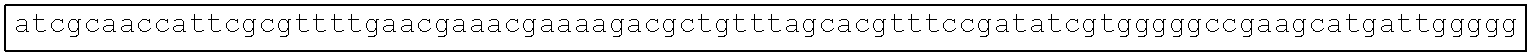

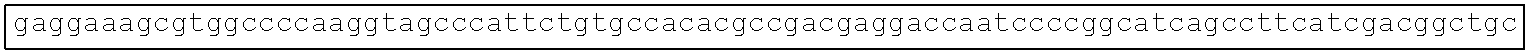

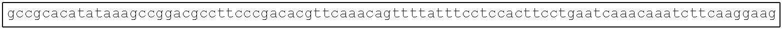

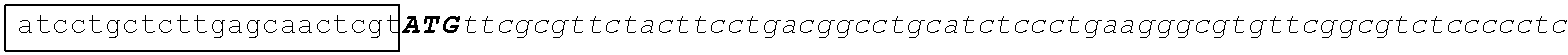

ctacaacggcctgggcctgacgccccagatgggctgggacaactggaacacgttcgcctgcgacgtctccgagcagctgctgc
tggacacggccgaccgcatctccgacctgggcctgaaggacatgggctacaagtacatcatcctggacgactgctggtcctcc
ggccgcgactccgacggcttcctggtcgccgacgagcagaagttccccaacggcatgggccacgtcgccgaccacctgcaca
acaactccttcctgttcggcatgtactcctccgcgggcgagtacacgtgcgccggctaccccggctccctgggccgcgaggagg
aggacgcccagttcttcgcgaacaaccgcgtggactacctgaagtacgacaactgctacaacaagggccagttcggcacgcc
cgagatacctaccaccgctacaaggccatgtccgacgccctgaacaagacgggccgccccatcttctactccagtgcaactg
gggccaggacctgaccttctactggggctccggcatcgcgaactcaggcgcatgtccggcgacgtcacggcggagttcacgc
gccccgactcccgctgcccctgcgacggcgacgagtacgactgcaagtacgccggcttccactgctccatcatgaacatcctga
acaaggccgcccccatgggccagaacgcgggcgtcggcggctggaacgacctggacaacctggaggtcggcgtcggcaac
ctgacggacgacgaggagaaggcgcacttctccatgtgggccatggtgaagtcccccctgatcatcggcgcgaacgtgaaca
acctgaaggcctcctcctactccatctactcccaggcgtccgtcatcgccatcaaccaggactccaacggcatccccgccacgcg
cgtctggcgctactacgtgtccgacacggacgagtacggccagggcgagatccagatgtggtccggccccctggacaacggc
gaccaggtcgtggcgctgctgaacggcggctccgtgtcccgccccatgaacacgaccctggaggagatcttcttcgactccaac
ctgggctccaagaagctgacctccacctgggacatctacgacctgtgggcgaaccgcgtcgacaactccacggcgtccgccat
cctgggccgcaacaagaccgccaccggcatcctgtacaacgccaccgagcagtcctacaaggacggcctgtccaagaacgac
acccgcctgttcggccagaagatcggctccctgtcccccaacgcgatcctgaacacgaccgtccccgcccacggcatcgcgttct
accgcctgcgcccctcctccTGAtacaacttattacgtattctgaccggcgctgatgtggcgcggacgccgtcgtactctttcagact
ttactcttgaggaattgaacctttctcgcttgctggcatgtaaacattggcgcaattaattgtgtgatgaagaaagggtggcacaaga
tggatcgcgaatgtacgagatcgacaacgatggtgattgttatgaggggccaaacctggctcaatcttgtcgcatgtccggcgcaat
gtgatccagcggcgtgactctcgcaacctggtagtgtgtgcgcaccgggtcgctttgattaaaactgatcgcattgccatcccgtcaa
ctcacaagcctactctagctcccattgcgcactcgggcgcccggctcgatcaatgttctgagcggagggcgaagcgtcaggaaatcg
tctcggcagctggaagcgcatggaatgcggagcggagatcgaatcaggatccttagggagcgacgagtgtgcgtgcggggctggc
gggagtgggacgccctcctcgctcctctctgttctgaacggaacaatcggccaccccgcgctacgcgccacgcatcgagcaacga
agaaaaccccccgatgataggttgcggtggctgccgggatatagatccggccgcacatcaaagggcccctccgccagagaagaa
gctcctttcccagcagactccttctgctgccaaaacacttctctgtccacagcaacaccaaaggatgaacagatcaacttgcgtctc
cgcgtagcttcctcggctagcgtgcttgcaacaggtccctgcactattatcttcctgctttcctctgaattatgcggcaggcgagcgct
cgctctggcgagcgctccttcgcgccgccctcgctgatcgagtgtacagtcaatgaatggtcctgggcgaagaacgagggaatttg
tgggtaaaacaagcatcgtctctcaggccccggcgcagtggccgttaaagtccaagaccgtgaccaggcagcgcagcgcgtccgt
gtgcgggccctgcctggcggctcggcgtgccaggctcgagagcagctccctcaggtcgccttggacggcctctgcgaggccggtga
gggcctgcaggagcgcctcgagcgtggcagtggcggtcgtatccgggtcgccggtcaccgcctgcgactcgccatccgaagagcg
tttaaac

Construct pSZ5654 was transformed into S5100. Primary transformants were clonally purified and screened under standard lipid production conditions at pH 5. Integration of pSZ5654 at the SAD2-1 locus was verified by DNA blot analysis. The fatty acid profiles and lipid titers of lead strains were assayed in 50-mL shake flasks (Table 21). 58754 was selected as the lead strain for additional rounds of genetic engineering. As shown in Table 21, C16:0 decreased from 17.6% to less than 6%, C18:0 increased from 4.3% to about 28%, C18:2 decreased from 5.8% to 1.3%.

TABLE 21
Fatty acid profiles of SAD2-1 ablation strains.

| Sample ID | S5100 | S8741 | S8742 | S8743 | S8744 | S8745 | S8746 | S8752 | S8753 | S8754 |
|---|---|---|---|---|---|---|---|---|---|---|
| C14:0 | 0.7 | 0.6 | 0.6 | 0.6 | 0.6 | 0.6 | 0.6 | 0.6 | 0.6 | 0.6 |
| C16:0 | 17.6 | 5.9 | 5.9 | 5.8 | 5.9 | 5.9 | 5.9 | 5.9 | 5.8 | 5.9 |
| C16:1 cis-9 | 0.4 | 0.0 | 0.0 | 0.0 | 0.0 | 0.0 | 0.0 | 0.0 | 0.0 | 0.1 |
| C18:0 | 4.3 | 28.2 | 28.1 | 27.7 | 27.8 | 27.4 | 28.2 | 28.3 | 28.3 | 28.1 |
| C18:1 | 69.8 | 60.1 | 60.2 | 60.6 | 60.5 | 60.9 | 60.0 | 60.0 | 60.0 | 60.0 |
| C18:2 | 5.8 | 1.3 | 1.3 | 1.3 | 1.3 | 1.3 | 1.3 | 1.3 | 1.2 | 1.3 |
| C18:3 α | 0.5 | 0.3 | 0.3 | 0.3 | 0.3 | 0.3 | 0.3 | 0.3 | 0.3 | 0.3 |
| C20:0 | 0.3 | 2.2 | 2.2 | 2.2 | 2.2 | 2.2 | 2.2 | 2.2 | 2.2 | 2.2 |
| saturates | 23.2 | 37.5 | 37.5 | 37.1 | 37.2 | 36.8 | 37.7 | 37.7 | 37.7 | 37.6 |
| lipid (g/L) | 13.5 | 12.8 | 12.5 | 12.5 | 12.5 | 12.3 | 12.3 | 12.3 | 12.4 | 12.3 |

### Construct Used for FATA-1 Knockout in S8754

The second intermediate strains were prepared by transformation of strain S8754 with integrative plasmid pSZ5868 (FATA-1vB::CpSAD1tp_GarmFATA1(G108A)_FLAG-PmSAD2-1:PmG3PDH-1-TcLPAT2-PmATP:CrTUB2-ScSUC2-PmPGH::FATA-1vC). This construct targeted ablation of allele 1 of the endogenous fatty acyl-ACP thioesterase gene (FATA-1), and contained expression modules for GarmFATA1 (G108A), encoding a variant of the Garcinia mangostana FATA1 thioesterase with improved activity, and TcLPAT2 encoding the Theobroma cacao lysophosphatidic acid acyltransferase (LPAAT). Deletion of one copy of FATA-1 reduced endogenous thioesterase activity, further reducing C16:0 accumulation. Expression of GarmFATA1(G108A) stimulated C18:0-ACP hydrolysis, further increasing C18:0. TcLPAT2 had superior specificity for transfer of C18:1 to the sn-2 position of triacylglycerides than the endogeneous LPAAT, leading to reduced accumulation of trisaturates. The second intermediate strains had increased C18:0 and lower C16:0 compared their parent, S8754. The S. cerevisiae SUC2 gene encoding a secreted sucrose invertase, served as a selectable marker as part of plasmid pSZ5868 and enabled the strain to grow on sucrose.

The sequence of the pSZ5868 transforming DNA is provided below. Relevant restriction sites in the construct are indicated in lowercase, bold and underlining and are 5′-3′ BspQI, PmeI, SpeI, AscI, ClaI, SacI, AvrII, NdeI, NsiI, AflII, KpnI, XbaI, MfeI, BamHI, BspQI and PmeI, respectively. BspQI and PmeI sites delimit the 5′ and 3′ ends of the transforming DNA. Proceeding in the 5′ to 3′ direction, bold, lowercase sequences represent FATA-1 5′ genomic DNA that permit targeted integration at the FATA-1 locus via homologous recombination. The initiator ATG of the sequence encoding the C. protothecoides SAD1 transit peptide (CpSAD1tp) is indicated by uppercase, bold italics, and the remainder of the CpSAD1tp sequence located between the ATG and the AscI site is indicated with lowercase, underlined italics. The GarmFATA1 (G108A) coding region is indicated by lowercase italics. A sequence encoding a 3× FLAG tag fused to the C-terminus of GarmFATA1(G108A) is represented by uppercase italics, and the TGA terminator codon is indicated with uppercase, bold italics. The P. moriformis SAD2-1 3′ UTR is indicated by lowercase underlined text. A spacer sequence is represented by lowercase text. The P. moriformis G3PDH-1 promoter, driving expression of the TcLPAT2 sequence is indicated by boxed text. The initiator ATG and terminator TGA codons of the TcLPAT2 gene are indicated by uppercase, bold italics, while the remainder of the coding region is represented with italics. Lowercase underlined text represents the P. moriformis ATP 3′ UTR. A second spacer sequence is represented by lowercase text. The C. reinhardtii TUB2 promoter driving the expression of the S. cerevisiae SUC2 gene is indicated by boxed text. The initiator ATG and terminator TGA for SUC2 are indicated by uppercase, bold italics while the coding region is indicated in lowercase italics. The P. moriformis PGH 3′ UTR is indicated by lowercase underlined text. The FATA-1 3′ genomic region indicated by bold, lowercase text.

SEQ ID NO: 127 Nucleotide sequence of transforming DNA contained in
pSZ5868
gaagagcgcccaatgtttaaacctcttttgctgcgtctcctcaggcttgggggcctccttgggcttgggtgccgccatgatctgcgcg
catcagagaaacgttgctggtaaaaaggagcgcccggctgcgcaatatatatataggcatgccaacacagcccaacctcactcg
ggagcccgtcccaccacccccaagtcgcgtgccttgacggcatactgctgcagaagcttcatgagaatgatgccgaacaagaggg
gcacgaggacccaatcccggacatccttgtcgataatgatctcgtgagtccccatcgtccgcccgacgctccggggagcccgccga
tgctcaagacgagagggccctcgaccaggaggggctggcccgggcgggcactggcgtcgaaggtgcgcccgtcgttcgcctgca
gtcctatgccacaaaacaagtcttctgacggggtgcgtttgctcccgtgcgggcaggcaacagaggtattcaccctggtcatgggg
agatcggcgatcgagctgggataagagatacggtcccgcgcaaggatcgctcatcctggtctgagccggacagtcattctggcaa
gcaatgacaacttgtcaggaccggaccgtgccatatatttctcacctagcgccgcaaaacctaacaatttgggagtcactgtgcca
ctgagttcgactggtagctgaatggagtcgctgctccactaaacgaattgtcagcaccgccagccggccgaggacccgagtcata

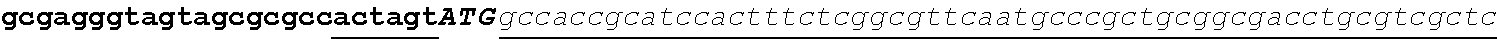

ggcgggctccgggccccggcgcccagcgaggcccctccccgtgcgcgggcgcgccatccccccccgcatcatcgtggtgtcctc
ctcctcctccaaggtgaaccccctgaagaccgaggccgtggtgtcctccggcctggccgaccgcctgcgcctgggctccctgacc
gaggacggcctgtcctacaaggagaagttcatcgtgcgctgctacgaggtgggcatcaacaagaccgccaccgtggagacc
atcgccaacctgctgcaggaggtgggctgcaaccacgcccagtccgtgggctactccaccgccggcttctccaccacccccacc
atgcgcaagctgcgcctgatctgggtgaccgcccgcatgcacatcgagatctacaagtaccccgcctggtccgacgtggtgga
gatcgagtcctggggccagggcgagggcaagatcggcacccgccgcgactggatcctgcgcgactacgccaccggccaggt
gatcggccgcgccacctccaagtgggtgatgatgaaccaggacacccgccgcctgcagaaggtggacgtggacgtgcgcga
cgagtacctggtgcactgcccccgcgagctgcgcctggccttccccgaggagaacaactcctccctgaagaagatctccaagct
ggaggacccctcccagtactccaagctgggcctggtgccccgccgcgccgacctggacatgaaccagcacgtgaacaacgtg
acctacatcggctgggtgctggagtccatgccccaggagatcatcgacacccacgagctgcagaccatcaccctggactaccg
ccgcgagtgccagcacgacgacgtggtggactccctgacctcccccgagccctccgaggacgccgaggccgtgttcaaccaca
acggcaccaacggctccgccaacgtgtccgccaacgaccacggctgccgcaacttcctgcacctgctgcgcctgtccggcaacg
gcctggagatcaaccgcggccgcaccgagtggcgcaagaagcccacccgcATGGACTACAAGGACCACGACGGCG

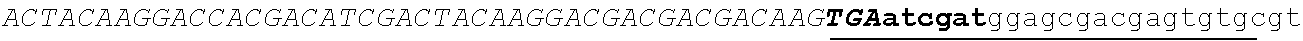

gcggggctggcgggagtgggacgccctcctcgctcctctctgttctgaacggaacaatcggccaccccgcgctacgcgccacgcatc
gagcaacgaagaaaaccccccgatgataggttgcggtggctgccgggatatagatccggccgcacatcaaagggcccctccgcca
gagaagaagctcctttcccagcagactccttctgctgccaaaacacttctctgtccacagcaacaccaaaggatgaacagatcaact
tgcgtctccgcgtagcttcctcggctagcgtgcttgcaacaggtccctgcactattatcttcctgctttcctctgaattatgcggcaggc
gagcgctcgctctggcgagcgctccttcgcgccgccctcgctgatcgagtgtacagtcaatgaatggtgagctccgcgtctcgaaca
gagcgcgcagaggaacgctgaaggtctcgcctctgtcgcacctcagcgcggcatacaccacaataaccacctgacgaatgcgcttg
gttcttcgtccattagcgaagcgtccggttcacacacgtgccacgttggcgaggtggcaggtgacaatgatcggtggagctgatggt

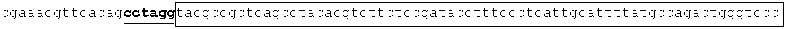

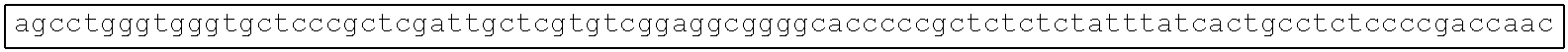

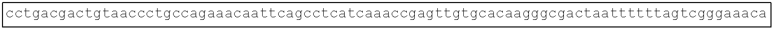

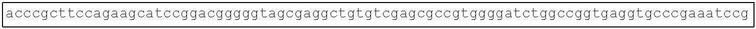

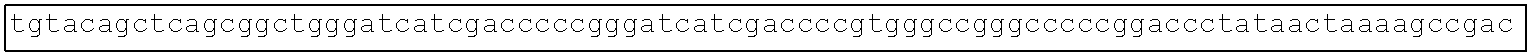

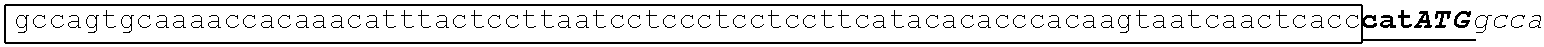

tcgccgccgccgccgtgatcgtgcccctgggcctgctgttcttcatctccggcctggtggtgaacctgatccaggccctgtgcttcg
tgctgatccgccccctgtccaagaacacctaccgcaagatcaaccgcgtggtggccgagctgctgtggctggagctgatctggc
tggtggactggtgggccggcgtgaagatcaaggtgttcatggaccccgagtccttcaacctgatgggcaaggagcacgccct
ggtggtggccaaccaccgctccgacatcgactggctggtgggctggctgctggcccagcgctccggctgcctgggctccgccct
ggccgtgatgaagaagtcctccaagttcctgcccgtgatcggctggtccatgtggttctccgagtacctgttcctggagcgctcct
gggccaaggacgagaacaccctgaaggccggcctgcagcgcctgaaggacttcccccgccccttctggctggccttcttcgtg
gagggcacccgcttcacccaggccaagttcctggccgcccaggagtacgccgcctcccagggcctgcccatcccccgcaacgt
gctgatcccccgcaccaagggcttcgtgtccgccgtgtcccacatgcgctccttcgtgcccgccatctacgacatgaccgtggcc
atccccaagtcctccccctcccccaccatgctgcgcctgttcaagggccagccctccgtggtgcacgtgcacatcaagcgctgcct
gatgaaggagctgcccgagaccgacgaggccgtggcccagtggtgcaaggacatgttcgtggagaaggacaagctgctgg
acaagcacatcgccgaggacaccttctccgaccagcccatgcaggacctgggccgccccatcaagtccctgctggtggtggcc
tcctgggcctgcctgatggcctacggcgccctgaagttcctgcagtgctcctccctgctgtcctcctggaagggcatcgccttcttc
ctggtgggcctggccatcgtgaccatcctgatgcacatcctgatcctgttctcccagtccgagcgctccacccccgccaaggtgg

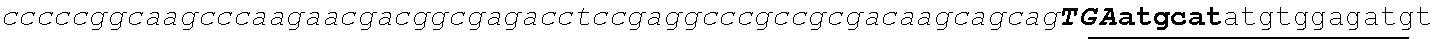

agggtggtcgactcgttggaggtgggtgtttttttttatcgagtgcgcggcgcggcaaacgggtccctttttatcgaggtgttcccaac
gccgcaccgccctcttaaaacaacccccaccaccacttgtcgaccttctcgtttgttatccgccacggcgccccggaggggcgtcgtc
tggccgcgcgggcagctgtatcgccgcgctcgctccaatggtgtgtaatcttggaaagataataatcgatggatgaggaggagagc
gtgggagatcagagcaaggaatatacagttggcacgaagcagcagcgtactaagctgtagcgtgttaagaaagaaaaactcgctg
ttaggctgtattaatcaaggagcgtatcaataattaccgaccctatacctttatctccaacccaatcgcggcttaaggatctaagtaa
gattcgaagcgctcgaccgtgccggacggactgcagccccatgtcgtagtgaccgccaatgtaagtgggctggcgtttccctgtacg
tgagtcaacgtcactgcacgcgcaccaccctctcgaccggcaggaccaggcatcgcgagatacagcgcgagccagacacggagtg

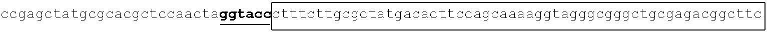

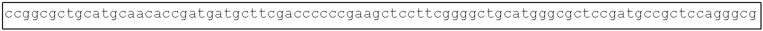

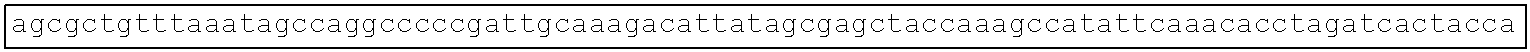

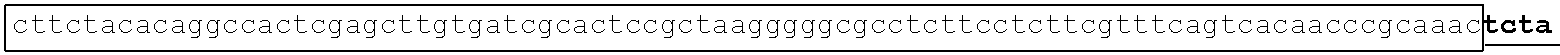

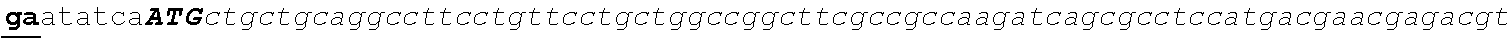

ccgaccgccccctggtgcacttcacccccaacaagggctggatgaacgaccccaacggcctgtggtacgacgagaaggacgc
caagtggcacctgtacttccagtacaacccgaacgacaccgtctgggggacgcccttgttctggggccacgccacgtccgacg
acctgaccaactgggaggaccagcccatcgccatcgccccgaagcgcaacgactccggcgccttctccggctccatggtggtg
gactacaacaacacctccggcttcttcaacgacaccatcgacccgcgccagcgctgcgtggccatctggacctacaacaccccg
gagtccgaggagcagtacatctcctacagcctggacggcggctacaccttcaccgagtaccagaagaaccccgtgctggccg
ccaactccacccagttccgcgacccgaaggtcttctggtacgagccctcccagaagtggatcatgaccgcggccaagtcccag
gactacaagatcgagatctactcctccgacgacctgaagtcctggaagctggagtccgcgttcgccaacgagggcttcctcgg
ctaccagtacgagtgccccggcctgatcgaggtccccaccgagcaggaccccagcaagtcctactgggtgatgttcatctccat
caaccccggcgccccggccggcggctccttcaaccagtacttcgtcggcagcttcaacggcacccacttcgaggccttcgacaa
ccagtcccgcgtggtggacttcggcaaggactactacgccctgcagaccttcttcaacaccgacccgacctacgggagcgccct
gggcatcgcgtgggcctccaactgggagtactccgccttcgtgcccaccaacccctggcgctcctccatgtccctcgtgcgcaag
ttctccctcaacaccgagtaccaggccaacccggagacggagctgatcaacctgaaggccgagccgatcctgaacatcagca
acgccggcccctggagccggttcgccaccaacaccacgttgacgaaggccaacagctacaacgtcgacctgtccaacagcac
cggcaccctggagttcgagctggtgtacgccgtcaacaccacccagacgatctccaagtccgtgttcgcggacctctccctctgg
ttcaagggcctggaggaccccgaggagtacctccgcatgggcttcgaggtgtccgcgtcctccttcttcctggaccgcgggaac
agcaaggtgaagttcgtgaaggagaacccctacttcaccaaccgcatgagcgtgaacaaccagcccttcaagagcgagaac
gacctgtcctactacaaggtgtacggcttgctggaccagaacatcctggagctgtacttcaacgacggcgacgtcgtgtccacc
aacacctacttcatgaccaccgggaacgccctgggctccgtgaacatgacgacgggggtggacaacctgttctacatcgaca

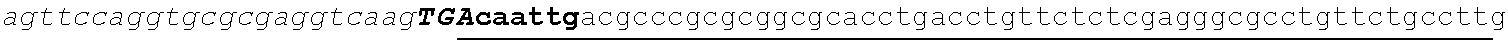

cgaaacaagcccctggagcatgcgtgcatgatcgtctctggcgccccgccgcgcggtttgtcgccctcgcgggcgccgcggccgcg
ggggcgcattgaaattgttgcaaaccccacctgacagattgagggcccaggcaggaaggcgttgagatggaggtacaggagtcaa
gtaactgaaagtttttatgataactaacaacaaagggtcgtttctggccagcgaatgacaagaacaagattccacatttccgtgtag
aggcttgccatcgaatgtgagcgggcgggccgcggacccgacaaaacccttacgacgtggtaagaaaaacgtggcgggcactgtc
cctgtagcctgaagaccagcaggagacgatcggaagcatcacagcacaggatcctgaggacagggtggttggctggatggggaa
acgctggtcgcgggattcgatcctgctgcttatatcctccctggaagcacacccacgactctgaagaagaaaacgtgcacacaca
caacccaaccggccgaatatttgcttccttatcccgggtccaagagagactgcgatgcccccctcaatcagcatcctcctccctgcc
gcttcaatcttccctgcttgcctgcgcccgcggtgcgccgtctgcccgcccagtcagtcactcctgcacaggccccttgtgcgcagtg
ctcctgtaccctttaccgctccttccattctgcgaggccccctattgaatgtattcgttgcctgtgtggccaagcgggctgctgggcgc
gccgccgtcgggcagtgctcggcgactttggcggaagccgattgttcttctgtaagccacgcgcttgctgctttgggaagagaagg
gggggggtactgaatggatgaggaggagaaggaggggtattggtattatctgagttggggaggcagggagagttggaaaatgt
aagtggcacgacgggcaaggagaatggtgagcatgtgcatggtgatgtcgttggtcgaggacgatcctgcacgcgtgtatctgat
gtagaatacggcaatcaccctagtctacatctataccttctccgtataacgccctttccaaatgccctcccgtttctctcctattcttg
atccacatgatgaccctggcactatttcaagggctggagaagagcgtttaaac

Construct pSZ5868 was transformed into 58754. Primary transformants were clonally purified and screened under standard lipid production conditions at pH 5. Integration of pSZ5868 at the FATA-1 locus was verified by DNA blot analysis. The fatty acid profiles and lipid titers of lead strains were assayed in 50-mL shake flasks (Table 22). 58813 was selected as the lead strain for the final round of genetic engineering. As shown in Table 22 as compared to strain S8754, C16:0 decreased from 5.9% to 3.4%, and C18:0 increased from 27.3% to about 45%. C18:2 increased slightly from 1.3% to about 1.6% due to the activity of the T. cacao LPAAT.

TABLE 22
Fatty acid profiles of FATA-1 ablation strains.

| Strain | S5100 | S8754 | S8813 | S8814 |
|---|---|---|---|---|
| C14:0 | 0.7 | 0.6 | 0.5 | 0.5 |
| C16:0 | 18.8 | 5.9 | 3.4 | 3.4 |
| C16:1 cis-9 | 0.5 | 0.0 | 0.0 | 0.0 |
| C18:0 | 4.0 | 27.3 | 45.3 | 44.8 |
| C18:1 | 68.3 | 60.9 | 45.9 | 46.3 |
| C18:2 | 6.3 | 1.3 | 1.5 | 1.6 |
| C18:3 α | 0.6 | 0.3 | 0.3 | 0.3 |
| C20:0 | 0.3 | 2.4 | 2.0 | 2.1 |
| saturates | 24.2 | 37.0 | 52.0 | 51.5 |
| lipid (g/L) | 12.7 | 11.9 | 11.9 | 11.9 |

### Constructs Used for FAD2 Knockout in S8813

The high-SOS strains were generated by transformation of strain S8813 with integrative plasmid pSZ6383 (FAD2-1vA::PmLDH1-AtTHIC-PmHSP90:PmSAD2-2v2-TcDGAT1-CvNR:PmSAD2-1v3-CpSAD1tp_GarmFATA1(G108A)_FLAG-PmSAD2-1::FAD2-1vB), plasmid pSZ6384 (FAD2-1vA::PmLDH1-AtTHIC-PmHSP90:PmSAD2-2v2-TcDGAT2-CvNR:PmSAD2-1v3-CpSAD1tp_GarmFATA1(G108A)_FLAG-PmSAD2-1::FAD2-1vB), or plasmid pSZ6377 (FAD2-1vA::PmLDH1-AtTHIC-PmHSP90: PmSAD2-1v3-CpSAD1tp_GarmFATA1(G108A)_FLAG-PmSAD2-1::FAD2-1vB). These constructs targeted ablation of allele 1 of the endogenous fatty acid desaturase 2 gene (FAD2-1), and contained expression modules for a second copy of GarmFATA1(G108A), and either TcDGAT1 encoding the Theobroma cacao diacylglycerol O-acyltransferase 1 (pSZ6383) or TcDGAT2 encoding the Theobroma cacao diacylglycerol O-acyltransferase 2 (pSZ6384). Deletion of one allele of FAD2 further reduced C18:2 accumulation. Expression of GarmFATA1(G108A) stimulated C18:0-ACP hydrolysis, further increasing C18:0. TcDGAT1 and TcDGAT2 had superior specificity for transfer of C18:0 to the sn-3 position of triacylglycerides than the endogeneous DGAT, leading to an increase in C18:0 and lipid titer, and a reduction in trisaturated TAGs. The final strains had higher C18:0, lower C16:0 and lower C18:2 than their parent, S8813. The Arabidopsis thaliana THIC gene (AtTHIC) catalyzes the conversion of 5-aminoimidazole ribotide (AIR) to 4-amino-5-hydroxymethylpyrimidine (HMP), providing the pyrimidine ring structure for the biosynthesis of thiamine. AtTHIC served as a selectable marker as part of plasmids pSZ6383 and pSZ6384, allowing the strains to grow in the absence of exogenous thiamine.

The sequence of the pSZ6383 transforming DNA is provided below. Relevant restriction sites in the construct are indicated in lowercase, bold and underlined text, and are 5′-3′ BspQI, KpnI, XbaI, SnaBI, BamHI, AvrII, SpeI, ClaI, AflII, EcoRI, SpeI, AscI, ClaI, SacI and BspQ I, respectively. BspQI sites delimit the 5′ and 3′ ends of the transforming DNA. Proceeding in the 5′ to 3′ direction, bold, lowercase sequences represent FAD2-1 5′ genomic DNA that permits targeted integration at the FAD2-1 locus via homologous recombination. The P. moriformis LDH1 promoter driving the expression of the Arabidopsis thaliana THIC gene is indicated by boxed text. The initiator ATG and terminator TGA for AtTHIC are indicated by uppercase, bold italics while the coding region is indicated in lowercase italics. The P. moriformis HSP90 3′ UTR is indicated by lowercase underlined text. A spacer sequence is represented by lowercase text. The P. moriformis SAD2-2 promoter, driving expression of the TcDGAT1 sequence is indicated by boxed text. The initiator ATG and terminator TGA codons of the TcDGAT1 gene are indicated by uppercase, bold italics, while the remainder of the coding region is represented with italics. Lowercase underlined text represents the C. vulgaris NR 3′ UTR. A second spacer sequence is represented by lowercase text. The P. moriformis SAD2-1 promoter, indicated by boxed italicized text, is utilized to drive the expression of the G. mangostana FATA1 gene. The initiator ATG of the sequence encoding the C. protothecoides SAD1 transit peptide (CpSAD1tp) is indicated by uppercase, bold italics, and the remainder of the CpSAD1tp sequence located between the ATG and the AscI site is indicated with lowercase, underlined italics. The GarmFATA1(G108A) coding region is indicated by lowercase italics. A sequence encoding a 3× FLAG tag fused to the C-terminus of GarmFATA1(G108A) is represented by uppercase italics, and the TGA terminator codon is indicated with uppercase, bold italics. The P. moriformis SAD2-1 3′ UTR is indicated by lowercase underlined text. The FAD2-1 3′ genomic region is indicated by bold, lowercase text.

SEQ ID NO: 128 Nucleotide sequence of transforming DNA contained in
pSZ6383
gctcttcgcgaaggtcattttccagaacaacgaccatggcttgtcttagcgatcgctcgaatgactgctagtgagtcgtacgctcga
cccagtcgctcgcaggagaacgcggcaactgccgagcttcggcttgccagtcgtgactcgtatgtgatcaggaatcattggcattg
gtagcattataattcggcttccgcgctgtttatgggcatggcaatgtctcatgcagtcgaccttagtcaaccaattctgggtggccag
ctccgggcgaccgggctccgtgtcgccgggcaccacctcctgccatgagtaacagggccgccctctcctcccgacgttggccaact
gaataccgtgtcttggggccctacatgatgggctgcctagtcgggcgggacgcgcaactgcccgcgcaatctgggacgtggtctga
atcctccaggcgggtttccccgagaaagaaagggtgccgatttcaaagcagagccatgtgccgggccctgtggcctgtgttggcgc
ctatgtagtcaccccccctcacccaattgtcgccagtttgcgcaatccataaactcaaaactgcagcttctgagctgcgctgttcaa

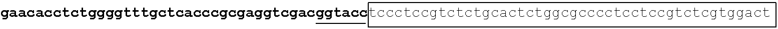

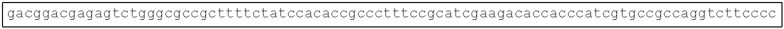

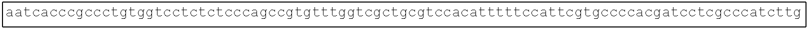

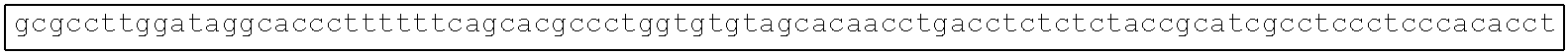

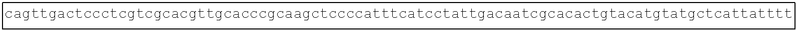

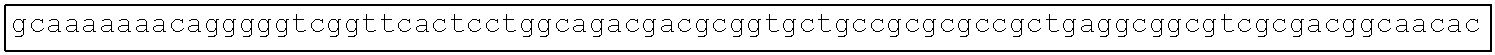

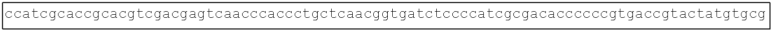

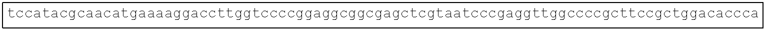

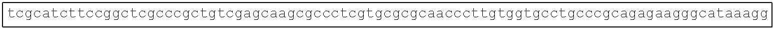

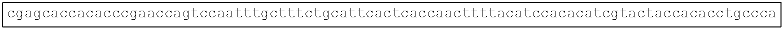

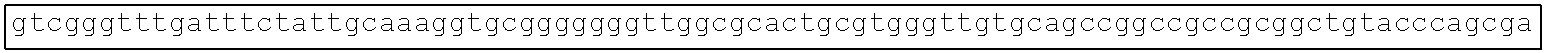

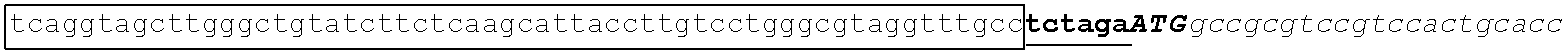

ctgatgtccgtggtctgcaacaacaagaaccactccgcccgccccaagctgcccaactcctccctgctgcccggcttcgacgtgg
tggtccaggccgcggccacccgcttcaagaaggagacgacgaccacccgcgccacgctgacgttcgacccccccacgaccaa
ctccgagcgcgccaagcagcgcaagcacaccatcgacccctcctcccccgacttccagcccatcccctccttcgaggagtgcttc
cccaagtccacgaaggagcacaaggaggtggtgcacgaggagtccggccacgtcctgaaggtgcccttccgccgcgtgcac
ctgtccggcggcgagcccgccttcgacaactacgacacgtccggcccccagaacgtcaacgcccacatcggcctggcgaagct
gcgcaaggagtggatcgaccgccgcgagaagctgggcacgccccgctacacgcagatgtactacgcgaagcagggcatcat
cacggaggagatgctgtactgcgcgacgcgcgagaagctggaccccgagttcgtccgctccgaggtcgcgcggggccgcgc
catcatcccctccaacaagaagcacctggagctggagcccatgatcgtgggccgcaagttcctggtgaaggtgaacgcgaac
atcggcaactccgccgtggcctcctccatcgaggaggaggtctacaaggtgcagtgggccaccatgtggggcgccgacacca
tcatggacctgtccacgggccgccacatccacgagacgcgcgagtggatcctgcgcaactccgcggtccccgtgggcaccgtc
cccatctaccaggcgctggagaaggtggacggcatcgcggagaacctgaactgggaggtgttccgcgagacgctgatcgag
caggccgagcagggcgtggactacttcacgatccacgcgggcgtgctgctgcgctacatccccctgaccgccaagcgcctgac
gggcatcgtgtcccgcggcggctccatccacgcgaagtggtgcctggcctaccacaaggagaacttcgcctacgagcactggg
acgacatcctggacatctgcaaccagtacgacgtcgccctgtccatcggcgacggcctgcgccccggctccatctacgacgcca
acgacacggcccagttcgccgagctgctgacccagggcgagctgacgcgccgcgcgtgggagaaggacgtgcaggtgatg
aacgagggccccggccacgtgcccatgcacaagatccccgagaacatgcagaagcagctggagtggtgcaacgaggcgcc
cttctacaccctgggccccctgacgaccgacatcgcgcccggctacgaccacatcacctccgccatcggcgcggccaacatcgg
cgccctgggcaccgccctgctgtgctacgtgacgcccaaggagcacctgggcctgcccaaccgcgacgacgtgaaggcgggc
gtcatcgcctacaagatcgccgcccacgcggccgacctggccaagcagcacccccacgcccaggcgtgggacgacgcgctgt
ccaaggcgcgcttcgagttccgctggatggaccagttcgcgctgtccctggaccccatgacggcgatgtccttccacgacgaga
cgctgcccgcggacggcgcgaaggtcgcccacttctgctccatgtgcggccccaagttctgctccatgaagatcacggaggac
atccgcaagtacgccgaggagaacggctacggctccgccgaggaggccatccgccagggcatggacgccatgtccgagga
gttcaacatcgccaagaagacgatctccggcgagcagcacggcgaggtcggcggcgagatctacctgcccgagtcctacgtc

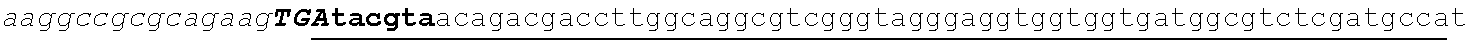

cgcacgcatccaacgaccgtatacgcatcgtccaatgaccgtcggtgtcctctctgcctccgttttgtgagatgtctcaggcttggtgc
atcctcgggtggccagccacgttgcgcgtcgtgctgcttgcctctcttgcgcctctgtggtactggaaaatatcatcgaggcccgttttt
ttgctcccatttcctttccgctacatcttgaaagcaaacgacaaacgaagcagcaagcaaagagcacgaggacggtgaacaagtct
gtcacctgtatacatctatttccccgcgggtgcacctactctctctcctgccccggcagagtcagctgccttacgtgacggatcccgcg
tctcgaacagagcgcgcagaggaacgctgaaggtctcgcctctgtcgcacctcagcgcggcatacaccacaataaccacctgacga
atgcgcttggttcttcgtccattagcgaagcgtccggttcacacacgtgccacgttggcgaggtggcaggtgacaatgatcggtgga

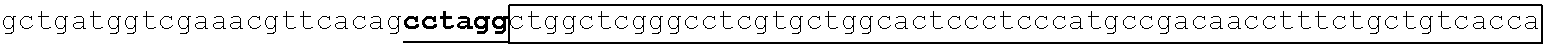

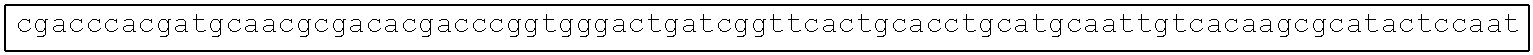

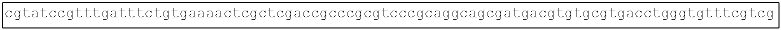

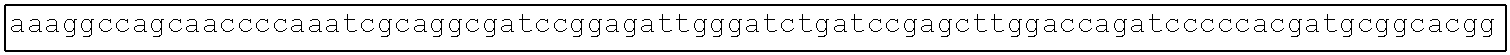

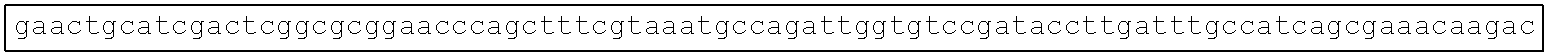

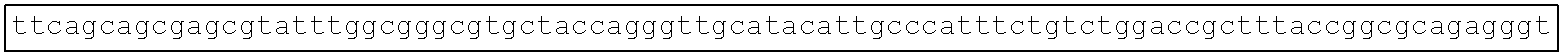

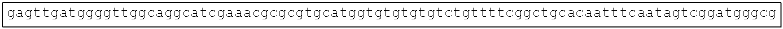

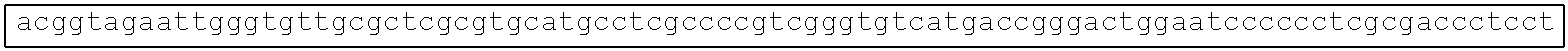

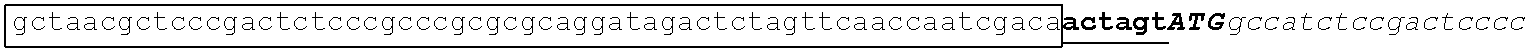

cgagatcctgggctccaccgccaccgtgacctcctcctcccactccgactccgacctgaacctgctgtccatccgccgccgcacct
ccaccaccgccgccgcccgcgcccccgaccgcgacgactccggcaacggcgaggccgtggacgaccgcgaccgcgtggagt
ccgccaacctgatgtccaacgtggccgagaacgccaacgagatgcccaactcctccgacacccgcttcacctaccgcccccgcg
tgcccgcccaccgccgcatcaaggagtcccccctgtcctccggcgccatcttcaagcagtcccacgccggcctgttcaacctgtgc
atcgtggtgctggtggccgtgaactcccgcctgatcatcgagaacctgatgaagtacggctggctgatccgctccggcttctggt
tctcctcccgctccctgtccgactggcccctgttcatgtgctgcctgaccctgcccatcttccccctggccgccttcgtggtggagaa
gctggtgcagcgcaactacatctccgagcccgtggtggtgttcctgcacgccatcatctccaccaccgccgtgctgtaccccgtg
atcgtgaacctgcgctgcgactccgccttcctgtccggcgtggccctgatgctgttcgcctgcatcgtgtggctgaagctggtgtc
ctacgcccacaccaacaacgacatgcgcgccctggccaagtccgccgagaagggcgacgtggacccctcctacgacgtgtcct
tcaagtccctggcctacttcatggtggcccccaccctgtgctaccagcagtcctacccccgcacccccgccgtgcgcaagtcctgg
gtggtgcgccagttcatcaagctgatcgtgttcaccggcctgatgggcttcatcatcgagcagtacatcaaccccatcgtgcag
aactcccagcaccccctgaagggcaacctgctgtacgccatcgagcgcgtgctgaagctgtccgtgcccaacctgtacgtgtgg
ctgtgcatgttctactgcttcttccacctgtggctgaacatcctggccgagctgctgcgcttcggcgaccgcgagttctacaagga
ctggtggaacgccaagaccgtggaggagtactggcgcatgtggaacatgcccgtgcacaagtggatggtgcgccacatctac
ttcccctgcctgcgcaacggcatccccaagggcgtggccatcgtgatcgccttcctggtgtccgccgtgttccacgagctgtgcat
cgccgtgccctgccacatgttcaagctgtgggccttcatcggcatcatgttccaggtgcccctggtgctgatcaccaactacctgc
aggacaagttccgctcctccatggtgggcaacatgatcttctggttcatcttctccatcctgggccagcccatgtgcgtgctgctgt

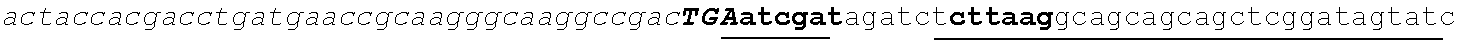

gacacactctggacgctggtcgtgtgatggactgttgccgccacacttgctgccttgacctgtgaatatccctgccgcttttatcaaac
agcctcagtgtgtttgatcttgtgtgtacgcgcttttgcgagttgctagctgcttgtgctatttgcgaataccacccccagcatccccttc
cctcgtttcatatcgcttgcatcccaaccgcaacttatctacgctgtcctgctatccctcagcgctgctcctgctcctgctcactgcccct
cgcacagccttggtttgggctccgcctgtattctcctggtactgcaacctgtaaaccagcactgcaatgctgatgcacgggaagtagt
gggatgggaacacaaatggacttaaggatctaagtaagattcgaagcgctcgaccgtgccggacggactgcagccccatgtcgta
gtgaccgccaatgtaagtgggctggcgtttccctgtacgtgagtcaacgtcactgcacgcgcaccaccctctcgaccggcaggacca
ggcatcgcgagatacagcgcgagccagacacggagtgccgagctatgcgcacgctccaactagatatcatgtggatgatgagcat

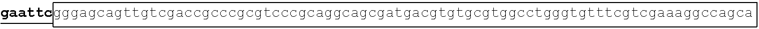

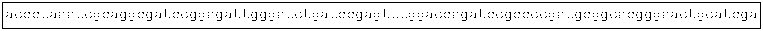

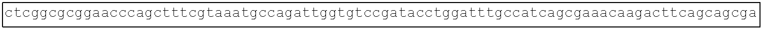

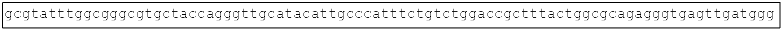

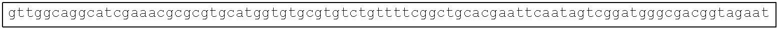

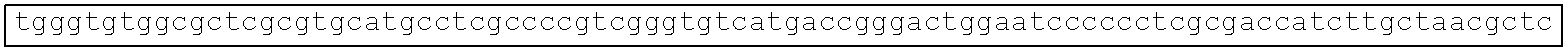

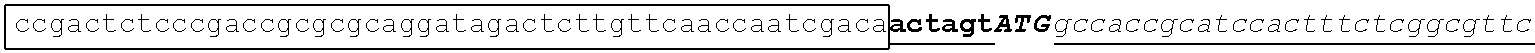

aatgcccgctgcggcgacctgcgtcgctcggcgggctccgggccccggcgcccagcgaggcccctccccgtgcgcgggcgcgc
catccccccccgcatcatcgtggtgtcctcctcctcctccaaggtgaaccccctgaagaccgaggccgtggtgtcctccggcctgg
ccgaccgcctgcgcctgggctccctgaccgaggacggcctgtcctacaaggagaagttcatcgtgcgctgctacgaggtgggc
atcaacaagaccgccaccgtggagaccatcgccaacctgctgcaggaggtgggctgcaaccacgcccagtccgtgggctact
ccaccgccggcttctccaccacccccaccatgcgcaagctgcgcctgatctgggtgaccgcccgcatgcacatcgagatctaca
agtaccccgcctggtccgacgtggtggagatcgagtcctggggccagggcgagggcaagatcggcacccgccgcgactgga
tcctgcgcgactacgccaccggccaggtgatcggccgcgccacctccaagtgggtgatgatgaaccaggacacccgccgcctg
cagaaggtggacgtggacgtgcgcgacgagtacctggtgcactgcccccgcgagctgcgcctggccttccccgaggagaaca
actcctccctgaagaagatctccaagctggaggacccctcccagtactccaagctgggcctggtgccccgccgcgccgacctgg
acatgaaccagcacgtgaacaacgtgacctacatcggctgggtgctggagtccatgccccaggagatcatcgacacccacga
gctgcagaccatcaccctggactaccgccgcgagtgccagcacgacgacgtggtggactccctgacctcccccgagccctccg
aggacgccgaggccgtgttcaaccacaacggcaccaacggctccgccaacgtgtccgccaacgaccacggctgccgcaactt
cctgcacctgctgcgcctgtccggcaacggcctggagatcaaccgcggccgcaccgagtggcgcaagaagcccacccgcAT
GGACTACAAGGACCACGACGGCGACTACAAGGACCACGACATCGACTACAAGGACGACGACGACAA

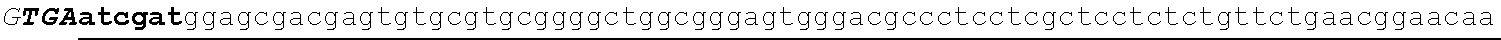

tcggccaccccgcgctacgcgccacgcatcgagcaacgaagaaaaccccccgatgataggttgcggtggctgccgggatatagat
ccggccgcacatcaaagggcccctccgccagagaagaagctcctttcccagcagactccttctgctgccaaaacacttctctgtcca
cagcaacaccaaaggatgaacagatcaacttgcgtctccgcgtagcttcctcggctagcgtgcttgcaacaggtccctgcactattat
cttcctgctttcctctgaattatgcggcaggcgagcgctcgctctggcgagcgctccttcgcgccgccctcgctgatcgagtgtacagt
caatgaatggtgagctcctcactcagcgcgcctgcgcggggatgcggaacgccgccgccgccttgtcttttgcacgcgcgactccgt
cgcttcgcgggtggcacccccattgaaaaaaacctcaattctgtttgtggaagacacggtgtacccccaaccacccacctgcacct
ctattattggtattattgacgcgggagcgggcgttgtactctacaacgtagcgtctctggttttcagctggctcccaccattgtaaatt
cttgctaaaatagtgcgtggttatgtgagaggtatggtgtaacagggcgtcagtcatgttggttttcgtgctgatctcgggcacaag
gcgtcgtcgacgtgacgtgcccgtgatgagagcaataccgcgctcaaagccgacgcatggcctttactccgcactccaaacgact
gtcgctcgtatttttcggatatctattttttaagagcgagcacagcgccgggcatgggcctgaaaggcctcgcggccgtgctcgtgg
tgggggccgcgagcgcgtggggcatcgcggcagtgcaccaggcgcagacggaggaacgcatggtgagtgcgcatcacaagatg
catgtcttgttgtctgtactataatgctagagcatcaccaggggcttagtcatcgcacctgctttggtcattacagaaattgcacaag
ggcgtcctccgggatgaggagatgtaccagctcaagctggagcggcttcgagccaagcaggagcgcggcgcatgacgacctacc
cacatgcgaagagc

The sequence of the pSZ6384 transforming DNA is provided below. Relevant restriction sites in the construct are indicated in lowercase, bold and underlined text, and are 5′-3′ BspQI, KpnI, XbaI, SnaBI, BamHI, AvrII, SpeI, ClaI, AflII, EcoRI, SpeI, AscI, ClaI, SacI and BspQ I, respectively. BspQI sites delimit the 5′ and 3′ ends of the transforming DNA. Proceeding in the 5′ to 3′ direction, bold, lowercase sequences represent FAD2-1 5′ genomic DNA that permits targeted integration at the FAD2-1 locus via homologous recombination. The P. moriformis LDH1 promoter driving the expression of the Arabidopsis thaliana THIC gene is indicated by boxed text. The initiator ATG and terminator TGA for AtTHIC are indicated by uppercase, bold italics while the coding region is indicated in lowercase italics. The P. moriformis HSP90 3′ UTR is indicated by lowercase underlined text. A spacer sequence is represented by lowercase text. The P. moriformis SAD2-2 promoter, driving expression of the TcDGAT2 sequence is indicated by boxed text. The initiator ATG and terminator TGA codons of the TcDGAT2 gene are indicated by uppercase, bold italics, while the remainder of the coding region is represented with italics. Lowercase underlined text represents the C. vulgaris NR 3′ UTR. A second spacer sequence is represented by lowercase text. The P. moriformis SAD2-1 promoter, indicated by boxed italicized text, is utilized to drive the expression of the G. mangostana FATA1 gene. The initiator ATG of the sequence encoding the C. protothecoides SAD1 transit peptide (CpSAD1tp) is indicated by uppercase, bold italics, and the remainder of the CpSAD1tp sequence located between the ATG and the AscI site is indicated with lowercase, underlined italics. The GarmFATA1(G108A) coding region is indicated by lowercase italics. A sequence encoding a 3× FLAG tag fused to the C-terminus of GarmFATA1(G108A) is represented by uppercase italics, and the TGA terminator codon is indicated with uppercase, bold italics. The P. moriformis SAD2-1 3′ UTR is indicated by lowercase underlined text. The FAD2-1 3′ genomic region is indicated by bold, lowercase text.

Nucleotide sequence of transforming DNA contained in pSZ6384
SEQ ID NO: 129

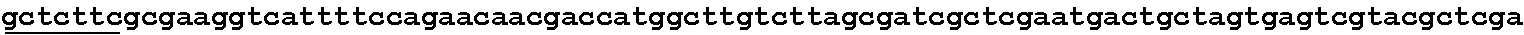

cccagtcgctcgcaggagaacgcggcaactgccgagcttcggcttgccagtcgtgactcgtatgtgatcaggaatcattggcattg
gtagcattataattcggcttccgcgctgtttatgggcatggcaatgtctcatgcagtcgaccttagtcaaccaattctgggtggccag
ctccgggcgaccgggctccgtgtcgccgggcaccacctcctgccatgagtaacagggccgccctctcctcccgacgttggccaact
gaataccgtgtcttggggccctacatgatgggctgcctagtcgggcgggacgcgcaactgcccgcgcaatctgggacgtggtctga
atcctccaggcgggtttccccgagaaagaaagggtgccgatttcaaagcagagccatgtgccgggccctgtggcctgtgttggcgc
ctatgtagtcaccccccctcacccaattgtcgccagtttgcgcaatccataaactcaaaactgcagcttctgagctgcgctgttcaa

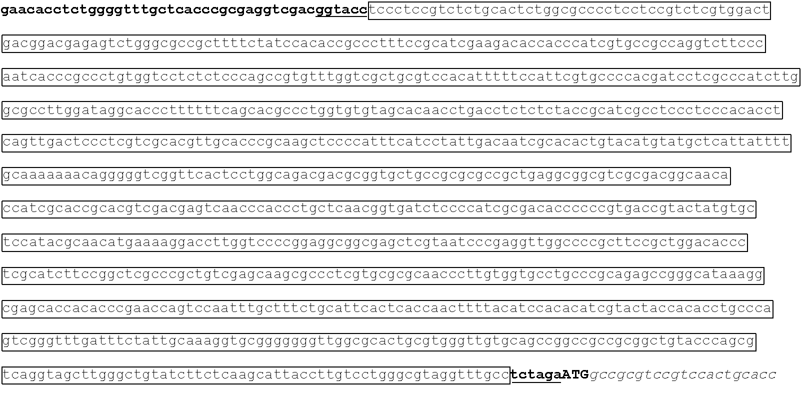

ctgatgtccgtggtctgcaacaacaagaaccactccgcccgccccaagctgcccaactcctccctgctgcccggcttcgacgtgg
tggtccaggccgcggccacccgcttcaagaaggagacgacgaccacccgcgccacgctgacgttcgacccccccacgaccaa
ctccgagcgcgccaagcagcgcaagcacaccatcgacccctcctcccccgacttccagcccatcccctccttcgaggagtgcttc
cccaagtccacgaaggagcacaaggaggtggtgcacgaggagtccggccacgtcctgaaggtgcccttccgccgcgtgcac
ctgtccggcggcgagcccgccttcgacaactacgacacgtccggcccccagaacgtcaacgcccacatcggcctggcgaagct
gcgcaaggagtggatcgaccgccgcgagaagctgggcacgccccgctacacgcagatgtactacgcgaagcagggcatcat
cacggaggagatgctgtactgcgcgacgcgcgagaagctggaccccgagttcgtccgctccgaggtcgcgcggggccgcgc
catcatcccctccaacaagaagcacctggagctggagcccatgatcgtgggccgcaagttcctggtgaaggtgaacgcgaac
atcggcaactccgccgtggcctcctccatcgaggaggaggtctacaaggtgcagtgggccaccatgtggggcgccgacacca
tcatggacctgtccacgggccgccacatccacgagacgcgcgagtggatcctgcgcaactccgcggtccccgtgggcaccgtc
cccatctaccaggcgctggagaaggtggacggcatcgcggagaacctgaactgggaggtgttccgcgagacgctgatcgag
caggccgagcagggcgtggactacttcacgatccacgcgggcgtgctgctgcgctacatccccctgaccgccaagcgcctgac
gggcatcgtgtcccgcggcggctccatccacgcgaagtggtgcctggcctaccacaaggagaacttcgcctacgagcactggg
acgacatcctggacatctgcaaccagtacgacgtcgccctgtccatcggcgacggcctgcgccccggctccatctacgacgcca
acgacacggcccagttcgccgagctgctgacccagggcgagctgacgcgccgcgcgtgggagaaggacgtgcaggtgatg
aacgagggccccggccacgtgcccatgcacaagatccccgagaacatgcagaagcagctggagtggtgcaacgaggcgcc
cttctacaccctgggccccctgacgaccgacatcgcgcccggctacgaccacatcacctccgccatcggcgcggccaacatcgg
cgccctgggcaccgccctgctgtgctacgtgacgcccaaggagcacctgggcctgcccaaccgcgacgacgtgaaggcgggc
gtcatcgcctacaagatcgccgcccacgcggccgacctggccaagcagcacccccacgcccaggcgtgggacgacgcgctgt
ccaaggcgcgcttcgagttccgctggatggaccagttcgcgctgtccctggaccccatgacggcgatgtccttccacgacgaga
cgctgcccgcggacggcgcgaaggtcgcccacttctgctccatgtgcggccccaagttctgctccatgaagatcacggaggac
atccgcaagtacgccgaggagaacggctacggctccgccgaggaggccatccgccagggcatggacgccatgtccgagga
gttcaacatcgccaagaagacgatctccggcgagcagcacggcgaggtcggcggcgagatctacctgcccgagtcctacgtc

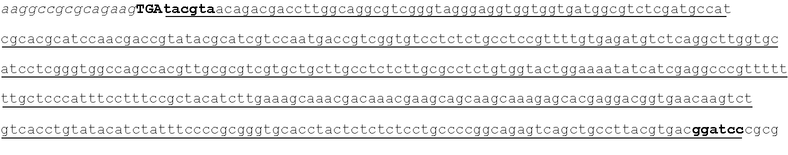

tctcgaacagagcgcgcagaggaacgctgaaggtctcgcctctgtcgcacctcagcgcggcatacaccacaataaccacctgacga
atgcgcttggttcttcgtccattagcgaagcgtccggttcacacacgtgccacgttggcgaggtggcaggtgacaatgatcggtgga

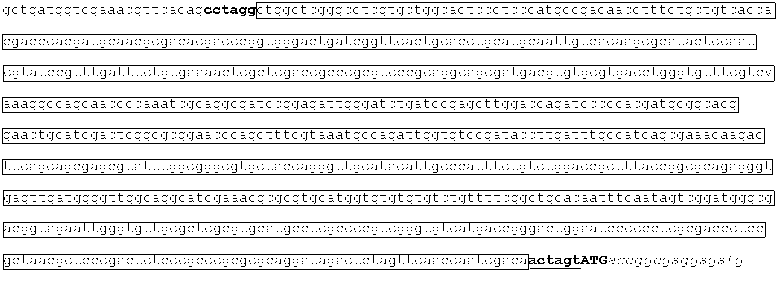

gaggagcgcaaggccaccggctaccgcgagttctccggccgccacgagttcccctccaacaccatgcacgccctgctggccat
gggcatctggctgggcgccatccacttcaacgccctgctgctgctgttctccttcctgttcctgcccttctccaagttcctggtggtgt
tcggcctgctgctgctgttcatgatcctgcccatcgacccctactccaagttcggccgccgcctgtcccgctacatctccaagcacg
cctgctcctacttccccatcaccctgcacgtggaggacatccacgccttccaccccgaccgcgcctacgtgttcggcttcgagccc
cactccgtgctgcccatcggcgtggtggccctggccgacctgaccggcttcatgcccctgcccaagatcaaggtgctggcctcct
ccgccgtgttctacacccccttcctgcgccacatctggacctggctgggcctgacccccgccaccaagaagaacttctcctccctg
ctggacgccggctactcctgcatcctggtgcccggcggcgtgcaggagaccttccacatggagcccggctccgagatcgccttc
ctgcgcgcccgccgcggcttcgtgcgcatcgccatggagatgggctcccccctggtgcccgtgttctgcttcggccagtcccacgt
gtacaagtggtggaagcccggcggcaagttctacctgcagttctcccgcgccatcaagttcacccccatcttcttctggggcatct
tcggctcccccctgccctaccagcaccccatgcacgtggtggtgggcaagcccatcgacgtgaagaagaacccccagcccatc
gtggaggaggtgatcgaggtgcacgaccgcttcgtggaggccctgcaggacctgttcgagcgccacaaggcccaggtgggc

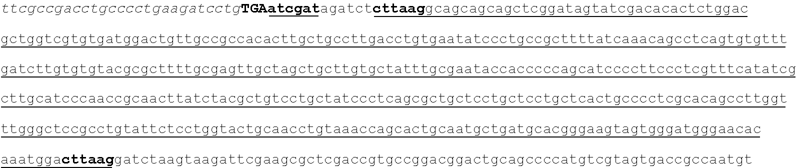

aagtgggctggcgtttccctgtacgtgagtcaacgtcactgcacgcgcaccaccctctcgaccggcaggaccaggcatcgcgagat

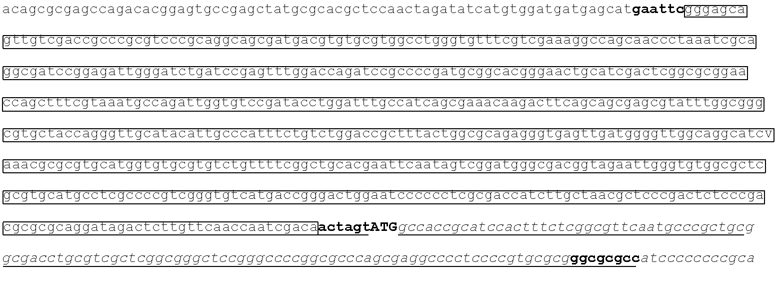

tcatcgtggtgtcctcctcctcctccaaggtgaaccccctgaagaccgaggccgtggtgtcctccggcctggccgaccgcctgcg
cctgggctccctgaccgaggacggcctgtcctacaaggagaagttcatcgtgcgctgctacgaggtgggcatcaacaagacc
gccaccgtggagaccatcgccaacctgctgcaggaggtgggctgcaaccacgcccagtccgtgggctactccaccgccggctt
ctccaccacccccaccatgcgcaagctgcgcctgatctgggtgaccgcccgcatgcacatcgagatctacaagtaccccgcctg
gtccgacgtggtggagatcgagtcctggggccagggcgagggcaagatcggcacccgccgcgactggatcctgcgcgacta
cgccaccggccaggtgatcggccgcgccacctccaagtgggtgatgatgaaccaggacacccgccgcctgcagaaggtgga
cgtggacgtgcgcgacgagtacctggtgcactgcccccgcgagctgcgcctggccttccccgaggagaacaactcctccctga
agaagatctccaagctggaggacccctcccagtactccaagctgggcctggtgccccgccgcgccgacctggacatgaacca
gcacgtgaacaacgtgacctacatcggctgggtgctggagtccatgccccaggagatcatcgacacccacgagctgcagacc
atcaccctggactaccgccgcgagtgccagcacgacgacgtggtggactccctgacctcccccgagccctccgaggacgccga
ggccgtgttcaaccacaacggcaccaacggctccgccaacgtgtccgccaacgaccacggctgccgcaacttcctgcacctgct
gcgcctgtccggcaacggcctggagatcaaccgcggccgcaccgagtggcgcaagaagcccacccgcATGGACTACAA

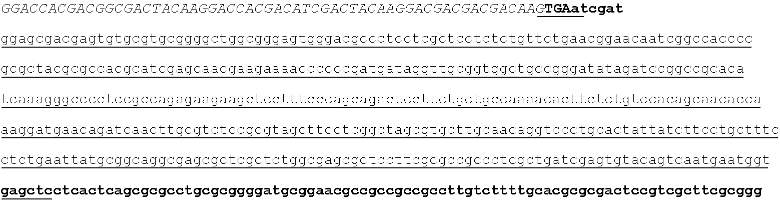

tggcacccccattgaaaaaaacctcaattctgtttgtggaagacacggtgtacccccaaccacccacctgcacctctattattggta
ttattgacgcgggagcgggcgttgtactctacaacgtagcgtctctggttttcagctggctcccaccattgtaaattcttgctaaaat
agtgcgtggttatgtgagaggtatggtgtaacagggcgtcagtcatgttggttttcgtgctgatctcgggcacaaggcgtcgtcgac
gtgacgtgcccgtgatgagagcaataccgcgctcaaagccgacgcatggcctttactccgcactccaaacgactgtcgctcgtatt
tttcggatatctattttttaagagcgagcacagcgccgggcatgggcctgaaaggcctcgcggccgtgctcgtggtgggggccgcg
agcgcgtggggcatcgcggcagtgcaccaggcgcagacggaggaacgcatggtgagtgcgcatcacaagatgcatgtcttgttg
tctgtactataatgctagagcatcaccaggggcttagtcatcgcacctgctttggtcattacagaaattgcacaagggcgtcctccg

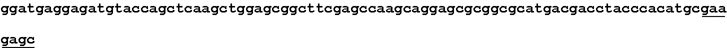

The sequence of the pSZ6377 transforming DNA is provided below. Relevant restriction sites in the construct are indicated in lowercase, bold and underlined text, and are 5′-3′ BspQI, KpnI, XbaI, SnaBI, BamHI, AvrII, SpeI, AscI, ClaI, SacI and BspQ I, respectively. BspQI sites delimit the 5′ and 3′ ends of the transforming DNA. Proceeding in the 5′ to 3′ direction, bold, lowercase sequences represent FAD2-1 5′ genomic DNA that permits targeted integration at the FAD2-1 locus via homologous recombination. The P. moriformis LDH1 promoter driving the expression of the Arabidopsis thaliana THIC gene is indicated by boxed text. The initiator ATG and terminator TGA for AtTHIC are indicated by uppercase, bold italics while the coding region is indicated in lowercase italics. The P. moriformis HSP90 3′ UTR is indicated by lowercase underlined text. A spacer sequence is represented by lowercase text. The P. moriformis SAD2-1 promoter, indicated by boxed italicized text, is utilized to drive the expression of the G. mangostana FATA1 gene. The initiator ATG of the sequence encoding the C. protothecoides SAD1 transit peptide (CpSAD1tp) is indicated by uppercase, bold italics, and the remainder of the CpSAD1tp sequence located between the ATG and the AscI site is indicated with lowercase, underlined italics. The GarmFATA1(G108A) coding region is indicated by lowercase italics. A sequence encoding a 3× FLAG tag fused to the C-terminus of GarmFATA1(G108A) is represented by uppercase italics, and the TGA terminator codon is indicated with uppercase, bold italics. The P. moriformis SAD2-1 3′ UTR is indicated by lowercase underlined text. The FAD2-1 3′ genomic region is indicated by bold, lowercase text.

Nucleotide sequence of transforming DNA contained in pSZ63 77
SEQ ID NO: 130
gctcttcgcgaaggtcattttccagaacaacgaccatggcttgtcttagcgatcgctcgaatgactgctagtgagtcgtacgctcga
cccagtcgctcgcaggagaacgcggcaactgccgagcttcggcttgccagtcgtgactcgtatgtgatcaggaatcattggcattg
gtagcattataattcggcttccgcgctgtttatgggcatggcaatgtctcatgcagtcgaccttagtcaaccaattctgggtggccag
ctccgggcgaccgggctccgtgtcgccgggcaccacctcctgccatgagtaacagggccgccctctcctcccgacgttggccaact
gaataccgtgtcttggggccctacatgatgggctgcctagtcgggcgggacgcgcaactgcccgcgcaatctgggacgtggtctga
atcctccaggcgggtttccccgagaaagaaagggtgccgatttcaaagcagagccatgtgccgggccctgtggcctgtgttggcgc
ctatgtagtcaccccccctcacccaattgtcgccagtttgcgcaatccataaactcaaaactgcagcttctgagctgcgctgttcaa

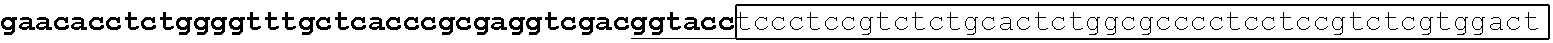

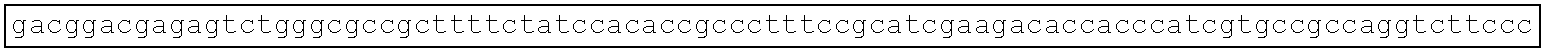

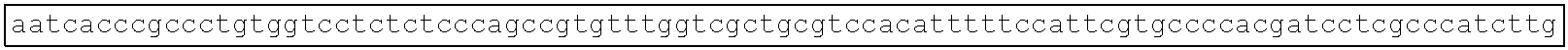

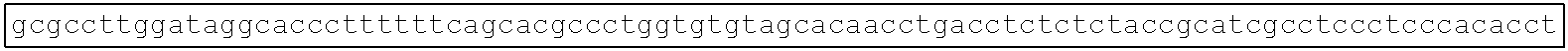

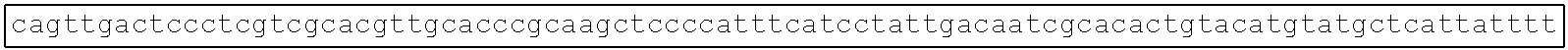

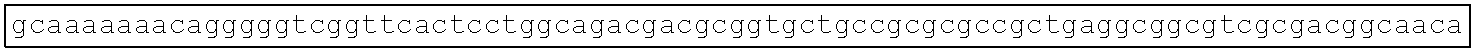

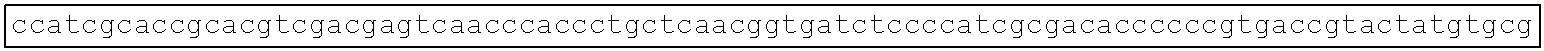

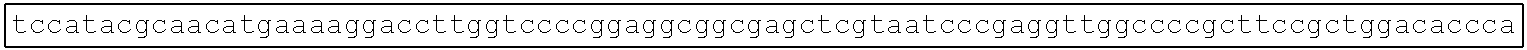

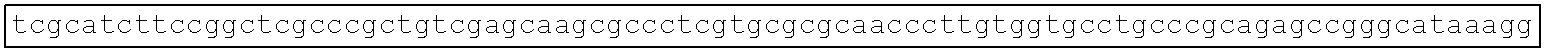

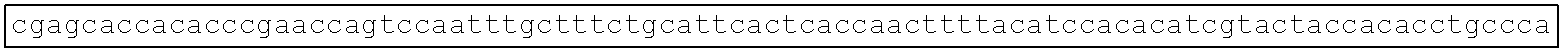

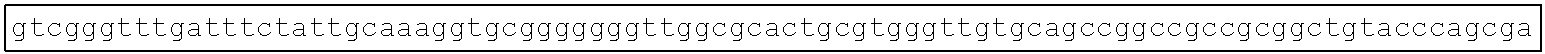

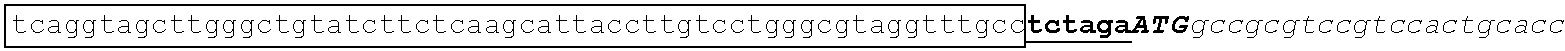

ctgatgtccgtggtctgcaacaacaagaaccactccgcccgccccaagctgcccaactcctccctgctgcccggcttcgacgtgg
tggtccaggccgcggccacccgcttcaagaaggagacgacgaccacccgcgccacgctgacgttcgacccccccacgaccaa
ctccgagcgcgccaagcagcgcaagcacaccatcgacccctcctcccccgacttccagcccatcccctccttcgaggagtgcttc
cccaagtccacgaaggagcacaaggaggtggtgcacgaggagtccggccacgtcctgaaggtgcccttccgccgcgtgcac
ctgtccggcggcgagcccgccttcgacaactacgacacgtccggcccccagaacgtcaacgcccacatcggcctggcgaagct
gcgcaaggagtggatcgaccgccgcgagaagctgggcacgccccgctacacgcagatgtactacgcgaagcagggcatcat
cacggaggagatgctgtactgcgcgacgcgcgagaagctggaccccgagttcgtccgctccgaggtcgcgcggggccgcgc
catcatcccctccaacaagaagcacctggagctggagcccatgatcgtgggccgcaagttcctggtgaaggtgaacgcgaac
atcggcaactccgccgtggcctcctccatcgaggaggaggtctacaaggtgcagtgggccaccatgtggggcgccgacacca
tcatggacctgtccacgggccgccacatccacgagacgcgcgagtggatcctgcgcaactccgcggtccccgtgggcaccgtc
cccatctaccaggcgctggagaaggtggacggcatcgcggagaacctgaactgggaggtgttccgcgagacgctgatcgag
caggccgagcagggcgtggactacttcacgatccacgcgggcgtgctgctgcgctacatccccctgaccgccaagcgcctgac
gggcatcgtgtcccgcggcggctccatccacgcgaagtggtgcctggcctaccacaaggagaacttcgcctacgagcactggg
acgacatcctggacatctgcaaccagtacgacgtcgccctgtccatcggcgacggcctgcgccccggctccatctacgacgcca
acgacacggcccagttcgccgagctgctgacccagggcgagctgacgcgccgcgcgtgggagaaggacgtgcaggtgatg
aacgagggccccggccacgtgcccatgcacaagatccccgagaacatgcagaagcagctggagtggtgcaacgaggcgcc
cttctacaccctgggccccctgacgaccgacatcgcgcccggctacgaccacatcacctccgccatcggcgcggccaacatcgg
cgccctgggcaccgccctgctgtgctacgtgacgcccaaggagcacctgggcctgcccaaccgcgacgacgtgaaggcgggc
gtcatcgcctacaagatcgccgcccacgcggccgacctggccaagcagcacccccacgcccaggcgtgggacgacgcgctgt
ccaaggcgcgcttcgagttccgctggatggaccagttcgcgctgtccctggaccccatgacggcgatgtccttccacgacgaga
cgctgcccgcggacggcgcgaaggtcgcccacttctgctccatgtgcggccccaagttctgctccatgaagatcacggaggac
atccgcaagtacgccgaggagaacggctacggctccgccgaggaggccatccgccagggcatggacgccatgtccgagga
gttcaacatcgccaagaagacgatctccggcgagcagcacggcgaggtcggcggcgagatctacctgcccgagtcctacgtc

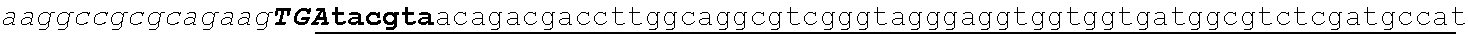

cgcacgcatccaacgaccgtatacgcatcgtccaatgaccgtcggtgtcctctctgcctccgttttgtgagatgtctcaggcttggtgc
atcctcgggtggccagccacgttgcgcgtcgtgctgcttgcctctcttgcgcctctgtggtactggaaaatatcatcgaggcccgttttt
ttgctcccatttcctttccgctacatcttgaaagcaaacgacaaacgaagcagcaagcaaagagcacgaggacggtgaacaagtct
gtcacctgtatacatctatttccccgcgggtgcacctactctctctcctgccccggcagagtcagctgccttacgtgacggatcccgcg
tctcgaacagagcgcgcagaggaacgctgaaggtctcgcctctgtcgcacctcagcgcggcatacaccacaataaccacctgacga
atgcgcttggttcttcgtccattagcgaagcgtccggttcacacacgtgccacgttggcgaggtggcaggtgacaatgatcggtgga

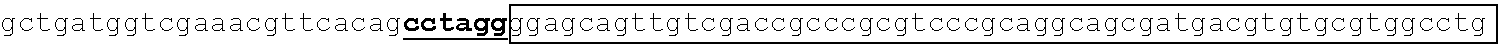

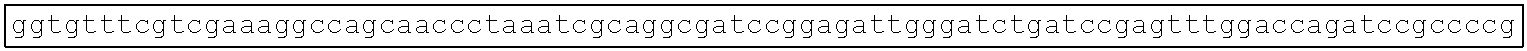

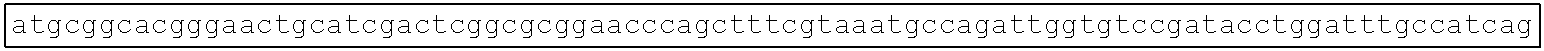

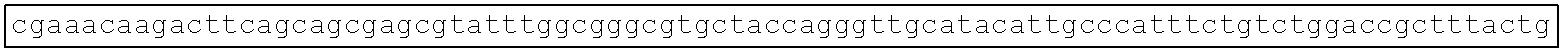

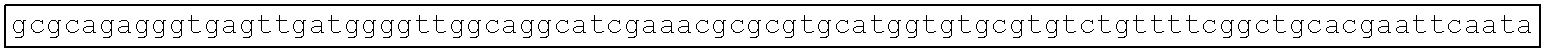

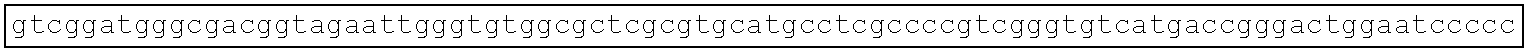

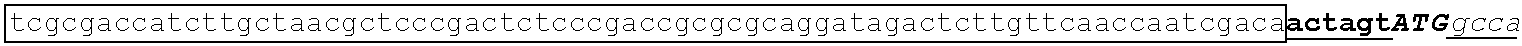

ccgcatccactttctcggcgttcaatgcccgctgcggcgacctgcgtcgctcggcgggctccgggccccggcgcccagcgaggc
ccctccccgtgcgcgggcgcgccatccccccccgcatcatcgtggtgtcctcctcctcctccaaggtgaaccccctgaagaccgag
gccgtggtgtcctccggcctggccgaccgcctgcgcctgggctccctgaccgaggacggcctgtcctacaaggagaagttcatc
gtgcgctgctacgaggtgggcatcaacaagaccgccaccgtggagaccatcgccaacctgctgcaggaggtgggctgcaac
cacgcccagtccgtgggctactccaccgccggcttctccaccacccccaccatgcgcaagctgcgcctgatctgggtgaccgccc
gcatgcacatcgagatctacaagtaccccgcctggtccgacgtggtggagatcgagtcctggggccagggcgagggcaaga
tcggcacccgccgcgactggatcctgcgcgactacgccaccggccaggtgatcggccgcgccacctccaagtgggtgatgatg
aaccaggacacccgccgcctgcagaaggtggacgtggacgtgcgcgacgagtacctggtgcactgcccccgcgagctgcgcc
tggccttccccgaggagaacaactcctccctgaagaagatctccaagctggaggacccctcccagtactccaagctgggcctg
gtgccccgccgcgccgacctggacatgaaccagcacgtgaacaacgtgacctacatcggctgggtgctggagtccatgcccca
ggagatcatcgacacccacgagctgcagaccatcaccctggactaccgccgcgagtgccagcacgacgacgtggtggactcc
ctgacctcccccgagccctccgaggacgccgaggccgtgttcaaccacaacggcaccaacggctccgccaacgtgtccgccaa
cgaccacggctgccgcaacttcctgcacctgctgcgcctgtccggcaacggcctggagatcaaccgcggccgcaccgagtggc
gcaagaagcccacccgcATGGACTACAAGGACCACGACGGCGACTACAAGGACCACGACATCGACTACA

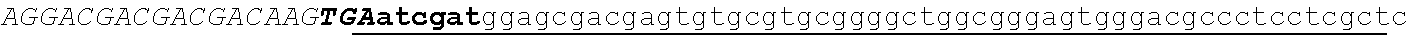

ctctctgttctgaacggaacaatcggccaccccgcgctacgcgccacgcatcgagcaacgaagaaaaccccccgatgataggttgc
ggtggctgccgggatatagatccggccgcacatcaaagggcccctccgccagagaagaagctcctttcccagcagactccttctgct
gccaaaacacttctctgtccacagcaacaccaaaggatgaacagatcaacttgcgtctccgcgtagcttcctcggctagcgtgcttg
caacaggtccctgcactattatcttcctgctttcctctgaattatgcggcaggcgagcgctcgctctggcgagcgctccttcgcgccgc
cctcgctgatcgagtgtacagtcaatgaatggtgagctcctcactcagcgcgcctgcgcggggatgcggaacgccgccgccgcctt
gtcttttgcacgcgcgactccgtcgcttcgcgggtggcacccccattgaaaaaaacctcaattctgtttgtggaagacacggtgtac
ccccaaccacccacctgcacctctattattggtattattgacgcgggagcgggcgttgtactctacaacgtagcgtctctggttttca
gctggctcccaccattgtaaattcttgctaaaatagtgcgtggttatgtgagaggtatggtgtaacagggcgtcagtcatgttggtt
ttcgtgctgatctcgggcacaaggcgtcgtcgacgtgacgtgcccgtgatgagagcaataccgcgctcaaagccgacgcatggcc
tttactccgcactccaaacgactgtcgctcgtatttttcggatatctattttttaagagcgagcacagcgccgggcatgggcctgaa
aggcctcgcggccgtgctcgtggtgggggccgcgagcgcgtggggcatcgcggcagtgcaccaggcgcagacggaggaacgcat
ggtgagtgcgcatcacaagatgcatgtcttgttgtctgtactataatgctagagcatcaccaggggcttagtcatcgcacctgcttt
ggtcattacagaaattgcacaagggcgtcctccgggatgaggagatgtaccagctcaagctggagcggcttcgagccaagcagg
agcgcggcgcatgacgacctacccacatgcgaagagc

Constructs pSZ6383, pSZ6384 and pSZ6377 were transformed into S8813. Primary transformants were clonally purified and screened under standard lipid production conditions at pH 5. Integration of pSZ6383 or pSZ6384 at the FAD2-1 locus was verified by DNA blot analysis. The fatty acid profiles, sn-2 profiles and lipid titers of lead strains were assayed in 50-mL shake flasks (Table 23). FAD2-1 ablation reduced C18:2 to <1% in most strains. Expression of a second copy of GarmFATA1(G108A) and TcDGAT1 (S8990, 58992, 58998 & S8999), or TcDGAT2 (S8994, 59000 & S9047) elevated C18:0 to >56%. The D5393-28 strain, expressing a second copy of GarmFATA1(G108A) without either of the cocoa DGAT genes (pSZ6377) had a similar fatty acid profile, but lower lipid titer. As shown in Table 23, as compared to strain S8813, for strains expressing either TcDGAT1 or TcDGAT2, C16:0 increased from 3.2% to 3.7%-4.0%, C18:0 increased from 45.8% to about 56%, C18:2 decreased from 1.4% to about 1.0%.

TABLE 23
Fatty acid profiles of FAD2-1 ablation strains.

| | Strain | | | | | | | | |
|---|---|---|---|---|---|---|---|---|---|
| | S8813 | D5393-28 | S8990 | S8992 | S8998 | S8999 | S8994 | S9000 | S9047 |
| C12:0 | 0.1 | 0.2 | 0.2 | 0.2 | 0.1 | 0.2 | 0.1 | 0.1 | 0.2 |
| C14:0 | 0.4 | 0.5 | 0.5 | 0.5 | 0.5 | 0.5 | 0.5 | 0.5 | 0.5 |
| C16:0 | 3.2 | 3.8 | 3.7 | 3.8 | 3.9 | 4.0 | 3.7 | 3.8 | 3.5 |
| C16:1 cis-7 | 0.1 | 0.1 | 0.1 | 0.1 | 0.1 | 0.1 | 0.1 | 0.1 | 0.1 |
| C16:1 cis-9 | 0.0 | 0.1 | 0.1 | 0.1 | 0.1 | 0.1 | 0.1 | 0.1 | 0.1 |
| C17:0 | 0.1 | 0.2 | 0.2 | 0.1 | 0.2 | 0.1 | 0.2 | 0.2 | 0.2 |
| C18:0 | 45.8 | 56.0 | 56.6 | 56.0 | 56.2 | 56.0 | 56.3 | 56.4 | 56.5 |
| C18:1 | 45.9 | 35.8 | 35.4 | 35.9 | 35.7 | 35.5 | 35.9 | 35.7 | 35.9 |
| C18:2 | 1.4 | 1.0 | 0.9 | 1.0 | 0.9 | 1.1 | 0.9 | 0.9 | 0.8 |
| C18:3 α | 0.3 | 0.3 | 0.3 | 0.2 | 0.3 | 0.2 | 0.2 | 0.3 | 0.3 |
| C20:0 | 2.0 | 1.6 | 1.6 | 1.5 | 1.6 | 1.5 | 1.5 | 1.5 | 1.5 |
| C22:0 | 0.2 | 0.2 | 0.2 | 0.2 | 0.2 | 0.2 | 0.2 | 0.2 | 0.2 |
| C24:0 | 0.1 | 0.1 | 0.1 | 0.1 | 0.1 | 0.1 | 0.1 | 0.1 | 0.1 |
| saturates | 52.1 | 62.6 | 63.1 | 62.6 | 62.9 | 62.8 | 62.8 | 62.9 | 62.7 |

Liquid chromatography and mass spectrometry were used to analyze the TAG composition of final strains. The strains accumulated 68-71% SOS, with trisaturates ranging from 2.5-2.8%. The D5393-28 strain, expressing a second copy of GarmFATA1(G108A) without either of the cocoa DGAT genes had similar SOS content but slightly higher trisaturates. The TAG composition of a typical Shea stearin and a sample of Kokum butter are shown for comparison

TABLE 24
LC/MS TAG profiles of FAD2-1 ablation strains.

| | Strain | | | | | | | | | |
|---|---|---|---|---|---|---|---|---|---|---|
| | | | | | | | | | Shea | Kokum |
| | D5393-28 | S8990 | S8992 | S8998 | S8999 | S8994 | S9000 | S9047 | stearin | butter |
| OOL | | | | | | | | | 0.4 | |
| LLS | | | | | | | | | 0.2 | |
| POL | | | | | | | | | 0.3 | |
| OOO | | | | | | | | | 1.3 | 1.7 |
| SOL | | | | | | | | | 1.0 | 0.4 |
| LaOS + MOP | 0.2 | 0.3 | 0.3 | 0.2 | 0.3 | 0.3 | 0.4 | 0.2 | | |
| OOP | 0.5 | 0.2 | 0.3 | 0.2 | 0.2 | 0.4 | 0.3 | 0.2 | 0.8 | 0.7 |
| PLS (+SLnS) | 0.6 | 0.7 | 0.7 | 0.7 | 0.7 | 0.6 | 0.6 | 0.4 | 0.6 | 0.3 |
| POP (+MOS) | 1.1 | 1.0 | 1.0 | 1.1 | 1.1 | 1.0 | 1.2 | 0.8 | 0.7 | 0.4 |
| OOS | 10.5 | 10.3 | 11.3 | 11.0 | 11.0 | 10.9 | 10.1 | 10.6 | 6.4 | 11.8 |
| SLS (+PLA) | 1.9 | 1.7 | 2.0 | 1.6 | 2.1 | 1.8 | 1.9 | 1.5 | 5.5 | 1.4 |
| POS | 8.4 | 8.5 | 8.4 | 8.7 | 8.9 | 8.4 | 10.0 | 7.7 | 6.3 | 4.8 |
| MaOS | | | | | | | | | | 0.3 |
| SOG | 0.4 | 0.5 | 0.5 | 0.6 | 0.3 | 0.5 | 0.4 | 0.5 | | |
| OOA | 0.5 | 0.3 | 0.4 | 0.4 | 0.4 | 0.4 | 0.4 | 0.4 | 0.2 | 0.2 |
| SOS (+POA) | 68.4 | 69.7 | 68.7 | 69.1 | 68.3 | 69.4 | 68.0 | 71.4 | 69.7 | 76.6 |
| SSP (+MSA) | 0.5 | 0.5 | 0.5 | 0.4 | 0.5 | 0.5 | 0.5 | 0.4 | 0.2 | |
| SOA + POB | 3.9 | 3.8 | 3.5 | 3.6 | 3.4 | 3.5 | 3.5 | 3.4 | 4.0 | 1.0 |
| SSS (+PSA) | 2.6 | 2.3 | 2.2 | 2.1 | 2.3 | 2.2 | 2.3 | 2.1 | 2.0 | 0.5 |
| SOB + LgOP + AOA | 0.4 | 0.2 | 0.2 | 0.3 | 0.3 | 0.3 | 0.3 | 0.3 | 0.4 | |
| SSA (+PBS) | | | | | | | | | 0.2 | |
| SOLg (+POHx) | | | | | | | | | 0.3 | |
| SUM (area %) | 99.8 | 99.9 | 99.8 | 99.9 | 99.8 | 99.9 | 100.0 | 99.9 | 100.0 | 100.0 |
| Sat-Sat-Sat | 3.1 | 2.8 | 2.7 | 2.5 | 2.7 | 2.7 | 2.8 | 2.5 | 2.4 | 0.5 |
| Sat-U-Sat | 84.9 | 85.9 | 84.7 | 85.3 | 85.1 | 85.0 | 86.0 | 85.8 | 87.5 | 84.7 |
| Sat-O-Sat | 82.4 | 83.5 | 82.0 | 82.9 | 82.3 | 82.6 | 83.4 | 83.9 | 81.4 | 83.1 |
| Sat-L-Sat | 2.5 | 2.4 | 2.6 | 2.3 | 2.8 | 2.4 | 2.6 | 1.9 | 6.1 | 1.6 |
| U-U-U/Sat | 11.8 | 11.3 | 12.4 | 12.2 | 12.0 | 12.2 | 11.3 | 11.7 | 10.6 | 14.8 |

La = laurate (C12:0),
M = myristate (C14:0),
P = palmitate (C16:0),
Ma = margarate (C17:0),
S = stearate (C18:0),
O = oleate (C18:1),
L = linoleate (C18:2),
Ln = α-linolenate (C18:3 α),
A = arachidate (C20:0),
G = (C20:1),
B = behenate (C22:0),
Lg = lignocerate (C24:0),
Hx = hexacosanoate (C26:0).
Sat = saturated,
U = unsaturated

### Example 8 Variant Brassica Napus Thioeserase

In this example, we demonstrate the modification of the enzyme specificity of a FATA thioesterase originally isolated from Brassica napus (BnOTE, accession CAA52070), by site directed mutagenesis targeting two amino acids positions D124 and D209).

To determine the impact of each amino acid substitution on the enzyme specificity of the BnOTE, the wild-type and the mutant BnOTE genes were cloned into a vector enabling expression and expressed in P. moriformis strain S8588. Strain S8588 is a strain in which the endogenous FATA1 allele has been disrupted and expresses a Prototheca moriformis KASII gene and sucrose invertase. Recombinant strains with FATA1 disruption and co-expression of P. moriformis KASII and invertase were previously disclosed in co-owned applications WO2012/106560 and WO2013/15898, herein incorporated by reference.

Strains that express wild type or mutant BnOTE enzymes, contructs pSZ6315, pSZ6316, pSZ6317, or pSZ6318 were expressed in S8588. In these constructs, the Saccharomyces carlsbergensis MEL1 gene (Accession no: AAA34770) was utilized as the selectable marker to introduce the wild-type and mutant BnOTE genes into the FAD2-2 locus of P. moriformis strain S8588 by homologous recombination using previously described transformation methods (biolistics). The constructs that have been expressed in S8588 are listed in Table 25.

TABLE 25
DNA lot# and plasmid ID of DNA constructs that
expressing wild-type and mutant BnOTE genes

| DNA | Solazyme | |
|---|---|---|
| Lot# | Plasmid | Construct |
| D5309 | pSZ6315 | FAD2-2::PmHXT1-ScarMEL1-PmPGK:PmSAD2-2 |
| | | V3-CpSADtp-BnOTE-PmSAD2-1 utr::FAD2-2 |
| D5310 | pSZ6316 | FAD2-2::PmHXT1-ScarMEL1-PmPGK:PmSAD2-2 |
| | | V3-CpSADtp-BnOTE(D124A)-PmSAD2-1 utr::FAD2-2 |
| D5311 | PSZ6317 | FAD2-2::PmHXT1-ScarMEL1-PmPGK:PmSAD2-2 |
| | | V3-CpSADtp-BnOTE(D209A)-PmSAD2-1 utr::FAD2-2 |
| D5312 | pSZ6318 | FAD2-2::PmHXT1-ScarMEL1-PmPGK:PmSAD2-2 |
| | | V3-CpSADtp-BnOTE(D124A, D209A)-PmSAD2-1 utr::FAD2-2 |

pSZ6315

The consruct psZ6315 can be written as FAD2-2::PmHXT1-ScarMEL1-PmPGK:PmSAD2-2 V3-CpSADtp-BnOTE-PmSAD2-1 utr::FAD2-2. The sequence of the pSZ6315 transforming DNA is provided below. Relevant restriction sites in pSZ6315 are indicated in lowercase, bold and underlining and are 5′-3′ SgrAI, Kpn I, SnaBI, AvrII, SpeI, AscI, ClaI, Sac I, SK respectively. SgrAI and Sbff sites delimit the 5′ and 3′ ends of the transforming DNA. Bold, lowercase sequences represent FAD2-2 genomic DNA that permit targeted integration at FAD2-2 locus via homologous recombination. Proceeding in the 5′ to 3′ direction, the P. moriformis HXT1 promoter driving the expression of the Saccharomyces carlsbergensis MEL1 gene is indicated by boxed text. The initiator ATG and terminator TGA for MEL1 gene are indicated by uppercase, bold italics while the coding region is indicated in lowercase italics. The P. moriformis PGK 3′ UTR is indicated by lowercase underlined text followed by the P. moriformis SAD2-2 V3 promoter, indicated by boxed italics text. The Initiator ATG and terminator TGA codons of the wild-type BnOTE are indicated by uppercase, bold italics, while the remainder of the coding region is indicated by bold italics in lower case. The three-nucleotide codon corresponding to the target amino acids, D124 and D209, are in lower case, italicized, bolded and wave underlined. The P. moriformis SAD2-1 3′UTR is again indicated by lowercase underlined text followed by the FAD2-2 genomic region indicated by bold, lowercase text.

Nucleotide sequence of transforming DNA contained in pSZ6315
SEQ ID NO: 131
caccggcgcgctgcttcgcgtgccgggtgcagcaatcagatccaagtctgacgacttgcgcgcacgcgccggatccttcaattccaaagtgtcg
tccgcgtgcgcttcttcgccttcgtcctcttgaacatccagcgacgcaagcgcagggcgctgggcggctggcgtcccgaaccggcctcggcgcac
gcggctgaaattgccgatgtcggcaatgtagtgccgctccgcccacctctcaattaagtttttcagcgcgtggttgggaatgatctgcgctcatg
gggcgaaagaaggggttcagaggtgctttattgttactcgactgggcgtaccagcattcgtgcatgactgattatacatacaaaagtacagctc
gcttcaatgccctgcgattcctactcccgagcgagcactcctctcaccgtcgggttgcttcccacgaccacgccggtaagagggtctgtggcctc
gcgcccctcgcgagcgcatctttccagccacgtctgtatgattttgcgctcatacgtctggcccgtcgaccccaaaatgacgggatcctgcataa
tatcgcccgaaatgggatccaggcattcgtcaggaggcgtcagccccgcgggagatgccggtcccgccgcattggaaaggtgtagagggggt

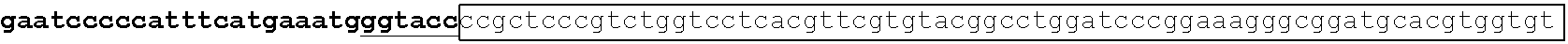

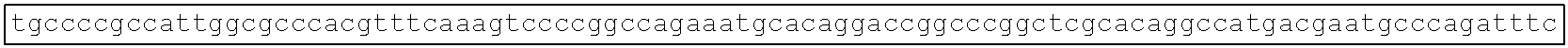

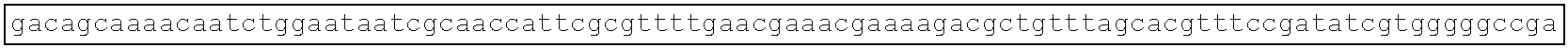

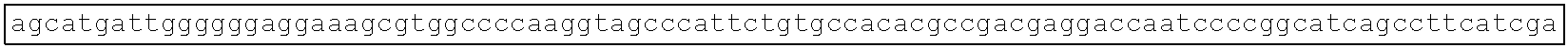

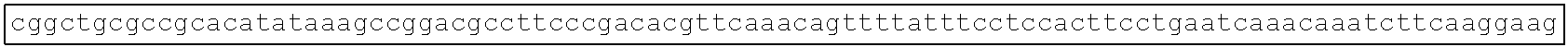

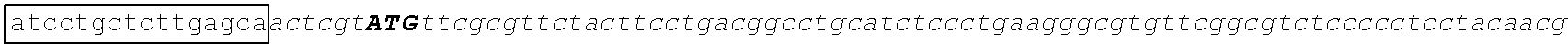

gcctgggcctgacgccccagatgggctgggacaactggaacacgttcgcctgcgacgtctccgagcagctgctgctggacacggccgacc
gcatctccgacctgggcctgaaggacatgggctacaagtacatcatcctggacgactgctggtcctccggccgcgactccgacggcttcctg
gtcgccgacgagcagaagttccccaacggcatgggccacgtcgccgaccacctgcacaacaactccttcctgttcggcatgtactcctccgc
gggcgagtacacgtgcgccggctaccccggctccctgggccgcgaggaggaggacgcccagttcttcgcgaacaaccgcgtggactacct
gaagtacgacaactgctacaacaagggccagttcggcacgcccgagatctcctaccaccgctacaaggccatgtccgacgccctgaacaa
gacgggccgccccatcttctactccctgtgcaactggggccaggacctgaccttctactggggctccggcatcgcgaactcctggcgcatgtc
cggcgacgtcacggcggagttcacgcgccccgactcccgctgcccctgcgacggcgacgagtacgactgcaagtacgccggcttccactgc
tccatcatgaacatcctgaacaaggccgcccccatgggccagaacgcgggcgtcggcggctggaacgacctggacaacctggaggtcgg
cgtcggcaacctgacggacgacgaggagaaggcgcacttctccatgtgggccatggtgaagtcccccctgatcatcggcgcgaacgtga
acaacctgaaggcctcctcctactccatctactcccaggcgtccgtcatcgccatcaaccaggactccaacggcatccccgccacgcgcgtct
ggcgctactacgtgtccgacacggacgagtacggccagggcgagatccagatgtggtccggccccctggacaacggcgaccaggtcgtg
gcgctgctgaacggcggctccgtgtcccgccccatgaacacgaccctggaggagatcttcttcgactccaacctgggctccaagaagctga
cctccacctgggacatctacgacctgtgggcgaaccgcgtcgacaactccacggcgtccgccatcctgggccgcaacaagaccgccaccg
gcatcctgtacaacgccaccgagcagtcctacaaggacggcctgtccaagaacgacacccgcctgttcggccagaagatcggctccctgtc

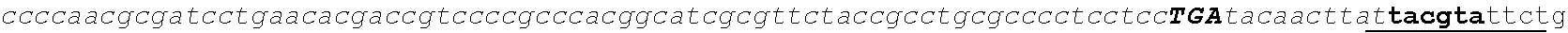

accggcgctgatgtggcgcggacgccgtcgtactctttcagactttactcttgaggaattgaacctttctcgcttgctggcatgtaaacattggcgc
aattaattgtgtgatgaagaaagggtggcacaagatggatcgcgaatgtacgagatcgacaacgatggtgattgttatgaggggccaaacctg
gctcaatcttgtcgcatgtccggcgcaatgtgatccagcggcgtgactctcgcaacctggtagtgtgtgcgcaccgggtcgctttgattaaaactg
atcgcattgccatcccgtcaactcacaagcctactctagctcccattgcgcactcgggcgcccggctcgatcaatgttctgagcggagggcgaag
cgtcaggaaatcgtctcggcagctggaagcgcatggaatgcggagcggagatcgaatcaggatcccgcgtctcgaacagagcgcgcagagga
acgctgaaggtctcgcctctgtcgcacctcagcgcggcatacaccacaataaccacctgacgaatgcgcttggttcttcgtccattagcgaagcgt

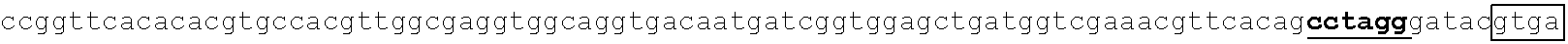

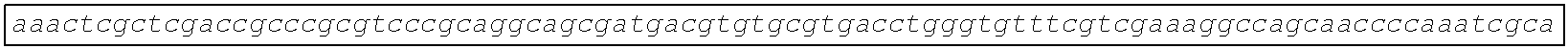

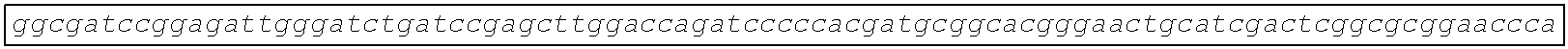

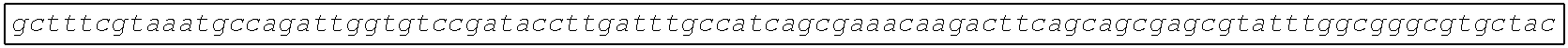

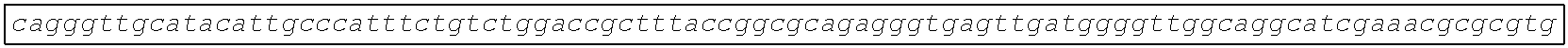

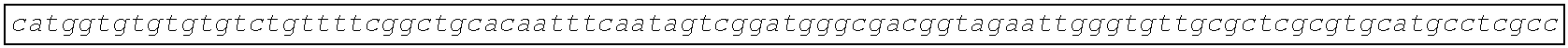

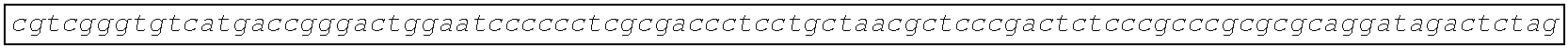

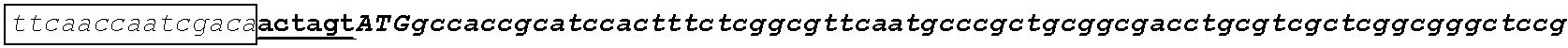

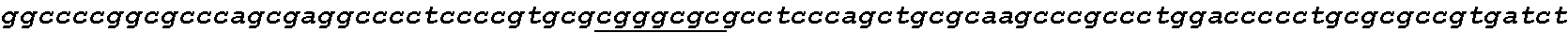

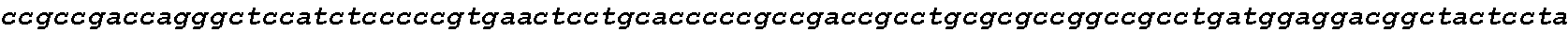

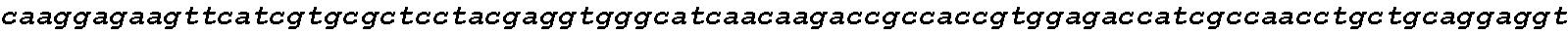

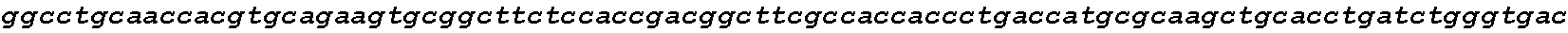

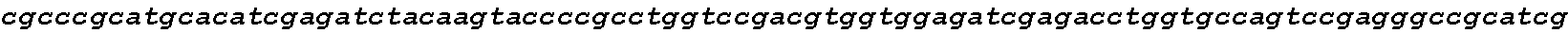

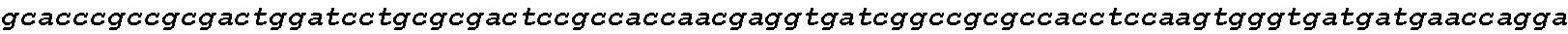

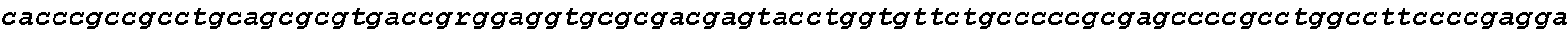

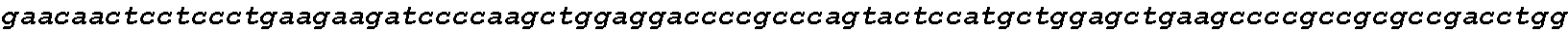

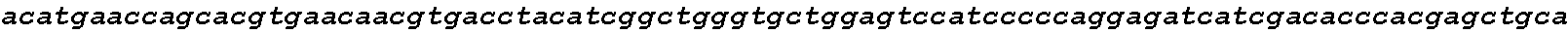

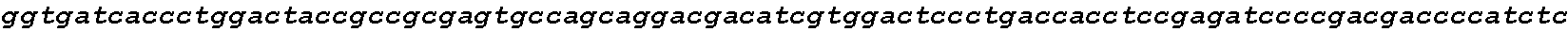

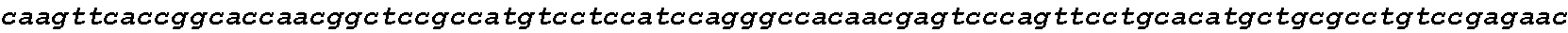

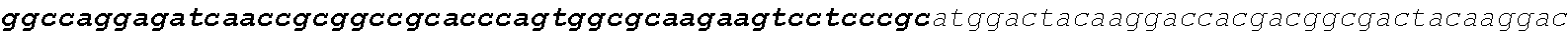

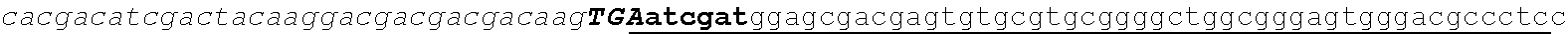

tcgctcctctctgttctgaacggaacaatcggccaccccgcgctacgcgccacgcatcgagcaacgaagaaaaccccccgatgataggttgcgg
tggctgccgggatatagatccggccgcacatcaaagggcccctccgccagagaagaagctcctttcccagcagactccttctgctgccaaaaca
cttctctgtccacagcaacaccaaaggatgaacagatcaacttgcgtctccgcgtagcttcctcggctagcgtgcttgcaacaggtccctgcacta
ttatcttcctgctttcctctgaattatgcggcaggcgagcgctcgctctggcgagcgctccttcgcgccgccctcgctgatcgagtgtacagtcaat
gaatggtgagctccgcgcctgcgcgaggacgcagaacaacgctgccgccgtgtatttgcacgcgcgactccggcgcttcgctggtggcacccc
cataaagaaaccctcaattctgtttgtggaagacacggtgtacccccacccacccacctgcacctctattattggtattattgacgcgggagtgg
gcgttgtaccctacaacgtagcttctctagttttcagctggctcccaccattgtaaattcatgctagaatagtgcgtggttatgtgagaggtatag
tgtgtctgagcagacggggcgggatgcatgtcgtggtggtgatctttggctcaaggcgtcgtcgacgtgacgtgcccgatcatgagagcaatac
cgcgctcaaagccgacgcatagcctttactccgcaatccaaacgactgtcgctcgtattttttggatatctattttaaagagcgagcacagcgcc
gggcatgggcctgaaaggcctcgcggccgtgctcgtggtgggggccgcgagcgcgtggggcatcgcggcagtgcaccaggcgcagacggag
gaacgcatggtgcgtgcgcaatataagatacatgtattgttgtcctgcagg
Nucleotide sequence of BnOTE (D124A) in pSZ6316
SEQ ID NO: 132

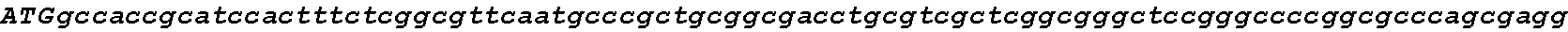

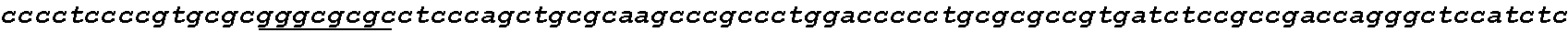

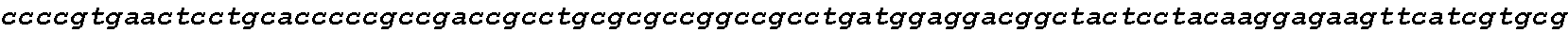

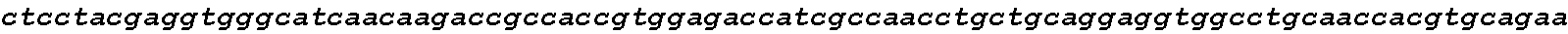

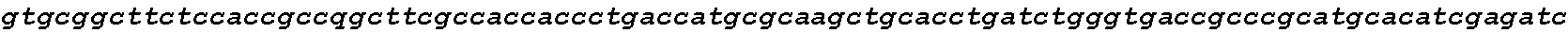

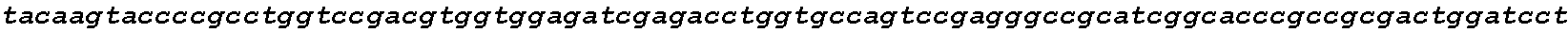

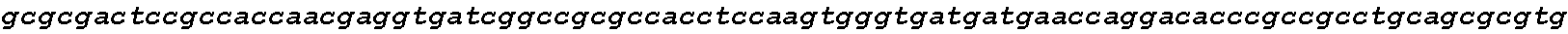

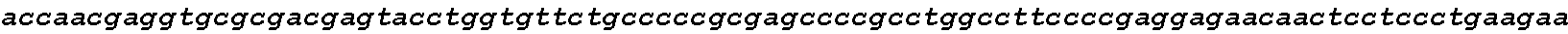

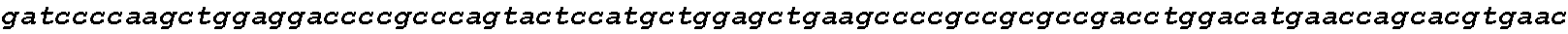

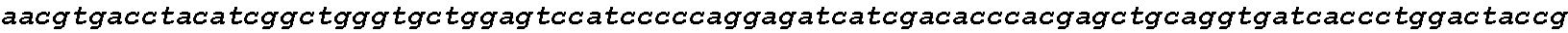

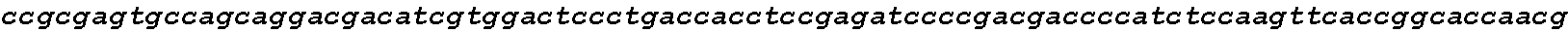

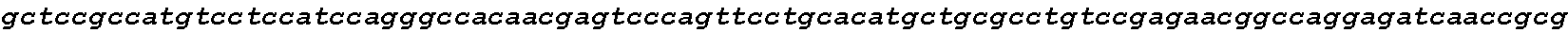

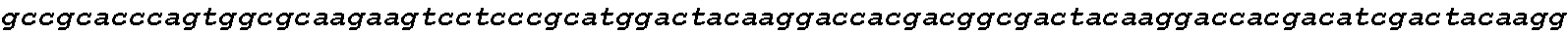

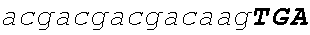

The sequence of the pSZ6317 transforming DNA is same as pSZ6315 except the D209A point mutation, the BnOTE D209A DNA sequence is provided below. The three-nucleotide codon corresponding to the target two amino acids, D124 and D209, are in lower case, italicized, bolded and wave underlined. pSZ6317 is written as FAD2-2::PmHXT1-ScarMEL1-PmPGK:PmSAD2-2 V3-CpSADtp-BnOTE (D209A)-PmSAD2-1 utr::FAD2-2

Nucleotide sequence of BnOTE (D209A) in pSZ6317:
SEQ ID NO: 133

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

atggactacaaggaccac
gacggcgactacaaggaccacgacatcgactacaaggacgacgacgaca
ag

[symbol]

The sequence of the pSZ6318 transforming DNA is same as pSZ6315 except two point mutations, D124A and D209A, the BnOTE (D124A, D209A) DNA sequence is provided below. The three-nucleotide codon corresponding to the target two amino acids, D124 and D209, are in lower case, italicized, bolded and wave underlined. pSZ6318 is written as FAD2-2::PmHXT1-ScarMEL1-PmPGK:PmSAD2-2 V3-CpSADtp-BnOTE (D124A, D209A)-PmSAD2-1 utr::FAD2-2

SEQ ID NO: 134 Nucleotide Sequence of BnOTE (D124A, D209A) in pSZ6318

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

atggactacaagga
ccacgacggcgactacaaggaccacgacatcgactacaaggacgacgac
gacaag

[symbol]

The DNA constructs containing the wild-type and mutant BnOTE genes were transformed into the parental strain S8588. Primary transformants were clonally purified and grown under standard lipid production conditions at pH5.0. The resulting profiles from representative clones arising from transformations with pSZ6315, pSZ6316, pSZ6317, and pSZ6318 into S8588 are shown in Table 26. The parental strain S8588 produces 5.4% C18:0, when transformed with the DNA cassette expressing wild-type BnOTE, the transgenic lines produce ˜11% C18:0. The BnOTE mutant (D124A) increased the amount of C18:0 by at least 2 fold compared to the wild-type protein. In contrast, the BnOTE D209A mutation appears to have no impact on the enzyme activity/specificity of the BnOTE thioesterase. Finally, expression of the BnOTE (D124A, D209A) resulted in very similar fatty acid profile to what we observed in the transformants from S8588 expressing BnOTE (D124A), again indicating that D209A has no significant impact on the enzyme activity.

TABLE 26
Fatty acid profiles in S8588 and derivative transgenic
lines transformed with wild-type and mutant BnOTE genes

| | | Fatty Acid Area % | | | |
|---|---|---|---|---|---|
| Transforming DNA | Sample ID | C16:0 | C18:0 | C18:1 | C18:2 |
| | pH5; S8588 (parental strain) | 3.00 | 5.43 | 81.75 | 6.47 |
| D5309, pSZ6315; | pH5; S8588, D5309-6; | 3.86 | 11.68 | 76.51 | 5.06 |
| wild-type BnOTE | pH5; S8588, D5309-2; | 3.50 | 11.00 | 77.80 | 4.95 |
| | pH5; S8588, D5309-9; | 3.51 | 10.72 | 78.03 | 5.00 |
| | pH5; S8588, D5309-10; | 3.55 | 10.69 | 78.06 | 4.96 |
| | pH5; S8588, D5309-11; | 3.61 | 10.69 | 78.05 | 4.95 |
| D5310, pSZ6316, | pH5; S8588, D5310-6; | 4.27 | 31.55 | 55.31 | 5.30 |
| BnOTE (D124A) | pH5; S8588, D5310-1; | 4.53 | 30.85 | 54.71 | 6.03 |
| | pH5; S8588, D5310-5; | 5.21 | 20.75 | 65.43 | 5.02 |
| | pH5; S8588, D5310-10; | 4.99 | 19.18 | 67.75 | 5.00 |
| | pH5; S8588, D5310-2; | 4.90 | 18.92 | 68.17 | 4.98 |
| D5311, pSZ6317, | pH5; S8588, D5311-3; | 3.50 | 11.90 | 76.95 | 4.98 |
| BnOTE (D209A) | pH5; S8588, D5311-4; | 3.63 | 11.35 | 77.44 | 4.94 |
| | pH5; S8588, D5311-14; | 3.47 | 11.23 | 77.68 | 4.98 |
| | pH5; S8588, D5311-10; | 3.60 | 11.20 | 77.53 | 5.00 |
| | pH5; S8588, D5311-12; | 3.53 | 11.12 | 77.59 | 5.09 |
| D5312, pSZ6318, | pH5; S8588, D5312-20 | 4.79 | 37.97 | 47.74 | 6.01 |
| BnOTE (D124A, | pH5; S8588, D5312-40; | 5.97 | 22.94 | 62.20 | 5.11 |
| D209A) | pH5; S8588, D5312-39; | 6.07 | 22.75 | 62.24 | 5.17 |
| | pH5; S8588, D5312-16; | 5.25 | 18.81 | 67.36 | 5.09 |
| | pH5; S8588, D5312-26; | 4.93 | 18.70 | 68.37 | 4.96 |

### Example 9 Variant Garcinia Mangostana Thioeserase

In this example, we demonstrate the ability to modify the activity and specificity of a FATA thioesterase originally isolated from Garcinia mangostana (GmFATA, accession 004792), using site directed mutagenesis targeting six amino acid positions within the enzyme and various combinations thereof. Facciotti et al (NatBiotech 1999) had previously altered three of the amino acids (G108, S111, V193). The remaining three amino acids targeted are L91, G96, and T156.

To test the impact of each mutation on the activity of the GmFATA, the wild-type and mutant genes were cloned into a vector enabling expression within the P. moriformis strain S3150. Table 27 summarizes the results from a three day lipid profile screen comparing the wild-type GmFATA with the 14 mutants. Three GmFATA mutants (DNA lot numbers D3998, D4000, D4003) increased the amount of C18:0 by at least 1.5 fold compared to the wild-type protein (DNA lot number D3997). D3998 and D4003 were mutations that had been described by Facciotti et al (NatBiotech 1999) as substitutions that increased the activity of the GmFATA. Strain S3150 expressing the mutations contained in DNA lot number D4000 was based on research at Solazyme which demonstrated this position influenced the activity of the FATB thioesterases. All of the constructs were codon optimized to reflect UTEX 1435 codon usage. Non-mutated GmFATA increases the fatty acid content of C18:0 and decreases the fatty acid content of C18:1 and C18:2. As can be seen in Table 27 the G90A mutant GmFATA increases the fatty acid content of C18:0 and decreases the fatty acid content of C18:1 and C18:2 when compared to the wild-type GmFATA.

TABLE 27

| Algal | | | | | | | |
|---|---|---|---|---|---|---|---|
| Strain | DNA # | GmFATA | C14:0 | C16:0 | C18:0 | C18:1 | C18:2 |
| P. moriformis | S3150 | | 1.63 | 29.82 | 3.08 | 55.95 | 7.22 |
| S3150 | D3997 | Wild-Type | 1.79 | 29.28 | 7.32 | 52.88 | 6.21 |
| | pSZ5083 | GmFATA | | | | | |
| | D3998 | S111A, | 1.84 | 28.88 | 11.19 | 49.08 | 6.21 |
| | pSZ5084 | V193A | | | | | |
| | D3999 | S111V, | 1.73 | 29.92 | 3.23 | 56.48 | 6.46 |
| | pSZ5085 | V193A | | | | | |
| | D4000 | G96A | 1.76 | 30.19 | 12.66 | 45.99 | 6.01 |
| | pSZ5086 | | | | | | |
| | D4001 | G96T | 1.82 | 30.60 | 3.58 | 55.50 | 6.28 |
| | pSZ5087 | | | | | | |
| | D4002 | G96V | 1.78 | 29.35 | 3.45 | 56.77 | 6.43 |
| | pSZ5088 | | | | | | |
| | D4003 | G108A | 1.77 | 29.06 | 12.31 | 47.86 | 6.08 |
| | pSZ5089 | | | | | | |
| | D4007 | G108V | 1.81 | 28.78 | 5.71 | 55.05 | 6.26 |
| | pSZ5093 | | | | | | |
| | D4004 | L91F | 1.76 | 29.60 | 6.97 | 53.04 | 6.13 |
| | pSZ5090 | | | | | | |
| | D4005 | L91K | 1.87 | 28.89 | 4.38 | 56.24 | 6.35 |
| | pSZ5091 | | | | | | |
| | D4006 | L91S | 1.85 | 28.06 | 4.81 | 56.45 | 6.47 |
| | pSZ5092 | | | | | | |
| | D4008 | T156F | 1.81 | 28.71 | 3.65 | 57.35 | 6.31 |
| | pSZ5094 | | | | | | |
| | D4009 | T156A | 1.72 | 29.66 | 5.44 | 54.54 | 6.26 |
| | pSZ5095 | | | | | | |
| | D4010 | T156K | 1.73 | 29.95 | 3.17 | 56.86 | 6.21 |
| | pSZ5096 | | | | | | |
| | D4011 | T156V | 1.80 | 29.17 | 4.97 | 55.44 | 6.27 |
| | pSZ5097 | | | | | | |

Nucleotide sequence of the GmFATA wild-type parental gene expression vector is shown below (D3997, pSZ5083). The plasmid pSZ5083 can be written as THI4a::CrTUB2-NeoR-PmPGH:PmSAD2-2Ver3-CpSAD1tp_GarmFATA1_FLAG-CvNR::THI4a. The 5′ and 3′ homology arms enabling targeted integration into the Thi4 locus are noted with lowercase; the CrTUB2 promoter is noted in uppercase italic which drives expression of the neomycin selection marker noted with lowercase italic followed by the PmPGH 3′UTR terminator highlighted in uppercase. The PmSAD2-1 promoter (noted in bold text) drives the expression of the GmFATA gene (noted with lowercase bold text) and is terminated with the CvNR 3′UTR noted in underlined, lower case bold. Restriction cloning sites and spacer DNA fragments are noted as underlined, uppercase plain lettering. The nucleotide sequence for all of the GmFATA constructs disclosed in this example is identical to that of pSZ5083 with the exception of the encoded GmFATA. The promoter, 3′UTR, selection marker and targeting arms are the same as described for pSZ5083. The individual GmFATA mutant sequences are shown below. The amino acid sequence of the unmutagenized GmFATA is showin in FIG. 1. The amino acid sequences of the altered GmFATA proteins are shown below.

pSZ5083
SEQ ID NO: 135
ccctcaactgcgacgctgggaaccttctccgggcaggcgatgtgcgtgggtttgcctccttg
gcacggctctacaccgtcgagtacgccatgaggcggtgatggctgtgtcggttgccacttcg
tccagagacggcaagtcgtccatcctctgcgtgtgtggcgcgacgctgcagcagtccctctg
cagcagatgagcgtgactttggccatttcacgcactcgagtgtacacaatccatttttctta
aagcaaatgactgctgattgaccagatactgtaacgctgatttcgctccagatcgcacagat
agcgaccatgttgctgcgtctgaaaatctggattccgaattcgaccctggcgctccatccat
gcaacagatggcgacacttgttacaattcctgtcacccatcggcatggagcaggtccactta
gattcccgatcacccacgcacatctcgctaatagtcattcgttcgtgtcttcgatcaatctc
aagtgagtgtgcatggatcttggttgacgatgcggtatgggtttgcgccgctggctgcaggg
tctgcccaaggcaagctaacccagctcctctccccgacaatactctcgcaggcaaagccggt
cacttgccttccagattgccaataaactcaattatggcctctgtcatgccatccatgggtct
gatgaatggtcacgctcgtgtcctgaccgttccccagcctctggcgtcccctgccccgccca
ccagcccacgccgcgcggcagtcgctgccaaggctgtctcggaGGTACCCTTTCTTGCGCTA
TGACACTTCCAGCAAAAGGTAGGGCGGGCTGCGAGACGGCTTCCCGGCGCTGCATGCAACAC
CGATGATGCTTCGACCCCCCGAAGCTCCTTCGGGGCTGCATGGGCGCTCCGATGCCGCTCCA
GGGCGAGCGCTGTTTAAATAGCCAGGCCCCCGATTGCAAAGACATTATAGCGAGCTACCAAA
GCCATATTCAAACACCTAGATCACTACCACTTCTACACAGGCCACTCGAGCTTGTGATCGCA
CTCCGCTAAGGGGGCGCCTCTTCCTCTTCGTTTCAGTCACAACCCGCAAACTCTAGAATATC
Aatgatcgagcaggacggcctccacgccggctcccccgccgcctgggtggagcgcctgttcg
gctacgactgggcccagcagaccatcggctgctccgacgccgccgtgttccgcctgtccgcc
cagggccgccccgtgctgttcgtgaagaccgacctgtccggcgccctgaacgagctgcagga
cgaggccgcccgcctgtcctggctggccaccaccggcgtgccctgcgccgccgtgctggacg
tggtgaccgaggccggccgcgactggctgctgctgggcgaggtgcccggccaggacctgctg
tcctcccacctggcccccgccgagaaggtgtccatcatggccgacgccatgcgccgcctgca
caccctggaccccgccacctgccccttcgaccaccaggccaagcaccgcatcgagcgcgccc
gcacccgcatggaggccggcctggtggaccaggacgacctggacgaggagcaccagggcctg
gcccccgccgagctgttcgcccgcctgaaggcccgcatgcccgacggcgaggacctggtggt
gacccacggcgacgcctgcctgcccaacatcatggtggagaacggccgcttctccggcttca
tcgactgcggccgcctgggcgtggccgaccgctaccaggacatcgccctggccacccgcgac
atcgccgaggagctgggcggcgagtgggccgaccgcttcctggtgctgtacggcatcgccgc
ccccgactcccagcgcatcgccttctaccgcctgctggacgagttcttctgaCAATTGACGC
CCGCGCGGCGCACCTGACCTGTTCTCTCGAGGGCGCCTGTTCTGCCTTGCGAAACAAGCCCC
TGGAGCATGCGTGCATGATCGTCTCTGGCGCCCCGCCGCGCGGTTTGTCGCCCTCGCGGGCG
CCGCGGCCGCGGGGGCGCATTGAAATTGTTGCAAACCCCACCTGACAGATTGAGGGCCCAGG
CAGGAAGGCGTTGAGATGGAGGTACAGGAGTCAAGTAACTGAAAGTTTTTATGATAACTAAC
AACAAAGGGTCGTTTCTGGCCAGCGAATGACAAGAACAAGATTCCACATTTCCGTGTAGAGG
CTTGCCATCGAATGTGAGCGGGCGGGCCGCGGACCCGACAAAACCCTTACGACGTGGTAAGA
AAAACGTGGCGGGCACIGTCCCTGTAGCCTGAAGACCAGCAGGAGACGATCGGAAGCATCAC
AGCACAGGATCCCGCGTCTCGAACAGAGCGCGCAGAGGAACGCTGAAGGTCTCGCCTCTGTC
GCACCTCAGCGCGGCATACACCACAATAACCACCTGACGAATGCGCTTGGTTCTTCGTCCAT
TAGCGAAGCGTCCGGTTCACACACGTGCCACGTTGGCGAGGTGGCAGGTGACAATGATCGGT
GGAGCTGATGGICGAAACGTTCACAGCCTAGGGATATCGTGAAAACTCGCTCGACCGCCCGC
GTCCCGCAGGCAGCGATGACGTGTGCGTGACCTGGGTGTTTCGTCGAAAGGCCAGCAACCCC
AAATCGCAGGCGATCCGGAGATTGGGATCTGATCCGAGCTTGGACCAGATCCCCCACGATGC
GGCACGGGAACTGCATCGACTCGGCGCGGAACCCAGCTTTCGTAAATGCCAGATTGGTGTCC
GATACCTTGATTTGCCATCAGCGAAACAAGACTTCAGCAGCGAGCGTATTTGGCGGGCGTGC
TACCAGGGTTGCATACATTGCCCATTTCTGTCTGGACCGCTTTACCGGCGCAGAGGGTGAGT
TGATGGGGTTGGCAGGCATCGAAACGCGCGTGCATGGTGTGTGTGTCTGTTTTCGGCTGCAC
AATTTCAATAGTCGGATGGGCGACGGTAGAATTGGGTGTTGCGCTCGCGTGCATGCCTCGCC
CCGTCGGGTGTCATGACCGGGACTGGAATCCCCCCTCGCGACCCTCCTGCTAACGCTCCCGA
CTCTCCCGCCCGCGCGCAGGATAGACTCTAGTTCAACCAATCGACAACTAGTatggccaccg
catccactttctcggcgttcaatgcccgctgcggcgacctgcgtcgctcggcgggctccggg
ccccggcgcccagcgaggcccctccccgtgcgcgggcgcgccatccccccccgcatcatcgt
ggtgtcctcctcctcctccaaggtgaaccccctgaagaccgaggccgtggtgtcctccggcc
tggccgaccgcctgcgcctgggctccctgaccgaggacggcctgtcctacaaggagaagttc
atcgtgcgctgctacgaggtgggcatcaacaagaccgccaccgtggagaccatcgccaacct
gctgcaggaggtgggctgcaaccacgcccagtccgtgggctactccaccggcggcttctcca
ccacccccaccatgcgcaagctgcgcctgatctgggtgaccgcccgcatgcacatcgagatc
tacaagtaccccgcctggtccgacgtggtggagatcgagtcctggggccagggcgagggcaa
gatcggcacccgccgcgactggatcctgcgcgactacgccaccggccaggtgatcggccgcg
ccacctccaagtgggtgatgatgaaccaggacacccgccgcctgcagaaggtggacgtggac
gtgcgcgacgagtacctggtgcactgcccccgcgagctgcgcctggccttccccgaggagaa
caactcctccctgaagaagatctccaagctggaggacccctcccagtactccaagctgggcc
tggtgccccgccgcgccgacctggacatgaaccagcacgtgaacaacgtgacctacatcggc
tgggtgctggagtccatgccccaggagatcatcgacacccacgagctgcagaccatcaccct
ggactaccgccgcgagtgccagcacgacgacgtggtggactccctgacctcccccgagccct
ccgaggacgccgaggccgtgttcaaccacaacggcaccaacggctccgccaacgtgtccgcc
aacgaccacggctgccgcaacttcctgcacctgctgcgcctgtccggcaacggcctggagat
caaccgcggccgcaccgagtggcgcaagaagcccacccgcatggactacaaggaccacgacg
gcgactacaaggaccacgacatcgactacaaggacgacgacgacaagtgaATCGATgcagca
gcagctcggatagtatcgacacactctggacgctggtcgtgtgatggactgttgccgccaca
cttgctgccttgacctgtgaatatccctgccgcttttatcaaacagcctcagtgtgtttgat
cttgtgtgtacgcgcttttgcgagttgctagctgcttgtgctatttgcgaataccaccccca
gcatccccttccctcgtttcatatcgcttgcatcccaaccgcaacttatctacgctgtcctg
ctatccctcagcgctgctcctgctcctgctcactgcccctcgcacagccttggtttgggctc
cgcctgtattctcctggtactgcaacctgtaaaccagcactgcaatgctgatgcacgggaag
tagtgggatgggaacacaaatggaAAGCTTGAGCTCcagcgccatgccacgccctttgatgg
cttcaagtacgattacggtgttggattgtgtgtttgttgcgtagtgtgcatggtttagaata
atacacttgatttcttgctcacggcaatctcggcttgtccgcaggttcaaccccatttcgga
gtctcaggtcagccgcgcaatgaccagccgctacttcaaggacttgcacgacaacgccgagg
tgagctatgtttaggacttgattggaaattgtcgtcgacgcatattcgcgctccgcgacagc
acccaagcaaaatgtcaagtgcgttccgatttgcgtccgcaggtcgatgttgtgatcgtcgg
cgccggatccgccggtctgtcctgcgcttacgagctgaccaagcaccctgacgtccgggtac
gcgagctgagattcgattagacataaattgaagattaaacccgtagaaaaatttgatggtcg
cgaaactgtgctcgattgcaagaaattgatcgtcctccactccgcaggtcgccatcatcgag
cagggcgttgctcccggcggcggcgcctggctggggggacagctgttctcggccatgtgtgt
acgtagaaggatgaatttcagctggttttcgttgcacagctgtttgtgcatgatttgtttca
gactattgttgaatgtttttagatttcttaggatgcatgatttgtctgcatgcgact
Amino acid sequence of Gm FATA wild-type parental gene;
D3997, pSZ5083. The algal transit peptide is underlined and the FLAG epitope tag is
uppercase bold
SEQ ID NO: 136
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Amino acid sequence of Gm FATA S111A, V193A mutant gene;
D3998, pSZ5084. The algal transit peptide is underlined, the FLAG epitope tag is
uppercase bold and the S111A, V193A residues are lower-case bold.
SEQ ID NO: 137
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFaTTPTMRKLRLIWVTARMHIEIYK
YPAWSDVVEIESWGQGEGKIGTRRDWILRDYATGQVIGRATSKWVMMNQDTRRLQKVDaDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Amino acid sequence of Gm FATA S111V, V193A mutant gene;
D3999, pSZ5085. The algal transit peptide is underlined, the FLAG epitope tag is
uppercase bold and the S111V, V193A residues are lower-case bold.
SEQ ID NO: 138
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFvTTPTMRKLRLIWVTARMHIEIYK
YPAWSDVVEIESWGQGEGKIGTRRDWILRDYATGQVIGRATSKWVMMNQDTRRLQKVDaDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Amino acid sequence of Gm FATA G96A mutant gene; D4000,
pSZ5086. The algal transit peptide is underlined, the FLAG epitope tag is uppercase
bold and the G96A residue is lower-case bold.
SEQ ID NO: 139
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVaCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Amino acid sequence of Gm FATA G96T mutant gene; D4001,
pSZ5087. The algal transit peptide is underlined, the FLAG epitope tag is uppercase
bold and the G96T residue is lower-case bold.
SEQ ID NO: 140
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVtCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Amino acid sequence of Gm FATA G96V mutant gene; D4002,
pSZ5088. The algal transit peptide is underlined, the FLAG epitope tag is uppercase
bold and the G96V residue is lower-case bold.
SEQ ID NO: 141
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVvCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Amino acid sequence of Gm FATA G108A mutant gene;
D4003, pSZ5089. The algal transit peptide is underlined, the FLAG epitope tag is
uppercase bold and the G108A residue is lower-case bold.
SEQ ID NO: 142
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTaGESTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Amino acid sequence of Gm FATA L91F mutant gene; D4004,
pSZ5090. The algal transit peptide is underlined, the FLAG epitope tag is uppercase
bold and the L91F residue is lower-case bold.
SEQ ID NO: 143
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANfLQEVGCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Amino acid sequence of Gm FATA L91K mutant gene; D4005,
pSZ5091. The algal transit peptide is underlined, the FLAG epitope tag is uppercase
bold and the L91K residue is lower-case bold
SEQ ID NO: 144
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANkLQEVGCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
FIG. 10. Amino acid sequence of Gm FATA L915 mutant
gene; D4006, pSZ5092. The algal transit peptide is underlined, the FLAG epitope tag is
uppercase bold and the L915 residue is lower-case bold
SEQ ID NO: 14
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANsLQEVGCNHAQSVGYSTGGFSTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Amino acid sequence of Gm FATA G108V mutant gene;
D4007, pSZ5093. The algal transit peptide is underlined, the FLAG epitope tag is
uppercase bold and the G108V residue is lower-case bold.
SEQ ID NO: 146
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTvGESTTPTMRKLRLIWVTARMHIEIYK
YPAWSDVVEIESWGQGEGKIGTRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Amino acid sequence of Gm FATA T156F mutant gene;
D4008, pSZ5094. The algal transit peptide is underlined, the FLAG epitope tag is
uppercase bold and the T156F residue is lower-case bold.
SEQ ID NO: 147
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGfRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Amino acid sequence of Gm FATA T156A mutant gene;
D4009, pSZ5095. The algal transit peptide is underlined, the FLAG epitope tag is
uppercase bold and the T156A residue is lower-case bold.
SEQ ID NO: 148
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGaRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Amino acid sequence of Gm FATA T156K mutant gene; D4010,
pSZ5096. The algal transit peptide is underlined, the FLAG epitope tag is uppercase
bold and the T156K residue is lower-case bold.
SEQ ID NO: 149
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGkRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Amino acid sequence of Gm FATA T156V mutant gene;
D4011, pSZ5097. The algal transit peptide is underlined, the FLAG epitope tag is
uppercase bold and the T156V residue is lower-case bold.
SEQ ID NO: 150
MATASTFSAFNARCGDLRRSAGSGPRRPARPLPVRGRAIPPRIIVVSSSSSKVNPLKTEAVVSSGLADRLRLGSL
TEDGLSYKEKFIVRCYEVGINKTATVETIANLLQEVGCNHAQSVGYSTGGFSTTPTMRKLRLIWVTARMHIEIYK
YPAWSDVVEIESWGQGEGKIGvRRDWILRDYATGQVIGRATSKWVMMNQDTRRLQKVDVDVRDEYLVHCPRELRL
AFPEENNSSLKKISKLEDPSQYSKLGLVPRRADLDMNQHVNNVTYIGWVLESMPQEIIDTHELQTITLDYRRECQ
HDDVVDSLTSPEPSEDAEAVENHNGTNGSANVSANDHGCRNFLHLLRLSGNGLEINRGRTEWRKKPTRMDYKDHD
GDYKDHDIDYKDDDDK
Nucleotide sequence of the GmFATA S111A, V193A mutant gene
(D3998, pSZ5084). The promoter, 3′UTR, selection marker and targeting arms are the same
as pSZ5083.
SEQ ID NO: 151
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttcgccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgcggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA S111V, V193A mutant gene
(D3999, pSZ5085). The promoter, 3′UTR, selection marker and targeting arms are the same
as pSZ5083.
SEQ ID NO: 152
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttcgtcaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgcggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA G96A mutant gene (D4000,
pSZ5086). The promoter, 3′UTR, selection marker and targeting arms are the same as
pSZ5083
SEQ ID NO: 153
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtggcgtgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA G96T mutant gene (D4001,
pSZ5087). The promoter, 3′UTR, selection marker and targeting arms are the same as
pSZ5083
SEQ ID NO: 154
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgacgtgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA G96V mutant gene (D4002,
pSZ5088). The promoter, 3′UTR, selection marker and targeting arms are the same as
pSZ5083.
SEQ ID NO: 155
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtggtgtgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA G108A mutant gene
(D4003, pSZ5089). The promoter, 3′UTR, selection marker and targeting arms are the same
as pSZ50836.
SEQ ID NO: 156
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccgcc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA L91F mutant gene (D4004,
pSZ5090). The promoter, 3′UTR, selection marker and targeting arms are the same as
pSZ5083
SEQ ID NO: 157
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacttcctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA L91K mutant gene (D4005,
pSZ5091). The promoter, 3′UTR, selection marker and targeting arms are the same as
pSZ5083.
SEQ ID NO: 158
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacaagctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA L91S mutant gene (D4006,
pSZ5092). The promoter, 3′UTR, selection marker and targeting arms are the same as
pSZ5083.
SEQ ID NO: 159
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaactcgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA G108V mutant gene
(D4007, pSZ5093). The promoter, 3′UTR, selection marker and targeting arms are the same
as pSZ5083.
SEQ ID NO: 160
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccgtc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcacccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA T156F mutant gene (D4008,
pSZ5094). The promoter, 3′UTR, selection marker and targeting arms are the same as
pSZ5083.
SEQ ID NO: 161
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcttccgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA T156A mutant gene (D4009,
pSZ5095). The promoter, 3′UTR, selection marker and targeting arms are the same as
pSZ5083
SEQ ID NO: 162
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcgcgcgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA T156K mutant gene (D4010,
pSZ5096). The promoter, 3′UTR, selection marker and targeting arms are the same as
pSZ5083.
SEQ ID NO: 163
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcaagcgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga
Nucleotide sequence of the GmFATA T156V mutant gene (D4011,
pSZ5097). The promoter, 3′UTR, selection marker and targeting arms are the same as
pSZ5083
SEQ ID NO: 164
atggccaccgcatccactttctcggcgttcaatgcccgctgcggcgacctgcgtcgctcggc
gggctccgggccccggcgcccagcgaggcccctccccgtgcgcgggcgcgccatcccccccc
gcatcatcgtggtgtcctcctcctcctccaaggtgaaccccctgaagaccgaggccgtggtg
tcctccggcctggccgaccgcctgcgcctgggctccctgaccgaggacggcctgtcctacaa
ggagaagttcatcgtgcgctgctacgaggtgggcatcaacaagaccgccaccgtggagacca
tcgccaacctgctgcaggaggtgggctgcaaccacgcccagtccgtgggctactccaccggc
ggcttctccaccacccccaccatgcgcaagctgcgcctgatctgggtgaccgcccgcatgca
catcgagatctacaagtaccccgcctggtccgacgtggtggagatcgagtcctggggccagg
gcgagggcaagatcggcgtgcgccgcgactggatcctgcgcgactacgccaccggccaggtg
atcggccgcgccacctccaagtgggtgatgatgaaccaggacacccgccgcctgcagaaggt
ggacgtggacgtgcgcgacgagtacctggtgcactgcccccgcgagctgcgcctggccttcc
ccgaggagaacaactcctccctgaagaagatctccaagctggaggacccctcccagtactcc
aagctgggcctggtgccccgccgcgccgacctggacatgaaccagcacgtgaacaacgtgac
ctacatcggctgggtgctggagtccatgccccaggagatcatcgacacccacgagctgcaga
ccatcaccctggactaccgccgcgagtgccagcacgacgacgtggtggactccctgacctcc
cccgagccctccgaggacgccgaggccgtgttcaaccacaacggcaccaacggctccgccaa
cgtgtccgccaacgaccacggctgccgcaacttcctgcacctgctgcgcctgtccggcaacg
gcctggagatcaaccgcggccgcaccgagtggcgcaagaagcccacccgcatggactacaag
gaccacgacggcgactacaaggaccacgacatcgactacaaggacgacgacgacaagtga

1. A recombinant vector construct or a host cell comprising nucleic acids that encodes a protein having acyltransferase activity, wherein the amino acid sequence of the acyltransferase has at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196.

2. The recombinant of claim 1, wherein the amino acid sequence of the protein comprises:
a. at least 96.3% identity to an acyltransferase of clade 1 of Table 5;
b. at least 93.9% identity to an acyltransferase of clade 2 of Table 5;
c. at least 86.5% identity to an acyltransferase of clade 3 of Table 5; or
d. at least 78.5% identity to an acyltransferase of clade 4 of Table 5.

3.-7. (canceled)

8. The nucleic acids of claim 1, wherein the nucleic acids encoding the acyltransferase are codon-optimized for expression in Prototheca or Chlorella, and wherein the coding sequence contains the most or second most preferred codon of Table 1 or Table 2 for at least 60% of the codons of the coding sequence, such that the codon-optimized sequence is more efficiently translated in Prototheca or Chlorella than a non-codon optimized sequence.

9.-10. (canceled)

11. The host cell of claim 8, wherein the cell is a microalgal cell, microbial cell or a plant cell, and wherein the fatty acid profile or the sn-2 profile of the host cell is altered by the expression of the nucleic acids.

12. The host cell of claim 11, wherein the microalgal cell is a Prototheca cell or a Chlorella cell.

13. The host cell of claim 12, wherein the cell is a Prototheca moriformis cell.

14. The recombinant vector construct or a host cell of claim 1, wherein the acyl transferase is a lysophosphatidic acid acyltransferase (LPAAT), glycerol phosphate acyltransferase (GPAT), diacyl glycerol acyltransferase (DGAT), lysophosphatidylcholine acyltransferase (LPCAT), or phospholipase A2 (PLA2).

15. The recombinant vector construct or a host cell of claim 14, wherein the acyl transferase is lysophosphatidic acid acyltransferase (LPAAT).

16. A method of cultivating a host cell, the host cell comprising recombinant nucleic acids encoding a protein having acyltransferase activity, wherein the amino acid sequence of the acyltransferase has at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196.

17. The method of claim 16, wherein the amino acid sequence of the protein comprises:
a. at least 96.3% identity to an acyltransferase of clade 1 of Table 5;
b. at least 93.9% identity to an acyltransferase of clade 2 of Table 5;
c. at least 86.5% identity to an acyltransferase of clade 3 of Table 5; or
d. at least 78.5% identity to an acyltransferase of clade 4 of Table 5.

18.-22. (canceled)

23. The method of claim 16, wherein the nucleic acids encoding the acyltransferase are codon-optimized for expression in Prototheca or Chlorella, and wherein the coding sequence contains the most or second most preferred codon of Table 1 or Table 2 for at least 60% of the codons of the coding sequence, such that the codon-optimized sequence is more efficiently translated in Prototheca or Chlorella than a non-codon optimized sequence.

24.-25. (canceled)

26. The method of claim 23, wherein the cell is a microalgal cell, microbial cell or a plant cell.

27. The method of claim 26, wherein the microalgal cell is a Prototheca cell or a Chlorella cell.

28. The method of claim 27, wherein the cell is a Prototheca moriformis cell.

29. The method of claim 16, wherein the acyl transferase is a lysophosphatidic acid acyltransferase (LPAAT), glycerol phosphate acyltransferase (GPAT), diacyl glycerol acyltransferase (DGAT), lysophosphatidylcholine acyltransferase (LPCAT), or phospholipase A2 (PLA2).

30. The method of claim 29, wherein the acyltransferase is lysophosphatidic acid acyltransferase (LPAAT).

31. A method of producing a triglyceride oil in a host cell, the host cell comprising recombinant nucleic acids encoding a protein having acyltransferase activity, wherein the amino acid sequence of the acyltransferase has at least 75%, 80%, 85%, 90%, 95%, 98%, 99%, or 100% identity to an acyltransferase of SEQ ID NOs: 23, 24, 25, 26, 27, 28, 29, 30, 31, 32, 33, 34, 35, 36, 37, 38, 39, 40, 42, 42, 43, 44, 45, 46, 47, 48, 49, 50, 52, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 169, 170, 171, 172, 173, 174, 175, 176, 177, 178, 179, 180, 181, 182, 183, 184, 185, 186, 187, 188, 189, 190, 191, 192, 193, 194, 195, or 196.

32. The method of claim 31, wherein the amino acid sequence of the protein comprises:
a. at least 96.3% identity to an acyltransferase of clade 1 of Table 5;
b. at least 93.9% identity to an acyltransferase of clade 2 of Table 5;
c. at least 86.5% identity to an acyltransferase of clade 3 of Table 5; or
d. at least 78.5% identity to an acyltransferase of clade 4 of Table 5.

33.-38. (canceled)

39. The method of claim 31, wherein the microalgal cell is a Prototheca cell or a Chlorella cell.

40. The method of claim 39, wherein the cell is a Prototheca moriformis cell.

41.-141. (canceled)
